(19) United States
(12) Patent Application Publication
(10) Pub. No.: US 2026/0357292 A1
(43) Pub. Date: Nov. 12, 2026

(54) NUCLEIC ACID REGULATORY ELEMENTS FOR GENE EXPRESSION IN THE MUSCLE AND METHODS OF USE

(71) Applicant: MEIRAGTX GENE REGULATION LIMITED, London (GB)

(72) Inventors: Alexandria Forbes, New York, NY (US); Josefa Sullivan, New York, NY (US); Chenjin Jin, New York, NY (US); Enrico Mossotto, New York, NY (US); Matthew During, Rowayton, CT (US); Ce Feng Liu, New York, NY (US); Dustin Lee, New York, NY (US); Jessica Schmerler, New York, NY (US)

(21) Appl. No.: 19/117,774

(22) PCT Filed: Oct. 11, 2023
(86) PCT No.: PCT/IB2023/000615

§ 371 Date: Apr. 2, 2025

Related U.S. Application Data
(60) Provisional application No. 63/379,138, filed on Oct. 11, 2022.
(60) Provisional application No. 63/496,554, filed on Apr. 17, 2023.

(51) Int. Cl. C12N 15/86

(52) U.S. Cl. CPC C12N 15/86; C12N 2750/14143; C12N 2830/50

(57) ABSTRACT

The application relates to nucleic acid regulatory elements that are able to enhance expression of genes constitutively in a variety of tissues, or in particular tissues including the muscle. The application further relates to methods employing these regulatory elements and uses of these elements. Expression cassettes and vectors containing these nucleic acid regulatory elements are also disclosed. These are particularly useful for applications using gene therapy.

### CROSS-REFERENCE TO RELATED APPLICATIONS

This application claims the benefit under 35 U.S.C. § 119 (e) of the earlier filing date of U.S. Provisional Patent Application Ser. No. 63/379,138, filed on Oct. 11, 2022 and U.S. Provisional Patent Application Ser. No. 63/496,554, filed on Apr. 17, 2023, which are hereby incorporated by reference in their entirety.

### REFERENCE TO AN ELECTRONIC SEQUENCE LISTING

The contents of the electronic sequence listing (SeqList-162027.54176.xml; Size: 290,976 bytes; and Date of Creation: Oct. 10, 2023) is herein incorporated by reference in its entirety.

### FIELD

The application relates to nucleic acid regulatory elements that are able to enhance expression of genes in a variety of tissues, and in particular tissues including in the muscle. The application further relates to methods employing these regulatory elements and uses of these elements. Expression cassettes and vectors containing these nucleic acid regulatory elements are also disclosed. These are particularly useful for applications using gene therapy.

### BACKGROUND

A promoter is a DNA region at which transcription of a gene is initiated. Since the promoter region controls when and where a gene of interest is expressed in an organism, promoters are crucial elements for regulating the level and specificity of transgene expression, particularly in the context of gene therapy.

The use of engineered nucleic acid regulatory elements (NAREs) (that may include various components, including, promoters, enhancers, etc.) that are particularly tailored to a given gene therapy provides a variety of benefits. To start, an optimized NARE allows for levels of gene expression that is desired for a specific therapeutic gene. Second, engineered NAREs with increased potency allow administration of smaller amounts of gene therapy vector, thus decreasing immune responses and associated safety risks. Third, for some gene therapies, it is desirable that gene expression is limited to a specific tissue or tissues. Accordingly, use of nucleic acid regulatory elements with tissue-specific expression can restrict unwanted transgene expression as well as facilitate persistent transgene expression in the tissue(s) or interest. Such tissue-specific NAREs can be used to eliminate the need for tissue-specific viral capsids used for gene delivery (or can be used in combination with tissue-specific viral capsids). Fourth, choosing an appropriate NARE also allows controlling the kinetics of gene expression, which in turn impact durability of gene therapy. Finally, it can be desirable to engineer NAREs with a reduced size (without sacrificing strength or specificity) to allow for efficient packaging of larger transgene cargo into viral vectors.

While efforts have been made to characterize and optimize NARE sequences through conventional low-throughput analyses (rational design) or newer high-throughput methodologies (e.g., MPRA), these approaches still require an expensive and time-consuming in vitro optimization. Accordingly, more efficient methods of engineering NAREs are needed as are nucleic acid regulatory elements with enhanced potency, reduced size, and/or tissue specificity.

### SUMMARY

Provided herein are nucleic acid regulatory elements (NAREs), including NAREs that are particularly suitable for the expression of an operably linked sequence (e.g., a protein or RNA coding sequence) in the muscle. Also provided herein are methods for the expression of a transgene that is operably linked to one or more of the nucleic acid regulatory elements disclosed herein.

In embodiments, provided is a polynucleotide sequence that comprises a sequence that is at least 80% identical to a NARE provided in Tables 4 or 6. In embodiments, provided is a polynucleotide sequence that comprises a sequence that is at least 90% identical to a NARE provided in in Tables 4 or 6. In embodiments, provided is a polynucleotide comprising a NARE provided in in Tables 4 or 6.

In embodiments, provided is a polynucleotide sequence that comprises a sequence that is at least 80% identical to a NARE selected from the group consisting of M1-M18, M20, M21, M23, M24, M32-M49, M51-M70, and M74-M120 (as listed in Table 6). In embodiments, provided is a polynucleotide sequence that comprises a sequence that is at least 90% identical to a NARE selected from the group consisting of M1-M18, M20, M21, M23, M24, M32-M49, M51-M70, and M74-M120. In embodiments, provided is a polynucleotide sequence that comprises a NARE selected from the group consisting of M1-M18, M20, M21, M23, M24, M32-M49, M51-M70, and M74-M120.

In embodiments, provided is a polynucleotide sequence that comprises a sequence that is at least 80% identical to a NARE selected from the group consisting of M21, M23-M28, M32, M34-M36, M51, M53, M55, M57, M59, M61, and M63 (as listed in Table 6). In embodiments, provided is a polynucleotide sequence that comprises a sequence that is at least 90% identical to a NARE selected from the group consisting of M21, M23-M28, M32, M34-M36, M51, M53, M55, M57, M59, M61, and M63. In embodiments, provided is a polynucleotide sequence that comprises a NARE selected from the group consisting of M21, M23-M28, M32, M34-M36, M51, M53, M55, M57, M59, M61, and M63.

In embodiments, provided is a polynucleotide sequence that comprises a sequence that is at least 80% identical to a NARE selected from the group consisting of M21 and M24. In embodiments, provided is a polynucleotide sequence that comprises a sequence that is at least 90% identical to a NARE selected from the group consisting of M21 and M24. In embodiments, provided is a polynucleotide sequence that comprises a NARE selected from the group consisting of M21 and M24.

In embodiments, provided is a polynucleotide sequence that comprises a sequence that is at least 80% identical to a NARE selected from the group consisting of M21, M24-M27, M30, and M32-M36 (as listed in Table 6). In embodiments, provided is a polynucleotide sequence that comprises a sequence that is at least 90% identical to a NARE selected from the group consisting of M21, M24-M27, M30, and M32-M36. In embodiments, provided is a polynucleotide sequence that comprises a NARE selected from the group consisting of M21, M24-M27, M30, and M32-M36.

In embodiments, provided is a polynucleotide sequence that comprises a sequence that is at least 80% identical to a NARE selected from the group consisting of M49, M51, M61, M98, M99, and M111-M115 (as listed in Table 6). In embodiments, provided is a polynucleotide sequence that comprises a sequence that is at least 90% identical to a NARE selected from the group consisting of M49, M51, M61, M98, M99, and M111-M115. In embodiments, provided is a polynucleotide sequence that comprises a NARE selected from the group consisting of M49, M51, M61, M98, M99, and M111-M115.

In embodiments, one or more NAREs disclosed herein is operably linked to a transgene.

In one aspect, provided is a NARE comprising: (i) a sequence that is at least 90% identical to SEQ ID NO:132; and (ii) a sequence that is at least 90% identical to any one of SEQ ID NOs: 156-163.

In some embodiments, provided is a NARE comprising: (i) a sequence that is at least 95% identical to SEQ ID NO:132; and (ii) a sequence that is at least 95% identical to any one of SEQ ID NOs: 156-163. In some embodiments, provided is a NARE comprising: (i) SEQ ID NO:132; and (ii) any one of SEQ ID NOs: 156-163.

In one aspect, provided is NARE comprising: (i) a sequence that is at least 90% identical to SEQ ID NO:164; (ii) a sequence that is at least 90% identical to SEQ ID NO:134; (iii) a sequence that is at least 90% identical to SEQ ID NO:135; and (vi) a sequence that is at least 90% identical to SEQ ID NOs: 148 or 149. In some embodiments, provided is a NARE comprising: (i) a sequence that is at least 95% identical to SEQ ID NO:164; (ii) a sequence that is at least 95% identical to SEQ ID NO:134; (iii) a sequence that is at least 95% identical to SEQ ID NO:135; and (vi) a sequence that is at least 95% identical to SEQ ID NOs: 148 or 149. In some embodiments, provided is a NARE comprising: (i) SEQ ID NO:164; (ii) SEQ ID NO: 134; (iii) SEQ ID NO:135; and (vi) SEQ ID NOs: 148 or 149.

In one aspect, provided is a NARE comprising: (i) a sequence that is at least 90% identical to SEQ ID NO:218; and (ii) a sequence that is at least 90% identical to any one of SEQ ID NOs: 148 or 149. In some embodiments, provided is a NARE comprising: (i) a sequence that is at least 95% identical to SEQ ID NO:218; and (ii) a sequence that is at least 95% identical to any one of SEQ ID NOs: 148 or 149. In some embodiments, provided is a NARE comprising: (i) SEQ ID NO:218; and (ii) SEQ ID NOs: 148 or 149.

In one aspect, provided is a NARE comprising: (i) a sequence that is at least 90% identical to any one of SEQ ID NOs: 133, 142, 165 or 217; (ii) optionally, a sequence that is at least 90% identical to SEQ ID NOs: 154 or 189; (iii) optionally, a sequence that is at least 90% identical to SEQ ID NO:220; and (iv) a sequence that is at least 90% identical to SEQ ID NO: 150.

In some embodiments, provided is a NARE comprising: (i) a sequence that is at least 95% identical to any one of SEQ ID NOs: 133, 142, 165 or 217; (ii) optionally, a sequence that is at least 95% identical to SEQ ID NOs: 154 or 189; (iii) optionally, a sequence that is at least 95% identical to SEQ ID NO:220; and (iv) a sequence that is at least 95% identical to SEQ ID NO:150.

In some embodiments, provided is a NARE comprising: (i) any one of SEQ ID NOs: 133, 142, 165 or 217; (ii) optionally, SEQ ID NOs: 154 or 189; (iii) optionally, SEQ ID NO: 220; and (iv) SEQ ID NO:150.

In one aspect, provided is a NARE comprising: (i) a sequence that is at least 90% identical to SEQ ID NO:131; (ii) a sequence that is at least 90% identical to SEQ ID NOs: 126 or 213; and (iii) optionally, a sequence that is at least 90% identical to SEQ ID NO:200.

In some embodiments, provided is a NARE comprising: (i) a sequence that is at least 95% identical to SEQ ID NO:131; (ii) a sequence that is at least 95% identical to SEQ ID NOs: 126 or 213; and (iii) optionally, a sequence that is at least 95% identical to SEQ ID NO: 200.

In some embodiments, provided is a NARE comprising: (i) SEQ ID NO:131; (ii) SEQ ID NOs: 126 or 213; and (iii) optionally, SEQ ID NO:200.

In some embodiments, provided is a NARE comprising: (i) a sequence that is at least 90% identical to SEQ ID NO:131; (ii) a sequence that is at least 90% identical to SEQ ID NO: 126; and (iii) optionally, a sequence that is at least 90% identical to SEQ ID NO:200.

In some embodiments, provided is a NARE comprising: (i) a sequence that is at least 95% identical to SEQ ID NO:131; (ii) a sequence that is at least 95% identical to SEQ ID NO: 126; and (iii) optionally, a sequence that is at least 95% identical to SEQ ID NO:200.

In some embodiments, provided is a NARE comprising: (i) SEQ ID NO:131; (ii) SEQ ID NO:126; and (iii) optionally, SEQ ID NO:200.

In one aspect, provided is a NARE comprising: (i) a sequence that is at least 90% identical to any one of SEQ ID NOs: 190-199; and (ii) a sequence that is at least 90% identical to SEQ ID NO:155.

In some embodiments, provided is a NARE comprising: (i) a sequence that is at least 95% identical to any one of SEQ ID NOs: 190-199; and (ii) a sequence that is at least 95% identical to SEQ ID NO:155.

In some embodiments, provided is a NARE comprising: (i) any one of SEQ ID NOs: 190-199; and (ii) SEQ ID NO:155.

In one aspect, provided is a NARE comprising: (i) a sequence that is at least 90% identical to any one of SEQ ID NOs: 190-199; and (ii) a sequence that is at least 90% identical to any one of SEQ ID NOs: 128-130.

In some embodiments, provided is a NARE comprising: (i) a sequence that is at least 95% identical to any one of SEQ ID NOs: 190-199; and (ii) a sequence that is at least 95% identical to any one of SEQ ID NOs: 128-130.

In some embodiments, provided is a NARE comprising: (i) any one of SEQ ID NOs: 190-199; and (ii) any one of SEQ ID NOs: 128-130.

In one aspect, provided is a NARE comprising: (i) a sequence that is at least 90% identical to any one of SEQ ID NOs: 128-130; and (ii) a sequence that is at least 90% identical to any one of SEQ ID NOs: 123-125, 127, 136-141, 143, 145-147, 152, 153, 209-212, 219, or 221.

In some embodiments, provided is a NARE comprising: (i) a sequence that is at least 95% identical to any one of SEQ ID NOs: 128-130; and (ii) a sequence that is at least 95% identical to any one of SEQ ID NOs: 123-125, 127, 136-141, 143, 145-147, 152, 153, 209-212, 219, or 221.

In some embodiments, provided is a NARE comprising: (i) any one of SEQ ID NOs: 128-130; and (ii) any one of SEQ ID NOs: 123-125, 127, 136-141, 143, 145-147, 152, 153, 209-212, 219, or 221.

In one aspect, provided is a NARE comprising: (i) a sequence that is at least 90% identical to SEQ ID NO:121; (ii) a sequence that is at least 90% identical to any one of SEQ ID NOs: 166-169, 171, or 172; and (iii) optionally, a sequence that is at least 90% identical to SEQ ID NOs: 122 or 154.

In some embodiments, provided is a NARE comprising: (i) a sequence that is at least 95% identical to SEQ ID NO:121; (ii) a sequence that is at least 95% identical to any one of SEQ ID NOs: 166-169, 171, or 172; and (iii) optionally, a sequence that is at least 95% identical to SEQ ID NOs: 122 or 154.

In some embodiments, provided is a NARE comprising: (i) SEQ ID NO:121; (ii) any one of SEQ ID NOs: 166-169, 171, or 172; and (iii) optionally, SEQ ID NOs: 122 or 154.

In some embodiments, provided is a NARE comprising: (i) a sequence that is at least 90% identical to SEQ ID NO:121; and (ii) a sequence that is at least 90% identical to any one of SEQ ID NOs: 166-169, 171, or 172.

In some embodiments, provided is a NARE comprising: (i) a sequence that is at least 95% identical to SEQ ID NO:121; and (ii) a sequence that is at least 95% identical to any one of SEQ ID NOs: 166-169, 171, or 172.

In some embodiments, provided is a NARE comprising: (i) SEQ ID NO:121; and (ii) SEQ ID NOs: 166-169, 171, or 172.

In some embodiments, provided is a NARE comprising: (i) a sequence that is at least 90% identical to SEQ ID NO:122 or 154; (ii) a sequence that is at least 90% identical to SEQ ID NO:121; and (iii) a sequence that is at least 90% identical to SEQ ID NO:167.

In some embodiments, provided is a NARE comprising: (i) a sequence that is at least 95% identical to SEQ ID NO:122 or 154; (ii) a sequence that is at least 95% identical to SEQ ID NO:121; and (iii) a sequence that is at least 90% identical to SEQ ID NO:167.

In some embodiments, provided is a NARE comprising: (i) SEQ ID NO: 122 or 154; (ii) SEQ ID NO:121; and (iii) SEQ ID NO:167.

In one aspect, provided is a NARE comprising: (i) a sequence that is at least 90% identical to SEQ ID NO:122; (ii) a sequence that is at least 90% identical to any one of SEQ ID NO: 173-179, or 188; and (iii) optionally, a sequence that is at least 90% identical to SEQ ID NO: 207.

In some embodiments, provided is a NARE comprising: (i) a sequence that is at least 95% identical to SEQ ID NO:122; (ii) a sequence that is at least 95% identical to any one of SEQ ID NO:173-179, or 188; and (iii) optionally, a sequence that is at least 95% identical to SEQ ID NO:207.

In some embodiments, provided is a NARE comprising: (i) SEQ ID NO:122; (ii) any one of SEQ ID NO:173-179, or 188; and (iii) optionally, SEQ ID NO:207.

In some embodiments, provided is a NARE comprising: (i) a sequence that is at least 90% identical to SEQ ID NO:122; and (ii) a sequence that is at least 90% identical to any one of SEQ ID NO: 173-179.

In some embodiments, provided is a NARE comprising: (i) a sequence that is at least 95% identical to SEQ ID NO:122; and (ii) a sequence that is at least 95% identical to any one of SEQ ID NO: 173-179.

The In some embodiments, provided is a NARE comprising: (i) SEQ ID NO:122; and (ii) any one of SEQ ID NO:173-179.

In one aspect, provided is a NARE comprising: (i) a sequence that is at least 90% identical to SEQ ID NO:184; and (ii) a sequence that is at least 90% identical to any one of SEQ ID NOs: 185-187.

In some embodiments, provided is a NARE comprising: a sequence that is at least 95% identical to SEQ ID NO:184; and (ii) a sequence that is at least 95% identical to any one of SEQ ID NOs: 185-187.

In some embodiments, provided is a NARE comprising: SEQ ID NO:184; and (ii) any one of SEQ ID NOs: 185-187.

In one aspect, provided is a NARE comprising: (i) a sequence that is at least 90% identical to SEQ ID NO:180; and (ii) a sequence that is at least 90% identical to any one of SEQ ID NOs: 181 or 182.

In some embodiments, provided is a NARE comprising: (i) a sequence that is at least 95% identical to SEQ ID NO:180; and (ii) a sequence that is at least 95% identical to any one of SEQ ID NOs: 181 or 182.

In some embodiments, provided is a NARE comprising: (i) SEQ ID NO: 180; and (ii) any one of SEQ ID NOs: 181 or 182.

In one aspect, provided is a NARE comprising: (i) a sequence that is at least 90% identical to SEQ ID NO:183; (ii) a sequence that is at least 90% identical to SEQ ID NO:182; and (iii) optionally, a sequence that is at least 90% identical to any one of SEQ ID NOs: 200, 201, 205, or 208.

In some embodiments, provided is a NARE comprising: (i) a sequence that is at least 95% identical to SEQ ID NO:183; (ii) a sequence that is at least 95% identical to SEQ ID NO: 182; and (iii) optionally, a sequence that is at least 95% identical to any one of SEQ ID NOs: 200, 201, 205, or 208.

In some embodiments, provided is a NARE comprising: (i) SEQ ID NO:183; (ii) SEQ ID NO:182; and (iii) optionally, any one of SEQ ID NOs: 200, 201, 205, or 208.

In some embodiments, provided is a NARE comprising: (i) a sequence that is at least 90% identical to SEQ ID NO:183; (ii) a sequence that is at least 90% identical to SEQ ID NO: 182; and (iii) a sequence that is at least 90% identical to any one of SEQ ID NOs: 200, 201, 205, or 208.

In some embodiments, provided is a NARE comprising: (i) a sequence that is at least 95% identical to SEQ ID NO:183; (ii) a sequence that is at least 95% identical to SEQ ID NO: 182; and (iii) a sequence that is at least 95% identical to any one of SEQ ID NOs: 200, 201, 205, or 208.

In some embodiments, provided is a NARE comprising: (i) SEQ ID NO:183; (ii) SEQ ID NO:182; and (iii) any one of SEQ ID NOs: 200, 201, 205, or 208.

In one aspect, provided is a NARE comprising: (i) a sequence that is at least 90% identical to SEQ ID NO:154 or 164; and (ii) a sequence that is at least 90% identical to any one of SEQ ID NOs: 128-130.

In some embodiments, provided is a NARE comprising: (i) a sequence that is at least 95% identical to SEQ ID NO:154 or 164; and (ii) a sequence that is at least 95% identical to any one of SEQ ID NOs: 128-130.

In some embodiments, provided is a NARE comprising: (i) SEQ ID NO: 154 or 164; and (ii) any one of SEQ ID NOs: 128-130.

In one aspect, provided is a NARE comprising: (i) a sequence that is at least 90% identical to SEQ ID NO:170; (ii) optionally, a sequence that is at least 90% identical to a SEQ ID NO:188; and (iii) a sequence that is at least 90% identical to SEQ ID NOs: 203 or 204.

In some embodiments, provided is a NARE comprising: (i) a sequence that is at least 95% identical to SEQ ID NO:170; (ii) optionally, a sequence that is at least 95% identical to a SEQ ID NO:188; and (iii) a sequence that is at least 95% identical to SEQ ID NOs: 203 or 204.

In some embodiments, provided is a NARE comprising: (i) SEQ ID NO: 170; (ii) optionally, SEQ ID NO:188; and (iii) SEQ ID NOs: 203 or 204.

In one aspect, provided is a NARE comprising:
(a) (i) a sequence that is at least 90% identical to SEQ ID NO:218; and (ii) a sequence that is at least 90% identical to SEQ ID NO:149;
(b) (i) a sequence that is at least 90% identical to SEQ ID NO:164; (ii) a sequence that is at least 90% identical to SEQ ID NO:134; (iii) a sequence that is at least 90% identical to SEQ ID NO:135; and (vi) SEQ ID NO:149;
(c) (i) a sequence that is at least 90% identical to SEQ ID NO:165; (ii) a sequence that is at least 90% identical to SEQ ID NO:154; (iii) a sequence that is at least 90% identical to SEQ ID NO:220; and (iv) a sequence that is at least 90% identical to SEQ ID NO: 150;
(d) (i) a sequence that is at least 90% identical to SEQ ID NO:142; and (ii) a sequence that is at least 90% identical to SEQ ID NO:150;
(e) (i) a sequence that is at least 90% identical to SEQ ID NO:217; (ii) a sequence that is at least 90% identical to SEQ ID NO:189; and (iii) a sequence that is at least 90% identical to SEQ ID NO:150;
(f) (i) a sequence that is at least 90% identical to SEQ ID NO:196; and (ii) a sequence that is at least 90% identical to SEQ ID NO:155;
(g) (i) a sequence that is at least 90% identical to SEQ ID NO:194; and (ii) a sequence that is at least 90% identical to SEQ ID NO:155; or
(h) (i) a sequence that is at least 90% identical to SEQ ID NO:184; and (ii) a sequence that is at least 90% identical to SEQ ID NO:187.

In some embodiments, provided is a NARE comprising:
(a) (i) a sequence that is at least 95% identical to SEQ ID NO:218; and (ii) a sequence that is at least 95% identical to SEQ ID NO:149;
(b) (i) a sequence that is at least 95% identical to SEQ ID NO:164; (ii) a sequence that is at least 95% identical to SEQ ID NO:134; (iii) a sequence that is at least 95% identical to SEQ ID NO:135; and (vi) SEQ ID NO:149;
(c) (i) a sequence that is at least 95% identical to SEQ ID NO:165; (ii) a sequence that is at least 95% identical to SEQ ID NO:154; (iii) a sequence that is at least 95% identical to SEQ ID NO:220; and (iv) a sequence that is at least 95% identical to SEQ ID NO: 150;
(d) (i) a sequence that is at least 95% identical to SEQ ID NO:142; and (ii) a sequence that is at least 95% identical to SEQ ID NO:150;
(e) (i) a sequence that is at least 95% identical to SEQ ID NO:217; (ii) a sequence that is at least 95% identical to SEQ ID NO:189; and (iii) a sequence that is at least 95% identical to SEQ ID NO:150;
(f) (i) a sequence that is at least 95% identical to SEQ ID NO:196; and (ii) a sequence that is at least 95% identical to SEQ ID NO:155;
(g) (i) a sequence that is at least 95% identical to SEQ ID NO:194; and (ii) a sequence that is at least 95% identical to SEQ ID NO:155; or
(h) (i) a sequence that is at least 95% identical to SEQ ID NO:184; and (ii) a sequence that is at least 95% identical to SEQ ID NO:187.

In some embodiments, provided is a NARE comprising:
(a) (i) SEQ ID NO:218; and (ii) SEQ ID NO:149;
(b) SEQ ID NO:164; (ii) SEQ ID NO:134; (iii) SEQ ID NO:135; and (vi) SEQ ID NO:149;
(c) (i) SEQ ID NO:165; (ii) SEQ ID NO:154; (iii) SEQ ID NO:220; and (iv) SEQ ID NO: 150;
(d) (i) SEQ ID NO:142; and (ii) SEQ ID NO:150;
(e) (i) SEQ ID NO:217; (ii) SEQ ID NO:189; and (iii) SEQ ID NO:150;
(f) (i) SEQ ID NO:196; and (ii) SEQ ID NO:155;
(g) (i) SEQ ID NO:194; and (ii) SEQ ID NO:155; or
(h) (i) SEQ ID NO:184; and (ii) SEQ ID NO:187.

In one aspect, provided is a NARE comprising a sequence that is at least 90% identical to any one of SEQ ID NOs: 1-18, 20, 21, 23, 24, 32-49, 51-70, or 74-120. In some embodiments, provided is a NARE comprising a sequence that is at least 95% identical to any one of SEQ ID NOs: 1-18, 20, 21, 23, 24, 32-49, 51-70, or 74-120. In some embodiments, provided is a NARE comprising any one of SEQ ID NOs: 1-18, 20, 21, 23, 24, 32-49, 51-70, or 74-120. In some embodiments, provided is a NARE comprising a sequence that is at least 90% identical to any one of SEQ ID NOs: 1-18 or 32. In some embodiments, provided is a NARE comprising a sequence that is at least 95% identical to any one of SEQ ID NOs: 1-18 or 32. In some embodiments, provided is a NARE comprising any one of SEQ ID NOs: 1-18 or 32.

In one aspect, provided is an expression construct comprising a NARE disclosed herein and an operatively linked transgene. In one embodiment, the expression construct further comprises a polyadenylation sequence.

In one aspect, provided is a vector comprising an expression construct disclosed herein. In one embodiment, the vector is a non-viral vector. In one embodiment, the vector is a viral vector. In one embodiment, the vector is an adeno-associated virus (AAV) vector. In one embodiment, the vector comprises a nucleic acid sequence comprising (i) the expression construct disclosed herein and (ii) one or more inverted terminal repeats (ITR). In one embodiment, the vector comprises a 5′ ITR and a 3′ ITR. In one embodiment, the 5′ ITR and a 3′ ITR are derived from AAV serotype AAV2.

In one aspect, provided is a cell comprising an expression construct or a vector disclosed herein. In some embodiments, the cells is a muscle cell.

In one aspect, provided is a pharmaceutical composition comprising (i) an expression construct disclosed herein or a vector disclosed herein and (ii) a pharmaceutically acceptable excipient.

In one aspect, provided is a method for expressing a transgene in a cell comprising an expression construct disclosed herein or a vector disclosed herein. In one aspect, provided is method for regulating transgene expression in a cell comprising an expression construct disclosed herein or a vector disclosed herein. In some embodiments, the cell is a muscle cell.

### BRIEF DESCRIPTION OF THE FIGURES

FIGS. 1A and 1B. Dual reporter design and measurement. FIG. 1a illustrates the design of the dual reporter construct. Candidate NAREs were cloned upstream (5′) of the mClover3 coding sequence. Each construct contains a constant region that includes a tdTomato transgene that is used as a normalization control. FIG. 1B provides an example of flow cytometry data obtained with the dual reporter system, in which mClover3 expression in tdTomato+ cells reflects the level of NARE activity.

FIGS. 2A, 2B, 2C and 2D. Generation of NAREs well suited for the expression of genes in the muscle. FIG. 2A: Example of nested NARE design (M24). Promoter tMCK was modified by adding the MTE/DPE modification. A synthetic HBG intron variant containing an intron-containing enhancer (SIE) and a modified human Desmin promoter was inserted downstream. FIG. 2B: Evolution of a strong, NARE, M24, with increased NARE strength of up to 7-fold as compared to NARE M22 in transfected C2C12 cells in vitro. FIG. 2C: NARE M24 was stronger than the reference promoter tMCK in cultured C2C12 myotubes. C2C12 myoblast cells were differentiated into myotubes and AAV8 transduction was performed at 10k MOI. Fluorescence of the iRFP713 reporter was measured on the indicated days. FIG. 2D: NARE M24 was stronger than the reference tMCK promoter and showed durable expression in primary human myotubes. Primary human myoblasts were differentiated into myotubes and AAV8 transduction was performed at 150 k MOI.

FIGS. 3A, 3B, 3C, 3D, 3E, and 3F. In vivo expression of NAREs M21 and M24. FIG. 3A: AAV8-iRFP713 comprising the tMCK promoter or the synthetic M24 NARE, respectively, was injected intramuscularly to male C57B16 mice at 8 weeks of age and imaged using the LICOR Pearl. FIG. 3B: Representative imaging data showing un-injected and injected hindlimbs for AAV8-tMCK and AAV8-M24, respectively, after 7, 10, or 14 days. FIG. 3C: Quantification of fluorescence showing that NARE M24 (top line) induced 8-fold higher expression as compared to the reference promoter tMCK (bottom line) (expression that is maintained out to 300 days post injection). FIG. 3D: AAV8-eGFP was injected into the gastrocnemius of male C57B16 mice at 8 weeks of age. After 4 weeks, the muscle was harvested and sectioned for measurement of native eGFP fluorescence. FIGS. 3E and 3F: Quantification (FIG. 3E) and representative images (FIG. 3F), scalebar 100 microns. NAREs M21 and M24 were stronger than reference promoters CAG or CK8e in the mouse gastrocnemius.

FIG. 4. Potency of selected NAREs in muscle cells. A library of NAREs was generated, the library containing NAREs from genes highly expressed in human skeletal muscle, whereby the NAREs were modified with various elements to increase NARE strength. NARE size ranged from 450 bp to 1.6 kb. Dual-Reporter plasmids with different NARE candidates were transiently transfected into C2C12 cells. GFP expression level in transfected (RFP+) cells was quantified by flow cytometry.

FIGS. 5A, 5B, 5C, 5D, 5E, 5F, 5G, 5H, 5I, 5J, 5K, 5L, 5M, 5N, 5O, and 5P. Potency of selected NAREs in muscle cells. FIGS. 9A, 9B, and 9C: Creation of synthetic enhancers via shuffling transcription factor (TF) motifs for increased expression in muscle. FIG. 5A: Diagram for creating enhancers placed upstream of a minimal promoter. Transcription factor binding sites (e.g., muscle-enriched sites: MPEX, MEF-1, MEF-2, MEF-3, MAPF and strong, ubiquitous sites: Trex, TEF, SRE, SP1, CREBH, GATA-2, CAATbox, AP-1, AP-2) were randomly ligated and inserted upstream of a minimal promoter (C6) and a mClover3 transgene to test enhancer function. FIG. 5B: Representative sequencing showed that the majority of the synthetic sequences were 200 bp or less. FIG. 5C: Relative expression from synthetic enhancers coupled with the promoter C6 normalized to the parent NARE in co-transfected C2C12 cells. Synthetic fragments increased expression from a minimal NARE up to 8-fold in mouse C2C12 muscle cells. FIG. 5D: Representative image of a particularly well-performing NARE identified by the screen (right) compared to the C6 promoter (left) in transfected C2C12 cells. FIG. 5E: Synthetic fragments identified above can be used in combination with other NAREs to generate potent, novel NAREs suitable for the expression in muscle. Expression of these NAREs was measured using the dual reporter assay in transfected C2C12 cells. FIG. 5F: Potency of NARE M51 compared to selected reference promoters in transfected C2C12 cells. FIG. 5G: Potency of CK8e-derived NARE M49 in transfected C2C12 cells. FIG. 5H: Potency of minMCK-derived NARE M51 in transfected C2C12 cells. FIG. 5I: Primary human myotubes were transduced with AAV8-miRFP713-WPRE-SV40polyA containing the indicated NAREs. FIG. 5J: Potency of ACTA1-derived NAREs as compared to indicated reference promoters using the dual reporter assay in C2C12 cells. FIG. 5K: Potency of hDes-derived NAREs as compared to indicated reference promoters using the dual reporter assay in C2C12 cells. FIG. 5L: Potency of tMCK-derived NAREs as compared to indicated reference promoters using the dual reporter assay in C2C12 myoblast cells.

### DETAILED DESCRIPTION

Nucleic acid regulatory elements (NAREs), including promoters, are essential components of a gene therapy that control the expression level and durability of expression of a therapeutic gene. NAREs can drive cell-specific expression of transgenes independent of, for example, capsid choice. Incorporation of stronger NAREs can increase potency and efficacy at lower viral vector doses, thereby potentially decreasing safety risks, immune responses, and reducing cost of vector production. Moreover, reducing NARE size while maintaining strength and specificity allows efficient packaging of larger transgenes or expression cassettes into AAV. The present application provides libraries of nucleic acid regulatory elements to improve the safety, efficacy, and durability of therapeutic transgene expression.

While many NAREs and particularly promoter sequences have been characterized/optimized through conventional low-throughput analyses (rational design) or newer high-throughput methodologies (e.g., MPRA), these approaches still require an expensive and time-consuming in vitro optimization. Here, using advanced artificial intelligence models, a convolutional neural network (CNN) was repurposed and optimized to predict NARE potency. A library of nucleic acid regulatory elements was computationally constructed by cloning all known enhancer elements reported in the ENCODE database upstream of a potent, compact constitutive promoter. Subsequently, the correct spacing between the particularly well-performing enhancer elements and the promoter sequence was optimized. In parallel, in silico saturated mutability was performed, whereby all possible point mutations within the NARE sequence were introduced and those with the best impact on the NARE's potency were selected. Particularly well-performing NARE's were produced and tested in vitro. While most of the nucleic acid regulatory elements exhibited better or equal performance than the original promoter, the best enhancer elements and point mutation were selected and combined in a second optimization round. After synthesizing and testing this new set in vitro, enhanced nucleic acid regulatory elements that exhibit increased potency were obtained.

### NAREs

Provided herein are NAREs that may include as part of their sequence a promoter and/or an enhancer. Traditionally, promoters are defined as DNA regions where transcription is initiated. Promoters include specific DNA motifs that transcription factors (TFs) and their complexes can access. On the other hand, enhancers are defined as DNA regions that amplify transcription initiation by directly interplaying with their target promoters. Likewise, the enhancer sequences, distal from their target promoters, contain DNA motifs that act as binding sites for TFs and cofactors. At times, the term promoter may be used as a shorthand to refer to a nucleic acid sequence that comprises multiple regulatory elements. CAG, for example, contains a CMV enhancer, β actin promoter region and intron, but may be referred to as a promoter. Specifically, the CAG promoter consists of (1) the cytomegalovirus (CMV) early enhancer element, (2) the promoter, the first exon and the first intron of chicken beta-actin gene, and (3) the splice acceptor of the rabbit beta-globin gene.

As used herein, the term “NARE” can refer to a promoter defined in the traditional sense as well as a combination of nucleic acid regulatory elements that comprises a promoter and/or other nucleic acid regulatory elements that modulate the expression of a gene that is operably linked to the element(s). A nucleic acid regulatory element may be, or include one or more of, e.g., promoters, enhancers, translation initiation signals, introns, and/or splicing enhancers.

As used herein, “transgene” refers to a gene (in particular the coding sequence of the gene) that is transferred into one or more cells of an organism, for example using a vector described herein. A transgene can encode a protein or RNA that is normally expressed in cells of the target organism, or may encode a protein or RNA from a different organism. The transgene may be integrated into the genome of the target cell, or may exist as part of an extrachromosomal expression construct.

As used herein, “operatively linked” refers to a first molecule joined to a second molecule, wherein the molecules are so arranged that the first molecule affects the function of the second molecule. The two molecules may or may not be part of a single contiguous molecule and may or may not be adjacent. For example, a NARE is operatively linked to a transcribable polynucleotide molecule if the NARE modulates transcription of the transcribable polynucleotide molecule of interest in a cell. Additionally, two portions of a transcription regulatory element are operatively linked to one another if they are joined such that the transcription-activating functionality of one portion is not adversely affected by the presence of the other portion. Two transcription regulatory elements may be operatively linked to one another by way of a linker nucleic acid (e.g., an intervening non-coding nucleic acid) or may be operatively linked to one another with no intervening nucleotides present.

Provided herein are engineered regulatory elements that can drive transgene expression in a variety of different tissues, including the muscle. The NARE engineering platform described herein has yielded numerous NAREs that can be used to drive the expression of numerous therapeutic transgenes.

Provided herein are NAREs particularly suitable to drive expression in the muscle. As used herein, NAREs starting with “M” are particularly useful for the expression of genes in the muscle. Muscle NAREs can be muscle-specific, meaning that they show significantly increased, or preferential, expression of an operably linked transgene in the muscle as compared to in other tissues.

Tables 4 and 6 provide NAREs particularly suitable to drive expression in the muscle.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE provided in Tables 4 and 6. In embodiments, provided is a polynucleotide comprising one or more NAREs provided in Tables 4 and 6.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. Methods and computer programs for determining both sequence identity and similarity are publicly available, including, but not limited to, the GCG program package (Devereux et al., Nucleic Acids Research 12:387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of M1-M18, M20, M21, M23, M24, M32-M49, M51-M70, and M74-M120 (as listed in Table 6). In embodiments, provided is a polynucleotide sequence that comprises a NARE selected from the group consisting of M1-M18, M20, M21, M23, M24, M32-M49, M51-M70, and M74-M120.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of M21, M23-M28, M32, M34-M36, M51, M53, M55, M57, M59, M61, and M63 (as listed in Table 6). In embodiments, provided is a polynucleotide sequence that comprises a NARE selected from the group consisting of M21, M23-M28, M32, M34-M36, M51, M53, M55, M57, M59, M61, and M63.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of M21 and M24. In embodiments, provided is a polynucleotide sequence that comprises a NARE selected from the group consisting of M21 and M24.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of M21, M24-M27, M30, and M32-M36 (as listed in Table 6). In embodiments, provided is a polynucleotide sequence that comprises a NARE selected from the group consisting of M21, M24-M27, M30, and M32-M36.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of M49, M51, M61, M98, M99, and M111-M115 (as listed in Table 6).

In embodiments, provided is a polynucleotide sequence that comprises a NARE selected from the group consisting of M49, M51, M61, M98, M99, and M111-M115.

Provided herein is a method for expression of a transgene in muscle cells, wherein the transgene is operably linked to a NARE disclosed herein particularly suitable to drive expression in the muscle.

Provided herein is a method for expression of a transgene in muscle cells, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a NARE provided herein. Provided herein is a method for expression of a transgene in muscle cells, wherein the transgene is operably linked to a NARE provided herein.

Provided herein is a method for expression of a transgene in muscle cells, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of M1-M18, M20, M21, M23, M24, M32-M49, M51-M70, and M74-M120. Provided herein is a method for expression of a transgene in muscle cells, wherein the transgene is operably linked to a NARE selected from the group consisting of M1-M18, M20, M21, M23, M24, M32-M49, M51-M70, and M74-M120.

In some embodiments, the NARE comprises a sequence that is at least 80% identical, at least 85% identical, at least 90% identical, at least 95% identical, at least 96% identical, at least 97% identical, at least 98% identical, or at least 99% identical to a sequence selected from the group consisting of SEQ ID NOs: 1-18, 20, 21, 23, 24, 32-49, 51-70, and 74-120.

In some embodiments, the NARE comprises a sequence selected from the group consisting of SEQ ID NOs: 1-18, 20, 21, 23, 24, 32-49, 51-70, and 74-120.

In some embodiments, the NARE comprises a sequence that is at least 80% identical, at least 85% identical, at least 90% identical, at least 95% identical, at least 96% identical, at least 97% identical, at least 98% identical, or at least 99% identical to a sequence selected from the group consisting of SEQ ID NOs: 1-18 and 32.

In some embodiments, the NARE comprises a sequence that is selected from the group consisting of SEQ ID NOs: 1-18 and 32.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 164; (ii) a sequence that is at least 80% identical, at least 85% identical, at least 90% identical, at least 95% identical, at least 96% identical, at least 97% identical, at least 98% identical, or at least 99% identical to SEQ ID NO:134; (iii) a sequence that is at least 80% identical, at least 85% identical, at least 90% identical, at least 95% identical, at least 96% identical, at least 97% identical, at least 98% identical, or at least 99% identical to SEQ ID NO: 135; and (vi) a sequence that is at least 80% identical, at least 85% identical, at least 90% identical, at least 95% identical, at least 96% identical, at least 97% identical, at least 98% identical, or at least 99% identical to SEQ ID NOs: 148 or 149.

In some embodiments, the NARE comprises: (i) SEQ ID NO: 164; (ii) SEQ ID NO: 134; (iii) SEQ ID NO:135; and (vi) SEQ ID NOs: 148 or 149.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 164; (ii) a sequence that is at least 80% identical, at least 85% identical, at least 90% identical, at least 95% identical, at least 96% identical, at least 97% identical, at least 98% identical, or at least 99% identical to SEQ ID NO:134; (iii) a sequence that is at least 80% identical, at least 85% identical, at least 90% identical, at least 95% identical, at least 96% identical, at least 97% identical, at least 98% identical, or at least 99% identical to SEQ ID NO: 135; and (vi) a sequence that is at least 80% identical, at least 85% identical, at least 90% identical, at least 95% identical, at least 96% identical, at least 97% identical, at least 98% identical, or at least 99% identical to SEQ ID NO:148.

In some embodiments, the NARE comprises: (i) SEQ ID NO:164; (ii) SEQ ID NO: 134; (iii) SEQ ID NO:135; and (vi) SEQ ID NO:148.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 164; (ii) a sequence that is at least 80% identical, at least 85% identical, at least 90% identical, at least 95% identical, at least 96% identical, at least 97% identical, at least 98% identical, or at least 99% identical to SEQ ID NO:134; (iii) a sequence that is at least 80% identical, at least 85% identical, at least 90% identical, at least 95% identical, at least 96% identical, at least 97% identical, at least 98% identical, or at least 99% identical to SEQ ID NO: 135; and (vi) a sequence that is at least 80% identical, at least 85% identical, at least 90% identical, at least 95% identical, at least 96% identical, at least 97% identical, at least 98% identical, or at least 99% identical to SEQ ID NO:149.

In some embodiments, the NARE comprises: (i) SEQ ID NO: 164; (ii) SEQ ID NO: 134; (iii) SEQ ID NO:135; and (vi) SEQ ID NO:149.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 218; and (ii) a sequence that is at least 80% identical, at least 85% identical, at least 90% identical, at least 95% identical, at least 96% identical, at least 97% identical, at least 98% identical, or at least 99% identical to SEQ ID NOs: 148 or 149.

In some embodiments, the NARE comprises: (i) SEQ ID NO:218; and (ii) SEQ ID NOs: 148 or 149.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 218; and (ii) a sequence that is at least 80% identical, at least 85% identical, at least 90% identical, at least 95% identical, at least 96% identical, at least 97% identical, at least 98% identical, or at least 99% identical to SEQ ID NO:148.

In some embodiments, the NARE comprises: (i) SEQ ID NO:218; and (ii) SEQ ID NO: 148.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 218; and (ii) a sequence that is at least 80% identical, at least 85% identical, at least 90% identical, at least 95% identical, at least 96% identical, at least 97% identical, at least 98% identical, or at least 99% identical to SEQ ID NO:149.

In some embodiments, the NARE comprises: (i) SEQ ID NO:218; and (ii) SEQ ID NO: 149.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 133, 142, 165 or 217; (ii) optionally, a sequence that is at least 80% identical, at least 85% identical, at least 90% identical, at least 95% identical, at least 96% identical, at least 97% identical, at least 98% identical, or at least 99% identical to SEQ ID NOs: 154 or 189; (iii) optionally, a sequence that is at least 80% identical, at least 85% identical, at least 90% identical, at least 95% identical, at least 96% identical, at least 97% identical, at least 98% identical, or at least 99% identical to SEQ ID NO:220; and (iv) a sequence that is at least 80% identical, at least 85% identical, at least 90% identical, at least 95% identical, at least 96% identical, at least 97% identical, at least 98% identical, or at least 99% identical to SEQ ID NO:150.

In some embodiments, the NARE comprises: (i) any one of SEQ ID NOs: 133, 142, 165 or 217; (ii) optionally, SEQ ID NOs: 154 or 189; (iii) optionally, SEQ ID NO:220; and (iv) SEQ ID NO:150.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 133; and (ii) a sequence that is at least 80% identical, at least 85% identical, at least 90% identical, at least 95% identical, at least 96% identical, at least 97% identical, at least 98% identical, or at least 99% identical to SEQ ID NO:150.

In some embodiments, the NARE comprises: (i) SEQ ID NO: 133; and (ii) SEQ ID NO: 150.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 165; (ii) a sequence that is at least 80% identical, at least 85% identical, at least 90% identical, at least 95% identical, at least 96% identical, at least 97% identical, at least 98% identical, or at least 99% identical to SEQ ID NO:154; (iii) a sequence that is at least 80% identical, at least 85% identical, at least 90% identical, at least 95% identical, at least 96% identical, at least 97% identical, at least 98% identical, or at least 99% identical to SEQ ID NO: 220; and (iv) a sequence that is at least 80% identical, at least 85% identical, at least 90% identical, at least 95% identical, at least 96% identical, at least 97% identical, at least 98% identical, or at least 99% identical to SEQ ID NO:150.

In some embodiments, the NARE comprises: (i) SEQ ID NO: 165; (ii) SEQ ID NO: 154; (iii) SEQ ID NO:220; and (iv) SEQ ID NO:150.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 142; and (ii) a sequence that is at least 80% identical, at least 85% identical, at least 90% identical, at least 95% identical, at least 96% identical, at least 97% identical, at least 98% identical, or at least 99% identical to SEQ ID NO:150.

In some embodiments, the NARE comprises: (i) SEQ ID NO: 142; and (ii) SEQ ID NO: 150.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 217; (ii) a sequence that is at least 80% identical, at least 85% identical, at least 90% identical, at least 95% identical, at least 96% identical, at least 97% identical, at least 98% identical, or at least 99% identical to SEQ ID NO:189; and (iii) a sequence that is at least 80% identical, at least 85% identical, at least 90% identical, at least 95% identical, at least 96% identical, at least 97% identical, at least 98% identical, or at least 99% identical to SEQ ID NO: 150.

In some embodiments, the NARE comprises: (i) SEQ ID NO:217; (ii) SEQ ID NO: 189; and (iii) SEQ ID NO:150.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 156-163.

In some embodiments, the NARE comprises: (i) SEQ ID NO: 132; and (ii) any one of SEQ ID NOs: 156-163.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:156.

In some embodiments, the NARE comprises: (i) SEQ ID NO: 132; and (ii) SEQ ID NO: 156.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:157.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:158.

In some embodiments, the NARE comprises: (i) SEQ ID NO: 132; and (ii) SEQ ID NO: 158.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO: 159.

In some embodiments, the NARE comprises: (i) SEQ ID NO:132; and (ii) SEQ ID NO: 159.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:160.

In some embodiments, the NARE comprises: (i) SEQ ID NO:132; and (ii) SEQ ID NO: 160.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:161.

In some embodiments, the NARE comprises: (i) SEQ ID NO:132; and (ii) SEQ ID NO: 161.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:162.

In some embodiments, the NARE comprises: (i) SEQ ID NO:132; and (ii) SEQ ID NO: 162.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 132; and (ii) a sequence that is at least 80% identical, at least 85% identical, at least 90% identical, at least 95% identical, at least 96% identical, at least 97% identical, at least 98% identical, or at least 99% identical to SEQ ID NO:163.

In some embodiments, the NARE comprises: (i) SEQ ID NO:132; and (ii) SEQ ID NO: 163.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 131; (ii) a sequence that is at least 80% identical, at least 85% identical, at least 90% identical, at least 95% identical, at least 96% identical, at least 97% identical, at least 98% identical, or at least 99% identical to SEQ ID NOs: 126 or 213; and (iii) optionally, a sequence that is at least 80% identical, at least 85% identical, at least 90% identical, at least 95% identical, at least 96% identical, at least 97% identical, at least 98% identical, or at least 99% identical to SEQ ID NO:200.

In some embodiments, the NARE comprises: (i) SEQ ID NO:131; (ii) SEQ ID NOs: 126 or 213; and (iii) optionally, SEQ ID NO:200.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 131; (ii) a sequence that is at least 80% identical, at least 85% identical, at least 90% identical, at least 95% identical, at least 96% identical, at least 97% identical, at least 98% identical, or at least 99% identical to SEQ ID NO:126; and (iii) optionally, a sequence that is at least 80% identical, at least 85% identical, at least 90% identical, at least 95% identical, at least 96% identical, at least 97% identical, at least 98% identical, or at least 99% identical to SEQ ID NO:200.

In some embodiments, the NARE comprises: (i) SEQ ID NO:131; (ii) SEQ ID NO: 126; and (iii) optionally, SEQ ID NO:200.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 131; and (ii) a sequence that is at least 80% identical, at least 85% identical, at least 90% identical, at least 95% identical, at least 96% identical, at least 97% identical, at least 98% identical, or at least 99% identical to SEQ ID NO: 126.

In some embodiments, the NARE comprises: (i) SEQ ID NO:131; and (ii) SEQ ID NO: 126.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 131; (ii) a sequence that is at least 80% identical, at least 85% identical, at least 90% identical, at least 95% identical, at least 96% identical, at least 97% identical, at least 98% identical, or at least 99% identical to SEQ ID NO:126; and (iii) a sequence that is at least 80% identical, at least 85% identical, at least 90% identical, at least 95% identical, at least 96% identical, at least 97% identical, at least 98% identical, or at least 99% identical to SEQ ID NO: 200.

In some embodiments, the NARE comprises: (i) SEQ ID NO:131; (ii) SEQ ID NO: 126; and (iii) SEQ ID NO:200.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 131; (ii) a sequence that is at least 80% identical, at least 85% identical, at least 90% identical, at least 95% identical, at least 96% identical, at least 97% identical, at least 98% identical, or at least 99% identical to SEQ ID NO:213; and (iii) a sequence that is at least 80% identical, at least 85% identical, at least 90% identical, at least 95% identical, at least 96% identical, at least 97% identical, at least 98% identical, or at least 99% identical to SEQ ID NO: 200.

In some embodiments, the NARE comprises: (i) SEQ ID NO:131; (ii) SEQ ID NO: 213; and (iii) SEQ ID NO:200.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 190-199; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) any one of SEQ ID NOs: 190-199; and (ii) SEQ ID NO:155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 196; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO:196; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO: 190; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 191; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO:191; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 192; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO:192; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 193; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO: 193; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 194; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO:194; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 195; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO: 195; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 197; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO:197; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 198; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO: 198; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 199; and (ii) a sequence that is at least 80% identical, at least 85% identical, at least 90% identical, at least 95% identical, at least 96% identical, at least 97% identical, at least 98% identical, or at least 99% identical to SEQ ID NO:155.

In some embodiments, the NARE comprises: (i) SEQ ID NO:199; and (ii) SEQ ID NO: 155.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 190-199; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) any one of SEQ ID NOs: 190-199; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 196; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:196; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:190; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 191; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:191; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 192; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:192; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 193; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO: 193; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 194; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO: 194; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 195; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:195; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 197; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO: 197; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 198; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:198; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 199; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO: 199; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 190-199; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) any one of SEQ ID NOs: 190-199; and (ii) SEQ ID NO:130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 196; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO: 196; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 190; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO: 190; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 191; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO: 191; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 192; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO:192; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 193; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO:193; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 194; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO: 194; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 195; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO:195; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 197; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO: 197; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 198; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO:198; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 199; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises: (i) SEQ ID NO: 199; and (ii) SEQ ID NO: 130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 123-125, 127, 136-141, 143, 145-147, 152, 153, 209-212, 219, and 221.

In some embodiments, the NARE comprises: (i) any one of SEQ ID NOs: 128-130; and (ii) any one of SEQ ID NOs: 123-125, 127, 136-141, 143, 145-147, 152, 153, 209-212, 219, or 221.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:137.

In some embodiments, the NARE comprises: (i) any one of SEQ ID NOs: 128-130; and (ii) SEQ ID NO:137.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:138.

In some embodiments, the NARE comprises: (i) any one of SEQ ID NOs: 128-130; and (ii SEQ ID NO:138.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:219.

In some embodiments, the NARE comprises: (i) any one of SEQ ID NOs: 128-130; and (ii) SEQ ID NO:219.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:212.

In some embodiments, the NARE comprises: (i) any one of SEQ ID NOs: 128-130; and (ii) SEQ ID NO:212.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:211.

In some embodiments, the NARE comprises: (i) any one of SEQ ID NOs: 128-130; and (ii) SEQ ID NO:211.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:143.

In some embodiments, the NARE comprises: (i) any one of SEQ ID NOs: 128-130; and (ii) SEQ ID NO:143.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:221.

In some embodiments, the NARE comprises: (i) any one of SEQ ID NOs: 128-130; and (ii) SEQ ID NO:221.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:123.

In some embodiments, the NARE comprises: (i) any one of SEQ ID NOs: 128-130; and (ii) SEQ ID NO:123.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:127.

In some embodiments, the NARE comprises: (i) any one of SEQ ID NOs: 128-130; and (ii) SEQ ID NO:127.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:139.

In some embodiments, the NARE comprises: (i) any one of SEQ ID NOs: 128-130; and (ii) SEQ ID NO:139.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:136.

In some embodiments, the NARE comprises: (i) any one of SEQ ID NOs: 128-130; and (ii) SEQ ID NO:136.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:141.

In some embodiments, the NARE comprises: (i) any one of SEQ ID NOs: 128-130; and (ii) SEQ ID NO:141.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:209.

In some embodiments, the NARE comprises: (i) any one of SEQ ID NOs: 128-130; and (ii) SEQ ID NO:209.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:124.

In some embodiments, the NARE comprises: (i) any one of SEQ ID NOs: 128-130; and (ii) SEQ ID NO:124.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:125.

In some embodiments, the NARE comprises: (i) any one of SEQ ID NOs: 128-130; and (ii) SEQ ID NO:125.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:152.

In some embodiments, the NARE comprises: (i) any one of SEQ ID NOs: 128-130; and (ii) SEQ ID NO:152.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:140.

In some embodiments, the NARE comprises: (i) any one of SEQ ID NOs: 128-130; and (ii) SEQ ID NO:140.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:145.

In some embodiments, the NARE comprises: (i) any one of SEQ ID NOs: 128-130; and (ii) SEQ ID NO:145.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:146.

In some embodiments, the NARE comprises: (i) any one of SEQ ID NOs: 128-130; and (ii) SEQ ID NO:146.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:147.

In some embodiments, the NARE comprises: (i) any one of SEQ ID NOs: 128-130; and (ii) SEQ ID NO:147.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:153.

In some embodiments, the NARE comprises: (i) any one of SEQ ID NOs: 128-130; and (ii) SEQ ID NO:153.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to any one of SEQ ID NOs: 128-130; and (ii) a sequence that is at least 80% identical, at least 85% identical, at least 90% identical, at least 95% identical, at least 96% identical, at least 97% identical, at least 98% identical, or at least 99% identical to SEQ ID NO:210.

In some embodiments, the NARE comprises: (i) any one of SEQ ID NOs: 128-130; and (ii) SEQ ID NO:210.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 123-125, 127, 136-141, 143, 145-147, 152, 153, 209-212, 219, or 221.

In some embodiments, the NARE comprises: (i) SEQ ID NO: 128; and (ii) any one of SEQ ID NOs: 123-125, 127, 136-141, 143, 145-147, 152, 153, 209-212, 219, or 221.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:137.

In some embodiments, the NARE comprises: (i) SEQ ID NO:128; and (ii) SEQ ID NO: 137.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:138.

In some embodiments, the NARE comprises: (i) SEQ ID NO: 128; and (ii SEQ ID NO: 138.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:219.

In some embodiments, the NARE comprises: (i) SEQ ID NO:128; and (ii) SEQ ID NO: 219.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:212.

In some embodiments, the NARE comprises: (i) SEQ ID NO:128; and (ii) SEQ ID NO: 212.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:211.

In some embodiments, the NARE comprises: (i) SEQ ID NO:128; and (ii) SEQ ID NO: 211.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:143.

In some embodiments, the NARE comprises: (i) SEQ ID NO:128; and (ii) SEQ ID NO: 143.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:221.

In some embodiments, the NARE comprises: (i) SEQ ID NO: 128; and (ii) SEQ ID NO: 221.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:123.

In some embodiments, the NARE comprises: (i) SEQ ID NO: 128; and (ii) SEQ ID NO: 123.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:127.

In some embodiments, the NARE comprises: (i) SEQ ID NO:128; and (ii) SEQ ID NO: 127.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:139.

In some embodiments, the NARE comprises: (i) SEQ ID NO: 128; and (ii) SEQ ID NO: 139.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:136.

In some embodiments, the NARE comprises: (i) SEQ ID NO: 128; and (ii) SEQ ID NO: 136.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:141.

In some embodiments, the NARE comprises: (i) SEQ ID NO:128; and (ii) SEQ ID NO: 141.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:209.

In some embodiments, the NARE comprises: (i) SEQ ID NO: 128; and (ii) SEQ ID NO: 209.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:124.

In some embodiments, the NARE comprises: (i) SEQ ID NO:128; and (ii) SEQ ID NO: 124.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:125.

In some embodiments, the NARE comprises: (i) SEQ ID NO:128; and (ii) SEQ ID NO: 125.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:152.

In some embodiments, the NARE comprises: (i) SEQ ID NO: 128; and (ii) SEQ ID NO: 152.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:140.

In some embodiments, the NARE comprises: (i) SEQ ID NO:128; and (ii) SEQ ID NO: 140.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:145.

In some embodiments, the NARE comprises: (i) SEQ ID NO: 128; and (ii) SEQ ID NO: 145.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:146.

In some embodiments, the NARE comprises: (i) SEQ ID NO: 128; and (ii) SEQ ID NO: 146.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:147.

In some embodiments, the NARE comprises: (i) a SEQ ID NO: 128; and (ii) SEQ ID NO: 147.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:153.

In some embodiments, the NARE comprises: (i) SEQ ID NO:128; and (ii) SEQ ID NO: 153.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 128; and (ii) a sequence that is at least 80% identical, at least 85% identical, at least 90% identical, at least 95% identical, at least 96% identical, at least 97% identical, at least 98% identical, or at least 99% identical to SEQ ID NO:210.

In some embodiments, the NARE comprises: (i) SEQ ID NO:128; and (ii) SEQ ID NO: 210.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 166-169, 171, or 172; and (iii) optionally, a sequence that is at least 80% identical, at least 85% identical, at least 90% identical, at least 95% identical, at least 96% identical, at least 97% identical, at least 98% identical, or at least 99% identical to SEQ ID NOs: 122 or 154

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 166-168, 171, 172; and (iii) optionally, a sequence that is at least 80% identical, at least 85% identical, at least 90% identical, at least 95% identical, at least 96% identical, at least 97% identical, at least 98% identical, or at least 99% identical to SEQ ID NOs: 122 or 154

In some embodiments, the NARE comprises: (ii) SEQ ID NO: 121; (iii) any one of SEQ ID NOs: 166-169, 171, 172; and (iii) optionally, SEQ ID NOs: 122 or 154;

In some embodiments, the NARE comprises: (i) SEQ ID NO:121; (ii) any one of SEQ ID NOs: 166-168, 171, 172; and (iii) optionally, SEQ ID NOs: 122 or 154

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 166-169, 171, or 172.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 166-168, 171, or 172.

In some embodiments, the NARE comprises: (i) SEQ ID NO:121; and (ii) any one of SEQ ID NOs: 166-169, 171, or 172.

In some embodiments, the NARE comprises: (i) SEQ ID NO: 121; and (ii) any one of SEQ ID NOs: 166-168, 171, or 172.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122 or 154; (ii) a sequence that is at least 80% identical, at least 85% identical, at least 90% identical, at least 95% identical, at least 96% identical, at least 97% identical, at least 98% identical, or at least 99% identical to any one of SEQ ID NO:121; and (iii) a sequence that is at least 80% identical, at least 85% identical, at least 90% identical, at least 95% identical, at least 96% identical, at least 97% identical, at least 98% identical, or at least 99% identical to SEQ ID NO: 167.

In some embodiments, the NARE comprises: (i) SEQ ID NO: 122 or 154; (ii) SEQ ID NO: 121; and (iii) SEQ ID NO:167.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to SEQ ID NO:166.

In some embodiments, the NARE comprises: (i) SEQ ID NO: 121; and (ii) SEQ ID NO: 166.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to SEQ ID NO:167.

In some embodiments, the NARE comprises: (i) SEQ ID NO:121; and (ii) SEQ ID NO: 167.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to SEQ ID NOs: 168 or 169.

In some embodiments, the NARE comprises: (i) SEQ ID NO:121; and (ii) SEQ ID NOs: 168 or 169.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to SEQ ID NO:168.

In some embodiments, the NARE comprises: (i) SEQ ID NO:121; and (ii) SEQ ID NO: 168.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to SEQ ID NO:171.

In some embodiments, the NARE comprises: (i) SEQ ID NO:121; and (ii) SEQ ID NO: 171.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 154; (ii) a sequence that is at least 80% identical, at least 85% identical, at least 90% identical, at least 95% identical, at least 96% identical, at least 97% identical, at least 98% identical, or at least 99% identical to SEQ ID NO:121; and (iii) a sequence that is at least 80% identical, at least 85% identical, at least 90% identical, at least 95% identical, at least 96% identical, at least 97% identical, at least 98% identical, or at least 99% identical to SEQ ID NO: 167.

In some embodiments, the NARE comprises: (i) SEQ ID NO:154; (ii) SEQ ID NO: 121; and (iii) SEQ ID NO:167.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; (ii) a sequence that is at least 80% identical, at least 85% identical, at least 90% identical, at least 95% identical, at least 96% identical, at least 97% identical, at least 98% identical, or at least 99% identical to SEQ ID NO:121; and (iii) a sequence that is at least 80% identical, at least 85% identical, at least 90% identical, at least 95% identical, at least 96% identical, at least 97% identical, at least 98% identical, or at least 99% identical to SEQ ID NO: 167.

In some embodiments, the NARE comprises: (i) SEQ ID NO: 122; (ii) SEQ ID NO: 121; and (iii) SEQ ID NO:167.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 121; and (ii) a sequence that is at least 80% identical, at least 85% identical, at least 90% identical, at least 95% identical, at least 96% identical, at least 97% identical, at least 98% identical, or at least 99% identical to SEQ ID NO:172.

In some embodiments, the NARE comprises: (i) SEQ ID NO:121; and (ii) SEQ ID NO: 172.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 173-179, or 188, and (iii) optionally, a sequence that is at least 80% identical, at least 85% identical, at least 90% identical, at least 95% identical, at least 96% identical, at least 97% identical, at least 98% identical, or at least 99% identical to SEQ ID NO:207.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; (ii) a sequence that is at least 80% identical, at least 85% identical, at least 90% identical, at least 95% identical, at least 96% identical, at least 97% identical, at least 98% identical, or at least 99% identical to any one of SEQ ID NOs: 173, 174, 176, 178, 179, or 188, and (iii) optionally, a sequence that is at least 80% identical, at least 85% identical, at least 90% identical, at least 95% identical, at least 96% identical, at least 97% identical, at least 98% identical, or at least 99% identical to SEQ ID NO:207.

In some embodiments, the NARE comprises: (i) SEQ ID NO:122; (ii) any one of SEQ ID NOs: 173-179, or 188, and (iii) optionally, SEQ ID NO:207.

In some embodiments, the NARE comprises: (i) SEQ ID NO:122; (ii) any one of SEQ ID NOs: 173, 174, 176, 178, 179, or 188, and (iii) optionally, SEQ ID NO:207.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 173-179.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 173, 174, 176, 178, or 179.

In some embodiments, the NARE comprises: (i) a SEQ ID NO:122; and (ii) any one of SEQ ID NOs: 173-179.

In some embodiments, the NARE comprises: (i) a SEQ ID NO:122; and (ii) any one of SEQ ID NOs: 173, 174, 176, 178, or 179.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NO:173.

In some embodiments, the NARE comprises: (i) SEQ ID NO:122; and (ii) SEQ ID NO: 173.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NOs: 174 or 175.

In some embodiments, the NARE comprises: (i) SEQ ID NO: 122; and (ii) SEQ ID NOs: 174 or 175.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NO:174.

In some embodiments, the NARE comprises: (i) SEQ ID NO: 122; and (ii) SEQ ID NO: 174.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NOs: 176 or 177.

In some embodiments, the NARE comprises: (i) SEQ ID NO:122; and (ii) SEQ ID NOs: 176 or 177.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NO:176.

In some embodiments, the NARE comprises: (i) SEQ ID NO:122; and (ii) SEQ ID NO: 176.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NO:178.

In some embodiments, the NARE comprises: (i) SEQ ID NO: 122; and (ii) SEQ ID NO: 178.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; and (ii) a sequence that is at least 80% identical, at least 85% identical, at least 90% identical, at least 95% identical, at least 96% identical, at least 97% identical, at least 98% identical, or at least 99% identical to SEQ ID NO:179.

In some embodiments, the NARE comprises: (i) SEQ ID NO: 122; and (ii) SEQ ID NO: 179.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; (ii) a sequence that is at least 80% identical, at least 85% identical, at least 90% identical, at least 95% identical, at least 96% identical, at least 97% identical, at least 98% identical, or at least 99% identical to SEQ ID NO:188; and (iii) a sequence that is at least 80% identical, at least 85% identical, at least 90% identical, at least 95% identical, at least 96% identical, at least 97% identical, at least 98% identical, or at least 99% identical to SEQ ID NO: 207.

In some embodiments, the NARE comprises: (i) SEQ ID NO:122; (ii) SEQ ID NO: 188; and (iii) SEQ ID NO:207.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 184; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 185-187.

In some embodiments, the NARE comprises: (i) SEQ ID NO:184; and (ii) any one of SEQ ID NOs: 185-187.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 184; and (ii) a sequence that is at least 80% identical, at least 85% identical, at least 90% identical, at least 95% identical, at least 96% identical, at least 97% identical, at least 98% identical, or at least 99% identical to SEQ ID NO:185.

In some embodiments, the NARE comprises: (i) SEQ ID NO:184; and (ii) SEQ ID NO: 185.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 184; and (ii) a sequence that is at least 80% identical, at least 85% identical, at least 90% identical, at least 95% identical, at least 96% identical, at least 97% identical, at least 98% identical, or at least 99% identical to SEQ ID NO:186.

In some embodiments, the NARE comprises: (i) SEQ ID NO:184; and (ii) SEQ ID NO: 186.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 184; and (ii) a sequence that is at least 80% identical, at least 85% identical, at least 90% identical, at least 95% identical, at least 96% identical, at least 97% identical, at least 98% identical, or at least 99% identical to SEQ ID NO:187.

In some embodiments, the NARE comprises: (i) SEQ ID NO:184; and (ii) SEQ ID NO: 187.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 180; and (ii) a sequence that is at least 80% identical, at least 85% identical, at least 90% identical, at least 95% identical, at least 96% identical, at least 97% identical, at least 98% identical, or at least 99% identical to SEQ ID NOs: 181 or 182.

In some embodiments, the NARE comprises: (i) SEQ ID NO: 180; and (ii) SEQ ID NOs: 181 or 182.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 180; and (ii) a sequence that is at least 80% identical, at least 85% identical, at least 90% identical, at least 95% identical, at least 96% identical, at least 97% identical, at least 98% identical, or at least 99% identical to SEQ ID NO:181.

In some embodiments, the NARE comprises: (i) SEQ ID NO:180; and (ii) SEQ ID NO: 181.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 180; and (ii) a sequence that is at least 80% identical, at least 85% identical, at least 90% identical, at least 95% identical, at least 96% identical, at least 97% identical, at least 98% identical, or at least 99% identical to SEQ ID NO:182.

In some embodiments, the NARE comprises: (i) SEQ ID NO:180; and (ii) SEQ ID NO: 182.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO:182; and (iii) optionally, a sequence that is at least 80% identical, at least 85% identical, at least 90% identical, at least 95% identical, at least 96% identical, at least 97% identical, at least 98% identical, or at least 99% identical to any one of SEQ ID NOs: 200, 201, 205, or 208.

In some embodiments, the NARE comprises: (i) SEQ ID NO:183; (ii) SEQ ID NO: 182; and (iii) optionally, any one of SEQ ID NOs: 200, 201, 205, or 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO:182; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 200, 201, 205, or 208.

In some embodiments, the NARE comprises: (i) SEQ ID NO: 183; (ii) SEQ ID NO: 182; and (iii) any one of SEQ ID NOs: 200, 201, 205, or 208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; and (ii) a sequence that is at least 80% identical, at least 85% identical, at least 90% identical, at least 95% identical, at least 96% identical, at least 97% identical, at least 98% identical, or at least 99% identical to SEQ ID NO:182.

In some embodiments, the NARE comprises: (i) SEQ ID NO:183; and (ii) SEQ ID NO: 182.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO:182; and (iii) a sequence that is at least 80% identical, at least 85% identical, at least 90% identical, at least 95% identical, at least 96% identical, at least 97% identical, at least 98% identical, or at least 99% identical to SEQ ID NO: 200.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO:182; and (iii) a sequence that is at least 80% identical, at least 85% identical, at least 90% identical, at least 95% identical, at least 96% identical, at least 97% identical, at least 98% identical, or at least 99% identical to SEQ ID NO: 205.

In some embodiments, the NARE comprises: (i) SEQ ID NO:183; (ii) SEQ ID NO: 182; and (iii)) SEQ ID NO:205.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO:182; and (iii) a sequence that is at least 80% identical, at least 85% identical, at least 90% identical, at least 95% identical, at least 96% identical, at least 97% identical, at least 98% identical, or at least 99% identical to SEQ ID NO: 208.

In some embodiments, the NARE comprises: (i) SEQ ID NO:183; (ii) SEQ ID NO: 182; and (iii)) SEQ ID NO:208.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 183; (ii) a sequence that is at least 80% identical, at least 85% identical, at least 90% identical, at least 95% identical, at least 96% identical, at least 97% identical, at least 98% identical, or at least 99% identical to SEQ ID NO:182; and (iii) a sequence that is at least 80% identical, at least 85% identical, at least 90% identical, at least 95% identical, at least 96% identical, at least 97% identical, at least 98% identical, or at least 99% identical to SEQ ID NO: 201.

In some embodiments, the NARE comprises: (i) SEQ ID NO: 183; (ii) SEQ ID NO: 182; and (iii)) SEQ ID NO:201.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 170; (ii) optionally, a sequence that is at least 80% identical, at least 85% identical, at least 90% identical, at least 95% identical, at least 96% identical, at least 97% identical, at least 98% identical, or at least 99% identical to SEQ ID NO:188; and (iii) a sequence that is at least 80% identical, at least 85% identical, at least 90% identical, at least 95% identical, at least 96% identical, at least 97% identical, at least 98% identical, or at least 99% identical to SEQ ID NOs: 203 or 204.

In some embodiments, the NARE comprises: (i) SEQ ID NO:170; (ii) optionally, a SEQ ID NO:188; and (iii) SEQ ID NOs: 203 or 204.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 170; and (ii) a sequence that is at least 80% identical, at least 85% identical, at least 90% identical, at least 95% identical, at least 96% identical, at least 97% identical, at least 98% identical, or at least 99% identical to SEQ ID NO:203.

In some embodiments, the NARE comprises: (i) SEQ ID NO:170; and (ii) SEQ ID NO: 203.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 122; (ii) a sequence that is at least 80% identical, at least 85% identical, at least 90% identical, at least 95% identical, at least 96% identical, at least 97% identical, at least 98% identical, or at least 99% identical to SEQ ID NO:188; and (iii) a sequence that is at least 80% identical, at least 85% identical, at least 90% identical, at least 95% identical, at least 96% identical, at least 97% identical, at least 98% identical, or at least 99% identical to SEQ ID NO: 204.

In some embodiments, the NARE comprises: (i) a SEQ ID NO:122; (ii) SEQ ID NO: 188; and (iii) SEQ ID NO:204.

In some embodiments, the NARE comprises: (i) at least one copy of a sequence that is at least 80% identical, at least 85% identical, at least 90% identical, at least 95% identical, at least 96% identical, at least 97% identical, at least 98% identical, or at least 99% identical to SEQ ID NOs: 154 or 164; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130. In some embodiments, the NARE comprises: (i) SEQ ID NOs: 154 or 164; and (ii) any one of SEQ ID NOs: 128-130.

In some embodiments, the NARE comprises: (i) at least one copy of a sequence that is at least 80% identical, at least 85% identical, at least 90% identical, at least 95% identical, at least 96% identical, at least 97% identical, at least 98% identical, or at least 99% identical to SEQ ID NO:154; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130. In some embodiments, the NARE comprises: (i) a first sequence that is at least 80% identical, at least 85% identical, at least 90% identical, at least 95% identical, at least 96% identical, at least 97% identical, at least 98% identical, or at least 99% identical to SEQ ID NO:154; (ii) a second sequence that is at least 80% identical, at least 85% identical, at least 90% identical, at least 95% identical, at least 96% identical, at least 97% identical, at least 98% identical, or at least 99% identical to SEQ ID NO:154; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) at least one copy of SEQ ID NO: 154; and (ii) any one of SEQ ID NOs: 128-130. In some embodiments, the NARE comprises: (i) a first sequence comprising SEQ ID NO:154; (ii) a second sequence comprising SEQ ID NO: 154; and (iii) any one of SEQ ID NOs: 128-130.

In some embodiments, the NARE comprises: (i) at least one copy of a sequence that is at least 80% identical, at least 85% identical, at least 90% identical, at least 95% identical, at least 96% identical, at least 97% identical, at least 98% identical, or at least 99% identical to SEQ ID NO:154; and (ii) a sequence that is at least 80% identical, at least 85% identical, at least 90% identical, at least 95% identical, at least 96% identical, at least 97% identical, at least 98% identical, or at least 99% identical to SEQ ID NO:128. In some embodiments, the NARE comprises: (i) a first sequence that is at least 80% identical, at least 85% identical, at least 90% identical, at least 95% identical, at least 96% identical, at least 97% identical, at least 98% identical, or at least 99% identical to SEQ ID NO:154; (ii) a second sequence that is at least 80% identical, at least 85% identical, at least 90% identical, at least 95% identical, at least 96% identical, at least 97% identical, at least 98% identical, or at least 99% identical to SEQ ID NO:154; and (iii) a sequence that is at least 80% identical, at least 85% identical, at least 90% identical, at least 95% identical, at least 96% identical, at least 97% identical, at least 98% identical, or at least 99% identical to SEQ ID NO:128.

In some embodiments, the NARE comprises: (i) at least one copy of SEQ ID NO: 154; and (ii) SEQ ID NO:128. In some embodiments, the NARE comprises: (i) a first sequence comprising SEQ ID NO:154; (ii) a second sequence comprising SEQ ID NO:154; and (iii) SEQ ID NO:128.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 164; and (ii) a sequence that is at least 80% identical, at least 85% identical, at least 90% identical, at least 95% identical, at least 96% identical, at least 97% identical, at least 98% identical, or at least 99% identical to any one of SEQ ID NOs: 128-130.

In some embodiments, the NARE comprises: (i) SEQ ID NO:164; and (ii) any one of SEQ ID NOs: 128-130.

In some embodiments, the NARE comprises: (i) a sequence that is at least 80% identical, at least 85% identical, at least 90% identical, at least 95% identical, at least 96% identical, at least 97% identical, at least 98% identical, or at least 99% identical to SEQ ID NO: 164; and (ii) a sequence that is at least 80% identical, at least 85% identical, at least 90% identical, at least 95% identical, at least 96% identical, at least 97% identical, at least 98% identical, or at least 99% identical to SEQ ID NO:129.

In some embodiments, the NARE comprises: (i) SEQ ID NO:164; and (ii) SEQ ID NO: 129.

In embodiments, the NARE comprises an intron or a portion of an intron. The intron or portion of an intron may be selected from: ACTA1 intron, ACTA1e intron, ACTC1 intron, ACTC1.3 intron, ACTC1e intron, ACTC1e-ACTC1p intron, ALDOA intron, APOC intron, APOC1 intron, ATF5 intron, CAMK2A intron, CBA intron, chimeric intron, CMV-rabbit beta globlin intron, CRYAB intron, DES.4 intron, EEF1A1 intron, EEF1B2 intron, EFla intron, FHL1 intron, FLOT1 intron, FXYD1 intron, GFAP intron, HBB intron, hCPE intron, hEfla2-intron, HPD intron, IFI27L2 intron, Murine IgG chimeric intron, MVM intron, MVMi-AATp intron, MVMi-SynE-mTTRp intron, Rabit beta globin intron, RBP4 intron, RPL26 intron, RPL27 intron, S100A6 intron, sEEF1A1 intron, SV40 intron, TMSB10 intron, and UCHL 1 intron.

In embodiments, the NARE comprises an enhancer or a portion of an enhancer. The enhancer or a portion of an enhancer may be selected from: ACTA1 enhancer, ACTC1 enhancer, CKM enhancer, CMV enhancer, CMV enhancer, MCK enhancer, mDES enhancer, mDES.1 enhancer, minCKM enhancer, minCKM2 enhancer, minDes enhancer, NRGN enhancer 1.1, NRGN enhancer 1.2, NRGN enhancer 2.1, NRGN enhancer 2.2, SV40 enhancer, and SV40 enhancer (SV40e).

In embodiments, the NARE comprises a UTR or a portion of a UTR. The UTR or a portion of a UTR may be selected from: CTNNB1 UTR, hEfla2-utr1, hEfla2-utr2, hNSE utr1, hNSE utr2, hSyn1_utr1, hSyn1_utr2, HTLV 5′ UTR, HTLV 5′UTR, L21 UTR, TMSB10_utr1, and TMSB10_utr2.

Also provided herein are NAREs used as references or controls. Some of the NAREs provide for constitutive expression of an operably linked transgene, that is, expression of the transgene is maintained at a constant level. Constitutive NAREs may drive expression of an operably linked transgene in a variety of cell types and tissues. As used herein, NAREs starting with “C” are constitutive NAREs. Constitutive NAREs disclosed herein may be useful for the expression of genes in the muscle.

### NARE Modifications

A person skilled in the art will appreciate that certain modifications can be made to the NAREs disclosed herein without eliminating the ability of the NARE to drive gene expression in the desired tissue/cell type. Various in vitro and in vivo methods of confirming that a modified NARE is still capable of driving gene expression are known in the art, including, but not limited to, the methods used herein. For example, the strength of a NARE may be assessed by operatively linking the NARE to a transgene encoding a protein and measuring transgene expression, for example by detecting the mRNA encoding the protein, or by measuring presence or activity of the protein, e.g., by ELISA, Western Blot, fluorescence, enzymatic activity of the protein, etc.

Provided herein are NAREs comprising one or more components. Provided herein are NAREs comprising one or more sequence components (or sequence variants thereof) listed in any one of Tables 4-6. Provided herein are NAREs comprising one or more sequence components (or sequence variants thereof) listed in Table 4. Provided herein is a NARE that comprises one or more components (or sequence variants thereof) of a full-length NARE provided in Table 6. Merely to illustrate in a non-limiting example, provided is a NARE that comprises one or more of the components (or sequence variants thereof) of NARE M37 (CK8emd2):(1) CK8e.1; and (2) md2. In embodiments, provided is a NARE that comprises one or more components (or sequence variants thereof) of a full-length NARE provided in Tables 4 or 6, whereby the components (or sequence variants thereof) are arranged from 5′ to 3′ as presented as in Table 6. In embodiments, provided is a NARE that comprises one or more components (or sequence variants thereof) of a full-length NARE provided in Table 6, whereby the components (or sequence variants thereof) are not arranged from 5′ to 3′ as presented as in Table 6, but in a different order.

In embodiments, in the NARE of Tables 4 or 6, one or more of the components have been replaced with a different sequence, including a component (or sequence variants thereof) from a different NARE of Tables 4 or 6. Further, also contemplated are NAREs that comprise components (or sequence variants thereof) derived from two or more NAREs disclosed Tables 4-6.

Moreover, contemplated are NARE variants that include sequence in addition (e.g., added at either end or inserted within the NARE sequence) to the elements of a NARE disclosed herein. Likewise, provided are NARE variants in which certain sequence has been removed from NAREs disclosed herein (e.g., as a terminal or internal deletion). In embodiments, provided are NAREs comprising one or more components without any additional nucleic acid sequence(s) joining the NARE's components. In embodiments, provided are NAREs comprising one or more components, whereby the individual components are connected by additional nucleic acid sequences. These additional nucleic acid sequences may or may not be relevant for expression of an operatively linked transgene. In embodiments, some of the components are directly linked, while other components are linked to other components via an intervening sequence. As such, contemplated are variants of the NAREs disclosed in Tables 4 or 6, wherein additional sequence has been added between the different components. Further contemplated are variants of the NAREs disclosed in Tables 4 or 6, which disclose the same components of a given NARE in Tables 4 or 6, but differ in the sequence(s) connecting the individual components.

### Nucleic Acid Constructs and Vectors

In one aspect, provided are nucleic acid constructs and vectors and their use for the introduction of a transgene or an expression construct into a cell. Nucleic acid constructs include expression constructs including plasmids. The term “expression construct” refers to a recombinant polynucleotide construct that includes a nucleic acid coding for an RNA capable of being transcribed in a cell. Methods for constructing expression constructs and plasmids through standard recombinant techniques are known in the art.

In some embodiments, the vectors comprise recombinant DNA constructs that include additional DNA elements, including DNA segments that provide for the replication of the DNA in a host cell and expression of the target gene in target cells at appropriate levels. “Vector,” as used herein, means a vehicle that comprises a polynucleotide to be delivered into a host cell, either in vitro, ex vivo or in vivo. Non-limiting examples of vectors include a recombinant plasmid, yeast artificial chromosome (YAC), mini chromosome, DNA mini-circle, or a virus (including virus derived sequences). A vector may also refer to a virion comprising a nucleic acid to be delivered into a host cell, either in vitro, ex vivo or in vivo. In some embodiments, a vector refers to a virion comprising a recombinant viral genome, wherein the viral genome comprises one or more ITRs and a transgene.

In one embodiment, the vector is a viral vector or a combination of multiple viral vectors. In one aspect, provided is a vector comprising any of the nucleic acid constructs disclosed herein.

Provided herein are nucleic acid constructs and vectors comprising a NARE disclosed herein operatively linked to a transgene. In embodiments, the nucleic acid constructs or vectors disclosed herein comprise additional regulatory elements, including, but not limited to, promoters, enhancers, translation initiation signals, introns, and/or splicing enhancers. In embodiments, the nucleic acid constructs or vectors disclosed herein comprise a polyadenylation sequence. In embodiments, the nucleic acid constructs or vectors disclosed herein comprise an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. In one embodiment, the IRES is located 3′ of the transgene.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The Rep reading frame encodes four proteins of molecular weight 78 kD, 68 kD, 52 kD, and 40 kD. These proteins function mainly in regulating AAV replication and rescue and integration of the AAV into a host cell's chromosomes. The Cap reading frame encodes three structural proteins of molecular weight 85 kD (VP 1), 72 kD (VP2), and 61 kD (VP3), which form the virion capsid. More than 80% of total proteins in AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, preferably the Rep and/or Cap genes, but retain functional flanking ITR sequences.

In some embodiments, the viral vector is an rAAV virion, which comprises an rAAV genome and one or more capsid proteins. In some embodiments, the rAAV genome comprises a nucleic acid construct disclosed herein.

In some embodiments, the viral vector disclosed herein comprises a nucleic acid comprising an AAV 5′ ITR and 3′ ITR located 5′ and 3′ of the sequence encoding a transgene, respectively. In embodiments, the transgene is UPF1, BDNF, or ATP7B. However, in certain embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to transgene. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, provided is a vector comprising a nucleic acid sequence comprising (i) a nucleic acid construct disclosed herein and (ii) one or more inverted terminal repeats (ITR). In one embodiment, the nucleic acid sequence comprises a 5′ITR and a 3′ ITR. In one embodiment, the 5′ ITR and a 3′ ITR are derived from adeno-associated virus (AAV) serotype AAV2.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments, the pseudotyped AAV is AAV2/7m8 (i.e., an AAV containing AAV2 ITRs and AAV7m8 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In some embodiments, the AAV vector contains two or more capsid proteins selected from different serotypes. In some embodiments, the AAV vector contains an rAAV2-retro and an AAVrh.10 capsid protein. In some embodiments, the AAV vector contains rAAV2-retro and AAVrh.10 capsid proteins respectively, in a ratio of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50. In some embodiments, the AAV vector contains AAVrh.10 and rAAV2-retro capsid proteins, respectively, in a ratio of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50.

In some embodiments, a mixture of (1) AAV vectors comprising rAAV2-retro and (2) AAV vectors comprising AAVrh. 10 is used. In some embodiments, a ratio of the (1) AAV vectors comprising rAAV2-retro and (2) AAV vectors comprising AAVrh. 10, respectively, of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50 is used. In some embodiments, a ratio of the (1) AAV vectors comprising AAVrh. 10 and (2) AAV vectors comprising rAAV2-retro, respectively, of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50 is used.

### Other Viral Vectors

Other viral vectors include adenoviral (AV) vectors, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Alternatively, other systems such as lentiviral vectors can be used. Lentiviral-based systems can transduce nondividing as well as dividing cells making them useful for applications targeting, for example, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the expression cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells include hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this disclosure are known to those with skill in nucleic acid manipulation and include genetic engineering, recombinant engineering, and synthetic techniques. See, e.g., Sambrook et al, “Molecular Cloning. A Laboratory Manual”, 2d ed., Cold Spring Harbor Laboratory, New York (1989), and Ausubel et al., Current Protocols in Molecular Biology, John Wiley & Sons, New York, 1989); and International Patent Publication No. WO 95/13598. Further, methods suitable for producing a rAAV cassette in an adenoviral capsid have been described in U.S. Pat. Nos. 5,856,152 and 5,871,982.

Briefly, in order to package the rAAV genome into a rAAV virion, a host cell is used that contains sequences necessary to express AAV rep and AAV cap or functional fragments thereof as well as helper genes essential for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection, and protoplast fusion. In one embodiment, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

The rep and cap sequences, along with their expression control sequences, may be supplied on a single vector, or each sequence may be supplied on its own vector. Preferably, the rep and cap sequences are supplied on the same vector. Alternatively, the rep and cap sequences may be supplied on a vector that contains other DNA sequences that are to be introduced into the host cells. Preferably, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this disclosure are conventional genetic engineering or recombinant engineering techniques such as those described in the references above. For example, the rAAV may be produced utilizing a triple transfection method using either the calcium phosphate method (Clontech) or Effectene reagent (Qiagen, Valencia, Calif.), according to manufacturer's instructions. See, also, Herzog et al, 1999, Nature Medic., 5(1):56-63, for the method used in the following examples, employing the plasmid with the transgene, a helper plasmid containing AAV rep and cap, and a plasmid supplying adenovirus helper functions of E2A, E4Orf6 and VA. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions can be produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art, preferably on a separate plasmid. Thereafter, the recombinant AAV virion which directs expression of the transgene is isolated from the cell or cell culture in the absence of contaminating helper virus or wildtype AAV.

Expression of a transgene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR; and the level of protein expression may be monitored by Western blotting, immunohistochemistry, enzyme-linked immunosorbent assay (ELISA), radioimmunoassay (RIA) or by the specific methods detailed below in the Examples.

### Cells

In embodiments, the nucleic acid constructs and vectors disclosed herein are used to deliver a NARE operatively linked to a transgene to a cell. Provided herein are cells comprising a NARE, a nucleic acid construct, or a vector disclosed herein. In embodiments, the cell is a muscle cell. The cell may be a mammalian cell. The cell may be a human cell. The cell may be isolated.

### Pharmaceutical Compositions

Provided herein are pharmaceutical compositions comprising a nucleic acid construct or vector disclosed herein and a pharmaceutically acceptable excipient.

The nucleic acid construct or vector disclosed herein is preferably assessed for contamination by conventional methods and then formulated into a pharmaceutical composition suitable for storage and/or administration to a patient.

Formulations of the nucleic acid constructs or vectors disclosed herein disclosed herein involve the use of a pharmaceutically and/or physiologically acceptable vehicle or carrier, particularly one suitable for subretinal injection, such as buffered saline or other buffers, e.g., HEPES, to maintain pH at appropriate physiological levels. The nucleic acid constructs or vectors disclosed herein can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilizers, buffers, antioxidants and/or other additives may be included, as required.

Pharmaceutical compositions comprising a nucleic acid construct or vector disclosed herein may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations of invention. Suitable preservatives for use in the formulation of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

For delayed release, nucleic acid construct or vector disclosed herein may be included in a pharmaceutical composition which is formulated for slow release, such as in microcapsules formed from biocompatible polymers or in liposomal carrier systems according to methods known in the art.

If a vector is to be stored long-term, it may be frozen in the presence of glycerol.

### Methods

Provided herein are methods for inducing expression of a transgene in a cell and/or a tissue. Provided herein is a method of inducing the expression of transgene, the method comprising providing a cell comprising a nucleic acid construct comprising a NARE disclosed herein operatively linked to a transgene and cultivating the cells under conditions allowing for the expression of the transgene. In embodiments, the cell is a muscle cell. Provided herein is a method for inducing expression of a transgene in vivo. Provided herein is a method for inducing expression of a transgene in vitro. Provided herein is a method for inducing expression of a transgene ex vivo.

Provided herein are methods of treating a disease in a subject in need thereof using nucleic acid constructs, vectors, and pharmaceutical compositions disclosed herein.

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with ALS without treatment. In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with disease without treatment. In some embodiments, treatment results in long term survival of a patient. As used herein, the term “long term survival” refers to a survival time or life expectancy longer than about 40 years, 45 years, 50 years, 55 years, 60 years, or longer.

Methods of administration include, but are not limited to, intracisternal magna, intracerebroventricular, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some instances, the nucleic acid construct or vector described herein is administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as silastic membranes, or fibers. In some situations, the nucleic acid construct or vector described herein is introduced into the central nervous system, circulatory system or gastrointestinal tract by any suitable route, including intraventricular injection, intrathecal injection, paraspinal injection, epidural injection, enema, and by injection adjacent to a peripheral nerve.

The compositions described herein can be administered as single administrations or as multiple administrations. Such compositions can be administered at regular intervals, depending on the nature, severity and extent of the subject's condition. In some embodiments, a therapeutically effective amount of the nucleic acid construct or vector is administered intrathecally periodically at regular intervals (e.g., once every year, once every six months, once every five months, once every three months, bimonthly (once every two months), monthly (once every month), biweekly (once every two weeks), or weekly).

The amount of the nucleic acid construct or vector described herein that is effective for treating disease can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner.

An effective amount of an rAAV carrying a nucleic acid sequence encoding a transgene (including, but not limited to UPF1, ATP7B, and BDNF) under the control of the NARE may, for example, range between about 1×10⁹ to about 1×10¹⁴ rAAV genome particles (vg)/kg body weight. A “genome particle” is defined herein as an AAV capsid that contains a single stranded DNA molecule that can be quantified with a sequence specific method (such as qPCR or ddPCR). In some embodiments, the rAAV is administered at about 1×10¹² to about 1×10¹³ rAAV vg/kg body weight. In some embodiments, the rAAV is administered at about 5×10¹¹ to about 5×10¹² to vg/mL of cerebrospinal fluid (CSF) volume. In some embodiments, the rAAV is administered at about 7.5×10¹³ to 7.5×10¹⁴ vg total per patient.

In some embodiments, the rAAV is administered to an animal at about 1×10¹¹ to about 1×10¹⁴ rAAV genome particles (vg)/kg body weight.

In some embodiments, the rAAV genome particles are provided in a volume of between about 20 μL to about 50 mL. In some embodiments, the rAAV genome particles are provided in a volume of between about 30 μL to about 30 mL. In some embodiments, the rAAV genome particles are provided in a volume of about 30, about 40, about 50, about 60, about 70, about 80, about 90, about 100, about 200, about 300, about 400, about 500, about 600, about 700, about 800, about 900, about 1000 μL. In some embodiments, the rAAV genome particles are provided in a volume of about 1, about 2, about 3, about 4, about 5, about 6, about 7, about 8, about 9, about 10, about 15, about 20, about 25, about 30, about 35, about 40, about 45, about or 50 mL.

Still other dosages in these ranges may be selected by the attending physician. It is to be understood that for any particular subject, specific dosage regimens can be adjusted over time according to the individual need and the professional judgment of the person administering or supervising the administration of the nucleic acid construct or vector and that dosage ranges set forth herein are exemplary only and are not intended to limit the scope or practice of the claimed invention.

The nucleic acid construct or vectors disclosed herein can also be advantageously provided to a cell ex vivo, followed by administration of the living cell to the subject. Methods for treating disease by implanting a cell that has been modified to express a recombinant protein are also well known. See, for example, U.S. Pat. No. 5,399,346, disclosing methods for introducing a nucleic acid into a primary human cell for introduction into a human. Although use of human cells for ex vivo therapy is preferred in some embodiments, other cells such as bacterial cells may be implanted in a subject's vasculature, continuously releasing a therapeutic agent. See, for example, U.S. Pat. Nos. 4,309,776 and 5,704,910.

This application is related to U.S. Provisional Application No. 63/379,109 entitled “UPF1 EXPRESSION CONSTRUCTS”, U.S. Provisional Application No. 63/379,113, entitled “ATP7B GENE THERAPY” and U.S. Provisional Application No. 63/379,114, entitled “BDNF GENE THERAPY” all filed on Oct. 11, 2022 by the same Applicant, and each of which is incorporated herein by reference in its entirety.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following Examples of specific embodiments are given. The following examples should not be read to limit or define the entire scope of the invention.

### EXAMPLES

### Example 1: NARE Design

### Materials and Methods

Potential NARE candidates were identified from genes that are highly expressed in humans in the target tissue (muscle). Promoter regions were then defined or cis-regulatory elements identified based on chromatin marks, accessibility, conservation, and other genome-wide datasets.

Motif ten element (MTE)/downstream promoter element (DPE) modifications were incorporated to increase transcription, as were various cellular, viral, or synthetic 5′ UTR motifs to increase transcription/translation. Intron choice was changed to include, for example, short cellular introns that naturally have a unique standalone transcriptional start site, which indicates the presence of a promoter within this particular intron. In addition, introns were inserted at different positions of the sequence, and/or introns were truncated while maintaining splicing elements. In addition, individual or multiple point mutations were introduced to increase expression or prevent inactivation. Also, different transcriptional enhancers were introduced upstream of the core promoter. Transcriptional motif insertions, replacements, and/or shuffling was employed in certain instances as was removal of CpG sequences to lower immunogenicity while maintaining promoter activity.

Gene regulation elements were also optimized by the following methods (i) highly expressed transcription factor binding sites (TFBS) were identified, shuffled and cloned upstream of core promoters; (ii) iterative mutation and scoring of promoters using artificial intelligence (AI) algorithms.

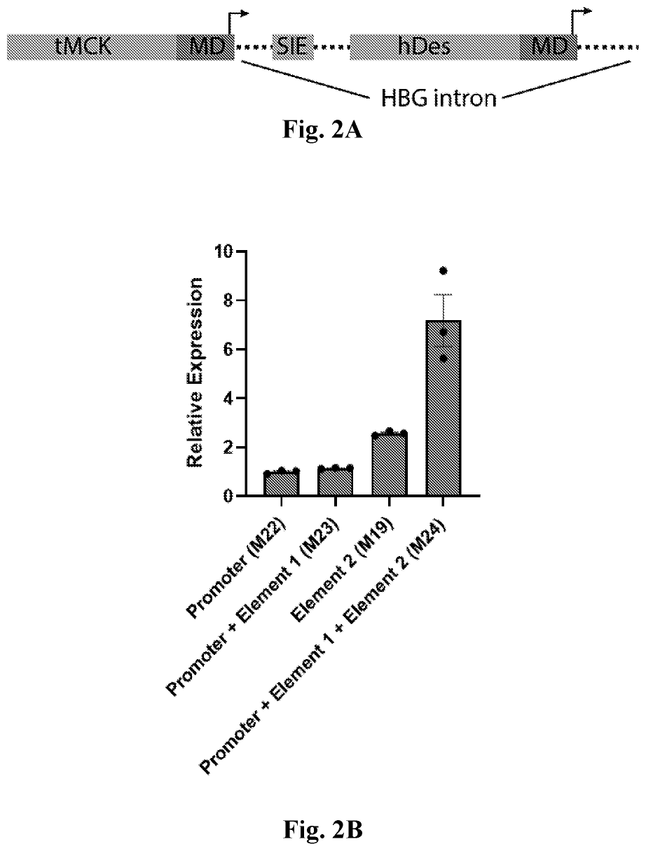

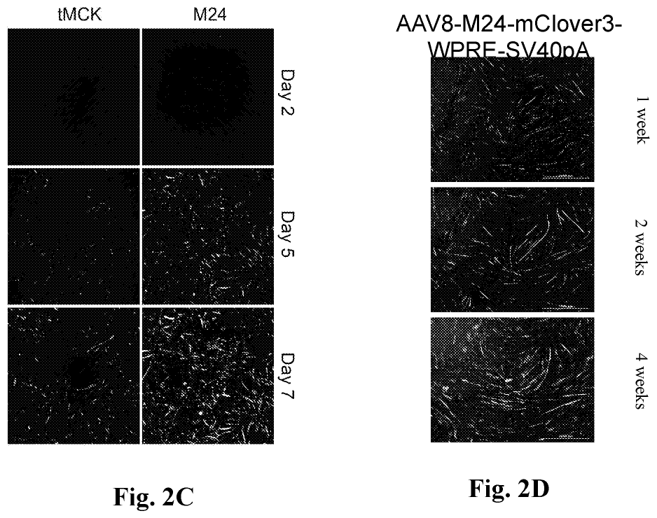

Further, nested NAREs were created (FIG. 2A). One or more promoters were cloned within the intron of one of the existing promoters while ensuring that placement does not create any unintended translational start sites. Dual, tri- and quad-promoters were created in this way allowing an increase in NARE strength while minimizing total length. For example, in FIG. 2A, tMCK was modified by adding the MTE/DPE modification. tMCK is a muscle creatine kinase (MCK) promoter, which was abbreviated by ligating triple tandem of MCK enhancer (206 bp) to its 87-bp basal promoter, generating the tMCK (720 bp) NARE. A synthetic HBG intron variant containing an enhancer (SIE) and a modified human Desmin promoter was inserted downstream. This nested promoter strategy allows for increased transcriptional output by having multiple promoters, and avoids or minimizes loss of expression due to promoter inactivation. The intronic location allows for multiple transcripts to be made and processed and prevents potential detrimental impact of a second promoter sequence on translation rates because it is spliced out in certain transcripts. In addition, this strategy allows for targeting multiple cell types with a single complex promoter and can confer tissue specificity to a normally constitutive promoter.

Generation and Screening of Synthetic Cis-regulatory elements (CREs) by Transcription Factor Shuffling

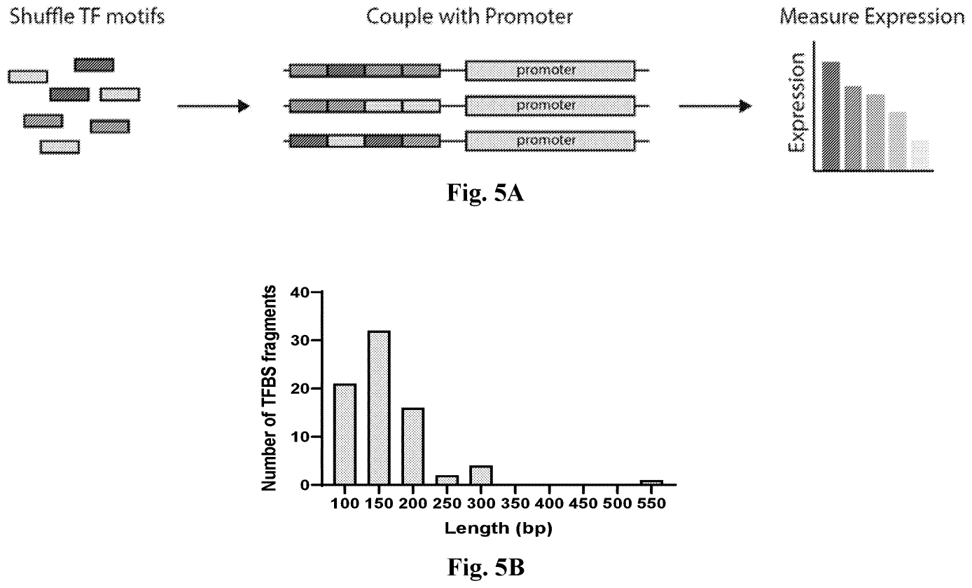

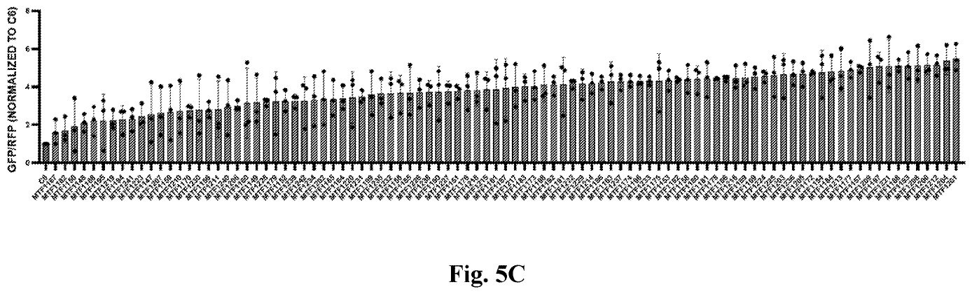

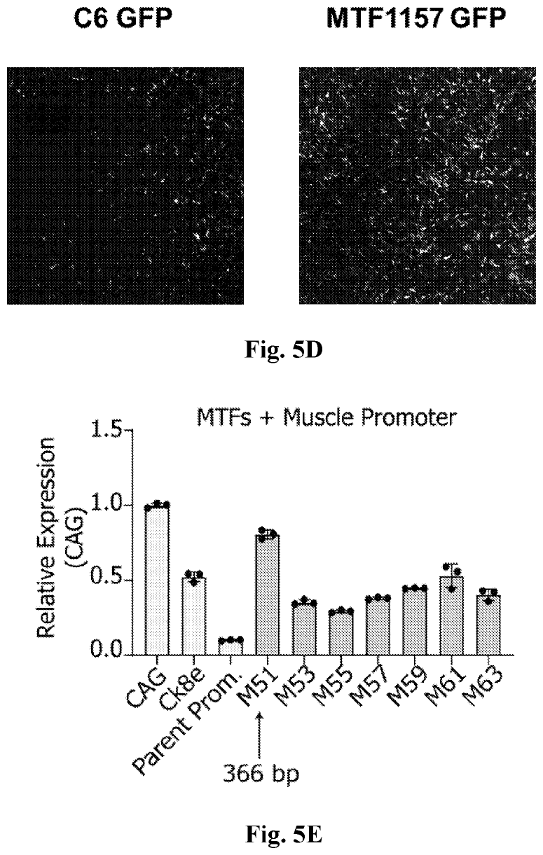

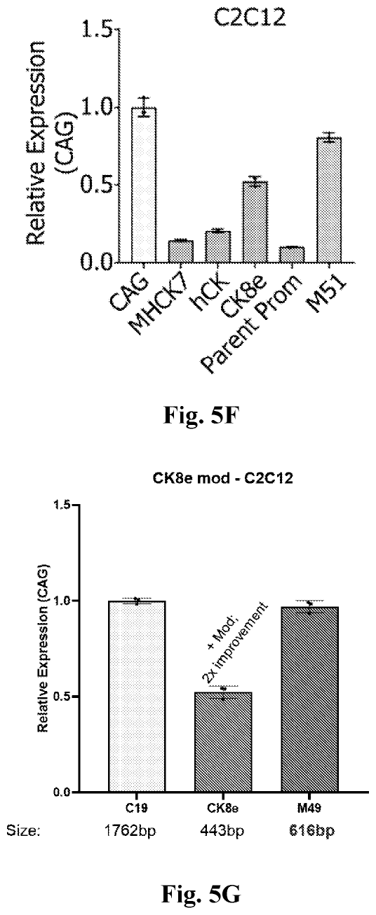

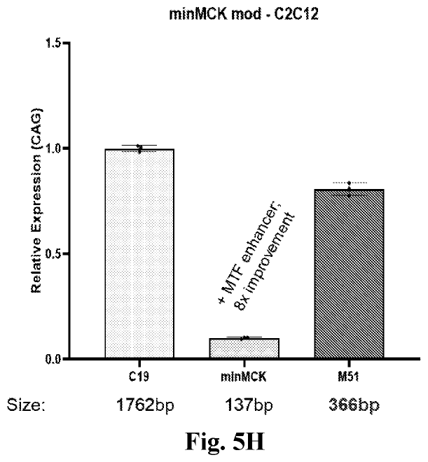

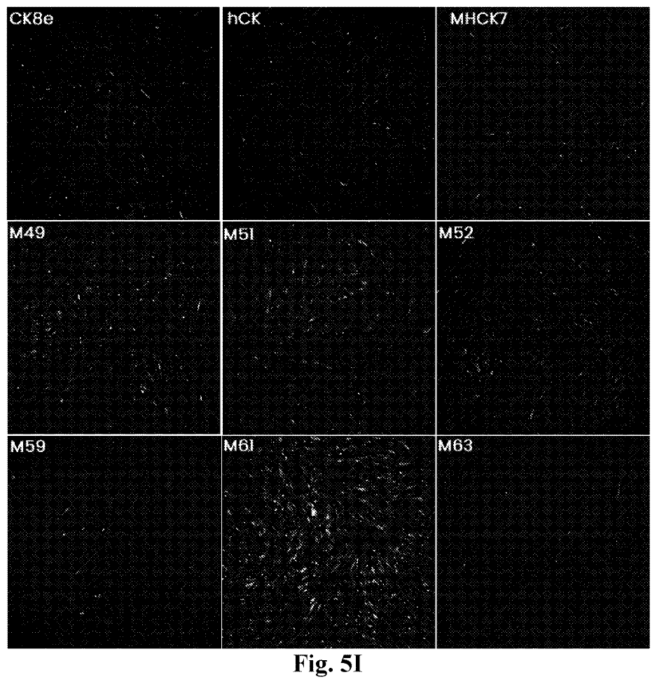

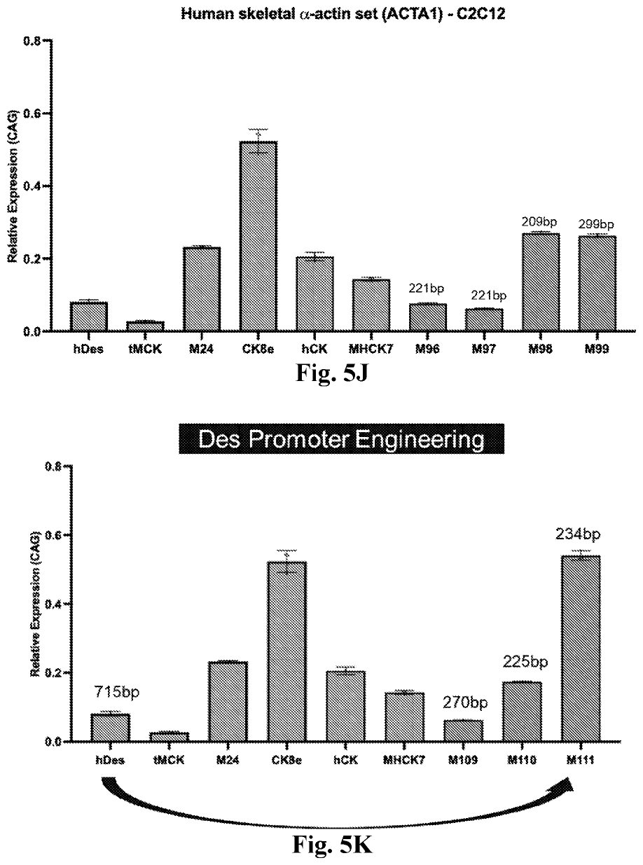

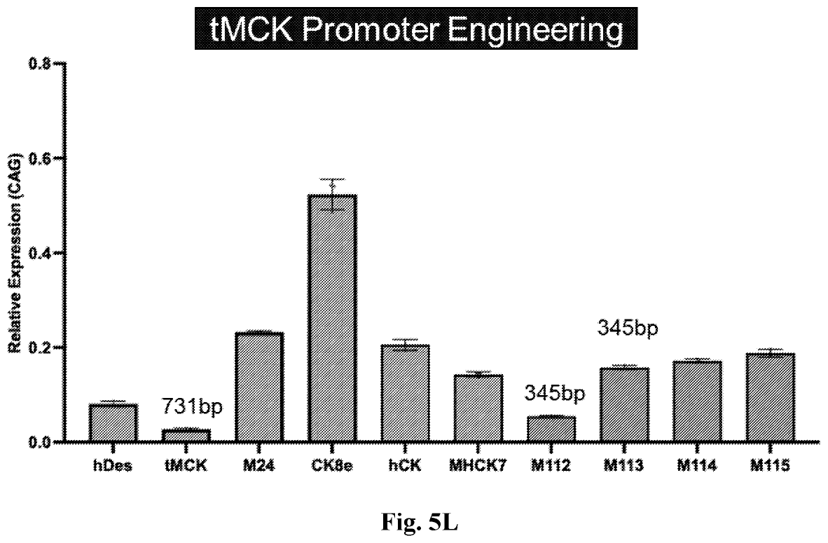

Binding site sequences of tissue-specific and highly expressed transcription factors for a given tissue were printed as a pool of single-stranded oligonucleotides (FIG. 5A). Oligonucleotides were annealed into double-stranded oligonucleotides and ligated together randomly overnight. Fragments were then ligated into an AAV plasmid upstream of the small, synthetic JeT promoter which drives expression of mClover3-WPRE3-synPolyA. This pooled library was transformed into cells, and individual colonies were mini-prepped to get sufficient DNA for transient transfection experiments.

Cells of interest were then transiently co-transfected with TFBS candidate plasmids and mCherry plasmid as a normalization control. After 48 hours, cells were imaged for mClover3 and mCherry fluorescence as described in Example 2. Images were analyzed automatically using CellProfiler to identify red fluorescent protein positive cells and to measure the intensity of green fluorescent protein from these cells. The GFP intensity of candidate TFBS NAREs relative to the JeT promoter alone was used to identify positive hits in this screen. Positive hits were then subcloned into the dual-reporter assay system to validate screening results with either JeT or other basal NAREs.

### Example 2: Dual-Reporter Protein Expression Assay

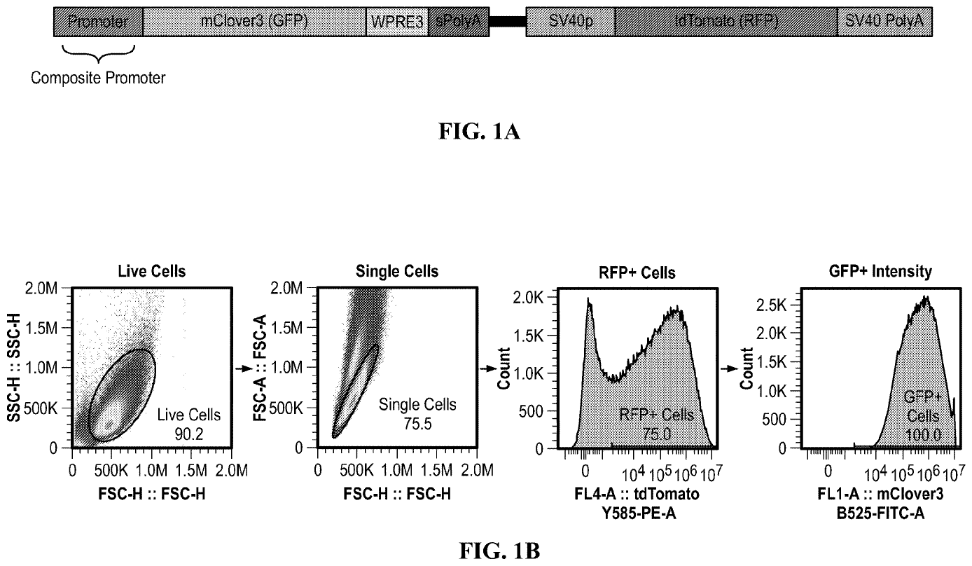

To test the ability of NARE candidates to drive gene expression, a dual reporter assay using flow cytometry was utilized. A diagram of the dual reporter expression construct and assay is provided in FIG. 1. Different synthetic candidate NARE sequences were cloned upstream of a transgene encoding mClover3 (a green/yellow fluorescent protein), which in turn was located upstream of a posttranscriptional regulatory element of woodchuck hepatitis virus (WPRE250) and a synthetic polyadenylation (polyA) signal. mClover3 fluorescence was used as a measure of NARE strength. The plasmid also contained a separate expression cassette (from 5′ to 3′: SV40 promoter-tdTomato (a red fluorescent protein)-SV40 polyA), which was the same in all NARE test constructs (FIG. 1). tdTomato fluorescence was used as an internal normalization control, allowing to account for variations in transfection efficiency.

Various cell lines were transiently transfected with dual reporter plasmids using Turbofect transfection reagent. After 48 hours, cells were trypsinized and resuspended for flow cytometry. Raw data was processed to select only live, single cells for analysis. A sample comprising untransfected cells was used to set the gate for tdTomato signal. This gate was then applied to all live single cells to identify the tdTomato-positive (and thus transfected) cells. Then, mClover3 fluorescence was determined for the tdTomato-positive population. Because transfection varied slightly between different experiments, the relative NARE potency was calculated by the ratio of mClover3 fluorescence to tdTomato fluorescence. Synthetic candidate NAREs were tested along with known constitutive and tissue-specific reference promoters such as CAG, CMV, or AAT.

### Example 3: Strength of Different NAREs in Muscle Cells (In Vitro)

### Materials and Methods

### Myotube Models

The strength of selected muscle specific NAREs was tested using three cellular models of myotubes. One model is the C2C12 immortalized cell line. C2C12 cells are murine skeletal muscle myoblasts derived from satellite cells of the thigh muscle of a two-month-old female C3H mouse. C2C12 cells were plated on Collagen I-coated vessels and grown in expansion media: DMEM (high glucose, no Sodium Pyruvate)+10% Fetal Bovine Serum+2 mM L-Glutamine+100 units/mL Penicillin and 100 μg/mL Streptomycin. After reaching confluence, the C2C12 cells underwent a media change with differentiation media: DMEM (high glucose, no Sodium Pyruvate)+2% Horse Serum+2 mM L-Glutamine+1 μM Insulin+100 units/mL Penicillin and 100 μg/mL Streptomycin. The C2C12 cells were cultured in differentiation media for about 7 days to form long, striated myotubes, which was the result of myoblastic fusion during myogenesis.

The second model used primary human skeletal myoblasts (Thermo Fisher Scientific), derived from biopsies of quadriceps of a human donor lot. The primary human skeletal myoblasts were plated on collagen I-coated vessels and thawed in differentiation media: DMEM (high glucose, no Sodium Pyruvate)+2% Horse Serum+2 mM L-Glutamine+1 μM Insulin+100 units/mL Penicillin and 100 μg/mL Streptomycin. The primary human skeletal myoblasts were cultured in differentiation media for 2-3 days to undergo rapid differentiation.

Alternatively, a third cellular model, human induced Pluripotent Stem Cell-derived myoblast, can be used to test NARE strength in myotubes. The hiPSC-derived myoblasts were plated on collagen I-coated vessels and grown in Skeletal Myoblast Medium (AMSBIO). After reaching confluence, the hiPSC-derived myoblasts underwent a media change with Myotube Medium or Myotube Fusion Medium (AMSBIO).

After successful differentiation of myoblasts into myotubes, the cells were AAV-transduced with synthetic candidate NAREs driving expression of fluorescent proteins such as mClover3 and mIRFP713. Candidate NAREs were tested along with known constitutive and tissue-specific reference promoters, such as CAG, CMV, tMCK, and creatine kinase 8 promoter (CK8e). With consistent Multiplicity of Infection in triplicate samples, the AAV candidates were diluted in differentiation media to replace the media containing the plated adherent myotubes. After 4 hours of incubation at 37 °C with 5% CO2, differentiation media supplemented with 200 nM doxorubicin was added at the same volume as the AAV-containing media to have a final concentration of 100 nM doxorubicin, as an agent to improve transduction efficiency. After 1-2 days of infection, the AAV- and doxorubicin-containing differentiation media was changed for regular differentiation media to remove the AAV and doxorubicin. Themyotubes' fluorescent protein expression was detected via Agilent's/BioTek's Cytation 5 Cell Imaging Multimode Reader. The myotubes' intensity of fluorescence is measured as an indicator of NARE strength, with synthetic candidate NAREs to be compared among the known reference promoters.

### Results

A library of NAREs suitable for the expression of genes in the muscle was developed.

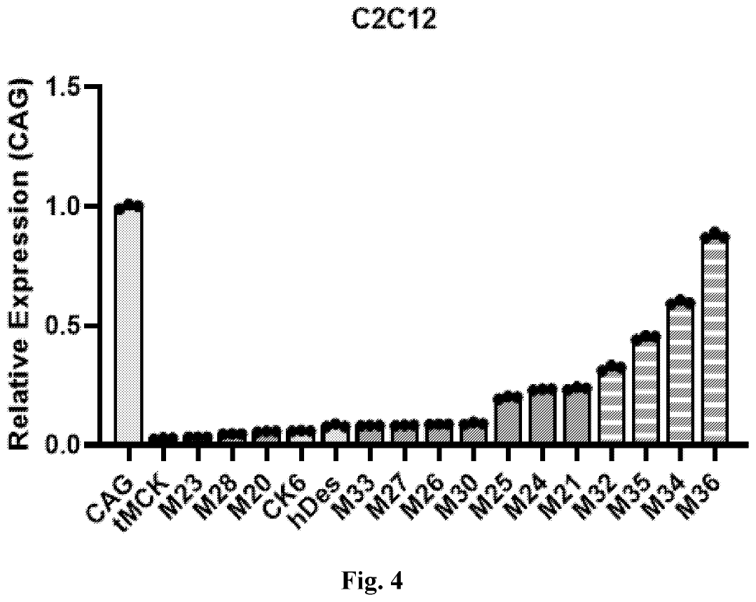

Some NAREs were derived from genes highly expressed in human skeletal muscle and were modified with various elements to increase NARE strength. The sizes of these NAREs ranged from 450 bp to 1.6 kb. Dual-Reporter plasmids with different NARE candidates were transiently transfected into C2C12 cells. GFP expression level in transfected (RFP+) cells was quantified by flow cytometry. Relative expression as compared to a CAG promoter is shown in FIG. 4.

Additional NAREs were also identified using a nested promoter design (see FIG. 2A for an example of nested promoter design and FIG. 2B for evolution of NARE M24). M24 is a stronger NARE than tMCK in cultured myotubes generated using C2C12 cells (FIG. 2C) and in primary human myotubes (FIG. 2D).

NAREs were also generated by creating synthetic enhancers via shuffling of transcription factor motifs (FIG. 5A). A majority of the synthetic sequences generated were 200 bp or less (FIG. 5B). Expression of a transgene encoding GFP under the control of the synthetic enhancers coupled with the promoter C6 in C2C12 was normalized to expression of the transgene encoding GFP under the control of the NARE alone (FIGS. 5C and 5D). Selected hits (MTF1291, MTF1173, MTF1186, MTF1197, MTF1209, MTF1211, MTF1242, MTF1295, MTF1304, MTF1308) were cloned into either the minCK promoter (M51, M53, M55, M57, M59, M61, M63, M65, M67, M69) or in upstream of the Ck8e promoter. (M52, M54, M56, M58, M60, M62, M64, M66, M68, M70).

The potency of selected NAREs in C2C12 cells is shown in FIGS. 5E and 5F.

The potency of CK8e-derived NARE M49, which is only 616 bp, as compared to the parent promoter CK8e (M31) in C2C12 cells is shown in FIG. 5G.

The potency of minMCK-derived NARE M51 (a 366 bp NARE that includes an additional enhancer as compared to the parent promoter minMCK (M50)) in C2C12 cells as compared M50 is shown in FIG. 5H. M51 is 1.5× stronger than the 443 bp CK8e promoter.

The potency of selected NAREs in C2C12 cells is shown in Table 1. The potency of selected NAREs in primary human myotubes is shown in FIG. 5I.

TABLE 1
Potency of indicated NAREs in C2C12 myoblasts.

| | | Size | Potency relative |
|---|---|---|---|
| NARE | Description | (bp) | to CK8e |
| M31 | CK8e | 443 | 1 |
| M49 | CK8e variant | 616 | 1.9 |
| M51 | MTFBS promoter | 366 | 1.5 |
| M52 | MTFBS promoter | 672 | 1.4 |
| M59 | MTFBS promoter | 267 | 0.9 |
| M61 | MTFBS promoter | 688 | 1.0 |
| M63 | MTFBS promoter | 264 | 0.8 |
| M71 | hCK | 614 | 0.4 |
| M73 | MHCK7 | 770 | 0.3 |

Plasmid transfections.

The potency of selected human skeletal a-actin gene (ACTA1)-derived NAREs in C2C12 cells is shown in FIG. 5J. Human desmin (hDes) promoter (M19), tMCK promoter (includes triple tandem copies of mouse MCK enhancer mouse MCK basal promoter) (M22), CK8e (M31), hCK promoter (M71), MHCK7 promoter (M73) served as controls. M98 and M99 are more potent than the hDes, tMCK, hCK M24, and MHCK7 promoters. The potency of selected NAREs is summarized in Table 2.

TABLE 2
Potency of ultra-compact NAREs in C2C12 myoblasts.

| | | | Potency | Potency | Potency | Potency |
|---|---|---|---|---|---|---|
| | Size | | relative to | relative to | relative to | relative to |
| NARE | (bp) | Family | tMCK | MHCK7 | hCK | CK8e |
| M98 | 209 | ACTA1 | 9.9 | 2 | 1.3 | 0.5 |
| M99 | 299 | ACTA1 | 9.6 | 2 | 1.3 | 0.5 |
| M104 | 234 | ACTC1 | 2.2 | 0.4 | 0.3 | 0.1 |
| M105 | 324 | ACTC1 | 2.2 | 0.4 | 0.3 | 0.1 |
| M106 | 351 | ACTC1 | 2.1 | 0.4 | 0.3 | 0.1 |
| M110 | 225 | DES | 6.4 | 1.2 | 0.8 | 0.3 |
| M111 | 234 | DES | 20 | 3.8 | 2.6 | 1 |
| M113 | 345 | MCK | 5.8 | 1.1 | 0.8 | 0.3 |
| M114 | 296 | MCK | 6.3 | 1.2 | 0.8 | 0.3 |

Plasmid transfection.
tMCK is 731 bp; MHCK7 is 770 bp; hCK is 661 bp; CK8e is 466 bp.

The potency of selected hDes-derived NAREs in C2C12 cells is shown in FIG. 5K. M111 (234 bp) is that is stronger than all tested muscle-specific NAREs.

The potency of selected tMCK-derived NAREs in C2C12 cells is shown in FIG. 5L.

### Example 4: Strength of Different NAREs in Muscle Cells (In Vivo)

### Materials and Methods

### Intramuscular Injections

Male C57BL6 mice, aged 6-8 weeks were kept in standard cages with ad libitum water, and were changed from the standard food chow to a purified chow containing no alfalfa to minimize background autofluorescence. Mice were anesthetized with 2-3% isoflurane with 1.5 LPM of oxygen. One leg for each mouse was shaved to remove all fur and cleaned with an alcohol prep pad, and the gastrocnemius was targeted using a 27G needle, being careful to avoid any blood vessels. Mice were allowed to recover in a clean warm cage and returned to their home cage after they were awake and mobile.

### Intravenous Injections

Male C57BL6 mice, aged 6-8 weeks were kept in standard cages with ad libitum water, and were changed from the standard food chow to a purified chow containing no alfalfa to minimize background autofluorescence. Mice were restrained and placed under a warming light to increase vasodilation and either tail vein was targeted using a 27G needle. Mice are allowed to recover in a clean warm cage and returned to their home cage after making sure they were not bleeding.

### Bioimaging Protocol

Mice were anesthetized using 2-3% isoflurane with 1.5 LPM oxygen and imaged using a LICOR Pearl System on the white channel and the 700 nm channel. Animals were imaged weekly for 5 weeks and then every two months for up to 12 months. Mice were allowed to recover in a clean warm cage and returned to their home cage after they were awake and mobile. Data analysis is performed using the LICOR Image Studio Software: a region of interest (ROI) was selected for background normalization on a part of the animal that had no fluorescence. An ROI encompassing the torso or both legs was drawn and normalized against the background ROI. The same area was used across animals on each timepoint to minimize artifacts in data collection. Absolute fluorescence was recorded for every imaging session.

eGFP/mClover3 Imaging of IM Injections

Four weeks after injection, animals were euthanized, and the gastrocnemius was dissected and drop-fixed overnight in 4% Paraformaldehyde in PBS at 4 °C. The next morning, muscles were changed to 15% sucrose in PBS and incubated at 4 °C overnight. Muscles were changed to 30% sucrose in PBS and incubated at 4 °C overnight. Muscles were then covered in OCT and allowed to equilibrate for 1 hour at RT in freezing molds. Samples were frozen on dry ice and then stored at −80 °C until being serially sliced for histology using the cryostat. Serial sections 25 μm thick were collected at 600 μm intervals to gain a full representation of expression across the muscle. After n=3 per group were sectioned, slides air-dried for 10 minutes to complete mounting process. Slides were then washed 3× with PBS and mounted with Prolong anti-fade with NucBlue and imaged on the BioTek imager to create tilescans of all slices

eGFP/mClover3 Imaging of IV Injections

Four weeks after injection, animals were euthanized and perfused with PBS followed by 4% Paraformaldehyde in PBS. Liver lobes were dissected and further fixed in 4% Paraformaldehyde in PBS for 1 hour at room temperature. Liver lobes were then changed to 15% sucrose in PBS and incubated at 4 °C overnight. The next morning, muscles were changed to 30% sucrose in PBS and incubated at 4 °C overnight. Liver lobes were then covered in OCT and allowed to equilibrate for 1 hour at RT in freezing molds. Samples were frozen on dry ice and then stored at −80 °C until being serially sliced for histology using the cryostat. Serial sections 25 μm thick were collected at 1000 μm intervals to gain a full representation of expression across the liver. After n=3 per group were sectioned, slides air-dried for 10 minutes to complete mounting process. Slides were then washed 3× with PBS and mounted with Prolong anti-fade with NucBlue and imaged on the BioTek imager to create tilescans of all slices.

### Western Blotting Tissue Prep

Four weeks after injection, mice were euthanized, and relevant tissues were dissected and snap frozen in liquid nitrogen. Frozen tissues were then cut and a 10 mg piece was lysed in RIPA buffer and sonicated. Lysate was spun down to remove insoluble materials and protein concentration was determined using BCA Concentration Assay. Protein is then diluted and normalized to 1 μg/ul using 4× LDS, 10× DTT, and lysis buffer. Samples were denatured at 70-80 °C for 10 min and stored at −20 °C. Samples were then analyzed via western blotting using a LICOR Odyssey Imager.

### Results

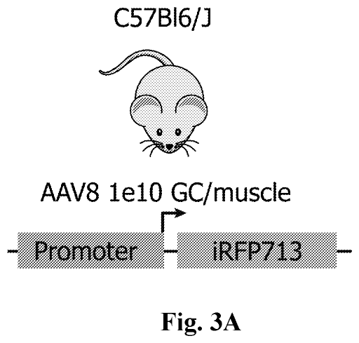

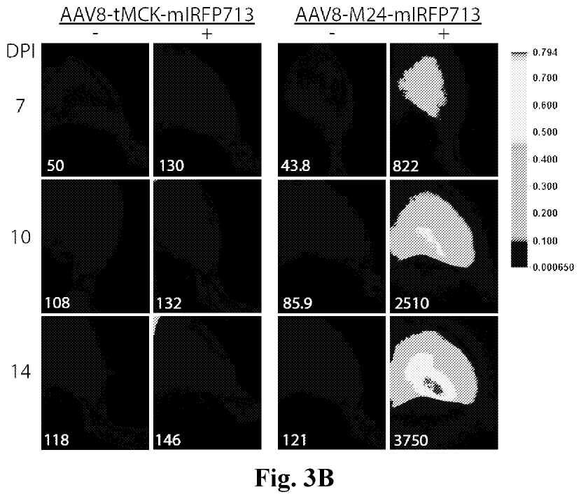

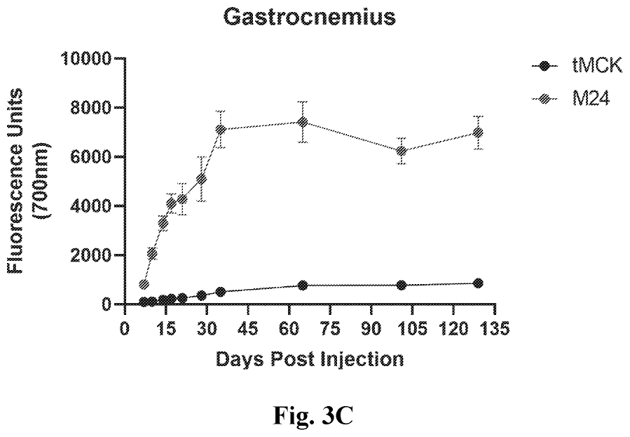

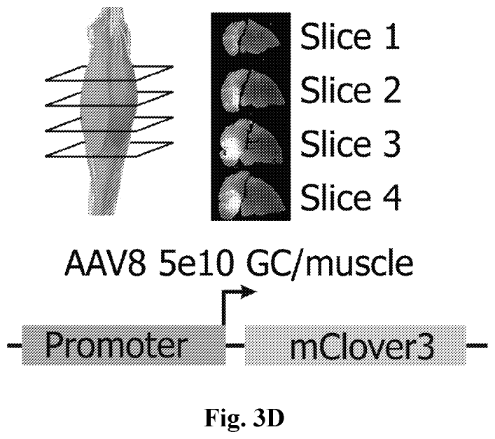

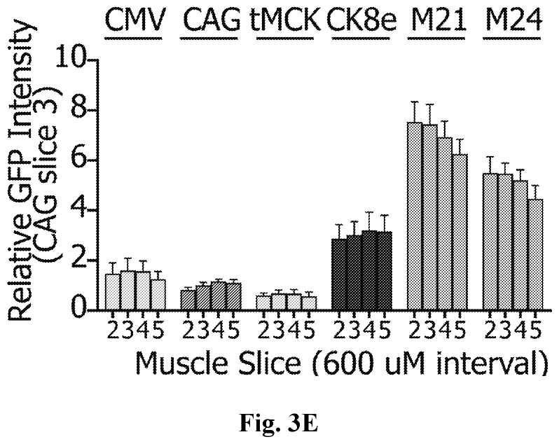

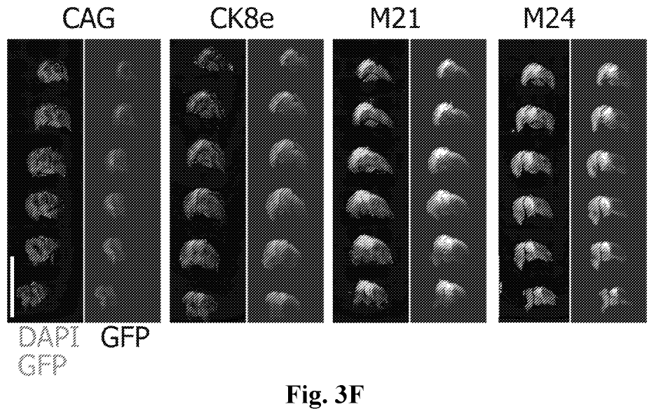

AAV8-iRFP713 under the control of tMCK or M24 NARE, respectively, was injected intramuscularly to male C57B16 mice at 8 weeks of age and imaged using the LICOR Pearl (FIGS. 3A and 3B). iRFP713 is a near-infrared fluorescent protein. NARE M24 has 8-fold higher expression than tMCK that is maintained out to 300 days post injection (FIG. 3C).

In a different experiment, AAV8-eGFP under the expression of NAREs CMV, CAG, tMCK, CK8e, M21, and M24, respectively, was injected into the gastrocnemius of male C57B16 mice at 8 weeks of age (FIG. 3D). After 4 weeks, the muscle was harvested and sectioned for measurement of native eGFP fluorescence. NAREs M21 and M24 show increased NARE strength as compared to CAG or CK8e in the mouse gastrocnemius (FIGS. 3E and 3F, Table 3).

TABLE 3
Description of expression constructs shown in FIG. 3E.

| NARE | ORF |
|---|---|
| CMV | mClover3-WPRE₂₅₀-sPolyA |
| CAG | eGFP-WPRE₂₅₀-bGHpolyA |
| tMCK | mClover3-WPRE-SV40polyA |
| CK8e | mClover3-WPRE-SV40polyA |
| M21 | mClover3-WPRE-SV40polyA |
| M24 | mClover3-WPRE-SV40polyA |

WPRE₂₅₀ activity is equivalent to WPRE.

An overview of NARE sequences can be found in Tables 4-6.

TABLE 4
NARE sequences.

| SEQ ID | |
|---|---|
| NO | NARE name |
| 1 | MPG |
| 2 | ACTA1 |
| 3 | CKM |
| 4 | MYL2 |
| 5 | MB |
| 6 | DES |
| 7 | TPM2 |
| 8 | TNNC2 |
| 9 | TNNC1 |
| 10 | TPT1 |
| 11 | TNNT1 |
| 12 | GAPDH |
| 13 | MT1X |
| 14 | MYLPF |
| 15 | MYL1 |
| 16 | MYH7 |
| 17 | CRYAB |
| 18 | TPM3 |
| 19 | hDES715 |
| 20 | mDESenh-CKMenh-CKMp-INR-MD- |
| | HBBi-SIE |
| 21 | mDESenh-CKMenh-CKMp-INR-MD- |
| | HBBi-SIE-hDES715p |
| 22 | tMCK |
| 23 | tMCK-MD-HBBi-SIE |
| 24 | SPc5-12a |
| 24 | tMCK-MD-HBBi-SIE-hDES715p |
| 26 | SPc5-12b |
| 27 | SP301a |
| 28 | SP301b |
| 29 | CK6 |
| 30 | CK8.3 |
| 31 | CK8e |
| 32 | CK8emd |
| 33 | CK8emd-hBG-SIE |
| 34 | mDESe-MCKe-TPT1p-MD-hBG-SIE |
| 35 | GAPDH-MD-hBG-SIE |
| 36 | SPc5e-MT1Xp-MD-hBG-SIE |
| 37 | CK8emd2 |
| 38 | CK8eMD2a |
| 39 | CK8eMD2b |
| 40 | CK8eMD2c |
| 41 | CK8eMD2d |
| 42 | CK8eMD2e |
| 43 | CK8eMD2f |
| 44 | CK8eMD2g |
| 45 | ttMCKe |
| 46 | mDes-CK8e |
| 47 | CK8e-BTImd2 |
| 48 | CK8e-BTImd2-MVMi |
| 49 | CK8e-sBTImd2-MVMi |
| 50 | MCKmin |
| 51 | MTF1291-MCKmin |
| 52 | MTF1291-CK8e |
| 53 | MTF1173-MCKmin |
| 54 | MTF1173-CK8e |
| 55 | MTF1186-MCKmin |
| 56 | MTF1186-CK8e |
| 57 | MTF1197-MCKmin |
| 58 | MTF1197-CK8e |
| 59 | MTF1209-MCKmin |
| 60 | MTF1209-CK8e |
| 61 | MTF1211-MCKmin |
| 62 | MTF1211-CK8e |
| 63 | MTF1242-MCKmin |
| 64 | MTF1242-CK8e |
| 65 | MTF1295-MCKmin |
| 66 | M66-MTF1295-CK8e |
| 67 | MTF1304-MCKmin |
| 68 | MTF1304-CK8e |
| 69 | MTF1308-MCKmin |
| 70 | MTF1308-CK8e |
| 71 | hCK |
| 72 | hCKplus |
| 73 | MHCK7 |
| 74 | CK8e-EEF1Ali |
| 75 | CK8e-EEF1B2i |
| 76 | CK8e-TMSB10i |
| 77 | CK8e-S100A6i |
| 78 | CK8e-RPL27i |
| 79 | CK8e-GFAPi |
| 80 | CK8e-UCHL1i |
| 81 | CK8e-ALDOAi |
| 82 | CK8e-CAMK2Ai |
| 83 | CK8e-FHL1i |
| 84 | CK8e-CRYABi |
| 85 | CK8e-FXYD1i |
| 86 | CK8e-RBP4i |
| 87 | CK8e-APOC1i |
| 88 | CK8e-ATF5i |
| 89 | CK8e-HDPi |
| 90 | CK8e-FLOT1i |
| 91 | CK8e-HBBi1 |
| 92 | CK8e-HBBi2 |
| 93 | CK8e-HBBi2xc |
| 94 | CK8e-IFI27L2i |
| 95 | CK8e-RPL26i |
| 96 | ACTA1.1 |
| 97 | ACTA1.2 |
| 98 | ACTA1.3 |
| 99 | ACTA1.4 |
| 100 | ACTA1.5 |
| 101 | ACTA1.6 |
| 102 | ACTC1.1 |
| 103 | ACTC1.2 |
| 104 | ACTC1.3 |
| 105 | ACTC1.4 |
| 106 | ACTC1.5 |
| 107 | ACTAC1.1 |
| 108 | ACTAC1.2 |
| 109 | DES.2 |
| 110 | DES.3 |
| 111 | DES.4 |
| 112 | CKM.1 |
| 113 | CKM.2 |
| 114 | CKM.3 |
| 115 | CKM.4 |
| 116 | CKM-ACTC |
| 117 | CKM-DES |
| 118 | CKM-ACTA |
| 119 | ATAC1.1b |
| 120 | ATAC1.2b |

TABLE 5
NARE components.

| SEQ ID | NARE name and NARE |
|---|---|
| NO | components |
| 121 | ACTA1 enhancer |
| 122 | ACTC1 enhancer |
| 123 | ALDOAi |
| 124 | APOC1i |
| 125 | ATF5i |
| 126 | BTImd2 |
| 127 | CAMK2Ai |
| 128 | CK8e promoter |
| 129 | CK8e promoter |
| 130 | CK8e |
| 131 | CK8e (partial) |
| 132 | CK8e.1 |
| 133 | CK8emd promoter |
| 134 | CKM enhancer |
| 135 | CKM promoter-INR-MD |
| 136 | CRYABi |
| 137 | EEF1A1i |
| 138 | EEF1B2i |
| 139 | FHL1i |
| 140 | FLOT1i |
| 141 | FXYD1i |
| 142 | GAPDHmd promoter |
| 143 | GFAPi |
| 144 | HBB intron-SIE |
| 145 | HBBi1 |
| 146 | HBBi2 |
| 147 | HBBi2xc |
| 148 | HBG/SIE intron |
| 149 | HBG/SIE/hDES715md intron |
| 150 | hBG-SIE intron |
| 151 | hDES715md promoter (part of |
| | HBG/SIE/hDES715md intron) |
| 152 | HDPi |
| 153 | IFI27L2i |
| 154 | MCK enhancer |
| 155 | MCKmin |
| 156 | md2 |
| 157 | md2a |
| 158 | md2b |
| 159 | md2c |
| 160 | md2d |
| 161 | md2e |
| 162 | md2f |
| 163 | md2g |
| 164 | mDES enhancer |
| 165 | mDES.1 enhancer |
| 166 | minACTA1.1 promoter |
| 167 | minACTA1.2 promoter |
| 168 | minACTA1.3 promoter variant 1 |
| 169 | minACTA1.3 promoter variant 3 |
| 170 | minACTA1.3 promoter variant 2 |
| 171 | minACTA1.4 promoter |
| 172 | minACTAC1.1 promoter |
| 173 | minACTC1 promoter |
| 174 | minACTC1.2 promoter |
| 175 | minACTC1.2 promoter (partial) |
| 176 | minACTC1.3 promoter |
| 177 | minACTC1.3 promoter (partial) |
| 178 | minACTC1.4 promoter |
| 179 | minACTC1.5 promoter |
| 180 | minCKM enhancer |
| 181 | minCKM.1 promoter |
| 182 | minCKM.2 promoter |
| 183 | minCKM2 enhancer |
| 184 | minDes enhancer |
| 185 | minDes promoter |
| 186 | minDes.3 promoter |
| 187 | minDes.4 promoter |
| 188 | miniACTC1.3 promoter (partial) |
| 189 | MT1Xmd promoter |
| 190 | MTF1173 |
| 191 | MTF1186 |
| 192 | MTF1197 |
| 193 | MTF1209 |
| 194 | MTF1211 |
| 195 | MTF1242 |
| 196 | MTF1291 |
| 197 | MTF1295 |
| 198 | MTF1304 |
| 199 | MTF1308 |
| 200 | MVM intron |
| 201 | MVM-ACTA1 intron |
| 202 | MVM-ACTA1e intron |
| 203 | MVM-ACTC1 intron variant 1 |
| 204 | MVM-ACTC1 intron variant 2 |
| 205 | MVM-ACTC1.3 intron |
| 206 | MVM-ACTC1e intron |
| 207 | MVM-ACTC1e-ACTC1p intron |
| 208 | MVM-DES.4 intron |
| 209 | RBP4i |
| 210 | RPL26i |
| 211 | RPL27i |
| 212 | S100A6i |
| 213 | sBTImd2 |
| 214 | SIE |
| 215 | SP-301 promoter |
| 216 | SPc5-12 promoter |
| 217 | SPc5e |
| 218 | tMCKmd promoter |
| 219 | TMSB10i |
| 220 | TPT1md promoter |
| 221 | UCHL1i |
| 222 | CK8e (without MCK enhancer |
| | sequence) |

TABLE 6
NAREs and NARE components. SN = SEQ ID NO.

| | | NARE | |
|---|---|---|---|
| | | name/ | |
| SN | Name | components | Sequence |
| 1 | M1 | MPG | CCTTTCAAAGCCTCAAGGGTGTTCTCATCCCCAGCAGTGGG |
| | | | ACAAACTGGGGACAGAGGAAGCCCCAGGCCCCCTCCTCTC |
| | | | ACCTGGCACCACTTTCTCTTGTCCTGGACCAGCCCGCCAGC |
| | | | CCACTCTGCAGGCCACAGTTGGAACTCTCATCCCACCGCTT |
| | | | CCAGCCTGTGGTGGGAGCATGAGACCACAAACCAGGAAGT |
| | | | GGGGGGGGGGAGTGCCCAGAGATTGCGCAACCCCCAGCA |
| | | | CTCAGTCCAGAACATCTGGAGAAGTGCCGGCCTCTCCACC |
| | | | TCCTGGGGCAGGGGCCAATCTGCTCTCCAAGTGATTCTCTT |
| | | | TCCTCATAAGAAAGGTGGGGATAAAAACACCGTGAGTTCT |
| | | | GCAACATCAGAATAAATATAACAACTGGGGCCTCAGGCTG |
| | | | TTCCCACAGGAAGGATGCAGATAAGCAGGGAGGGTGTGG |
| | | | AATCACATCCAGGGCTGCTTTATAAACACGGATTGATGAC |
| | | | ACATTATGCCCCCACCCCGGGAAGCGGGATGGCCCCTCCC |
| | | | TCCCCAGGAAGTCCTGGCTGGGGCCCATGGGGGTGGGACG |
| | | | GCTGGTCAGAGGAGGAGAGGCAGTTGGAGACCAGGTCCT |
| | | | GCCCTGTTAGCCTTTCTGTAGGACCAGGTGTCACACTCATC |
| | | | TGAGGGCTCCAGACACAGGGCCTGTGCGTCTGTCCTGCCC |
| | | | AGCGTCTCCCACCTGACGCGGCCAGGTGCCCAGTTGCTCTT |
| | | | CAAGGCCTTTTCAGGTGCTCCTTTCCAGGACCAGGGTCACA |
| | | | GAAGTCAAAGCTCAGGAAAAGCCCCTGATTGTGCCTGCCA |
| | | | CAGTGGAGGGGCCCTGCAGACCCAGATAAACCTTCATGGG |
| | | | GCTGCCTTGGGCCGTGGGAGCCAGCAGTGTGCCCAGCTCC |
| | | | ACCGTCCCTGCCCCTCCCTAGGGGCTGCCTGCGCTCCAGCC |
| | | | AGCATCCCTGGGCGGAAATCCTTCCTTCCCCCGACCAGGC |
| | | | CAGGGGCGGGGGCCTCCGCCCTGGATAAGGACTCCCCGGC |
| | | | CCACTCCGGACCAGGGCTGGGGCGGCCTCCCAGGCGCTCA |
| | | | CTCCGCTGGCACCCCACCGGAAAACACGTCTGCGGCCCGC |
| | | | CCCCTCCCCCAAAGCACGACCACTCCGCCCGGGCCCCCTCT |
| | | | GGGGCCGCCAGGCGACCAGGGCGCAGGCGGGGGCGGACA |
| | | | GCTCATTGGGAGGGGCGCCGGGGCACAGTGCGGGGCTCGC |
| | | | CCCACCCCCAGGTGCCCCTTCCCCGCTCTCGCCTCGCAGGC |
| | | | ACCGCATCGGGCCCGGGAATCGGTCCGGACCTGGCGGTGG |
| | | | GCGCTGGGAAGAGGATCCACCTCCACGTGGCCCGCCCCGC |
| | | | CCCGGGGGCGCAGCCAGTTCCCGGCGCTCACTGCCCCCCT |
| | | | TCTCCCGGCTTCCGTCCCCTTCTGCGCAGGCGCCGCTCCGC |
| | | | CCCGGTCCTAGGGGTGCTTCCGTGGTCGGCGGCTGCTGGG |
| | | | CTCCGCGCCGGGGTCCGAGTCCCACGAAGCCCCGGCCCGA |
| | | | GGGATCCGCCACC |
| 2 | M2 | ACTA1 | CCCGAAGAGGAGTTGAAATATTGGAAGTCAGCAGTCAGGC |
| | | | ACCTTCCCGAGCGCCCAGGGCGCTCAGAGTGGACATGGTT |
| | | | GGGGAGGCCTTTGGGACAGGTGCGGTTCCCGGAGCGCAGG |
| | | | CGCACACATGCACCCACCGGCGAACGCGGTGACCCTCGCC |
| | | | CCACCCCATCCCCTCCGGCGGGCAACTGGGTCGGGTCAGG |
| | | | AGGGGCAAACCCGCTAGGGAGACACTCCATATACGGCCCG |
| | | | GCCCGCGTTACCTGGGACCGGGCCAACCCGCTCCTTCTTTG |
| | | | GTCAACGCAGGGGACCCGGGCGGGGGCCCAGGCCGCGAA |
| | | | CCGGCCGAGGGAGGGGGCTCTAGTGCCCAACACCCAAATA |
| | | | TGGCTCGAGAAGGGCAGCGACATTCCTGCGGGGTGGCGCG |
| | | | GAGGGAATGCCCGCGGGCTATATAAAACCTGAGCAGAGG |
| | | | GACAAGCGGCCACCGCAGCGGACAGCGCCAAGTGAAGCC |
| | | | TCGCTTCCCCTCCGCGGCGACCGGATCCGCCACC |
| 3 | M3 | CKM | GTAGGGGTGAGGGCCCAGGGGGCACAGGGGGAGCCGAGG |
| | | | GCCACAGGAAGGGCTGGTGGCTGAAGGAGACTCAGGGGC |
| | | | CAGGGGACGGTGGCTTCTACGTGCTTGGGACGTTCCCAGC |
| | | | CACCGTCCCATGTTCCCGGCGGGGGGCCAGCTGTCCCCAC |
| | | | CGCCAGCCCAACTCAGCACTTGGTCAGGGTATCAGCTTGG |
| | | | TGGGGGGGCGTGAGCCCAGCCCCTGGGGCGGCTCAGCCCA |
| | | | TACAAGGCCATGGGGCTGGGCGCAAAGCATGCCTGGGTTC |
| | | | AGGGTGGGTATGGTGCGGGAGCAGGGAGGTGAGAGGCTC |
| | | | AGCTGCCCTCCAGAACTCCTCCCTGGGGACAACCCCTCCC |
| | | | AGCCAATAGCACAGCCTAGGTCCCCCTATATAAGGCCACG |
| | | | GCTGCTGGCCCTTCCTTTGGGTCAGTGTCACCTCCAGGATA |
| | | | CAGACAGCCCCCCTTCAGCCCAGCCCAGCCAGGTACTGCA |
| | | | CGGGGCGGGAATCTGGGTGGGGGGATCCGCCACC |
| 4 | M4 | MYL2 | GGAGATGACATATGAGCAAAGATGCAGTGAAGGAGGTGA |
| | | | AGGAAGGAGCCGTGCGATGACTGACAGAAAGACATTCCA |
| | | | GGTAGAGGGCACACAGGTGCAAAGACCCTGAGGCCAGAT |
| | | | CCAGGCTGATAAAACAGAGCATTTTAGCAGTCTCCTCTCCC |
| | | | TGCCATTTTTTTTCTCAAAATTGACAAGCACAAGTGTCCCC |
| | | | GGCCCAAGCACCGCAGAGAGCGCGCAGCATCTCTCCCCGT |
| | | | GACCATGACCCAGCTACTGCCTCTTTAACCTTGAATGCCTT |
| | | | TTTGGGGGCTCACGTGTCACCCAGTGGCGAGTGAGCCACC |
| | | | CTTACTTCAGAAGAACGGCATGGGGTGGGGGGGCCTTAGG |
| | | | TGGTGCCCGCCTCACCTATGACTGCCAAAAGCGGTCATGG |
| | | | GGTTATTTTTAAACATGGGGAGGAAGTATTTATTGTTCCTG |
| | | | GGCTGCAGAGAGCTGGGCGGAGTGTGGAATTCTTCTCGGG |
| | | | AGGCAGTGCTGGGTCCTTTGGATCCGCCACC |
| 5 | M5 | MB | TGGCAAAGGGGCAGAAGGTGCGTGAGAGGTTGCGAATGG |
| | | | CCAGGACTGTCCTGGGGCCAGCCGGGGCACCTGGTGGCCA |
| | | | AGCTTAGAAACATGACAGGTCCTCTTGGGAGGGCTGACCG |
| | | | CAGGGAGCGTTGGGTTTCAGGCTGCTGGCGTCGGCTTCTGT |
| | | | GGTGCCCTTTCTGTCGGCTATGAGAGTCCAGACAGTGCCC |
| | | | AACCTCCTCCCCTTCTTTCCACACGCACAACCACCCCACCC |
| | | | CCTGTGGCCTGAGCTGTCCTGCCTCGCCACAATGGCACCTG |
| | | | CCCTAAAATAGCTTCCCATGTGAGGGCTAGAGAAAGGAAA |
| | | | AGATTAGACCCTCCCTGGATGAGAGAGAGAAAGTGAAGG |
| | | | AGGGCAGGGGAGGGGGACAGCGAGCCATTGAGCGATCTT |
| | | | TGTCAAGCATCCCAGAAGGTATAAAAACGCCCTTGGGACC |
| | | | AGGCAGCCTCAAACCCCAGCTGTTGGGGCCAGGACACCCA |
| | | | GTGAGCCCATACTTGCTCTTGGATCCGCCACC |
| 6 | M6 | DES | TCCTCAGGTACCCCCTGCCCCCCACAGCTCCTCTCCTGTGC |
| | | | CTTGTTTCCCAGCCATGCGTTCTCCTCTATAAATACCCGCT |
| | | | CTGGTATTTGGGGTTGGCAGCTGTTGCTGCCAGGGAGATG |
| | | | GTTGGGTTGACATGCGGCTCCTGACAAAACACAAACCCCT |
| | | | GGTGTGTGTGGGCGTGGGTGGTGTGAGTAGGGGGATGAAT |
| | | | CAGGGAGGGGGCGGGGGACCCAGGGGGCAGGAGCCACAC |
| | | | AAAGTCTGTGCGGGGGTGAGAAATGACCCAAAGTCTCTCC |
| | | | TTGACCAAAAAGGGGACCCTCAAACTAAAGGGAAGCCTCT |
| | | | CTTCTGCTGTCTCCCCTGACCCCACTCCCCCCCACCCCAGG |
| | | | ACGAGGAGATAACCAGGGCTGAAAGAGGCCCGCCTGGGG |
| | | | GCTGCAGACATGCTTGCTGCCTGCCCTGGCGAAGGATTGG |
| | | | CAGGCTTGCCCGTCACAGGACCCCCGCTGGCTGACTCAGG |
| | | | GGCGCAGGCCTCTTGCGGGGGAGCTGGCCTCCCCGCCCCC |
| | | | ACGGCCACGGGCCGCCCTTTCCTGGCAGGACAGCGGGATC |
| | | | TTGCAGCTGTCAGGGGAGGGGAGGCGGGGGCTGATGTCAG |
| | | | GAGGGATACAAATAGTGCCGACGGCTGGGGGCCCTGTCTC |
| | | | CCCTCGCCGCATCCACTCTCCGGCCGGCCGCCTGCCCGCCG |
| | | | CCTCCTCCGTGCGCCCGCCAGCCTCGCCCGCGCCGGATCCG |
| | | | CCACC |
| 7 | M7 | TPM2 | GCTCCGCCTCCCGAGAGACATTTAATCCGGGGGGATTTGC |
| | | | AGGAAACTTCTAAATTAAGGGTAGCGGCTGCTGCAGCTGA |
| | | | GGGGGGGCACGCCGGTCCCTGCGCCCGGGCAGCTGCCGTG |
| | | | AGCTCACGCCCCGAAATAGCCCCAGGGGCCCCAGCCGCAG |
| | | | CTGCCACTGGGTCCGGCTGTCACTCAGAGGAAGCACGGAG |
| | | | CCCCCAGCCCAAGGGTCCCCTCCCCTTCGCATCGCCGGGGT |
| | | | TTTTCCAGCCGACCGTCGGCCACTTTTTCCTCCGACGGCTG |
| | | | GCAGGGAAGAGGGGGATGGGGGCGGGACCCCAAGGGAGG |
| | | | CGGTCCCCAGTGGGTGGGCGAAGGGGGCGGCCGCACCCCC |
| | | | CGGCCGGGCCGTGCTTCTGCCCCTACAAGGTTTGGGCCGA |
| | | | GGTGGGGGAGGGTCCTGGTTGCCGGCCCCGCCCGGTCCCT |
| | | | CCCCGCCTTTTAGGCGCCCGCGTGGCCGGGACGTCCCAGT |
| | | | CCCGCTCCGTCCTCCTCGCCTGCCACCGGTGCACCCAGTCC |
| | | | GCTCACCCAGCCCAGTCCGTCCGGTCCTCACCGCCTGCCGG |
| | | | CCGGCCCACCCCCCACCGGATCCGCCACC |
| 8 | M8 | TNNC2 | CCACTGCATTCCAGCCTGGGTGACAGAGTGAGAGTCTGTC |
| | | | TCAAAAACAAAACCCCAAAAAACCAAAAAACAATAAAAA |
| | | | CTAAATTGGAATCCCATGGGTCTCCCTCACCCTTTGGACCC |
| | | | TGCCTGCCTGGGGGAGGCCTTATTTAGCTTGGCCCAGCCCT |
| | | | GCTGAGCTTTCCTTGGGAATGTCTATATATAGGGGAAGGG |
| | | | GGCAGCCCAGTTGCCACTGTCCATCTGCATTCCTTGGTGTC |
| | | | CAGCCCCCCTTTCCTGGCCCCACTGCCCCTTACCATGGTTC |
| | | | AAAGACGGGACATTCCTCTTGCCAAGCACCAACGCTGTGT |
| | | | ACCCAGCACAAGCTAGGTGACTTATACCCGGGTTCCCATTT |
| | | | ACCATGGTCTCTCTGTGAGGGAGACCCCACAGACAGGTCC |
| | | | AATCCCACAGATGAAGAAATGGCTCAGAGAGGCACTGCTA |
| | | | CCGGCCCAAGGTCACGCCACCTGCTTGGAGGCTTAAATTT |
| | | | CTCTGCAAGGGCCCTTGGCTAAATTAGGTAATGGGTTCAG |
| | | | ACTGTGGGAGGGGTGGGACTCGCTGACCCCAGGATCTGAT |
| | | | TGGGCAGGGTCTCCAGTGCTGGGGAGCAGGGAGGTGGGA |
| | | | GGGGAGGGTGCCCCTACAAATCCCGGGGGCTAGAGCAGG |
| | | | CCAGGTCATCTTTGGGTGGTGGAGTGCAAAGGAGGCGACC |
| | | | TGCAACAGAGGAGTCCCGGTCACCAGGATCCGCCACC |
| 9 | M9 | TNNC1 | TGGGTCAGTCACTTCCTCCTTCACCAGTTCCACAGCATCTT |
| | | | ACTATGAGCTTGGCATTCGAGGCTTCTCTTGGCAGGGCCCT |
| | | | GCACTCCTAGCCTCTCCTTGCACATTGCACCCCCATTCCAG |
| | | | AGAGGTTTAGTTAAAGGCGGGGGTTACCAAGTCAGTCAGA |
| | | | TCTTGGGCAAGTCACCACTCCTCCAGAGCCTCAGTTTCCTT |
| | | | ATCTGGAAAGTGGAGGTCATGGCAACCCGCCAACCTGGTT |
| | | | GGATGGGAGCCTGAGCTGTTGTGTTGCACCTTGCCTGGGG |
| | | | CCCACGACTTTGTAGCTCCTGTCCTGCACTGGGCTTATGTT |
| | | | TTCATTCATTCCAGAAACCTTTTCAGAGAGTCCCTTTGGGG |
| | | | AGTGTGGGGGACAGGAGGGAAAGAAACCTGGTCCTTGTA |
| | | | GCCGTTCGTCTGCTCCCTGCCCTGGGCAGAGGACGTGGGG |
| | | | ACTCAGGCCAGCCTGAGATCACTGGGACCAGAGGAGGGG |
| | | | CTGGAGGATACTACACGCAGGGGTGGGCTGGGCTGGGCTG |
| | | | GGCTGGGCCAGGAATGCAGCGGGGCAGGGCTATTTAAGTC |
| | | | AAGGGCCGGCTGGCAACCCCAGCAAGCTGTCCTGTGAGCC |
| | | | GGGATCCGCCACC |
| 10 | M10 | TPT1 | CCTCCCACGCTGCGCGCGCACCTCCCCGCCCCCACCCCTAC |
| | | | CCGCTGGCGTGCCCAGTGGAACGGAGCCTTGTGTCTCCGC |
| | | | CTCAAGTCCCCGGATGCTCACCTCCCCGACTCGCCCCCGCT |
| | | | GTGGCCCCGCCCCCGCGCGGCTCTTCGTGCCACGTCACCGC |
| | | | CTGCGTCGCTTCCGGAGGCGCAGCGGGCGATGACGTAGAG |
| | | | GGACGTGCCCTCTATATGAGGTTGGGGAGCGGCTGAGTCG |
| | | | GCCTTTTCCGCCCGCTCCCCCCTCCCCCCGAGCGCCGCTCC |
| | | | GGCTGCACCGCGCTCGCTCCGAGTTTCAGGCTCGTGCTAA |
| | | | GCTAGCGCCGTCGTCGTCTCCCTTCAGTCGGATCCGCCACC |
| 11 | M11 | TNNT1 | TCAAGGTCATGGCTTGGCTGGGAAGCGGGAGGGGGATACC |
| | | | AAAAAAGATCAGCGTTAGATGGAGGTGGGGAGGGAGGGT |
| | | | AGGTTTCACCCTAATTGACACCCCACCCTCCCCCTCCCCCT |
| | | | CCCCACACCTGCTTCCCGGCTTTAGTGCCTGTTGTCACCAG |
| | | | GATGGAATCCTGGCGGCAGACAAAGGGGTGGGGGTGGGA |
| | | | GGAGGAGACAAAAAGATGGACAGAGACAGAGGAGGAGA |
| | | | CTAAGAAGTAGAGACAGACACACAGAGACACACAGATGG |
| | | | AGAGCCACAGAGACAACAGAAATGCAGGGGCGGGGCTCC |
| | | | TGGGCCATGCAGATGGGGTTGGCATGGGGTTGGGGAGGGG |
| | | | CCCACGGAGGCTTCTGGACTCTGAGTGTGGAGCCAGGGGC |
| | | | AGCAGGGCTGTCTCTGAACTCCCAGGCTTGTGTTTGAGCA |
| | | | AAGGAATTCTCTCCCCCTCCCTCTCCCCTCGGGGGGGGCCC |
| | | | GAGCCTCCAGCTATAAACTCCCCGGAGCTTCAGTGCCCTC |
| | | | AGCAAGGCTCAGCCTCAAGAGGATCCGCCACC |
| 12 | M12 | GAPDH | GAGGACACAGCCTGGCTCTGGGTCCCAGCAGGAAAGGCA |
| | | | ATCCCAGAAAGGCAGGGTCAGGGACTGGAGTCCTGTGGGT |
| | | | GCTTTTTAAGCAAAGATTATCACCAGGCAGGCTAAACTTA |
| | | | GCAACCGGCTTTTAGCTAGAAGGGCAGGGGGCTGGTGTCA |
| | | | GGTTATGCTGGGCCAGCAAAGAGGCCCGGGATCCCCCTCC |
| | | | CATGCACCTGCTGATGGGCCAAGCAGTGAAAGGGAGTCCC |
| | | | TGGCTCCTGGCTCGCCTGCACGTCCCAGGGGGGGAGGGA |
| | | | CTTCCGCCCTCACGTCCCGCTCTTCGCAGGTTTCCAGATCC |
| | | | TGTCCTCCGCCTGGGGCCCCAGCCCGGAGAGAGCCGCTGG |
| | | | TGCACACAGGGCCGGGATTGTCTGCCCTAATTATCAGGTC |
| | | | CAGGCCCCCTCCCCCTCAAGCCGCCTCCCGAGCCTCCTTCC |
| | | | TCTAGATGGGGAGGGGGAAGTGGGGGCTGGGAAGGAACC |
| | | | ACGGGCCCCCGCCCGAGGCCCATGGGCCCCTCCTCTTTCCC |
| | | | GCCTCTCAGCCTTTGAAAGAAAGAAAGGGGAGGGGGCAG |
| | | | GTCCCAAAGTCCTCCTGTTTCATCCAAGCGTGTAAGGGTCC |
| | | | CCGTCCTTGACTCCCTAGTGTCCTGCTGCCCACAGTCCAGT |
| | | | CCTGGGAACCAGCACCGATCACCTCCCATCGGGCCAATCT |
| | | | CAGTCCCTTCCCCCCTACGTCGGGGCCCACACGCTCGGTGC |
| | | | GTGCCCAGTTGAACCAGGCGGCTGCGGAAAAAAAAAAGC |
| | | | GGGGAGAAAGTAGGGCCCGGCTACTAGCGGTTTTACGGGC |
| | | | GCACGTAGCTCAGGCCTCAAGACCTTGGGCTGGGACTGGC |
| | | | TGAGCCTGGCGGGAGGCGGGGTCCGAGTCACCGCCTGCCG |
| | | | CCGCGCCCCCGGTTTCTATAAATTGAGCCCGCAGCCTCCCG |
| | | | CTTCGCTCTCTGCTCCTCCTGTTCGACAGTCAGCCGCATCT |
| | | | TCTTTTGCGTCGCCAGGTGAAGACGGGCGGGGATCCGCCA |
| | | | CC |
| 13 | M13 | MT1X | AAAGTGACTAAAAGGAAGGAAAAAACCTAAAATGTTTCC |
| | | | AAGTGGTGGGGTGTATGGGGAGAAATGTGCCCATTCAGCC |
| | | | TCTCAAGGGGGCAGAGCCCTGTGTCTGTTCTGTCCCTAGAC |
| | | | CGAGGTGGGGACGGGGACAGGGACCGGCAATGCAGGAAA |
| | | | TCCCTGCCTCTCATTTGCACACTTGGGAAATGCAAAGCTAC |
| | | | ACACAGTGGAGCGGGGGCTGGGGGTGTGTAGACCGCCGG |
| | | | GATTCCGAGTTTCCCGGCACGCCTAGGAGAGGGAGAGGCA |
| | | | GGCAATGTCAGGGAAATTGGGCAGGCAAGACGCCAGGGA |
| | | | CGCCACGTACTGCCAGGTTCTCAACGAGGTGGAGCCAAAG |
| | | | GGGCAGGCCCCGCGGTGCGCCCGGCGCTGGGCTCACGGGT |
| | | | TGCTGCACCCGGCCCAGGATCGCGGGCGGTGCAGACTCAG |
| | | | CAGGGGGGGGTGCAAGGACGAGGCGGGGCCTCTGCGCCC |
| | | | GGCCCTCTTCCCGGACTATAAAGAGAGCCGCCGGCTTCTG |
| | | | GGCTCCACCACGCTTTTCATCTGTCCCGCTGCGTGTTTTCCT |
| | | | CTTGATCGGGAACTCCTGCTTCTCCTTGCCGGATCCGCCAC |
| | | | C |
| 14 | M14 | MYLPF | TAAAAAAAAACCTGAACTCCTGGCCTCAAGCAATCCTCCT |
| | | | GCCTCAGCTTCCCAAAGTGCTGGGATTACAGGTGTGAACC |
| | | | ACTGCACCCAGACGTTTACTGGGTTTAAATCTGAGCCCACC |
| | | | CCACTCAGGGGTACAGGATCTGGTTCATTTATCTCCCAGAA |
| | | | TGATGCCTTGGGTTGCGTCCCCTCTAAACTCTGGCACATTC |
| | | | CAGCCCCTCTTTGGGAGAAAGAACATCTCTTCTTGCCCAGG |
| | | | CCCTAGTGAGACATGCATCGCAAGACATGCTCCCCCCTTCC |
| | | | TCCCCGCTGGGCTCCAGCTGCTGCCAGCCCTCCAAGAAAG |
| | | | GAGAGGCCCTGAGTTGGGCTATTTTGGTATCTGGGGTGGG |
| | | | CACCCGCAGGGCTAAGGTTACCTTGGTATGTTAAGGGCTC |
| | | | CCTGGGGCAGGACTATATAACCCCAGAGGGACTGCCCCAT |
| | | | GCTGACTCCTTGCTTCTTTCCAGCCGGAGCCGCTGCCTTGC |
| | | | CCCCCGGAGACTGGATCCGCCACC |
| 15 | M15 | MYL1 | TAAAATAAAATAAAGTAGTTAAAAAAAAATCTATCAGCTT |
| | | | TCCTGCCAAAATAAAAAAAATTATAAAGATAACAAGGAG |
| | | | GGAAGATGATGAAAAGCGGATTGAAAGTGCGCTCTCTGAA |
| | | | CTTCTCCTGCTATAGCAGTTGCCAGCTCTTCTGCCATCAAA |
| | | | CAAATAGGAAGCTTCTTAAAGGAAGATGCTCCAAGAGAAC |
| | | | ATCTTGGAAATAAAGACCATAAAAGTACCGGCAGGCTTCC |
| | | | ATCTCACTATGGCTGTCTACTCCAAGGTTCTGGTCATCTAA |
| | | | AAATAGCTCTCAGGGTACAGATCCATCTTTCCCTTTGCCCT |
| | | | AAGAAAGCTAAAGAACTCTCCAAGGGGTGTGGCAACTTAT |
| | | | CTCTGAAACCTGATGCTAGCTGTGAGGTCAAAGCTTGCCC |
| | | | AGAAATAAAAGGAAGCCTCAGCCAGGGATGACCCCACTC |
| | | | AGGGACCGGAGCAGCCCTCAACTCACTCTTCAGCTTCCCT |
| | | | GCTGTGTTGCAGCCCAGCCGCGGATCCGCCACC |
| 16 | M16 | MYH7 | CACTGTCCCTGTGACCTCAACACATTGCCTGCTGGTCCAAG |
| | | | GAAATCAGTTTATTGTTCTTGCCACCTGGCTAGGAAATGTC |
| | | | CTTCCAGCTGCCCCAGTCTAGCTGCCTCACCCTGGGGCCAT |
| | | | GCCCCCAACTCTGTCCTACCCTTTCCAGTTGGAACACTGTG |
| | | | GAAATGCGGCCAGGCCATCAGGGGCCTCGCACTCTCCTGC |
| | | | CAGCTGTGGAATGTGAGGCCTGGCCTGGGAGATATTTTTG |
| | | | CTGCACTTTGAGCCACCCCGCCCCCTGGAACTCAGACCCTG |
| | | | CACAGTCCATGCCATAACAATGACGACCACTTCCAATTGTT |
| | | | TCCTAGCTGGAGAGGCGGGGAGGGGAGCACTGTTTGGGAA |
| | | | GGGGGGGAGCCTGGGGGAAATGCTTCTAGTGACAACAGCC |
| | | | CTTTCTAAATCCGGCTAGGGACTGGGTGCCGTTGGGGGTG |
| | | | GGGGTGCCCTGCTGCCCCATATATACAGCCCCTGAGACCA |
| | | | GGTCTGGCTCCACAGCTCTGTCCTGCTCTGTGTCTTTCCCT |
| | | | GCTGCTCTCAGGTAGGAGCGGGATCCGCCACC |
| 17 | M17 | CRYAB | GACTCACAAAGAGTTAATGTCCCTGGGGCTCAGCCTAGGA |
| | | | AGATTCCAGTCCCTGCCCAGGCCCAAGATAGTTGCTGGCC |
| | | | TGATTCCCCTGGCATTCAGGACTGGAAAGGAGGAGGAGGG |
| | | | GCACACTACGCCGGCTCCCATCCTCCCCCCACCCCGCGTGC |
| | | | CTGCTTGGGATTCCTGACTCTGTACCAGCTTCAGAGAACAG |
| | | | GGGTGGGGGTGGGTGCCATTGGGTGTGGACAGAAAGCTAG |
| | | | TGAAACAAGACCATGACAAGTCACTGGCCGGCTCAGACGT |
| | | | GTTTGTGTCTCTCTTTTCTTAGCTCAGTGAGTACTGGGTAT |
| | | | GTGTCACATTGCCAAATCCCGGATCACAAGTCTCCATGAA |
| | | | CTGCTGGTGAGCTAGGATAATAAAACCCCTGACATCACCA |
| | | | TTCCAGAAGCTTCACAAGACTGCATATATAAGGGGCTGGC |
| | | | TGTAGCTGCAGCTGAAGGAGCTGACCAGCCAGCTGACCCC |
| | | | TCACACTCACCTAGGATCCGCCACC |
| 18 | M18 | TPM3 | CCGACTACCTGCAAAGGGGCCAACCATTGTGGGCAGCCAG |
| | | | CCTGGCGGGAGAGGCTAGGGCACGAAGCGCCGCCCAGTTA |
| | | | CCATGGTAACACTCCCTCGTCCCAACCCCGTCCCAGTTTAC |
| | | | TGGCCCAAGGTAAGGCCGGGAGCCGTACTCCAGGGCGACT |
| | | | GGGAGGATCCACTCCCTGCCGCGGCTCTCCCTCCAGTGGA |
| | | | CTGCCTTCCCGCCTGCCTGGCGGTGGGAGGGCGCGGCAGA |
| | | | GGGACGTCACATCCGGGCGGGTTGGTGAGTTCCGGTATTT |
| | | | CAGGGCGTAGCAGGCGGAAGTAAGGGTGAGAGGAGGCTG |
| | | | CAACGCCGAGCGGAGGAGGCAGGAACCGGAGCGCGAGCA |
| | | | GTAGCTGGGTGGGCACCATGGCTGGGATCACCACCATCGA |
| | | | GGCGGTGAAGCGGGATCCGCCACC |
| 19 | M19 | hDES715 | CACCCATGCCTCCTCAGGTACCCCCTGCCCCCCACAGCTCC |
| | | | TCTCCTGTGCCTTGTTTCCCAGCCATGCGTTCTCCTCTATAA |
| | | | ATACCCGCTCTGGTATTTGGGGTTGGCAGCTGTTGCTGCCA |
| | | | GGGAGATGGTTGGGTTGACATGCGGCTCCTGACAAAACAC |
| | | | AAACCCCTGGTGTGTGTGGGCGTGGGTGGTGTGAGTAGGG |
| | | | GGATGAATCAGGGAGGGGGCGGGGGACCCAGGGGGCAGG |
| | | | AGCCACACAAAGTCTGTGCGGGGGGGGAGCGCACATAGC |
| | | | AATTGGAAACTGAAAGCTTATCAGACCCTTTCTGGAAATC |
| | | | AGCCCACTGTTTATAAACTTGAGGCCCCACCCTCGACGGA |
| | | | GATAACCAGGGCTGAAAGAGGCCCGCCTGGGGGCTGCAG |
| | | | ACATGCTTGCTGCCTGCCCTGGCGAAGGATTGGCAGGCTT |
| | | | GCCCGTCACAGGACCCCCGCTGGCTGACTCAGGGGCGCAG |
| | | | GCCTCTTGCGGGGGAGCTGGCCTCCCCGCCCCCACGGCCA |
| | | | CGGGCCGCCCTTTCCTGGCAGGACAGCGGGATCTTGCAGC |
| | | | TGTCAGGGGAGGGGAGGCGGGGGCTGATGTCAGGAGGGA |
| | | | TACAAATAGTGCCGACGGCTGGGGGCCCTGTCTCCCCTCG |
| | | | CCGCATCCACTCTCCGGCCGGCCGCCTGCCCGCCGCCTCCT |
| | | | CCGTGCGCCCGCCAGCCTCGCCCGCGCCGTCACC |
| 20 | M20 | mDESenh- | AGCTTGTCGCTGTCAGCCTTCCTTGACACCTCTGTCTCCTC |
| | | CKMenh- | AGGTGCCTGGCTCCCAGTCCCCAGAACGCCTCTCCTGTACC |
| | | CKMp- | TTGCTTCCTAGCTGGGCCTTTCCTTCTCCTCTATAAATACCA |
| | | INR-MD- | GCTCTGGTATTTCGCCTTGGCAGCTGTTGCTGCTAGGGAGA |
| | | HBBi-SIE | CGGCTGGCTTGACACCACTACGGGTCTAGGCTGCCCATGT |
| | | | AAGGAGGCAAGGCCTGGGGACACCCGAGATGCCTGGTTAT |
| | | | AATTAACCCAGACATGTGGCTGCCCCCCCCCCCCAACACC |
| | | | TGCTGCCTGAGCCTCACCCCCACCCCGGTGCCTGGGTCTTA |
| | | | GGCTCTGTACACCATGGAGGAGAAGCTCGCTCTAAAAATA |
| | | | ACCCTGTCCCTGGTGGATCATCAAGGCTGTGGGGGACTGA |
| | | | GGGCAGGCTGTAACAGGCTTGGGGGCCAGGGCTTATACGT |
| | | | GCCTGGGACTCCCAAAGTATTACTGTTCCATGTTCCCGGCG |
| | | | AAGGGCCAGCTGTCCCCCGCCAGCTAGACTCAGCACTTAG |
| | | | TTTAGGAACCAGTGAGCAAGTCAGCCCTTGGGGCAGCCCA |
| | | | TACAAGGCCATGGGGCTGGGCAAGCTGCACGCCTGGGTCC |
| | | | GGGGTGGGCACGGTGCCCGGGCAACGAGCTGAAAGCTCAT |
| | | | CTACTCTCAGGGGCCCCTCCCTGGGGACAGCCCCTCCTGGC |
| | | | TAGTCACACCCTGTAGGCTCCTCTATATAACCCAGGGGCA |
| | | | CAGGGGCTGCCCTCAGTCCGCCTGGAGACCTCGAGCCGAG |
| | | | TGGTCGTCTCAGGAGCCGGGCAGGTTGGTATCAAGGTTAC |
| | | | AAGCAGCCCACCTGTCCCAGTGCTGACTTAGTGCAAGGCG |
| | | | AGCCAGCAAGGAGGGAGGACAGGTGGCAGTGGGGGGTGA |
| | | | GGAGCATCTAAAAATAGCCACTCTTGGGTTTCTGATAGGC |
| | | | ACTGACTCTCTCTGCCTATTGGTCTATTTTCCCACCCTTAGG |
| | | | CTGCTAAAGGCCGCCACC |
| 164 | | mDES | AGCTTGTCGCTGTCAGCCTTCCTTGACACCTCTGTCTCCTC |
| | | enhancer | AGGTGCCTGGCTCCCAGTCCCCAGAACGCCTCTCCTGTACC |
| | | | TTGCTTCCTAGCTGGGCCTTTCCTTCTCCTCTATAAATACCA |
| | | | GCTCTGGTATTTCGCCTTGGCAGCTGTTGCTGCTAGGGAGA |
| | | | CGGCTGGCTTGACA |
| 134 | | CKM | CCACTACGGGTCTAGGCTGCCCATGTAAGGAGGCAAGGCC |
| | | enhancer | TGGGGACACCCGAGATGCCTGGTTATAATTAACCCAGACA |
| | | | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATC |
| 135 | | CKM | ATCAAGGCTGTGGGGGACTGAGGGCAGGCTGTAACAGGCT |
| | | promoter- | TGGGGGCCAGGGCTTATACGTGCCTGGGACTCCCAAAGTA |
| | | INR-MD | TTACTGTTCCATGTTCCCGGCGAAGGGCCAGCTGTCCCCCG |
| | | | CCAGCTAGACTCAGCACTTAGTTTAGGAACCAGTGAGCAA |
| | | | GTCAGCCCTTGGGGCAGCCCATACAAGGCCATGGGGCTGG |
| | | | GCAAGCTGCACGCCTGGGTCCGGGGTGGGCACGGTGCCCG |
| | | | GGCAACGAGCTGAAAGCTCATCTACTCTCAGGGGCCCCTC |
| | | | CCTGGGGACAGCCCCTCCTGGCTAGTCACACCCTGTAGGC |
| | | | TCCTCTATATAACCCAGGGGCACAGGGGCTGCCCTCAGTC |
| | | | CGCCTGGAGACCTCGAGCCGAGTGGTCGT |
| 148 | | HBG/SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE (part | CAGCCCACCTGTCCCAGTGCTGACTTAGTGCAAGGCGAGC |
| | | of HBG/SIE | CAGCAAGGAGGGAGGACAGGTGGCAGTGGGGGGTGAGGA |
| | | intron | GCATCTAAAAATAGCC |
| | | sequence) | |
| 21 | M21 | mDESenh- | AGCTTGTCGCTGTCAGCCTTCCTTGACACCTCTGTCTCCTC |
| | | CKMenh- | AGGTGCCTGGCTCCCAGTCCCCAGAACGCCTCTCCTGTACC |
| | | CKMp- | TTGCTTCCTAGCTGGGCCTTTCCTTCTCCTCTATAAATACCA |
| | | INR-MD- | GCTCTGGTATTTCGCCTTGGCAGCTGTTGCTGCTAGGGAGA |
| | | HBBi-SIE- | CGGCTGGCTTGACACCACTACGGGTCTAGGCTGCCCATGT |
| | | hDES715p | AAGGAGGCAAGGCCTGGGGACACCCGAGATGCCTGGTTAT |
| | | | AATTAACCCAGACATGTGGCTGCCCCCCCCCCCCAACACC |
| | | | TGCTGCCTGAGCCTCACCCCCACCCCGGTGCCTGGGTCTTA |
| | | | GGCTCTGTACACCATGGAGGAGAAGCTCGCTCTAAAAATA |
| | | | ACCCTGTCCCTGGTGGATCATCAAGGCTGTGGGGGACTGA |
| | | | GGGCAGGCTGTAACAGGCTTGGGGGCCAGGGCTTATACGT |
| | | | GCCTGGGACTCCCAAAGTATTACTGTTCCATGTTCCCGGCG |
| | | | AAGGGCCAGCTGTCCCCCGCCAGCTAGACTCAGCACTTAG |
| | | | TTTAGGAACCAGTGAGCAAGTCAGCCCTTGGGGCAGCCCA |
| | | | TACAAGGCCATGGGGCTGGGCAAGCTGCACGCCTGGGTCC |
| | | | GGGGTGGGCACGGTGCCCGGGCAACGAGCTGAAAGCTCAT |
| | | | CTACTCTCAGGGGCCCCTCCCTGGGGACAGCCCCTCCTGGC |
| | | | TAGTCACACCCTGTAGGCTCCTCTATATAACCCAGGGGCA |
| | | | CAGGGGCTGCCCTCAGTCCGCCTGGAGACCTCGAGCCGAG |
| | | | TGGTCGTCTCAGGAGCCGGGCAGGTTGGTATCAAGGTTAC |
| | | | AAGCAGCCCACCTGTCCCAGTGCTGACTTAGTGCAAGGCG |
| | | | AGCCAGCAAGGAGGGAGGACAGGTGGCAGTGGGGGGTGA |
| | | | GGAGCATCTAAAAATAGCCCACCCATGCCTCCTCAGGTAC |
| | | | CCCCTGCCCCCCACAGCTCCTCTCCTGTGCCTTGTTTCCCA |
| | | | GCCATGCGTTCTCCTCTATAAATACCCGCTCTGGTATTTGG |
| | | | GGTTGGCAGCTGTTGCTGCCAGGGAGATGGTTGGGTTGAC |
| | | | ATGCGGCTCCTGACAAAACACAAACCCCTGGTGTGTGTGG |
| | | | GCGTGGGTGGTGTGAGTAGGGGGATGAATCAGGGAGGGG |
| | | | GCGGGGGACCCAGGGGGCAGGAGCCACACAAAGTCTGTG |
| | | | CGGGGGTGGGAGCGCACATAGCAATTGGAAACTGAAAGC |
| | | | TTATCAGACCCTTTCTGGAAATCAGCCCACTGTTTATAAAC |
| | | | TTGAGGCCCCACCCTCGACGGAGATAACCAGGGCTGAAAG |
| | | | AGGCCCGCCTGGGGGCTGCAGACATGCTTGCTGCCTGCCC |
| | | | TGGCGAAGGATTGGCAGGCTTGCCCGTCACAGGACCCCCG |
| | | | CTGGCTGACTCAGGGGCGCAGGCCTCTTGCGGGGGAGCTG |
| | | | GCCTCCCCGCCCCCACGGCCACGGGCCGCCCTTTCCTGGCA |
| | | | GGACAGCGGGATCTTGCAGCTGTCAGGGGAGGGGAGGCG |
| | | | GGGGCTGATGTCAGGAGGGATATATAAAGTGCCGACGGCT |
| | | | GGGGGCCCTTCAGTCCGCCTGGAGACCTCGAGCCGAGTGG |
| | | | TCGTACTCTTGGGTTTCTGATAGGCACTGACTCTCTCTGCC |
| | | | TATTGGTCTATTTTCCCACCCTTAGGCTGCTAAAGGCCGCC |
| | | | ACC |
| 164 | | mDES | AGCTTGTCGCTGTCAGCCTTCCTTGACACCTCTGTCTCCTC |
| | | enhancer | AGGTGCCTGGCTCCCAGTCCCCAGAACGCCTCTCCTGTACC |
| | | | TTGCTTCCTAGCTGGGCCTTTCCTTCTCCTCTATAAATACCA |
| | | | GCTCTGGTATTTCGCCTTGGCAGCTGTTGCTGCTAGGGAGA |
| | | | CGGCTGGCTTGACA |
| 134 | | CKM | CCACTACGGGTCTAGGCTGCCCATGTAAGGAGGCAAGGCC |
| | | enhancer | TGGGGACACCCGAGATGCCTGGTTATAATTAACCCAGACA |
| | | | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATC |
| 135 | | CKM | ATCAAGGCTGTGGGGGACTGAGGGCAGGCTGTAACAGGCT |
| | | promoter- | TGGGGGCCAGGGCTTATACGTGCCTGGGACTCCCAAAGTA |
| | | INR-MD | TTACTGTTCCATGTTCCCGGCGAAGGGCCAGCTGTCCCCCG |
| | | | CCAGCTAGACTCAGCACTTAGTTTAGGAACCAGTGAGCAA |
| | | | GTCAGCCCTTGGGGCAGCCCATACAAGGCCATGGGGCTGG |
| | | | GCAAGCTGCACGCCTGGGTCCGGGGTGGGCACGGTGCCCG |
| | | | GGCAACGAGCTGAAAGCTCATCTACTCTCAGGGGCCCCTC |
| | | | CCTGGGGACAGCCCCTCCTGGCTAGTCACACCCTGTAGGC |
| | | | TCCTCTATATAACCCAGGGGCACAGGGGCTGCCCTCAGTC |
| | | | CGCCTGGAGACCTCGAGCCGAGTGGTCGT |
| 149 | | HBG/SIE/ | GGGCAGGTTGGTATCAAGGTTACAAGCAGCCCACCTGTCC |
| | | hDES715md | CAGTGCTGACTTAGTGCAAGGCGAGCCAGCAAGGAGGGA |
| | | intron | GGACAGGTGGCAGTGGGGGGTGAGGAGCATCTAAAAATA |
| | | | GCCCACCCATGCCTCCTCAGGTACCCCCTGCCCCCCACAGC |
| | | | TCCTCTCCTGTGCCTTGTTTCCCAGCCATGCGTTCTCCTCTA |
| | | | TAAATACCCGCTCTGGTATTTGGGGTTGGCAGCTGTTGCTG |
| | | | CCAGGGAGATGGTTGGGTTGACATGCGGCTCCTGACAAAA |
| | | | CACAAACCCCTGGTGTGTGTGGGCGTGGGTGGTGTGAGTA |
| | | | GGGGGATGAATCAGGGAGGGGGCGGGGGACCCAGGGGGC |
| | | | AGGAGCCACACAAAGTCTGTGCGGGGGTGGGAGCGCACA |
| | | | TAGCAATTGGAAACTGAAAGCTTATCAGACCCTTTCTGGA |
| | | | AATCAGCCCACTGTTTATAAACTTGAGGCCCCACCCTCGAC |
| | | | GGAGATAACCAGGGCTGAAAGAGGCCCGCCTGGGGGCTG |
| | | | CAGACATGCTTGCTGCCTGCCCTGGCGAAGGATTGGCAGG |
| | | | CTTGCCCGTCACAGGACCCCCGCTGGCTGACTCAGGGGCG |
| | | | CAGGCCTCTTGCGGGGGAGCTGGCCTCCCCGCCCCCACGG |
| | | | CCACGGGCCGCCCTTTCCTGGCAGGACAGCGGGATCTTGC |
| | | | AGCTGTCAGGGGAGGGGAGGCGGGGGCTGATGTCAGGAG |
| | | | GGATATATAAAGTGCCGACGGCTGGGGGCCCTTCAGTCCG |
| | | | CCTGGAGACCTCGAGCCGAGTGGTCGTACTCTTGGGTTTCT |
| | | | GATAGGCACTGACTCTCTCTGCCTATTGGTCTATTTTCCCA |
| | | | CCCTTAGGCTGCT |
| 214 | | SIE (part | CAGCCCACCTGTCCCAGTGCTGACTTAGTGCAAGGCGAGC |
| | | of HBG/SIE/ | CAGCAAGGAGGGAGGACAGGTGGCAGTGGGGGGTGAGGA |
| | | hDES715md | GCATCTAAAAATAGCC |
| | | intron) | |
| 151 | | hDES715md | CACCCATGCCTCCTCAGGTACCCCCTGCCCCCCACAGCTCC |
| | | promoter | TCTCCTGTGCCTTGTTTCCCAGCCATGCGTTCTCCTCTATAA |
| | | (part of | ATACCCGCTCTGGTATTTGGGGTTGGCAGCTGTTGCTGCCA |
| | | HBG/SIE/ | GGGAGATGGTTGGGTTGACATGCGGCTCCTGACAAAACAC |
| | | hDES715md | AAACCCCTGGTGTGTGTGGGCGTGGGTGGTGTGAGTAGGG |
| | | intron) | GGATGAATCAGGGAGGGGGCGGGGGACCCAGGGGGCAGG |
| | | | AGCCACACAAAGTCTGTGCGGGGGTGGGAGCGCACATAGC |
| | | | AATTGGAAACTGAAAGCTTATCAGACCCTTTCTGGAAATC |
| | | | AGCCCACTGTTTATAAACTTGAGGCCCCACCCTCGACGGA |
| | | | GATAACCAGGGCTGAAAGAGGCCCGCCTGGGGGCTGCAG |
| | | | ACATGCTTGCTGCCTGCCCTGGCGAAGGATTGGCAGGCTT |
| | | | GCCCGTCACAGGACCCCCGCTGGCTGACTCAGGGGCGCAG |
| | | | GCCTCTTGCGGGGGAGCTGGCCTCCCCGCCCCCACGGCCA |
| | | | CGGGCCGCCCTTTCCTGGCAGGACAGCGGGATCTTGCAGC |
| | | | TGTCAGGGGAGGGGAGGCGGGGGCTGATGTCAGGAGGGA |
| | | | TATATAAAGTGCCGACGGCTGGGGGCCCTTCAGTCCGCCT |
| | | | GGAGACCTCGAGCCGAGTGGTCGT |
| 22 | M22 | tMCK | CCACTACGGGTCTAGGCTGCCCATGTAAGGAGGCAAGGCC |
| | | | TGGGGACACCCGAGATGCCTGGTTATAATTAACCCAGACA |
| | | | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATCCCCACTACGGGTCTAGGCTGCCCATGTAAGGAGGCA |
| | | | AGGCCTGGGGACACCCGAGATGCCTGGTTATAATTAACCC |
| | | | AGACATGTGGCTGCCCCCCCCCCCCAACACCTGCTGCCTG |
| | | | AGCCTCACCCCCACCCCGGTGCCTGGGTCTTAGGCTCTGTA |
| | | | CACCATGGAGGAGAAGCTCGCTCTAAAAATAACCCTGTCC |
| | | | CTGGTGGATCCCCACTACGGGTCTAGGCTGCCCATGTAAG |
| | | | GAGGCAAGGCCTGGGGACACCCGAGATGCCTGGTTATAAT |
| | | | TAACCCAGACATGTGGCTGCCCCCCCCCCCCAACACCTGCT |
| | | | GCCTGAGCCTCACCCCCACCCCGGTGCCTGGGTCTTAGGCT |
| | | | CTGTACACCATGGAGGAGAAGCTCGCTCTAAAAATAACCC |
| | | | TGTCCCTGGTGGATCCCTCCCTGGGGACAGCCCCTCCTGGC |
| | | | TAGTCACACCCTGTAGGCTCCTCTATATAACCCAGGGGCA |
| | | | CAGGGGCTGCCCCCGGGTCACC |
| 23 | M23 | tMCK-MD- | CCACTACGGGTCTAGGCTGCCCATGTAAGGAGGCAAGGCC |
| | | HBBi-SIE | TGGGGACACCCGAGATGCCTGGTTATAATTAACCCAGACA |
| | | | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATCCCCACTACGGGTCTAGGCTGCCCATGTAAGGAGGCA |
| | | | AGGCCTGGGGACACCCGAGATGCCTGGTTATAATTAACCC |
| | | | AGACATGTGGCTGCCCCCCCCCCCCAACACCTGCTGCCTG |
| | | | AGCCTCACCCCCACCCCGGTGCCTGGGTCTTAGGCTCTGTA |
| | | | CACCATGGAGGAGAAGCTCGCTCTAAAAATAACCCTGTCC |
| | | | CTGGTGGATCCCCACTACGGGTCTAGGCTGCCCATGTAAG |
| | | | GAGGCAAGGCCTGGGGACACCCGAGATGCCTGGTTATAAT |
| | | | TAACCCAGACATGTGGCTGCCCCCCCCCCCCAACACCTGCT |
| | | | GCCTGAGCCTCACCCCCACCCCGGTGCCTGGGTCTTAGGCT |
| | | | CTGTACACCATGGAGGAGAAGCTCGCTCTAAAAATAACCC |
| | | | TGTCCCTGGTGGATCCCTCCCTGGGGACAGCCCCTCCTGGC |
| | | | TAGTCACACCCTGTAGGCTCCTCTATATAACCCAGGGGCA |
| | | | CAGGGGCTGCCCTCAGTCCGCCTGGAGACCTCGAGCCGAG |
| | | | TGGTCGTCTCAGGAGCCGGGCAGGTTGGTATCAAGGTTAC |
| | | | AAGCAGCCCACCTGTCCCAGTGCTGACTTAGTGCAAGGCG |
| | | | AGCCAGCAAGGAGGGAGGACAGGTGGCAGTGGGGGGTGA |
| | | | GGAGCATCTAAAAATAGCCACTCTTGGGTTTCTGATAGGC |
| | | | ACTGACTCTCTCTGCCTATTGGTCTATTTTCCCACCCTTAGG |
| | | | CTGCTAAAGGCCGCCACC |
| 218 | | tMCKmd | CCACTACGGGTCTAGGCTGCCCATGTAAGGAGGCAAGGCC |
| | | promoter | TGGGGACACCCGAGATGCCTGGTTATAATTAACCCAGACA |
| | | | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATCCCCACTACGGGTCTAGGCTGCCCATGTAAGGAGGCA |
| | | | AGGCCTGGGGACACCCGAGATGCCTGGTTATAATTAACCC |
| | | | AGACATGTGGCTGCCCCCCCCCCCCAACACCTGCTGCCTG |
| | | | AGCCTCACCCCCACCCCGGTGCCTGGGTCTTAGGCTCTGTA |
| | | | CACCATGGAGGAGAAGCTCGCTCTAAAAATAACCCTGTCC |
| | | | CTGGTGGATCCCCACTACGGGTCTAGGCTGCCCATGTAAG |
| | | | GAGGCAAGGCCTGGGGACACCCGAGATGCCTGGTTATAAT |
| | | | TAACCCAGACATGTGGCTGCCCCCCCCCCCCAACACCTGCT |
| | | | GCCTGAGCCTCACCCCCACCCCGGTGCCTGGGTCTTAGGCT |
| | | | CTGTACACCATGGAGGAGAAGCTCGCTCTAAAAATAACCC |
| | | | TGTCCCTGGTGGATCCCTCCCTGGGGACAGCCCCTCCTGGC |
| | | | TAGTCACACCCTGTAGGCTCCTCTATATAACCCAGGGGCA |
| | | | CAGGGGCTGCCCTCAGTCCGCCTGGAGACCTCGAGCCGAG |
| | | | TGGTCGTCTCAGGAGCC |
| 148 | | HBG/SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE (part | CAGCCCACCTGTCCCAGTGCTGACTTAGTGCAAGGCGAGC |
| | | of HBG/SIE | CAGCAAGGAGGGAGGACAGGTGGCAGTGGGGGGTGAGGA |
| | | intron) | GCATCTAAAAATAGCC |
| 24 | M24 | tMCK-MD- | CCACTACGGGTCTAGGCTGCCCATGTAAGGAGGCAAGGCC |
| | | HBBi-SIE- | TGGGGACACCCGAGATGCCTGGTTATAATTAACCCAGACA |
| | | hDES715p | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATCCCCACTACGGGTCTAGGCTGCCCATGTAAGGAGGCA |
| | | | AGGCCTGGGGACACCCGAGATGCCTGGTTATAATTAACCC |
| | | | AGACATGTGGCTGCCCCCCCCCCCCAACACCTGCTGCCTG |
| | | | AGCCTCACCCCCACCCCGGTGCCTGGGTCTTAGGCTCTGTA |
| | | | CACCATGGAGGAGAAGCTCGCTCTAAAAATAACCCTGTCC |
| | | | CTGGTGGATCCCCACTACGGGTCTAGGCTGCCCATGTAAG |
| | | | GAGGCAAGGCCTGGGGACACCCGAGATGCCTGGTTATAAT |
| | | | TAACCCAGACATGTGGCTGCCCCCCCCCCCCAACACCTGCT |
| | | | GCCTGAGCCTCACCCCCACCCCGGTGCCTGGGTCTTAGGCT |
| | | | CTGTACACCATGGAGGAGAAGCTCGCTCTAAAAATAACCC |
| | | | TGTCCCTGGTGGATCCCTCCCTGGGGACAGCCCCTCCTGGC |
| | | | TAGTCACACCCTGTAGGCTCCTCTATATAACCCAGGGGCA |
| | | | CAGGGGCTGCCCTCAGTCCGCCTGGAGACCTCGAGCCGAG |
| | | | TGGTCGTCTCAGGAGCCGGGCAGGTTGGTATCAAGGTTAC |
| | | | AAGCAGCCCACCTGTCCCAGTGCTGACTTAGTGCAAGGCG |
| | | | AGCCAGCAAGGAGGGAGGACAGGTGGCAGTGGGGGGTGA |
| | | | GGAGCATCTAAAAATAGCCACCCATGCCTCCTCAGGTACC |
| | | | CCCTGCCCCCCACAGCTCCTCTCCTGTGCCTTGTTTCCCAG |
| | | | CCATGCGTTCTCCTCTATAAATACCCGCTCTGGTATTTGGG |
| | | | GTTGGCAGCTGTTGCTGCCAGGGAGATGGTTGGGTTGACA |
| | | | TGCGGCTCCTGACAAAACACAAACCCCTGGTGTGTGTGGG |
| | | | CGTGGGTGGTGTGAGTAGGGGGATGAATCAGGGAGGGGG |
| | | | CGGGGGACCCAGGGGGCAGGAGCCACACAAAGTCTGTGC |
| | | | GGGGGTGGGAGCGCACATAGCAATTGGAAACTGAAAGCTT |
| | | | ATCAGACCCTTTCTGGAAATCAGCCCACTGTTTATAAACTT |
| | | | GAGGCCCCACCCTCGACGGAGATAACCAGGGCTGAAAGA |
| | | | GGCCCGCCTGGGGGCTGCAGACATGCTTGCTGCCTGCCCT |
| | | | GGCGAAGGATTGGCAGGCTTGCCCGTCACAGGACCCCCGC |
| | | | TGGCTGACTCAGGGGCGCAGGCCTCTTGCGGGGGAGCTGG |
| | | | CCTCCCCGCCCCCACGGCCACGGGCCGCCCTTTCCTGGCAG |
| | | | GACAGCGGGATCTTGCAGCTGTCAGGGGAGGGGAGGCGG |
| | | | GGGCTGATGTCAGGAGGGATATATAAAGTGCCGACGGCTG |
| | | | GGGGCCCTTCAGTCCGCCTGGAGACCTCGAGCCGAGTGGT |
| | | | CGTACTCTTGGGTTTCTGATAGGCACTGACTCTCTCTGCCT |
| | | | ATTGGTCTATTTTCCCACCCTTAGGCTGCTAAAGGCCGCCA |
| | | | CC |
| 218 | | tMCKmd | CCACTACGGGTCTAGGCTGCCCATGTAAGGAGGCAAGGCC |
| | | promoter | TGGGGACACCCGAGATGCCTGGTTATAATTAACCCAGACA |
| | | | TGTGGCTGCCCCCCCCCCCCAACACCTGCTGCCTGAGCCTC |
| | | | ACCCCCACCCCGGTGCCTGGGTCTTAGGCTCTGTACACCAT |
| | | | GGAGGAGAAGCTCGCTCTAAAAATAACCCTGTCCCTGGTG |
| | | | GATCCCCACTACGGGTCTAGGCTGCCCATGTAAGGAGGCA |
| | | | AGGCCTGGGGACACCCGAGATGCCTGGTTATAATTAACCC |
| | | | AGACATGTGGCTGCCCCCCCCCCCCAACACCTGCTGCCTG |
| | | | AGCCTCACCCCCACCCCGGTGCCTGGGTCTTAGGCTCTGTA |
| | | | CACCATGGAGGAGAAGCTCGCTCTAAAAATAACCCTGTCC |
| | | | CTGGTGGATCCCCACTACGGGTCTAGGCTGCCCATGTAAG |
| | | | GAGGCAAGGCCTGGGGACACCCGAGATGCCTGGTTATAAT |
| | | | TAACCCAGACATGTGGCTGCCCCCCCCCCCCAACACCTGCT |
| | | | GCCTGAGCCTCACCCCCACCCCGGTGCCTGGGTCTTAGGCT |
| | | | CTGTACACCATGGAGGAGAAGCTCGCTCTAAAAATAACCC |
| | | | TGTCCCTGGTGGATCCCTCCCTGGGGACAGCCCCTCCTGGC |
| | | | TAGTCACACCCTGTAGGCTCCTCTATATAACCCAGGGGCA |
| | | | CAGGGGCTGCCCTCAGTCCGCCTGGAGACCTCGAGCCGAG |
| | | | TGGTCGTCTCAGGAGCC |
| 149 | | HBG/SIE/ | GGGCAGGTTGGTATCAAGGTTACAAGCAGCCCACCTGTCC |
| | | hDES715md | CAGTGCTGACTTAGTGCAAGGCGAGCCAGCAAGGAGGGA |
| | | intron | GGACAGGTGGCAGTGGGGGGTGAGGAGCATCTAAAAATA |
| | | | GCCCACCCATGCCTCCTCAGGTACCCCCTGCCCCCCACAGC |
| | | | TCCTCTCCTGTGCCTTGTTTCCCAGCCATGCGTTCTCCTCTA |
| | | | TAAATACCCGCTCTGGTATTTGGGGTTGGCAGCTGTTGCTG |
| | | | CCAGGGAGATGGTTGGGTTGACATGCGGCTCCTGACAAAA |
| | | | CACAAACCCCTGGTGTGTGTGGGCGTGGGTGGTGTGAGTA |
| | | | GGGGGATGAATCAGGGAGGGGGCGGGGGACCCAGGGGGC |
| | | | AGGAGCCACACAAAGTCTGTGCGGGGGGGGAGCGCACA |
| | | | TAGCAATTGGAAACTGAAAGCTTATCAGACCCTTTCTGGA |
| | | | AATCAGCCCACTGTTTATAAACTTGAGGCCCCACCCTCGAC |
| | | | GGAGATAACCAGGGCTGAAAGAGGCCCGCCTGGGGGCTG |
| | | | CAGACATGCTTGCTGCCTGCCCTGGCGAAGGATTGGCAGG |
| | | | CTTGCCCGTCACAGGACCCCCGCTGGCTGACTCAGGGGCG |
| | | | CAGGCCTCTTGCGGGGGAGCTGGCCTCCCCGCCCCCACGG |
| | | | CCACGGGCCGCCCTTTCCTGGCAGGACAGCGGGATCTTGC |
| | | | AGCTGTCAGGGGAGGGGAGGCGGGGGCTGATGTCAGGAG |
| | | | GGATATATAAAGTGCCGACGGCTGGGGGCCCTTCAGTCCG |
| | | | CCTGGAGACCTCGAGCCGAGTGGTCGTACTCTTGGGTTTCT |
| | | | GATAGGCACTGACTCTCTCTGCCTATTGGTCTATTTTCCCA |
| | | | CCCTTAGGCTGCT |
| 214 | | SIE (part | CAGCCCACCTGTCCCAGTGCTGACTTAGTGCAAGGCGAGC |
| | | of HBG/SIE/ | CAGCAAGGAGGGAGGACAGGTGGCAGTGGGGGGTGAGGA |
| | | hDES715md | GCATCTAAAAATAGCC |
| | | intron) | |
| 151 | | hDES715md | CACCCATGCCTCCTCAGGTACCCCCTGCCCCCCACAGCTCC |
| | | promoter | TCTCCTGTGCCTTGTTTCCCAGCCATGCGTTCTCCTCTATAA |
| | | (part of | ATACCCGCTCTGGTATTTGGGGTTGGCAGCTGTTGCTGCCA |
| | | HBG/SIE/ | GGGAGATGGTTGGGTTGACATGCGGCTCCTGACAAAACAC |
| | | hDES715md | AAACCCCTGGTGTGTGTGGGCGTGGGTGGTGTGAGTAGGG |
| | | intron) | GGATGAATCAGGGAGGGGGCGGGGGACCCAGGGGGCAGG |
| | | | AGCCACACAAAGTCTGTGCGGGGGTGGGAGCGCACATAGC |
| | | | AATTGGAAACTGAAAGCTTATCAGACCCTTTCTGGAAATC |
| | | | AGCCCACTGTTTATAAACTTGAGGCCCCACCCTCGACGGA |
| | | | GATAACCAGGGCTGAAAGAGGCCCGCCTGGGGGCTGCAG |
| | | | ACATGCTTGCTGCCTGCCCTGGCGAAGGATTGGCAGGCTT |
| | | | GCCCGTCACAGGACCCCCGCTGGCTGACTCAGGGGCGCAG |
| | | | GCCTCTTGCGGGGGAGCTGGCCTCCCCGCCCCCACGGCCA |
| | | | CGGGCCGCCCTTTCCTGGCAGGACAGCGGGATCTTGCAGC |
| | | | TGTCAGGGGAGGGGAGGCGGGGGCTGATGTCAGGAGGGA |
| | | | TATATAAAGTGCCGACGGCTGGGGGCCCTTCAGTCCGCCT |
| | | | GGAGACCTCGAGCCGAGTGGTCGT |
| 25 | M25 | SPc5-12a | GCCGTCCGCCTTCGGCACCATCCTCACGACACCCAAATAT |
| | | | GGCGACGGGTGAGGAATGGTGGGGAGTTATTTTTAGAGCG |
| | | | GTGAGGAATGGTGGGCAGGCAGCAGGTGTTGGCGCTCTAA |
| | | | AAATAACTCCCGGGAGTTATTTTTAGAGCGGAGGAATGGT |
| | | | GGACACCCAAATATGGCGACGGTTCCTCACCCGTCGCCAT |
| | | | ATTTGGGTGTCCGCCCTCGGCCGGGGCCGCATTCCTGGGG |
| | | | GCCGGGCGGCGCTCCCGCCCGCCTCGATAAAAGGCTCCGG |
| | | | GGCCGGCGGCGGCCCACGAGCTACCCGGAGGAGCGGGAG |
| | | | GCGGCCACC |
| 26 | M26 | SPc5-12b | GCCGTCCGCCTTCGGCACCATCCTCACGACACCCAAATAT |
| | | | GGCGACGGGTGAGGAATGGTGGGGAGTTATTTTTAGAGCG |
| | | | GTGAGGAATGGTGGGCAGGCAGCAGGTGTTGGCGCTCTAA |
| | | | AAATAACTCCCGGGAGTTATTTTTAGAGCGGAGGAATGGT |
| | | | GGACACCCAAATATGGCGACGGTTCCTCACCCGTCGCCAT |
| | | | ATTTGGGTGTCCGCCCTCGGCCGGGGCCGCATTCCTGGGG |
| | | | GCCGGGCGGCGCTCCCGCCCGCCTCTATATAACCCAGGGG |
| | | | CACAGGGGCTGCCCTCAGTCCGCCTGGAGACCTCGAGCCG |
| | | | AGTGGTCGTCTCAGGAGCCGGGCAGGTTGGTATCAAGGTT |
| | | | ACAAGCAGCCCACCTGTCCCAGTGCTGACTTAGTGCAAGG |
| | | | CGAGCCAGCAAGGAGGGAGGACAGGTGGCAGTGGGGGGT |
| | | | GAGGAGCATCTAAAAATAGCCACTCTTGGGTTTCTGATAG |
| | | | GCACTGACTCTCTCTGCCTATTGGTCTATTTTCCCACCCTTA |
| | | | GGCTGCTAAAGGCCGCCACC |
| 216 | | SPc5-12 | CGGCCGTCCGCCTTCGGCACCATCCTCACGACACCCAAAT |
| | | promoter | ATGGCGACGGGTGAGGAATGGTGGGGAGTTATTTTTAGAG |
| | | | CGGTGAGGAATGGTGGGCAGGCAGCAGGTGTTGGCGCTCT |
| | | | AAAAATAACTCCCGGGAGTTATTTTTAGAGCGGAGGAATG |
| | | | GTGGACACCCAAATATGGCGACGGTTCCTCACCCGTCGCC |
| | | | ATATTTGGGTGTCCGCCCTCGGCCGGGGCCGCATTCCTGGG |
| | | | GGCCGGGCGGCGCTCCCGCCCGCCTCTATATAACCCAGGG |
| | | | GCACAGGGGCTGCCCTCAGTCCGCCTGGAGACCTCGAGCC |
| | | | GAGTGGTCGT |
| 144 | | HBB intron- | CTCAGGAGCCGGGCAGGTTGGTATCAAGGTTACAAGCAGC |
| | | SIE | CCACCTGTCCCAGTGCTGACTTAGTGCAAGGCGAGCCAGC |
| | | | AAGGAGGGAGGACAGGTGGCAGTGGGGGGTGAGGAGCAT |
| | | | CTAAAAATAGCCACTCTTGGGTTTCTGATAGGCACTGACTC |
| | | | TCTCTGCCTATTGGTCTATTTTCCCACCCTTAGGCTGCTAA |
| | | | AGGCCGCCACC |
| 214 | | SIE | CAGCCCACCTGTCCCAGTGCTGACTTAGTGCAAGGCGAGC |
| | | | CAGCAAGGAGGGAGGACAGGTGGCAGTGGGGGGTGAGGA |
| | | | GCATCTAAAAATAGCC |
| 27 | M27 | SP301a | TGGTTGCTGACTAATTGAGATGCATGCTTTGCATACTTCTG |
| | | | CCTGCTGGGGAGCCTGGGGACTTTCCACACCGGGAGTTAT |
| | | | TTTTAGAGCGATATGGCGACGGCCACTTGGCAGTACATCA |
| | | | ATTGGAAAGTCCCGTTAGTGCTATTGGCTGGCCAGCCAAT |
| | | | AGCTGGTTGCTGACTAATTGAGATGCATGCTTTGCATACTT |
| | | | CTGCCTGCTGGGGAGCCTGGGGACTTTCCACACCGTGAGG |
| | | | AATGGTGCCAGGCATCTCGGGTTTGATGTACTGCCAAGCC |
| | | | AGCCAATAGCCGCTCTAAAAATAACTCCCCCGTCGCCATA |
| | | | TTTGGGTGTCCGCTCTAAAAATAACTCCCGGCCGTCGCCAT |
| | | | ATGGGCCCCTCCCTGGGGACAGCCCCTTGGAAAGTCCCGT |
| | | | TAGTGCCAGGCGGGCCATTTACCGTCGCTCTAAAAATAAC |
| | | | TCCCTAGGGAAACCTGAAGCCCCCATTGACGTCAATGGGG |
| | | | CAGGTTTTTTATATAGTCCGGCTTCAGGTTTCCCTACAAGG |
| | | | CCTGGGGACAACCCGAGATGCCTGGCAGGTTTTTTATATA |
| | | | GTCCACCAAGTCAGCA |
| 28 | M28 | SP301b | TGGTTGCTGACTAATTGAGATGCATGCTTTGCATACTTCTG |
| | | | CCTGCTGGGGAGCCTGGGGACTTTCCACACCGGGAGTTAT |
| | | | TTTTAGAGCGATATGGCGACGGCCACTTGGCAGTACATCA |
| | | | ATTGGAAAGTCCCGTTAGTGCTATTGGCTGGCCAGCCAAT |
| | | | AGCTGGTTGCTGACTAATTGAGATGCATGCTTTGCATACTT |
| | | | CTGCCTGCTGGGGAGCCTGGGGACTTTCCACACCGTGAGG |
| | | | AATGGTGCCAGGCATCTCGGGTTTGATGTACTGCCAAGCC |
| | | | AGCCAATAGCCGCTCTAAAAATAACTCCCCCGTCGCCATA |
| | | | TTTGGGTGTCCGCTCTAAAAATAACTCCCGGCCGTCGCCAT |
| | | | ATGGGCCCCTCCCTGGGGACAGCCCCTTGGAAAGTCCCGT |
| | | | TAGTGCCAGGCGGGCCATTTACCGTCGCTCTAAAAATAAC |
| | | | TCCCTAGGGAAACCTGAAGCCCCCATTGACGTCAATGGGG |
| | | | CAGGTTTTTTATATAGTCCGGCTTCAGGTTTCCCTACAAGG |
| | | | CCTGGGGACAACCCGAGATGCCTGGCAGGTTTTTTATATA |
| | | | ACCCAGGGGCACAGGGGCTGCCCTCAGTCCGCCTGGAGAC |
| | | | CTCGAGCCGAGTGGTCGTCTCAGGAGCCGGGCAGGTTGGT |
| | | | ATCAAGGTTACAAGCAGCCCACCTGTCCCAGTGCTGACTT |
| | | | AGTGCAAGGCGAGCCAGCAAGGAGGGAGGACAGGTGGCA |
| | | | GTGGGGGGTGAGGAGCATCTAAAAATAGCCACTCTTGGGT |
| | | | TTCTGATAGGCACTGACTCTCTCTGCCTATTGGTCTATTTTC |
| | | | CCACCCTTAGGCTGCTAAAGGCCGCCACC |
| 215 | | SP-301 | TGGTTGCTGACTAATTGAGATGCATGCTTTGCATACTTCTG |
| | | promoter | CCTGCTGGGGAGCCTGGGGACTTTCCACACCGGGAGTTAT |
| | | | TTTTAGAGCGATATGGCGACGGCCACTTGGCAGTACATCA |
| | | | ATTGGAAAGTCCCGTTAGTGCTATTGGCTGGCCAGCCAAT |
| | | | AGCTGGTTGCTGACTAATTGAGATGCATGCTTTGCATACTT |
| | | | CTGCCTGCTGGGGAGCCTGGGGACTTTCCACACCGTGAGG |
| | | | AATGGTGCCAGGCATCTCGGGTTTGATGTACTGCCAAGCC |
| | | | AGCCAATAGCCGCTCTAAAAATAACTCCCCCGTCGCCATA |
| | | | TTTGGGTGTCCGCTCTAAAAATAACTCCCGGCCGTCGCCAT |
| | | | ATGGGCCCCTCCCTGGGGACAGCCCCTTGGAAAGTCCCGT |
| | | | TAGTGCCAGGCGGGCCATTTACCGTCGCTCTAAAAATAAC |
| | | | TCCCTAGGGAAACCTGAAGCCCCCATTGACGTCAATGGGG |
| | | | CAGGTTTTTTATATAGTCCGGCTTCAGGTTTCCCTACAAGG |
| | | | CCTGGGGACAACCCGAGATGCCTGGCAGGTTTTTTATATA |
| | | | ACCCAGGGGCACAGGGGCTGCCCTCAGTCCGCCTGGAGAC |
| | | | CTCGAGCCGAGTGGTCGT |
| 150 | | hBG-SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE | CAGCCCACCTGTCCCAGTGCTGACTTAGTGCAAGGCGAGC |
| | | | CAGCAAGGAGGGAGGACAGGTGGCAGTGGGGGGTGAGGA |
| | | | GCATCTAAAAATAGCC |
| 29 | M29 | CK6 | CACTACGGGTCTAGGCTGCCCATGTAAGGAGGCAAGGCCT |
| | | | GGGGACACCCGAGATGCCTGGTTATAATTAACCCAGACAT |
| | | | GTGGCTGCCCCCCCCCCCCAACACCTGCTGCCTGAGCCTCA |
| | | | CCCCCACCCCGGTGCCTGGGTCTTAGGCTCTGTACACCATG |
| | | | GAGGAGAAGCTCGCTCTAAAAATAACCCTGTCCCTGGTGG |
| | | | ATCCATCAAGGCTGTGGGGGACTGAGGGCAGGCTGTAACA |
| | | | GGCTTGGGGGCCAGGGCTTATACGTGCCTGGGACTCCCAA |
| | | | AGTATTACTGTTCCATGTTCCCGGCGAAGGGCCAGCTGTCC |
| | | | CCCGCCAGCTAGACTCAGCACTTAGTTTAGGAACCAGTGA |
| | | | GCAAGTCAGCCCTTGGGGCAGCCCATACAAGGCCATGGGG |
| | | | CTGGGCAAGCTGCACGCCTGGGTCCGGGGTGGGCACGGTG |
| | | | CCCGGGCAACGAGCTGAAAGCTCATCTACTCTCAGGGGCC |
| | | | CCTCCCTGGGGACAGCCCCTCCTGGCTAGTCACACCCTGTA |
| | | | GGCTCCTCTATATAACCCAGGGGCACAGGGGCTGCCCCCG |
| | | | GGTCACCGCCACC |
| 30 | M30 | CK8.3 | CACTACGGGTCTAGGCTGCCCATGTAAGGAGGCAAGGCCT |
| | | | GGGGACACCCGAGATGCCTGGTTATAATTAACCCAGACAT |
| | | | GTGGCTGCCCCCCCCCCCCAACACCTGCTGCCCTCTAAAAA |
| | | | TAACCCTGTCCCTGGTGGATCCACACTACGGGTCTAGGCTG |
| | | | CCCATGTAAGGAGGCAAGGCCTGGGGACACCCGAGATGCC |
| | | | TGGTTATAATTAACCCAGACATGTGGCTGCCCCCCCCCCCC |
| | | | AACACCTGCTGCCCTCTAAAAATAACCCTGTCCCTGGTGG |
| | | | ATCCACACTACGGGTCTAGGCTGCCCATGTAAGGAGGCAA |
| | | | GGCCTGGGGACACCCGAGATGCCTGGTTATAATTAACCCA |
| | | | GACATGTGGCTGCCCCCCCCCCCCAACACCTGCTGCCCTCT |
| | | | AAAAATAACCCTGTCCCTGGTGGATCCATCAAGGCTGTGG |
| | | | GGGACTGAGGGCAGGCTGTAACAGGCTTGGGGGCCAGGG |
| | | | CTTATACGTGCCTGGGACTCCCAAAGTATTACTGTTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCCCGGGTCACCACCACCTCC |
| | | | ACAGCACAGACAGACACTCAGGAGCCAGCCAGCCGCCAC |
| | | | C |
| 31 | M31 | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 32 | M32 | CK8emd | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCAGTCCGCCTGGAGACCT |
| | | | CGAGCCGAGTGGTCGTCTCAGGAGCCAGCCAGCCGCCACC |
| 33 | M33 | CK8emd- | TGCCCATGTAAGGAGGCAAGGCCTGGGGACACCCGAGATG |
| | | hBG-SIE | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCAGTCCGCCTGGAGACCT |
| | | | CGAGCCGAGTGGTCGTCTCAGGAGCCAGCCAGCCGGGCAG |
| | | | GTTGGTATCAAGGTTACAAGCAGCCCACCTGTCCCAGTGC |
| | | | TGACTTAGTGCAAGGCGAGCCAGCAAGGAGGGAGGACAG |
| | | | GTGGCAGTGGGGGGTGAGGAGCATCTAAAAATAGCCACTC |
| | | | TTGGGTTTCTGATAGGCACTGACTCTCTCTGCCTATTGGTC |
| | | | TATTTTCCCACCCTTAGGCTGCTAAAGGCCGCCACC |
| 154 | | MCK | TGCCCATGTAAGGAGGCAAGGCCTGGGGACACCCGAGATG |
| | | enhancer | CCTGGTTATAATTAACCCAGACATGTGGCTGCCCCCCCCCC |
| | | part of | CCAACACCTGCTGCCCTCTAAAAATAACCCTG |
| | | CK8emd | |
| | | promoter) | |
| 133 | | CK8emd | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCAGTCCGCCTGGAGACCT |
| | | | CGAGCCGAGTGGTCGT |
| 150 | | hBG-SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE (part | CAGCCCACCTGTCCCAGTGCTGACTTAGTGCAAGGCGAGC |
| | | of hBG-SIE | CAGCAAGGAGGGAGGACAGGTGGCAGTGGGGGGTGAGGA |
| | | intron) | GCATCTAAAAATAGCC |
| 34 | M34 | mDESe- | TCGCTGTCAGCCTTCCTTGACACCTCTGTCTCCTCAGGTGC |
| | | MCKe- | CTGGCTCCCAGTCCCCAGAACGCCTCTCCTGTACCTTGCTT |
| | | TPT1p- | CCTAGCTGGGCCTTTCCTTCTCCTCTATAAATACCAGCTCT |
| | | MD-hBG- | GGTATTTCGCCTTGGCAGCTGTTGCCCATGTAAGGAGGCA |
| | | SIE | AGGCCTGGGGACACCCGAGATGCCTGGTTATAATTAACCC |
| | | | AGACATGTGGCTGCCCCCCCCCCCCAACACCTGCTGCCCTC |
| | | | TAAAAATAACCCTGCCTCCCACGCTGCGCGCGCACCTCCC |
| | | | CGCCCCCACCCCTACCCGCTGGCGTGCCCAGTGGAACGGA |
| | | | GCCTTGTGTCTCCGCCTCAAGTCCCCGGATGCTCACCTCCC |
| | | | CGACTCGCCCCCGCTGTGGCCCCGCCCCCGCGCGGCTCTTC |
| | | | GTGCCACGTCACCGCCTGCGTCGCTTCCGGAGGCGCAGCG |
| | | | GGCGATGACGTAGAGGGACGTGCCCTCTATATAAGGTTGG |
| | | | GGAACAGGGGCTGCCCTCAGTCCGCCTGGAGACCTCGAGC |
| | | | CGAGTGGTCGTGCGGCTGAGTCGGCCTTTTCCGCCCGCTCC |
| | | | CCCCTCCCCCCGAGCGCCGCTCCGGCTGCACCGCGCTCGCT |
| | | | CCGAGTTTCAGGCTCGTGCTAAGCTAGCGCCGTCGTCGTCT |
| | | | CCCTTCAGTCCTCAGGAGCCGGGCAGGTTGGTATCAAGGT |
| | | | TACAAGCAGCCCACCTGTCCCAGTGCTGACTTAGTGCAAG |
| | | | GCGAGCCAGCAAGGAGGGAGGACAGGTGGCAGTGGGGGG |
| | | | TGAGGAGCATCTAAAAATAGCCACTCTTGGGTTTCTGATA |
| | | | GGCACTGACTCTCTCTGCCTATTGGTCTATTTTCCCACCCTT |
| | | | AGGCTGCTAAAGGCCGCCACC |
| 165 | | mDES.1 | TCGCTGTCAGCCTTCCTTGACACCTCTGTCTCCTCAGGTGC |
| | | enhancer | CTGGCTCCCAGTCCCCAGAACGCCTCTCCTGTACCTTGCTT |
| | | | CCTAGCTGGGCCTTTCCTTCTCCTCTATAAATACCAGCTCT |
| | | | GGTATTTCGCCTTGGCAGCTGT |
| 154 | | MCK | TGCCCATGTAAGGAGGCAAGGCCTGGGGACACCCGAGATG |
| | | enhancer | CCTGGTTATAATTAACCCAGACATGTGGCTGCCCCCCCCCC |
| | | | CCAACACCTGCTGCCCTCTAAAAATAACCCTG |
| 220 | | TPT1md | CCTCCCACGCTGCGCGCGCACCTCCCCGCCCCCACCCCTAC |
| | | promoter | CCGCTGGCGTGCCCAGTGGAACGGAGCCTTGTGTCTCCGC |
| | | | CTCAAGTCCCCGGATGCTCACCTCCCCGACTCGCCCCCGCT |
| | | | GTGGCCCCGCCCCCGCGCGGCTCTTCGTGCCACGTCACCGC |
| | | | CTGCGTCGCTTCCGGAGGCGCAGCGGGCGATGACGTAGAG |
| | | | GGACGTGCCCTCTATATAAGGTTGGGGAACAGGGGCTGCC |
| | | | CTCAGTCCGCCTGGAGACCTCGAGCCGAGTGGTCGTGCGG |
| | | | CTGAGTCGGCCTTTTCCGCCCGCTCCCCCCTCCCCCCGAGC |
| | | | GCCGCTCCGGCTGCACCGCGCTCGCTCCGAGTTTCAGGCTC |
| | | | GTGCTAAGCTAGCGCCGTCGTCGTCTCCCTTCAGTC |
| 150 | | hBG-SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE (part | CAGCCCACCTGTCCCAGTGCTGACTTAGTGCAAGGCGAGC |
| | | of hBG-SIE | CAGCAAGGAGGGAGGACAGGTGGCAGTGGGGGGTGAGGA |
| | | intron) | GCATCTAAAAATAGCC |
| 35 | M35 | GAPDH- | GAGGACACAGCCTGGCTCTGGGTCCCAGCAGGAAAGGCA |
| | | MD-hBG- | ATCCCAGAAAGGCAGGGTCAGGGACTGGAGTCCTGTGGGT |
| | | SIE | GCTTTTTAAGCAAAGATTATCACCAGGCAGGCTAAACTTA |
| | | | GCAACCGGCTTTTAGCTAGAAGGGCAGGGGGCTGGTGTCA |
| | | | GGTTATGCTGGGCCAGCAAAGAGGCCCGGGATCCCCCTCC |
| | | | CATGCACCTGCTGATGGGCCAAGCAGTGAAAGGGAGTCCC |
| | | | TGGCTCCTGGCTCGCCTGCACGTCCCAGGGCGGGGAGGGA |
| | | | CTTCCGCCCTCACGTCCCGCTCTTCGCAGGTTTCCAGATCC |
| | | | TGTCCTCCGCCTGGGGCCCCAGCCCGGAGAGAGCCGCTGG |
| | | | TGCACACAGGGCCGGGATTGTCTGCCCTAATTATCAGGTC |
| | | | CAGGCCCCCTCCCCCTCAAGCCGCCTCCCGAGCCTCCTTCC |
| | | | TCTAGATGGGGAGGGGGAAGTGGGGGCTGGGAAGGAACC |
| | | | ACGGGCCCCCGCCCGAGGCCCATGGGCCCCTCCTCTTTCCC |
| | | | GCCTCTCAGCCTTTGAAAGAAAGAAAGGGGAGGGGGCAG |
| | | | GTCCCAAAGTCCTCCTGTTTCATCCAAGCGTGTAAGGGTCC |
| | | | CCGTCCTTGACTCCCTAGTGTCCTGCTGCCCACAGTCCAGT |
| | | | CCTGGGAACCAGCACCGATCACCTCCCATCGGGCCAATCT |
| | | | CAGTCCCTTCCCCCCTACGTCGGGGCCCACACGCTCGGTGC |
| | | | GTGCCCAGTTGAACCAGGCGGCTGCGGAAAAAAAAAAGC |
| | | | GGGGAGAAAGTAGGGCCCGGCTACTAGCGGTTTTACGGGC |
| | | | GCACGTAGCTCAGGCCTCAAGACCTTGGGCTGGGACTGGC |
| | | | TGAGCCTGGCGGGAGGCGGGGTCCGAGTCACCGCCTGCCG |
| | | | CCGCGCCCCCGGTTTCTATAAATTGAGCCCGCAGCCTCCCG |
| | | | CTTCAGTCCGCCTGGAGACCTCGAGCCGAGTGGTCGTATCT |
| | | | TCTTTTGCGTCGCCAGGTGAAGACGGGCGGGGGCAGGTTG |
| | | | GTATCAAGGTTACAAGCAGCCCACCTGTCCCAGTGCTGAC |
| | | | TTAGTGCAAGGCGAGCCAGCAAGGAGGGAGGACAGGTGG |
| | | | CAGTGGGGGGTGAGGAGCATCTAAAAATAGCCACTCTTGG |
| | | | GTTTCTGATAGGCACTGACTCTCTCTGCCTATTGGTCTATTT |
| | | | TCCCACCCTTAGGCTGCTAAAGGCCGCCACC |
| 142 | | GAPDHmd | GAGGACACAGCCTGGCTCTGGGTCCCAGCAGGAAAGGCA |
| | | promoter | ATCCCAGAAAGGCAGGGTCAGGGACTGGAGTCCTGTGGGT |
| | | | GCTTTTTAAGCAAAGATTATCACCAGGCAGGCTAAACTTA |
| | | | GCAACCGGCTTTTAGCTAGAAGGGCAGGGGGCTGGTGTCA |
| | | | GGTTATGCTGGGCCAGCAAAGAGGCCCGGGATCCCCCTCC |
| | | | CATGCACCTGCTGATGGGCCAAGCAGTGAAAGGGAGTCCC |
| | | | TGGCTCCTGGCTCGCCTGCACGTCCCAGGGCGGGGAGGGA |
| | | | CTTCCGCCCTCACGTCCCGCTCTTCGCAGGTTTCCAGATCC |
| | | | TGTCCTCCGCCTGGGGCCCCAGCCCGGAGAGAGCCGCTGG |
| | | | TGCACACAGGGCCGGGATTGTCTGCCCTAATTATCAGGTC |
| | | | CAGGCCCCCTCCCCCTCAAGCCGCCTCCCGAGCCTCCTTCC |
| | | | TCTAGATGGGGAGGGGGAAGTGGGGGCTGGGAAGGAACC |
| | | | ACGGGCCCCCGCCCGAGGCCCATGGGCCCCTCCTCTTTCCC |
| | | | GCCTCTCAGCCTTTGAAAGAAAGAAAGGGGAGGGGGCAG |
| | | | GTCCCAAAGTCCTCCTGTTTCATCCAAGCGTGTAAGGGTCC |
| | | | CCGTCCTTGACTCCCTAGTGTCCTGCTGCCCACAGTCCAGT |
| | | | CCTGGGAACCAGCACCGATCACCTCCCATCGGGCCAATCT |
| | | | CAGTCCCTTCCCCCCTACGTCGGGGCCCACACGCTCGGTGC |
| | | | GTGCCCAGTTGAACCAGGCGGCTGCGGAAAAAAAAAAGC |
| | | | GGGGAGAAAGTAGGGCCCGGCTACTAGCGGTTTTACGGGC |
| | | | GCACGTAGCTCAGGCCTCAAGACCTTGGGCTGGGACTGGC |
| | | | TGAGCCTGGCGGGAGGCGGGGTCCGAGTCACCGCCTGCCG |
| | | | CCGCGCCCCCGGTTTCTATAAATTGAGCCCGCAGCCTCCCG |
| | | | CTTCAGTCCGCCTGGAGACCTCGAGCCGAGTGGTCGTATCT |
| | | | TCTTTTGCGTCGCCAGGTGAAGACGGGCGG |
| 150 | | hBG-SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE (part | CAGCCCACCTGTCCCAGTGCTGACTTAGTGCAAGGCGAGC |
| | | of hBG-SIE | CAGCAAGGAGGGAGGACAGGTGGCAGTGGGGGGTGAGGA |
| | | intron) | GCATCTAAAAATAGCC |
| 36 | M36 | SPc5e- | CGGCCGTCCGCCTTCGGCACCATCCTCACGACACCCAAAT |
| | | MT1Xp- | ATGGCGACGGGTGAGGAATGGTGGGGAGTTATTTTTAGAG |
| | | MD-hBG- | CGGTGAGGAATGGTGGGCAGGCAGCAGGTGTTGGCGCTCT |
| | | SIE | AAAAATAACTCCCGGGAGTTATTTTTAGAGCGGAGGAATG |
| | | | GTGGACACCCAAATATGGCGACGGTTCCTCACCCGTCGCC |
| | | | ATATTTGGGTGTCCGCCCTCGGCCGGGGCCGCATTCCTGGG |
| | | | GGCCGGGCGGCGCTCCCGCCCGCCTCAAAGTGACTAAAAG |
| | | | GAAGGAAAAAACCTAAAATGTTTCCAAGTGGTGGGGTGTA |
| | | | TGGGGAGAAATGTGCCCATTCAGCCTCTCAAGGGGGCAGA |
| | | | GCCCTGTGTCTGTTCTGTCCCTAGACCGAGGTGGGGACGG |
| | | | GGACAGGGACCGGCAATGCAGGAAATCCCTGCCTCTCATT |
| | | | TGCACACTTGGGAAATGCAAAGCTACACACAGTGGAGCGG |
| | | | GGGCTGGGGGTGTGTAGACCGCCGGGATTCCGAGTTTCCC |
| | | | GGCACGCCTAGGAGAGGGAGAGGCAGGCAATGTCAGGGA |
| | | | AATTGGGCAGGCAAGACGCCAGGGACGCCACGTACTGCCA |
| | | | GGTTCTCAACGAGGTGGAGCCAAAGGGGCAGGCCCCGCG |
| | | | GTGCGCCCGGCGCTGGGCTCACGGGTTGCTGCACCCGGCC |
| | | | CAGGATCGCGGGCGGTGCAGACTCAGCAGGGGGGGGTGC |
| | | | AAGGACGAGGCGGGGCCTCTGCGCCCGGCCCTCTTCCCGG |
| | | | ACTATAAAAAGAGCCGCCGGCTTCTGGGCTCAGTTCGCCT |
| | | | GGAGACCTCGAGCCGAGTGGTCGTCGCTTTTCATCTGTCCC |
| | | | GCTGCGTGTTTTCCTCTTGATCGGGAACTCCTGCTTCTCCTT |
| | | | GCCGGGCAGGTTGGTATCAAGGTTACAAGCAGCCCACCTG |
| | | | TCCCAGTGCTGACTTAGTGCAAGGCGAGCCAGCAAGGAGG |
| | | | GAGGACAGGTGGCAGTGGGGGGTGAGGAGCATCTAAAAA |
| | | | TAGCCACTCTTGGGTTTCTGATAGGCACTGACTCTCTCTGC |
| | | | CTATTGGTCTATTTTCCCACCCTTAGGCTGCTAAAGGCCGC |
| | | | CACC |
| 217 | | SPc5e | CGGCCGTCCGCCTTCGGCACCATCCTCACGACACCCAAAT |
| | | | ATGGCGACGGGTGAGGAATGGTGGGGAGTTATTTTTAGAG |
| | | | CGGTGAGGAATGGTGGGCAGGCAGCAGGTGTTGGCGCTCT |
| | | | AAAAATAACTCCCGGGAGTTATTTTTAGAGCGGAGGAATG |
| | | | GTGGACACCCAAATATGGCGACGGTTCCTCACCCGTCGCC |
| | | | ATATTTGGGTGTCCGCCCTCGGCCGGGGCCGCATTCCTGGG |
| | | | GGCCGGGCGGCGCTCCCGCCCGCCTC |
| 189 | | MT1Xmd | AAAGTGACTAAAAGGAAGGAAAAAACCTAAAATGTTTCC |
| | | promoter | AAGTGGTGGGGTGTATGGGGAGAAATGTGCCCATTCAGCC |
| | | | TCTCAAGGGGGCAGAGCCCTGTGTCTGTTCTGTCCCTAGAC |
| | | | CGAGGTGGGGACGGGGACAGGGACCGGCAATGCAGGAAA |
| | | | TCCCTGCCTCTCATTTGCACACTTGGGAAATGCAAAGCTAC |
| | | | ACACAGTGGAGCGGGGGCTGGGGGTGTGTAGACCGCCGG |
| | | | GATTCCGAGTTTCCCGGCACGCCTAGGAGAGGGAGAGGCA |
| | | | GGCAATGTCAGGGAAATTGGGCAGGCAAGACGCCAGGGA |
| | | | CGCCACGTACTGCCAGGTTCTCAACGAGGTGGAGCCAAAG |
| | | | GGGCAGGCCCCGCGGTGCGCCCGGCGCTGGGCTCACGGGT |
| | | | TGCTGCACCCGGCCCAGGATCGCGGGCGGTGCAGACTCAG |
| | | | CAGGGGCGGGTGCAAGGACGAGGCGGGGCCTCTGCGCCC |
| | | | GGCCCTCTTCCCGGACTATAAAAAGAGCCGCCGGCTTCTG |
| | | | GGCTCAGTTCGCCTGGAGACCTCGAGCCGAGTGGTCGTCG |
| | | | CTTTTCATCTGTCCCGCTGCGTGTTTTCCTCTTGATCGGGAA |
| | | | CTCCTGCTTCTCCTTGCC |
| 150 | | hBG-SIE | GGGCAGGTTGGTATCAAGGTTACAAGCAGCCCACCTGTCC |
| | | intron | CAGTGCTGACTTAGTGCAAGGCGAGCCAGCAAGGAGGGA |
| | | | GGACAGGTGGCAGTGGGGGGTGAGGAGCATCTAAAAATA |
| | | | GCCACTCTTGGGTTTCTGATAGGCACTGACTCTCTCTGCCT |
| | | | ATTGGTCTATTTTCCCACCCTTAGGCTGCT |
| 214 | | SIE (part | CAGCCCACCTGTCCCAGTGCTGACTTAGTGCAAGGCGAGC |
| | | of hBG-SIE | CAGCAAGGAGGGAGGACAGGTGGCAGTGGGGGGTGAGGA |
| | | intron) | GCATCTAAAAATAGCC |
| 37 | M37 | CK8emd2 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGAATTCC |
| | | | GAACGCAACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 156 | | md2 | TCATTCGTCTCTTGACCCCCGCCATACACGGTTCGCCTTCG |
| | | | CCTCAGGAGCCAGCCAGCC |
| 38 | M38 | CK8eMD2a | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGACCCCC |
| | | | GAACGCAACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 157 | | md2a | TCATTCGTCTCTTGACCCCCGAACGCAACGGTTCGCCTTCG |
| | | | CCTCAGGAGCCAGCCAGCC |
| 39 | M39 | CK8eMD2b | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGAATTCC |
| | | | GCCAGCAACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 158 | | md2b | TCATTCGTCTCTTGAATTCCGCCAGCAACGGTTCGCCTTCG |
| | | | CCTCAGGAGCCAGCCAGCC |
| 40 | M40 | CK8eMD2c | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGAATTCC |
| | | | GAACTACACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 159 | | md2c | TCATTCGTCTCTTGAATTCCGAACTACACGGTTCGCCTTCG |
| | | | CCTCAGGAGCCAGCCAGCC |
| 41 | M41 | CK8eMD2d | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGACCCCC |
| | | | GCCAGCAACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 160 | | md2d | TCATTCGTCTCTTGACCCCCGCCAGCAACGGTTCGCCTTCG |
| | | | CCTCAGGAGCCAGCCAGCC |
| 42 | M42 | CK8eMD2e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGACCCCC |
| | | | GAACTACACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 161 | | md2e | TCATTCGTCTCTTGACCCCCGAACTACACGGTTCGCCTTCG |
| | | | CCTCAGGAGCCAGCCAGCC |
| 43 | M43 | CK8eMD2f | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGAATTCC |
| | | | GCCATACACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 162 | | md2f | TCATTCGTCTCTTGAATTCCGCCATACACGGTTCGCCTTCG |
| | | | CCTCAGGAGCCAGCCAGCC |
| 44 | M44 | CK8eMD2g | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCGTCTCTTGACCCCC |
| | | | GCCATACACGGTTCGCCTTCGCCTCAGGAGCCAGCCAGCC |
| | | | GCCACC |
| 132 | | CK8e.1 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCC |
| 163 | | md2g | TCATTCGTCTCTTGACCCCCGCCATACACGGTTCGCCTTCG |
| | | | CCTCAGGAGCCAGCCAGCC |
| 45 | M45 | ttMCKe | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGTGCCCATGT |
| | | | AAGGAGGCAAGGCCTGGGGACACCCGAGATGCCTGGTTAT |
| | | | AATTAACCCAGACATGTGGCTGCCCCCCCCCCCCAACACC |
| | | | TGCTGCCCTCTAAAAATAACCCTGTGCCCATGTAAGGAGG |
| | | | CAAGGCCTGGGGACACCCGAGATGCCTGGTTATAATTAAC |
| | | | CCAGACATGTGGCTGCCCCCCCCCCCCAACACCTGCTGCCC |
| | | | TCTAAAAATAACCCTGCATTCCATGTTCCCGGCGAAGGGC |
| | | | CAGCTGTCCCCCGCCAGCTAGACTCAGCACTTAGTTTAGG |
| | | | AACCAGTGAGCAAGTCAGCCCTTGGGGCAGCCCATACAAG |
| | | | GCCATGGGGCTGGGCAAGCTGCACGCCTGGGTCCGGGGTG |
| | | | GGCACGGTGCCCGGGCAACGAGCTGAAAGCTCATCTACTC |
| | | | TCAGGGGCCCCTCCCTGGGGACAGCCCCTCCTGGCTAGTC |
| | | | ACACCCTGTAGGCTCCTCTATATAACCCAGGGGCACAGGG |
| | | | GCTGCCCTCATTCTACCACCACCTCCACAGCACAGACAGA |
| | | | CACTCAGGAGCCAGCCAGCCGCCACC |
| 154 | | MCK | TGCCCATGTAAGGAGGCAAGGCCTGGGGACACCCGAGATG |
| | | enhancer | CCTGGTTATAATTAACCCAGACATGTGGCTGCCCCCCCCCC |
| | | | CCAACACCTGCTGCCCTCTAAAAATAACCCTG |
| 154 | | MCK | TGCCCATGTAAGGAGGCAAGGCCTGGGGACACCCGAGATG |
| | | enhancer 1 | CCTGGTTATAATTAACCCAGACATGTGGCTGCCCCCCCCCC |
| | | | CCAACACCTGCTGCCCTCTAAAAATAACCCTG |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | (MCK | CCAACACCTGCTGCCCTCTAAAAATAACCCTGCATTCCATG |
| | | enhancer | TTCCCGGCGAAGGGCCAGCTGTCCCCCGCCAGCTAGACTC |
| | | sequence | AGCACTTAGTTTAGGAACCAGTGAGCAAGTCAGCCCTTGG |
| | | underlined) | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 222 | | CK8e | CATTCCATGTTCCCGGCGAAGGGCCAGCTGTCCCCCGCCA |
| | | (without | GCTAGACTCAGCACTTAGTTTAGGAACCAGTGAGCAAGTC |
| | | MCK | AGCCCTTGGGGCAGCCCATACAAGGCCATGGGGCTGGGCA |
| | | enhancer | AGCTGCACGCCTGGGTCCGGGGTGGGCACGGTGCCCGGGC |
| | | sequence) | AACGAGCTGAAAGCTCATCTACTCTCAGGGGCCCCTCCCT |
| | | | GGGGACAGCCCCTCCTGGCTAGTCACACCCTGTAGGCTCC |
| | | | TCTATATAACCCAGGGGCACAGGGGCTGCCCTCATTCTAC |
| | | | CACCACCTCCACAGCACAGACAGACACTCAGGAGCCAGCC |
| | | | AGCC |
| 46 | M46 | mDes- | AGCTTGTCGCTGTCAGCCTTCCTTGACACCTCTGTCTCCTC |
| | | CK8e | AGGTGCCTGGCTCCCAGTCCCCAGAACGCCTCTCCTGTACC |
| | | | TTGCTTCCTAGCTGGGCCTTTCCTTCTCCTCTATAAATACCA |
| | | | GCTCTGGTATTTCGCCTTGGCAGCTGTTGCTGCTAGGGAGA |
| | | | CGGCTGGCTTGACACCACTACGGGTCTAGGCTGCCCATGT |
| | | | AAGGAGGCAAGGCCTGGGGACACCCGAGATGCCTGGTTAT |
| | | | AATTAACCCAGACATGTGGCTGCCCCCCCCCCCCAACACC |
| | | | TGCTGCCCTCTAAAAATAACCCTGCATTCCATGTTCCCGGC |
| | | | GAAGGGCCAGCTGTCCCCCGCCAGCTAGACTCAGCACTTA |
| | | | GTTTAGGAACCAGTGAGCAAGTCAGCCCTTGGGGCAGCCC |
| | | | ATACAAGGCCATGGGGCTGGGCAAGCTGCACGCCTGGGTC |
| | | | CGGGGTGGGCACGGTGCCCGGGCAACGAGCTGAAAGCTC |
| | | | ATCTACTCTCAGGGGCCCCTCCCTGGGGACAGCCCCTCCTG |
| | | | GCTAGTCACACCCTGTAGGCTCCTCTATATAACCCAGGGG |
| | | | CACAGGGGCTGCCCTCATTCTACCACCACCTCCACAGCAC |
| | | | AGACAGACACTCAGGAGCCAGCCAGCCGCCACC |
| 164 | | mDes | AGCTTGTCGCTGTCAGCCTTCCTTGACACCTCTGTCTCCTC |
| | | enhancer | AGGTGCCTGGCTCCCAGTCCCCAGAACGCCTCTCCTGTACC |
| | | | TTGCTTCCTAGCTGGGCCTTTCCTTCTCCTCTATAAATACCA |
| | | | GCTCTGGTATTTCGCCTTGGCAGCTGTTGCTGCTAGGGAGA |
| | | | CGGCTGGCTTGACA |
| 129 | | CK8e | CCACTACGGGTCTAGGCTGCCCATGTAAGGAGGCAAGGCC |
| | | promoter | TGGGGACACCCGAGATGCCTGGTTATAATTAACCCAGACA |
| | | | TGTGGCTGCCCCCCCCCCCCAACACCTGCTGCCCTCTAAAA |
| | | | ATAACCCTGCATTCCATGTTCCCGGCGAAGGGCCAGCTGT |
| | | | CCCCCGCCAGCTAGACTCAGCACTTAGTTTAGGAACCAGT |
| | | | GAGCAAGTCAGCCCTTGGGGCAGCCCATACAAGGCCATGG |
| | | | GGCTGGGCAAGCTGCACGCCTGGGTCCGGGGTGGGCACGG |
| | | | TGCCCGGGCAACGAGCTGAAAGCTCATCTACTCTCAGGGG |
| | | | CCCCTCCCTGGGGACAGCCCCTCCTGGCTAGTCACACCCTG |
| | | | TAGGCTCCTCTATATAACCCAGGGGCACAGGGGCTGCCCT |
| | | | CATTCTACCACCACCTCCACAGCACAGACAGACACTCAGG |
| | | | AGCCAGCCAGCC |
| 47 | M47 | CK8e- | TGCCCATGTAAGGAGGCAAGGCCTGGGGACACCCGAGATG |
| | | BTImd2 | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGCGGCGCCTATATAAG |
| | | | GTGTGGGCGGGTGTGGCACAGCTAGTTCCGTCGCAGCCGG |
| | | | CGAGCCGAACGGTCGTCCGCGGATCCGCCACC |
| 131 | | CK8e | TGCCCATGTAAGGAGGCAAGGCCTGGGGACACCCGAGATG |
| | | (partial) | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAG |
| 126 | | BTImd2 | CGGCGCCTATATAAGGTGTGGGCGGGTGTGGCACAGCTAG |
| | | | TTCCGTCGCAGCCGGCGAGCCGAACGGTCGT |
| 48 | M48 | CK8e- | TGCCCATGTAAGGAGGCAAGGCCTGGGGACACCCGAGATG |
| | | BTImd2- | CCTGGTTATAATTAACCCAGACATGTGGCTGCCCCCCCCCC |
| | | MVMi | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGCGGCGCCTATATAAG |
| | | | GTGTGGGCGGGTGTGGCACAGCTAGTTCCGTCGCAGCCGG |
| | | | CGAGCCGAACGGTCGTTGAGCCAAGAGGTAAGGGTTTAAG |
| | | | GGATGGTTGGTTGGTGGGGTATTAATGTTTAATTACCTGGA |
| | | | GCACCTGCCTGAAATCACTTTTTTTCAGGTTGGCCGCGGAT |
| | | | CCGCCACC |
| 131 | | CK8e | TGCCCATGTAAGGAGGCAAGGCCTGGGGACACCCGAGATG |
| | | (partial) | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAG |
| 126 | | BTImd2 | CGGCGCCTATATAAGGTGTGGGCGGGTGTGGCACAGCTAG |
| | | | TTCCGTCGCAGCCGGCGAGCCGAACGGTCGT |
| 200 | | MVM intron | AAGAGGTAAGGGTTTAAGGGATGGTTGGTTGGTGGGGTAT |
| | | | TAATGTTTAATTACCTGGAGCACCTGCCTGAAATCACTTTT |
| | | | TTTCAGGTTGG |
| 49 | M49 | CK8e- | TGCCCATGTAAGGAGGCAAGGCCTGGGGACACCCGAGATG |
| | | sBTImd2- | CCTGGTTATAATTAACCCAGACATGTGGCTGCCCCCCCCCC |
| | | MVMi | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGGCGAAGCCGGTCC |
| | | | CCGCCCCGCGGGGCCCCGCCCCGCGTGCGCCGTTCCGAAA |
| | | | GTTGCTTTCTAGATCTCGCCCCGCCCCGGCGGCGCCTATAT |
| | | | AAGGTGTGGGCGGGTGTGGCACAGCTAGTTCCGTCGCAGC |
| | | | CGGCGAGCCGAACGGTCGTTGAGCCAAGAGGTAAGGGTTT |
| | | | AAGGGATGGTTGGTTGGTGGGGTATTAATGTTTAATTACCT |
| | | | GGAGCACCTGCCTGAAATCACTTTTTTTCAGGTTGGCCGCG |
| | | | GATCCGCCACC |
| 131 | | CK8e | TGCCCATGTAAGGAGGCAAGGCCTGGGGACACCCGAGATG |
| | | (partial) | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAG |
| 213 | | sBTImd2 | GGCGAAGCCGGTCCCCGCCCCGCGGGGCCCCGCCCCGCGT |
| | | | GCGCCGTTCCGAAAGTTGCTTTCTAGATCTCGCCCCGCCCC |
| | | | GGCGGCGCCTATATAAGGTGTGGGCGGGTGTGGCACAGCT |
| | | | AGTTCCGTCGCAGCCGGCGAGCCGAACGGTCGTTGAGCC |
| 200 | | MVM intron | AAGAGGTAAGGGTTTAAGGGATGGTTGGTTGGTGGGGTAT |
| | | | TAATGTTTAATTACCTGGAGCACCTGCCTGAAATCACTTTT |
| | | | TTTCAGGTTGG |
| 50 | M50 | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACC |
| 51 | M51 | MTF1291- | TAATTCAAGGCCTGGGGACAAATTCTGCATTCCTACTGAAT |
| | | MCKmin | TGGGCCCCTCCCTGGGGACAGCCCCAATTAGTGGGGGCGG |
| | | | GGCAGAATTCAAGGCCTGGGGACAAATTCTGCATTCCTAC |
| | | | TGAATTACCAAGTCAGCAAATTCTGCCAAATATGGACTAA |
| | | | TTCTGCTAAAAATAACTCCACTGAATTCTGCCAAATATGGA |
| | | | CTAATTCTGCCAAATATGGACTAATTACCTCCCTGGGGACA |
| | | | GCCCCTCCTGGCTAGTCACACCCTGTAGGCTCCTCTATATA |
| | | | ACCCAGGGGCACAGGGGCTGCCCTCATTCTACCACCACCT |
| | | | CCACAGCACAGACAGACACTCAGGAGCCAGCCAGCCGCC |
| | | | ACC |
| 196 | | MTF1291 | TAATTCAAGGCCTGGGGACAAATTCTGCATTCCTACTGAAT |
| | | | TGGGCCCCTCCCTGGGGACAGCCCCAATTAGTGGGGGCGG |
| | | | GGCAGAATTCAAGGCCTGGGGACAAATTCTGCATTCCTAC |
| | | | TGAATTACCAAGTCAGCAAATTCTGCCAAATATGGACTAA |
| | | | TTCTGCTAAAAATAACTCCACTGAATTCTGCCAAATATGGA |
| | | | CTAATTCTGCCAAATATGGACTAATTA |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 52 | M52 | MTF1291- | TAATTCAAGGCCTGGGGACAAATTCTGCATTCCTACTGAAT |
| | | CK8e | TGGGCCCCTCCCTGGGGACAGCCCCAATTAGTGGGGGCGG |
| | | | GGCAGAATTCAAGGCCTGGGGACAAATTCTGCATTCCTAC |
| | | | TGAATTACCAAGTCAGCAAATTCTGCCAAATATGGACTAA |
| | | | TTCTGCTAAAAATAACTCCACTGAATTCTGCCAAATATGGA |
| | | | CTAATTCTGCCAAATATGGACTAATTATGCCCATGTAAGG |
| | | | AGGCAAGGCCTGGGGACACCCGAGATGCCTGGTTATAATT |
| | | | AACCCAGACATGTGGCTGCCCCCCCCCCCCAACACCTGCT |
| | | | GCCCTCTAAAAATAACCCTGCATTCCATGTTCCCGGCGAA |
| | | | GGGCCAGCTGTCCCCCGCCAGCTAGACTCAGCACTTAGTTT |
| | | | AGGAACCAGTGAGCAAGTCAGCCCTTGGGGCAGCCCATAC |
| | | | AAGGCCATGGGGCTGGGCAAGCTGCACGCCTGGGTCCGGG |
| | | | GTGGGCACGGTGCCCGGGCAACGAGCTGAAAGCTCATCTA |
| | | | CTCTCAGGGGCCCCTCCCTGGGGACAGCCCCTCCTGGCTA |
| | | | GTCACACCCTGTAGGCTCCTCTATATAACCCAGGGGCACA |
| | | | GGGGCTGCCCTCATTCTACCACCACCTCCACAGCACAGAC |
| | | | AGACACTCAGGAGCCAGCCAGCCGCCACC |
| 196 | | MTF1291 | TAATTCAAGGCCTGGGGACAAATTCTGCATTCCTACTGAAT |
| | | | TGGGCCCCTCCCTGGGGACAGCCCCAATTAGTGGGGGCGG |
| | | | GGCAGAATTCAAGGCCTGGGGACAAATTCTGCATTCCTAC |
| | | | TGAATTACCAAGTCAGCAAATTCTGCCAAATATGGACTAA |
| | | | TTCTGCTAAAAATAACTCCACTGAATTCTGCCAAATATGGA |
| | | | CTAATTCTGCCAAATATGGACTAATTA |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 53 | M53 | MTF1173- | TATTCTGCATTCCTACTGAATTCAAGGCCTGGGGACAAATT |
| | | MCKmin | TAACCAGGCATCTCGGGTGTCCAATTGGGGCTGTCCCCAG |
| | | | GGAGGGGCCCAATTAGTCCATATTTGGCAGAATTCCAGGC |
| | | | ATATCGGGTAATTAGTGGGGGCGGGGCAGAATTTAGGGAA |
| | | | ACCTGAAGCCAATTCCTCCCTGGGGACAGCCCCTCCTGGCT |
| | | | AGTCACACCCTGTAGGCTCCTCTATATAACCCAGGGGCAC |
| | | | AGGGGCTGCCCTCATTCTACCACCACCTCCACAGCACAGA |
| | | | CAGACACTCAGGAGCCAGCCAGCCGCCACC |
| 190 | | MTF1173 | TATTCTGCATTCCTACTGAATTCAAGGCCTGGGGACAAATT |
| | | | TAACCAGGCATCTCGGGTGTCCAATTGGGGCTGTCCCCAG |
| | | | GGAGGGGCCCAATTAGTCCATATTTGGCAGAATTCCAGGC |
| | | | ATATCGGGTAATTAGTGGGGGCGGGGCAGAATTTAGGGAA |
| | | | ACCTGAAGCCAATT |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 54 | M54 | MTF1173- | TATTCTGCATTCCTACTGAATTCAAGGCCTGGGGACAAATT |
| | | CK8e | TAACCAGGCATCTCGGGTGTCCAATTGGGGCTGTCCCCAG |
| | | | GGAGGGGCCCAATTAGTCCATATTTGGCAGAATTCCAGGC |
| | | | ATATCGGGTAATTAGTGGGGGCGGGGCAGAATTTAGGGAA |
| | | | ACCTGAAGCCAATTTGCCCATGTAAGGAGGCAAGGCCTGG |
| | | | GGACACCCGAGATGCCTGGTTATAATTAACCCAGACATGT |
| | | | GGCTGCCCCCCCCCCCCAACACCTGCTGCCCTCTAAAAATA |
| | | | ACCCTGCATTCCATGTTCCCGGCGAAGGGCCAGCTGTCCCC |
| | | | CGCCAGCTAGACTCAGCACTTAGTTTAGGAACCAGTGAGC |
| | | | AAGTCAGCCCTTGGGGCAGCCCATACAAGGCCATGGGGCT |
| | | | GGGCAAGCTGCACGCCTGGGTCCGGGGTGGGCACGGTGCC |
| | | | CGGGCAACGAGCTGAAAGCTCATCTACTCTCAGGGGCCCC |
| | | | TCCCTGGGGACAGCCCCTCCTGGCTAGTCACACCCTGTAG |
| | | | GCTCCTCTATATAACCCAGGGGCACAGGGGCTGCCCTCAT |
| | | | TCTACCACCACCTCCACAGCACAGACAGACACTCAGGAGC |
| | | | CAGCCAGCCGCCACC |
| 190 | | MTF1173 | TATTCTGCATTCCTACTGAATTCAAGGCCTGGGGACAAATT |
| | | | TAACCAGGCATCTCGGGTGTCCAATTGGGGCTGTCCCCAG |
| | | | GGAGGGGCCCAATTAGTCCATATTTGGCAGAATTCCAGGC |
| | | | ATATCGGGTAATTAGTGGGGGCGGGGCAGAATTTAGGGAA |
| | | | ACCTGAAGCCAATT |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 55 | M55 | MTF1186- | TAATTTTGCTAAAAATAACTCCAGTGAATCCAGTAGGAAT |
| | | MCKmin | GCAGAATTATATGGTGCCGGCCAATTCTGCTAAAAATAAC |
| | | | TCCACTGAATTTAGGGAAACCTGAAGCCAATTTAGGGAAA |
| | | | CTTGAAGCCAATTCAGGGGAGTTATTTTTAGCAGAATTCA |
| | | | GTGGAGTTATTTTTAGCAGAATTCTGCCCCGCCCCCACTAA |
| | | | TTCCTCCCTGGGGACAGCCCCTCCTGGCTAGTCACACCCTG |
| | | | TAGGCTCCTCTATATAACCCAGGGGCACAGGGGCTGCCCT |
| | | | CATTCTACCACCACCTCCACAGCACAGACAGACACTCAGG |
| | | | AGCCAGCCAGCCGCCACC |
| 191 | | MTF1186 | TAATTTTGCTAAAAATAACTCCAGTGAATCCAGTAGGAAT |
| | | | GCAGAATTATATGGTGCCGGCCAATTCTGCTAAAAATAAC |
| | | | TCCACTGAATTTAGGGAAACCTGAAGCCAATTTAGGGAAA |
| | | | CTTGAAGCCAATTCAGGGGAGTTATTTTTAGCAGAATTCA |
| | | | GTGGAGTTATTTTTAGCAGAATTCTGCCCCGCCCCCACTAA |
| | | | TT |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 56 | M56 | MTF1186- | TAATTTTGCTAAAAATAACTCCAGTGAATCCAGTAGGAAT |
| | | CK8e | GCAGAATTATATGGTGCCGGCCAATTCTGCTAAAAATAAC |
| | | | TCCACTGAATTTAGGGAAACCTGAAGCCAATTTAGGGAAA |
| | | | CTTGAAGCCAATTCAGGGGAGTTATTTTTAGCAGAATTCA |
| | | | GTGGAGTTATTTTTAGCAGAATTCTGCCCCGCCCCCACTAA |
| | | | TTTGCCCATGTAAGGAGGCAAGGCCTGGGGACACCCGAGA |
| | | | TGCCTGGTTATAATTAACCCAGACATGTGGCTGCCCCCCCC |
| | | | CCCCAACACCTGCTGCCCTCTAAAAATAACCCTGCATTCCA |
| | | | TGTTCCCGGCGAAGGGCCAGCTGTCCCCCGCCAGCTAGAC |
| | | | TCAGCACTTAGTTTAGGAACCAGTGAGCAAGTCAGCCCTT |
| | | | GGGGCAGCCCATACAAGGCCATGGGGCTGGGCAAGCTGC |
| | | | ACGCCTGGGTCCGGGGTGGGCACGGTGCCCGGGCAACGAG |
| | | | CTGAAAGCTCATCTACTCTCAGGGGCCCCTCCCTGGGGAC |
| | | | AGCCCCTCCTGGCTAGTCACACCCTGTAGGCTCCTCTATAT |
| | | | AACCCAGGGGCACAGGGGCTGCCCTCATTCTACCACCACC |
| | | | TCCACAGCACAGACAGACACTCAGGAGCCAGCCAGCCGCC |
| | | | ACC |
| 191 | | MTF1186 | TAATTTTGCTAAAAATAACTCCAGTGAATCCAGTAGGAAT |
| | | | GCAGAATTATATGGTGCCGGCCAATTCTGCTAAAAATAAC |
| | | | TCCACTGAATTTAGGGAAACCTGAAGCCAATTTAGGGAAA |
| | | | CTTGAAGCCAATTCAGGGGAGTTATTTTTAGCAGAATTCA |
| | | | GTGGAGTTATTTTTAGCAGAATTCTGCCCCGCCCCCACTAA |
| | | | TT |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 57 | M57 | MTF1197- | TAGGCCTGGGGACAAATTCAAGGCCTGGGGACAAATTTGC |
| | | MCKmin | TGACTTGGTAATTTGTCCCCAGGCCTTGAATTGGCTTCAGG |
| | | | TTTCCCTAAATTCTGCACCTGACTAATTCTGCTAAAAATAA |
| | | | CTCCACTGAATTTAGGGAAACCTGAAGCCAATTGCTATTG |
| | | | GCTGGAATTTAGGGAAACCTGAAGCCAATTCCAGGCATAT |
| | | | CGGGTAATTCTGCCAAATATGGACTAATTCCTCCCTGGGG |
| | | | ACAGCCCCTCCTGGCTAGTCACACCCTGTAGGCTCCTCTAT |
| | | | ATAACCCAGGGGCACAGGGGCTGCCCTCATTCTACCACCA |
| | | | CCTCCACAGCACAGACAGACACTCAGGAGCCAGCCAGCCG |
| | | | CCACC |
| 192 | | MTF1197 | TAGGCCTGGGGACAAATTCAAGGCCTGGGGACAAATTTGC |
| | | | TGACTTGGTAATTTGTCCCCAGGCCTTGAATTGGCTTCAGG |
| | | | TTTCCCTAAATTCTGCACCTGACTAATTCTGCTAAAAATAA |
| | | | CTCCACTGAATTTAGGGAAACCTGAAGCCAATTGCTATTG |
| | | | GCTGGAATTTAGGGAAACCTGAAGCCAATTCCAGGCATAT |
| | | | CGGGTAATTCTGCCAAATATGGACTAATT |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 58 | M58 | MTF1197- | TAGGCCTGGGGACAAATTCAAGGCCTGGGGACAAATTTGC |
| | | CK8e | TGACTTGGTAATTTGTCCCCAGGCCTTGAATTGGCTTCAGG |
| | | | TTTCCCTAAATTCTGCACCTGACTAATTCTGCTAAAAATAA |
| | | | CTCCACTGAATTTAGGGAAACCTGAAGCCAATTGCTATTG |
| | | | GCTGGAATTTAGGGAAACCTGAAGCCAATTCCAGGCATAT |
| | | | CGGGTAATTCTGCCAAATATGGACTAATTTGCCCATGTAA |
| | | | GGAGGCAAGGCCTGGGGACACCCGAGATGCCTGGTTATAA |
| | | | TTAACCCAGACATGTGGCTGCCCCCCCCCCCCAACACCTGC |
| | | | TGCCCTCTAAAAATAACCCTGCATTCCATGTTCCCGGCGAA |
| | | | GGGCCAGCTGTCCCCCGCCAGCTAGACTCAGCACTTAGTTT |
| | | | AGGAACCAGTGAGCAAGTCAGCCCTTGGGGCAGCCCATAC |
| | | | AAGGCCATGGGGCTGGGCAAGCTGCACGCCTGGGTCCGGG |
| | | | GTGGGCACGGTGCCCGGGCAACGAGCTGAAAGCTCATCTA |
| | | | CTCTCAGGGGCCCCTCCCTGGGGACAGCCCCTCCTGGCTA |
| | | | GTCACACCCTGTAGGCTCCTCTATATAACCCAGGGGCACA |
| | | | GGGGCTGCCCTCATTCTACCACCACCTCCACAGCACAGAC |
| | | | AGACACTCAGGAGCCAGCCAGCCGCCACC |
| 192 | | MTF1197 | TAGGCCTGGGGACAAATTCAAGGCCTGGGGACAAATTTGC |
| | | | TGACTTGGTAATTTGTCCCCAGGCCTTGAATTGGCTTCAGG |
| | | | TTTCCCTAAATTCTGCACCTGACTAATTCTGCTAAAAATAA |
| | | | CTCCACTGAATTTAGGGAAACCTGAAGCCAATTGCTATTG |
| | | | GCTGGAATTTAGGGAAACCTGAAGCCAATTCCAGGCATAT |
| | | | CGGGTAATTCTGCCAAATATGGACTAATT |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 59 | M59 | MTF1209- | TAATTGGCTTCAGGTTTCCCTAAATTGGCTTCAGGTTTCCC |
| | | MCKmin | TAAATTCCAGCCAATAGCAATTTGCTGACTTGGTAATTCTG |
| | | | CTAAAAATAACTCCACTGAATTTAACCAGGCATCTCGGGT |
| | | | GTCCAATTCCTCCCTGGGGACAGCCCCTCCTGGCTAGTCAC |
| | | | ACCCTGTAGGCTCCTCTATATAACCCAGGGGCACAGGGGC |
| | | | TGCCCTCATTCTACCACCACCTCCACAGCACAGACAGACA |
| | | | CTCAGGAGCCAGCCAGCCGCCACC |
| 193 | | MTF1209 | TAATTGGCTTCAGGTTTCCCTAAATTGGCTTCAGGTTTCCC |
| | | | TAAATTCCAGCCAATAGCAATTTGCTGACTTGGTAATTCTG |
| | | | CTAAAAATAACTCCACTGAATTTAACCAGGCATCTCGGGT |
| | | | GTCCAATT |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 60 | M60 | MTF1209- | TAATTGGCTTCAGGTTTCCCTAAATTGGCTTCAGGTTTCCC |
| | | CK8e | TAAATTCCAGCCAATAGCAATTTGCTGACTTGGTAATTCTG |
| | | | CTAAAAATAACTCCACTGAATTTAACCAGGCATCTCGGGT |
| | | | GTCCAATTTGCCCATGTAAGGAGGCAAGGCCTGGGGACAC |
| | | | CCGAGATGCCTGGTTATAATTAACCCAGACATGTGGCTGC |
| | | | CCCCCCCCCCCAACACCTGCTGCCCTCTAAAAATAACCCTG |
| | | | CATTCCATGTTCCCGGCGAAGGGCCAGCTGTCCCCCGCCA |
| | | | GCTAGACTCAGCACTTAGTTTAGGAACCAGTGAGCAAGTC |
| | | | AGCCCTTGGGGCAGCCCATACAAGGCCATGGGGCTGGGCA |
| | | | AGCTGCACGCCTGGGTCCGGGGTGGGCACGGTGCCCGGGC |
| | | | AACGAGCTGAAAGCTCATCTACTCTCAGGGGCCCCTCCCT |
| | | | GGGGACAGCCCCTCCTGGCTAGTCACACCCTGTAGGCTCC |
| | | | TCTATATAACCCAGGGGCACAGGGGCTGCCCTCATTCTAC |
| | | | CACCACCTCCACAGCACAGACAGACACTCAGGAGCCAGCC |
| | | | AGCCGCCACC |
| 193 | | MTF1209 | TAATTGGCTTCAGGTTTCCCTAAATTGGCTTCAGGTTTCCC |
| | | | TAAATTCCAGCCAATAGCAATTTGCTGACTTGGTAATTCTG |
| | | | CTAAAAATAACTCCACTGAATTTAACCAGGCATCTCGGGT |
| | | | GTCCAATT |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 61 | M61 | MTF1211- | TAATTGGACACCCGAGATGCCTGGTTAAATTCAGTGGAGT |
| | | MCKmin | TATTTTTAGCAGAATTATATGGCGACGGCCAATTATATGGC |
| | | | GACGGCCAATTCCAGGCATATCGGGTAATTTGCTGACTTG |
| | | | GTAATTTGTCCCCAGGCCTTGAATTCTGCCCCGCCCCCACT |
| | | | AATTAGTCCATATTTGGCAGAATTTATTACCCGATATACGT |
| | | | GGAATTCCAGGCATATCGGGTAATTTGCTGACTTGGTAATT |
| | | | TAACCAGGCATCTCGGGTGTCCAATTGCTATTGGCTGGAAT |
| | | | TTGTCCCCAGGCCTTGAATTCTGCCAAATATGGACTAATTC |
| | | | AGTAGGAATGCAGAATTTGTCCCCAGGCCTTGAATTCCAG |
| | | | GCATATCGGGTAATAATTGGCCGTCGCCATATAATTCCAG |
| | | | GCATATCGGGTAATTCTGCATTCCTACTGAATTTGTCCCCA |
| | | | GGCCTTGAATTAGTCCATATTTGGCAGAATTTAGGGAAAC |
| | | | CTGAAGCCAATTAGTCCATATTTGGCAGAATTACCAAGTC |
| | | | AGCAAATTCTGCACCTGACTAATTCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 194 | | MTF1211 | TAATTGGACACCCGAGATGCCTGGTTAAATTCAGTGGAGT |
| | | | TATTTTTAGCAGAATTATATGGCGACGGCCAATTATATGGC |
| | | | GACGGCCAATTCCAGGCATATCGGGTAATTTGCTGACTTG |
| | | | GTAATTTGTCCCCAGGCCTTGAATTCTGCCCCGCCCCCACT |
| | | | AATTAGTCCATATTTGGCAGAATTTATTACCCGATATACGT |
| | | | GGAATTCCAGGCATATCGGGTAATTTGCTGACTTGGTAATT |
| | | | TAACCAGGCATCTCGGGTGTCCAATTGCTATTGGCTGGAAT |
| | | | TTGTCCCCAGGCCTTGAATTCTGCCAAATATGGACTAATTC |
| | | | AGTAGGAATGCAGAATTTGTCCCCAGGCCTTGAATTCCAG |
| | | | GCATATCGGGTAATAATTGGCCGTCGCCATATAATTCCAG |
| | | | GCATATCGGGTAATTCTGCATTCCTACTGAATTTGTCCCCA |
| | | | GGCCTTGAATTAGTCCATATTTGGCAGAATTTAGGGAAAC |
| | | | CTGAAGCCAATTAGTCCATATTTGGCAGAATTACCAAGTC |
| | | | AGCAAATTCTGCACCTGACTAATT |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 62 | M62 | MTF1211- | TAATTGGACACCCGAGATGCCTGGTTAAATTCAGTGGAGT |
| | | CK8e | TATTTTTAGCAGAATTATATGGCGACGGCCAATTATATGGC |
| | | | GACGGCCAATTCCAGGCATATCGGGTAATTTGCTGACTTG |
| | | | GTAATTTGTCCCCAGGCCTTGAATTCTGCCCCGCCCCCACT |
| | | | AATTAGTCCATATTTGGCAGAATTTATTACCCGATATACGT |
| | | | GGAATTCCAGGCATATCGGGTAATTTGCTGACTTGGTAATT |
| | | | TAACCAGGCATCTCGGGTGTCCAATTGCTATTGGCTGGAAT |
| | | | TTGTCCCCAGGCCTTGAATTCTGCCAAATATGGACTAATTC |
| | | | AGTAGGAATGCAGAATTTGTCCCCAGGCCTTGAATTCCAG |
| | | | GCATATCGGGTAATAATTGGCCGTCGCCATATAATTCCAG |
| | | | GCATATCGGGTAATTCTGCATTCCTACTGAATTTGTCCCCA |
| | | | GGCCTTGAATTAGTCCATATTTGGCAGAATTTAGGGAAAC |
| | | | CTGAAGCCAATTAGTCCATATTTGGCAGAATTACCAAGTC |
| | | | AGCAAATTCTGCACCTGACTAATTTGCCCATGTAAGGAGG |
| | | | CAAGGCCTGGGGACACCCGAGATGCCTGGTTATAATTAAC |
| | | | CCAGACATGTGGCTGCCCCCCCCCCCCAACACCTGCTGCCC |
| | | | TCTAAAAATAACCCTGCATTCCATGTTCCCGGCGAAGGGC |
| | | | CAGCTGTCCCCCGCCAGCTAGACTCAGCACTTAGTTTAGG |
| | | | AACCAGTGAGCAAGTCAGCCCTTGGGGCAGCCCATACAAG |
| | | | GCCATGGGGCTGGGCAAGCTGCACGCCTGGGTCCGGGGTG |
| | | | GGCACGGTGCCCGGGCAACGAGCTGAAAGCTCATCTACTC |
| | | | TCAGGGGCCCCTCCCTGGGGACAGCCCCTCCTGGCTAGTC |
| | | | ACACCCTGTAGGCTCCTCTATATAACCCAGGGGCACAGGG |
| | | | GCTGCCCTCATTCTACCACCACCTCCACAGCACAGACAGA |
| | | | CACTCAGGAGCCAGCCAGCCGCCACC |
| 194 | | MTF1211 | TAATTGGACACCCGAGATGCCTGGTTAAATTCAGTGGAGT |
| | | | TATTTTTAGCAGAATTATATGGCGACGGCCAATTATATGGC |
| | | | GACGGCCAATTCCAGGCATATCGGGTAATTTGCTGACTTG |
| | | | GTAATTTGTCCCCAGGCCTTGAATTCTGCCCCGCCCCCACT |
| | | | AATTAGTCCATATTTGGCAGAATTTATTACCCGATATACGT |
| | | | GGAATTCCAGGCATATCGGGTAATTTGCTGACTTGGTAATT |
| | | | TAACCAGGCATCTCGGGTGTCCAATTGCTATTGGCTGGAAT |
| | | | TTGTCCCCAGGCCTTGAATTCTGCCAAATATGGACTAATTC |
| | | | AGTAGGAATGCAGAATTTGTCCCCAGGCCTTGAATTCCAG |
| | | | GCATATCGGGTAATAATTGGCCGTCGCCATATAATTCCAG |
| | | | GCATATCGGGTAATTCTGCATTCCTACTGAATTTGTCCCCA |
| | | | GGCCTTGAATTAGTCCATATTTGGCAGAATTTAGGGAAAC |
| | | | CTGAAGCCAATTAGTCCATATTTGGCAGAATTACCAAGTC |
| | | | AGCAAATTCTGCACCTGACTAATT |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 63 | M63 | MTF1242- | TAATTAGTCAGGTGCAGAATTCCAGCCAATAGCAATTAGT |
| | | MCKmin | GGGGGCGGGGCAGAATTGGGGCTGTCCCCAGGGAGGGGC |
| | | | CCAATTCAGTGGAGTTATTTTTAGCAGAATTACCAAGTCAG |
| | | | CAAATTACCTCCCTGGGGACAGCCCCTCCTGGCTAGTCAC |
| | | | ACCCTGTAGGCTCCTCTATATAACCCAGGGGCACAGGGGC |
| | | | TGCCCTCATTCTACCACCACCTCCACAGCACAGACAGACA |
| | | | CTCAGGAGCCAGCCAGCCGCCACC |
| 195 | | MTF1242 | TAATTAGTCAGGTGCAGAATTCCAGCCAATAGCAATTAGT |
| | | | GGGGGCGGGGCAGAATTGGGGCTGTCCCCAGGGAGGGGC |
| | | | CCAATTCAGTGGAGTTATTTTTAGCAGAATTACCAAGTCAG |
| | | | CAAATTA |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 64 | M64 | MTF1242- | TAATTAGTCAGGTGCAGAATTCCAGCCAATAGCAATTAGT |
| | | CK8e | GGGGGCGGGGCAGAATTGGGGCTGTCCCCAGGGAGGGGC |
| | | | CCAATTCAGTGGAGTTATTTTTAGCAGAATTACCAAGTCAG |
| | | | CAAATTATGCCCATGTAAGGAGGCAAGGCCTGGGGACACC |
| | | | CGAGATGCCTGGTTATAATTAACCCAGACATGTGGCTGCC |
| | | | CCCCCCCCCCAACACCTGCTGCCCTCTAAAAATAACCCTGC |
| | | | ATTCCATGTTCCCGGCGAAGGGCCAGCTGTCCCCCGCCAG |
| | | | CTAGACTCAGCACTTAGTTTAGGAACCAGTGAGCAAGTCA |
| | | | GCCCTTGGGGCAGCCCATACAAGGCCATGGGGCTGGGCAA |
| | | | GCTGCACGCCTGGGTCCGGGGTGGGCACGGTGCCCGGGCA |
| | | | ACGAGCTGAAAGCTCATCTACTCTCAGGGGCCCCTCCCTG |
| | | | GGGACAGCCCCTCCTGGCTAGTCACACCCTGTAGGCTCCTC |
| | | | TATATAACCCAGGGGCACAGGGGCTGCCCTCATTCTACCA |
| | | | CCACCTCCACAGCACAGACAGACACTCAGGAGCCAGCCAG |
| | | | CCGCCACC |
| 195 | | MTF1242 | TAATTAGTCAGGTGCAGAATTCCAGCCAATAGCAATTAGT |
| | | | GGGGGCGGGGCAGAATTGGGGCTGTCCCCAGGGAGGGGC |
| | | | CCAATTCAGTGGAGTTATTTTTAGCAGAATTACCAAGTCAG |
| | | | CAAATTA |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 65 | M65 | MTF1295- | TAATTAGTCCATATTTGGCAGAATTACCAAGTCAGCAAATT |
| | | MCKmin | TAGGGAAACCTGAAGCCAATTCAGTGGAGTTATTTTTAGC |
| | | | AGAATTGGGCCCCTCCCTGGGGACAGCCCCAATTCCAGGC |
| | | | ATATCGGGTAATTCAGTAGGAATGCAGAATTCAGTGGAGT |
| | | | TATTTTTAGCAGAATTCTGCCAAATATGGACTAATTCCAGC |
| | | | CAATAGCAATTACCTCCCTGGGGACAGCCCCTCCTGGCTA |
| | | | GTCACACCCTGTAGGCTCCTCTATATAACCCAGGGGCACA |
| | | | GGGGCTGCCCTCATTCTACCACCACCTCCACAGCACAGAC |
| | | | AGACACTCAGGAGCCAGCCAGCCGCCACC |
| 197 | | MTF1295 | TAATTAGTCCATATTTGGCAGAATTACCAAGTCAGCAAATT |
| | | | TAGGGAAACCTGAAGCCAATTCAGTGGAGTTATTTTTAGC |
| | | | AGAATTGGGCCCCTCCCTGGGGACAGCCCCAATTCCAGGC |
| | | | ATATCGGGTAATTCAGTAGGAATGCAGAATTCAGTGGAGT |
| | | | TATTTTTAGCAGAATTCTGCCAAATATGGACTAATTCCAGC |
| | | | CAATAGCAATTA |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 66 | M66 | M66- | TAATTAGTCCATATTTGGCAGAATTACCAAGTCAGCAAATT |
| | | MTF1295- | TAGGGAAACCTGAAGCCAATTCAGTGGAGTTATTTTTAGC |
| | | CK8e | AGAATTGGGCCCCTCCCTGGGGACAGCCCCAATTCCAGGC |
| | | | ATATCGGGTAATTCAGTAGGAATGCAGAATTCAGTGGAGT |
| | | | TATTTTTAGCAGAATTCTGCCAAATATGGACTAATTCCAGC |
| | | | CAATAGCAATTATGCCCATGTAAGGAGGCAAGGCCTGGGG |
| | | | ACACCCGAGATGCCTGGTTATAATTAACCCAGACATGTGG |
| | | | CTGCCCCCCCCCCCCAACACCTGCTGCCCTCTAAAAATAAC |
| | | | CCTGCATTCCATGTTCCCGGCGAAGGGCCAGCTGTCCCCCG |
| | | | CCAGCTAGACTCAGCACTTAGTTTAGGAACCAGTGAGCAA |
| | | | GTCAGCCCTTGGGGCAGCCCATACAAGGCCATGGGGCTGG |
| | | | GCAAGCTGCACGCCTGGGTCCGGGGTGGGCACGGTGCCCG |
| | | | GGCAACGAGCTGAAAGCTCATCTACTCTCAGGGGCCCCTC |
| | | | CCTGGGGACAGCCCCTCCTGGCTAGTCACACCCTGTAGGC |
| | | | TCCTCTATATAACCCAGGGGCACAGGGGCTGCCCTCATTCT |
| | | | ACCACCACCTCCACAGCACAGACAGACACTCAGGAGCCAG |
| | | | CCAGCCGCCACC |
| 197 | | MTF1295 | TAATTAGTCCATATTTGGCAGAATTACCAAGTCAGCAAATT |
| | | | TAGGGAAACCTGAAGCCAATTCAGTGGAGTTATTTTTAGC |
| | | | AGAATTGGGCCCCTCCCTGGGGACAGCCCCAATTCCAGGC |
| | | | ATATCGGGTAATTCAGTAGGAATGCAGAATTCAGTGGAGT |
| | | | TATTTTTAGCAGAATTCTGCCAAATATGGACTAATTCCAGC |
| | | | CAATAGCAATTA |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 67 | M67 | MTF1304- | TAATTAGTCCATATTTGGCAGAATTTGTCCCCAGGCCTTGA |
| | | MCKmin | ATTCAGTAGGAATGCAGAATTCAGTAGGAATGCAGAATTT |
| | | | AACCAGGCATCTCGGGTGTCCAATTACCTCCCTGGGGACA |
| | | | GCCCCTCCTGGCTAGTCACACCCTGTAGGCTCCTCTATATA |
| | | | ACCCAGGGGCACAGGGGCTGCCCTCATTCTACCACCACCT |
| | | | CCACAGCACAGACAGACACTCAGGAGCCAGCCAGCCGCC |
| | | | ACC |
| 198 | | MTF1304 | TAATTAGTCCATATTTGGCAGAATTTGTCCCCAGGCCTTGA |
| | | | ATTCAGTAGGAATGCAGAATTCAGTAGGAATGCAGAATTT |
| | | | AACCAGGCATCTCGGGTGTCCAATTA |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 68 | M68 | MTF1304- | TAATTAGTCCATATTTGGCAGAATTTGTCCCCAGGCCTTGA |
| | | CK8e | ATTCAGTAGGAATGCAGAATTCAGTAGGAATGCAGAATTT |
| | | | AACCAGGCATCTCGGGTGTCCAATTATGCCCATGTAAGGA |
| | | | GGCAAGGCCTGGGGACACCCGAGATGCCTGGTTATAATTA |
| | | | ACCCAGACATGTGGCTGCCCCCCCCCCCCAACACCTGCTG |
| | | | CCCTCTAAAAATAACCCTGCATTCCATGTTCCCGGCGAAG |
| | | | GGCCAGCTGTCCCCCGCCAGCTAGACTCAGCACTTAGTTTA |
| | | | GGAACCAGTGAGCAAGTCAGCCCTTGGGGCAGCCCATACA |
| | | | AGGCCATGGGGCTGGGCAAGCTGCACGCCTGGGTCCGGGG |
| | | | TGGGCACGGTGCCCGGGCAACGAGCTGAAAGCTCATCTAC |
| | | | TCTCAGGGGCCCCTCCCTGGGGACAGCCCCTCCTGGCTAGT |
| | | | CACACCCTGTAGGCTCCTCTATATAACCCAGGGGCACAGG |
| | | | GGCTGCCCTCATTCTACCACCACCTCCACAGCACAGACAG |
| | | | ACACTCAGGAGCCAGCCAGCCGCCACC |
| 198 | | MTF1304 | TAATTAGTCCATATTTGGCAGAATTTGTCCCCAGGCCTTGA |
| | | | ATTCAGTAGGAATGCAGAATTCAGTAGGAATGCAGAATTT |
| | | | AACCAGGCATCTCGGGTGTCCAATTA |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 69 | M69 | MTF1308- | TAATTGCTATTGGCTGGAATTATATGGCGACGGCCAATTG |
| | | MCKmin | GGGCTGTCCCCAGGGAGGGGCCCAATTCAAGGCCTGGGGA |
| | | | CAAATTACCTCCCTGGGGACAGCCCCTCCTGGCTAGTCAC |
| | | | ACCCTGTAGGCTCCTCTATATAACCCAGGGGCACAGGGGC |
| | | | TGCCCTCATTCTACCACCACCTCCACAGCACAGACAGACA |
| | | | CTCAGGAGCCAGCCAGCCGCCACC |
| 199 | | MTF1308 | TAATTGCTATTGGCTGGAATTATATGGCGACGGCCAATTG |
| | | | GGGCTGTCCCCAGGGAGGGGCCCAATTCAAGGCCTGGGGA |
| | | | CAAATTA |
| 155 | | MCKmin | CCTCCCTGGGGACAGCCCCTCCTGGCTAGTCACACCCTGTA |
| | | | GGCTCCTCTATATAACCCAGGGGCACAGGGGCTGCCCTCA |
| | | | TTCTACCACCACCTCCACAGCACAGACAGACACTCAGGAG |
| | | | CCAGCCAGCCGCCACC |
| 70 | M70 | MTF1308- | TAATTGCTATTGGCTGGAATTATATGGCGACGGCCAATTG |
| | | CK8e | GGGCTGTCCCCAGGGAGGGGCCCAATTCAAGGCCTGGGGA |
| | | | CAAATTATGCCCATGTAAGGAGGCAAGGCCTGGGGACACC |
| | | | CGAGATGCCTGGTTATAATTAACCCAGACATGTGGCTGCC |
| | | | CCCCCCCCCCAACACCTGCTGCCCTCTAAAAATAACCCTGC |
| | | | ATTCCATGTTCCCGGCGAAGGGCCAGCTGTCCCCCGCCAG |
| | | | CTAGACTCAGCACTTAGTTTAGGAACCAGTGAGCAAGTCA |
| | | | GCCCTTGGGGCAGCCCATACAAGGCCATGGGGCTGGGCAA |
| | | | GCTGCACGCCTGGGTCCGGGGTGGGCACGGTGCCCGGGCA |
| | | | ACGAGCTGAAAGCTCATCTACTCTCAGGGGCCCCTCCCTG |
| | | | GGGACAGCCCCTCCTGGCTAGTCACACCCTGTAGGCTCCTC |
| | | | TATATAACCCAGGGGCACAGGGGCTGCCCTCATTCTACCA |
| | | | CCACCTCCACAGCACAGACAGACACTCAGGAGCCAGCCAG |
| | | | CCGCCACC |
| 199 | | MTF1308 | TAATTGCTATTGGCTGGAATTATATGGCGACGGCCAATTG |
| | | | GGGCTGTCCCCAGGGAGGGGCCCAATTCAAGGCCTGGGGA |
| | | | CAAATTA |
| 130 | | CK8e | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCCGCCAC |
| | | | C |
| 71 | M71 | hCK | GAATTCGGTACCCCACTACGGGTTTAGGCTGCCCATGTAA |
| | | | GGAGGCAAGGCCTGGGGACACCCGAGATGCCTGGTTATAA |
| | | | TTAACCCAGACATGTGGCTGCCCCCCCCCCCCCCAACACCT |
| | | | GCTGCCTCTAAAAATAACCCTGTCCCTGGTGGATCCCCTGC |
| | | | ATGCGAAGATCTTCGAACAAGGCTGTGGGGGACTGAGGGC |
| | | | AGGCTGTAACAGGCTTGGGGGCCAGGGCTTATACGTGCCT |
| | | | GGGACTCCCAAAGTATTACTGTTCCATGTTCCCGGCGAAG |
| | | | GGCCAGCTGTCCCCCGCCAGCTAGACTCAGCACTTAGTTTA |
| | | | GGAACCAGTGAGCAAGTCAGCCCTTGGGGCAGCCCATACA |
| | | | AGGCCATGGGGCTGGGCAAGCTGCACGCCTGGGTCCGGGG |
| | | | TGGGCACGGTGCCCGGGCAACGAGCTGAAAGCTCATCTGC |
| | | | TCTCAGGGGCCCCTCCCTGGGGACAGCCCCTCCTGGCTAGT |
| | | | CACACCCTGTAGGCTCCTCTATATAACCCAGGGGCACAGG |
| | | | GGCTGCCCTCATTCTACCACCACCTCCACAGCACAGACAG |
| | | | ACACTCAGGAGCCAGCCAGCGTCGAGCGGCCGATCCGCCA |
| | | | CCCCGCCACC |
| 72 | M72 | hCKplus | GAATTCGGTACCCCACTACGGGTCTAGGCTGCCCATGTAA |
| | | | GGAGGCAAGGCCTGGGGACACCCGAGATGCCTGGTTATAA |
| | | | TTAACCCCAACACCTGCTGCCCCCCCCCCCCCAACACCTGC |
| | | | TGCCTCTAAAAATAACCCTGTCCCTGGTGGATCCCCTGCAT |
| | | | GCCCCACTACGGGTTTAGGCTGCCCATGTAAGGAGGCAAG |
| | | | GCCTGGGGACACCCGAGATGCCTGGTTATAATTAACCCAG |
| | | | ACATGTGGCTGCCCCCCCCCCCCCCAACACCTGCTGCCTCT |
| | | | AAAAATAACCCTGTCCCTGGTGGATCCCCTGCATGCGAAG |
| | | | ATCTTCGAACAAGGCTGTGGGGGACTGAGGGCAGGCTGTA |
| | | | ACAGGCTTGGGGGCCAGGGCTTATACGTGCCTGGGACTCC |
| | | | CAAAGTATTACTGTTCCATGTTCCCGGCGAAGGGCCAGCT |
| | | | GTCCCCCGCCAGCTAGACTCAGCACTTAGTTTAGGAACCA |
| | | | GTGAGCAAGTCAGCCCTTGGGGCAGCCCATACAAGGCCAT |
| | | | GGGGCTGGGCAAGCTGCACGCCTGGGTCCGGGGTGGGCAC |
| | | | GGTGCCCGGGCAACGAGCTGAAAGCTCATCTGCTCTCAGG |
| | | | GGCCCCTCCCTGGGGACAGCCCCTCCTGGCTAGTCACACC |
| | | | CTGTAGGCTCCTCTATATAACCCAGGGGCACAGGGGCTGC |
| | | | CCTCATTCTACCACCACCTCCACAGCACAGACAGACACTC |
| | | | AGGAGCCAGCCAGCGTCGAGCGGCCGATCCGCCACCCCGC |
| | | | CACC |
| 73 | M73 | MHCK7 | TAGAAGCTGCATGTCTAAGCTAGACCCTTCAGATTAAAAA |
| | | | TAACTGAGGTAAGGGCCTGGGTAGGGGAGGTGGTGTGAG |
| | | | ACGCTCCTGTCTCTCCTCTATCTGCCCATCGGCCCTTTGGG |
| | | | GAGGAGGAATGTGCCCAAGGACTAAAAAAAGGCCATGGA |
| | | | GCCAGAGGGGCGAGGGCAACAGACCTTTCATGGGCAAAC |
| | | | CTTGGGGCCCTGCTGTCTAGCATGCCCCACTACGGGTCTAG |
| | | | GCTGCCCATGTAAGGAGGCAAGGCCTGGGGACACCCGAG |
| | | | ATGCCTGGTTATAATTAACCCAGACATGTGGCTGCCCCCCC |
| | | | CCCCCCAACACCTGCTGCCTCTAAAAATAACCCTGTCCCTG |
| | | | GTGGATCCCCTGCATGCGAAGATCTTCGAACAAGGCTGTG |
| | | | GGGGACTGAGGGCAGGCTGTAACAGGCTTGGGGGCCAGG |
| | | | GCTTATACGTGCCTGGGACTCCCAAAGTATTACTGTTCCAT |
| | | | GTTCCCGGCGAAGGGCCAGCTGTCCCCCGCCAGCTAGACT |
| | | | CAGCACTTAGTTTAGGAACCAGTGAGCAAGTCAGCCCTTG |
| | | | GGGCAGCCCATACAAGGCCATGGGGCTGGGCAAGCTGCAC |
| | | | GCCTGGGTCCGGGGTGGGCACGGTGCCCGGGCAACGAGCT |
| | | | GAAAGCTCATCTGCTCTCAGGGGCCCCTCCCTGGGGACAG |
| | | | CCCCTCCTGGCTAGTCACACCCTGTAGGCTCCTCTATATAA |
| | | | CCCAGGGGCACAGGGGCTGCCCTCATTCTACCACCACCTC |
| | | | CACAGCACCCGCCACC |
| 74 | M74 | CK8e- | TGTACGGGCCAGATATACGCGTTTGCCCATGTAAGGAGGC |
| | | EEF1A1i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCGGGAGAGCGGGCGGGTGAG |
| | | | TCACCCACACAAAGGAAAAGGGCCTTTCCGTCCTCAGCCG |
| | | | TCGCTTCATGTGACTCCACGGAGTACCGGGCGCCGTCCAG |
| | | | GCACCTCGATTAGTTCTCGAGCTTTTGGAGTACGTCGTCTT |
| | | | TAGGTTGGGGGGAGGGGTTTTATGCGATGGAGTTTCCCCA |
| | | | CACTGAGTGGGTGGAGACTGAAGTTAGGCCAGCTTGGCAC |
| | | | TTGATGTAATTCTCCTTGGAATTTGCCCTTTTTGAGTTTGGA |
| | | | TCTTGGTTCATTCTCAAGCCTCAGACAGTGGTTCAAAGTTT |
| | | | TTTTCTTCCATTTCAGGTGTCGTGAAAACTAGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 137 | M75 | EEF1A1i | CGGGAGAGCGGGCGGGTGAGTCACCCACACAAAGGAAAA |
| | | | GGGCCTTTCCGTCCTCAGCCGTCGCTTCATGTGACTCCACG |
| | | | GAGTACCGGGCGCCGTCCAGGCACCTCGATTAGTTCTCGA |
| | | | GCTTTTGGAGTACGTCGTCTTTAGGTTGGGGGGAGGGGTTT |
| | | | TATGCGATGGAGTTTCCCCACACTGAGTGGGTGGAGACTG |
| | | | AAGTTAGGCCAGCTTGGCACTTGATGTAATTCTCCTTGGAA |
| | | | TTTGCCCTTTTTGAGTTTGGATCTTGGTTCATTCTCAAGCCT |
| | | | CAGACAGTGGTTCAAAGTTTTTTTCTTCCATTTCAGGTGTC |
| | | | GTGAAAACTA |
| 75 | | CK8e- | TGTACGGGCCAGATATACGCGTTTGCCCATGTAAGGAGGC |
| | | EEF1B2i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCTCGGGAGCCGTGGAGGTACGA |
| | | | ACTTAAGACATGCCTATTTTATTAATTTACTTCCAAACGCA |
| | | | ACGAAAGGTCCATGGACAATTTGTGGGCCATTTAATTCAG |
| | | | GGCCCCCAATTCGTACGTGGAGAAGTGGGAATGCAAAAGT |
| | | | ACTTTGACCTTTAACCTTCGGTCCGGCGCGGTGGAGGGAA |
| | | | ACGCCTCCGTCTCTATATAAGGAATTTTCCGGTCTCTTCGG |
| | | | GTCCTTTTTCCTCTCTTCAGCGTGGGGCGCCCACAATTTGC |
| | | | GCGCTCTCTTTCTGCTGCTCCCCAGCTCTCGGATACAGCCG |
| | | | ACACCGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 138 | | EEF1B2i | TCGGGAGCCGTGGAGGTACGAACTTAAGACATGCCTATTT |
| | | | TATTAATTTACTTCCAAACGCAACGAAAGGTCCATGGACA |
| | | | ATTTGTGGGCCATTTAATTCAGGGCCCCCAATTCGTACGTG |
| | | | GAGAAGTGGGAATGCAAAAGTACTTTGACCTTTAACCTTC |
| | | | GGTCCGGCGCGGTGGAGGGAAACGCCTCCGTCTCTATATA |
| | | | AGGAATTTTCCGGTCTCTTCGGGTCCTTTTTCCTCTCTTCAG |
| | | | CGTGGGGCGCCCACAATTTGCGCGCTCTCTTTCTGCTGCTC |
| | | | CCCAGCTCTCGGATACAGCCGACACC |
| 76 | M76 | CK8e- | TGTACGGGCCAGATATACGCGTTTGCCCATGTAAGGAGGC |
| | | TMSB10i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCGAACGAGACTGCACGGTGAGT |
| | | | GCGGCGCCGGGGCGGGGGGCCCACCCAGGGTGTGGTCGG |
| | | | ATCCGGTGCACCGGGCGGGCGCGCCGCAACCGCGACAGGC |
| | | | GCCCTTCTCGGACCGGACGCAGGGGCCGGCGACCACGCCC |
| | | | TGGGACCGAGAAGAGGGGTGCGGGACGCGCCCAGATCCT |
| | | | CGGCCTTGGGGCTGCTCGGCACGCCTTGGCGCGAGTGCCA |
| | | | CGTCGAGAGGCGTCGGCGGGGAGCGCGGAAGGGGACGCT |
| | | | GGCCCCCAGGCCCAGGTCAAGCGCCTTGGTTTGCCCACTA |
| | | | GGATTGTTTTAAGAAAAGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 219 | | TMSB10i | GAACGAGACTGCACGGTGAGTGCGGCGCCGGGGCGGGGG |
| | | | GCCCACCCAGGGTGTGGTCGGATCCGGTGCACCGGGCGGG |
| | | | CGCGCCGCAACCGCGACAGGCGCCCTTCTCGGACCGGACG |
| | | | CAGGGGCCGGCGACCACGCCCTGGGACCGAGAAGAGGGG |
| | | | TGCGGGACGCGCCCAGATCCTCGGCCTTGGGGCTGCTCGG |
| | | | CACGCCTTGGCGCGAGTGCCACGTCGAGAGGCGTCGGCGG |
| | | | GGAGCGCGGAAGGGGACGCTGGCCCCCAGGCCCAGGTCA |
| | | | AGCGCCTTGGTTTGCCCACTAGGATTGTTTTAAGAAAA |
| 77 | M77 | CK8e- | TGTACGGGCCAGATATACGCGTTTGCCCATGTAAGGAGGC |
| | | S100A6i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCGCGATTGTGCGTTGTGTAAGC |
| | | | CACGGCGCAGGGTGGGGCGCGGGCGGGACTTGGGCGGGC |
| | | | GGGGTGGGCTTGGCCGAGCTGGCCTCCGGGGCACCGACCG |
| | | | CTATAAGGCCAGTCGGACTGCGACACAGCCCATCCCCTCG |
| | | | ACCGCTCGCGTCGCATTTGGCCGCCTCCCTACCGGTGAGTT |
| | | | CTCTCCAGGAGCCCTGGGTACTTGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 212 | | S100A6i | GCGATTGTGCGTTGTGTAAGCCACGGCGCAGGGTGGGGCG |
| | | | CGGGCGGGACTTGGGCGGGCGGGGTGGGCTTGGCCGAGCT |
| | | | GGCCTCCGGGGCACCGACCGCTATAAGGCCAGTCGGACTG |
| | | | CGACACAGCCCATCCCCTCGACCGCTCGCGTCGCATTTGGC |
| | | | CGCCTCCCTACCGGTGAGTTCTCTCCAGGAGCCCTGGGTAC |
| | | | TT |
| 78 | M78 | CK8e- | TGTACGGGCCAGATATACGCGTTTGCCCATGTAAGGAGGC |
| | | RPL27i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCGCCTGAAGGGCGGGACAGGA |
| | | | AGTGAGGTCACTCCTCACCGGGGTGAAAGGTTAGCGGAAG |
| | | | TGTCCTTCTTTCCTTTTTGCTGGTAGGGCCGGGTGGTTGCT |
| | | | GCCGGTAAGTAGAAGCTTGGGTTGAATCTTTCAATCCGCT |
| | | | GCCATCCGCGGCTTGGGGGTCGAAGGTCCGGGCGGCGCCG |
| | | | GGTGCATTCCTTCCCTAGGTCTCTGGCGGCTACGTATCCGA |
| | | | TCCTGTCCTGCGAGCAGGGCCCTCGGGCGGCCTTGACCAA |
| | | | GGCGACAGTGAACACAGTCTGAAGTTCCGGCCCAAGACCT |
| | | | GGGCTTGCTGGCAGGAGTCAGCTCTGGGCATCTTTGGCTTT |
| | | | ACGGATTTTTAAGTGGCCCTTTCTCCTTGCTCTCTGCAGAA |
| | | | AGGGGCAAGTTCAGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 211 | | RPL27i | GCCTGAAGGGCGGGACAGGAAGTGAGGTCACTCCTCACCG |
| | | | GGGTGAAAGGTTAGCGGAAGTGTCCTTCTTTCCTTTTTGCT |
| | | | GGTAGGGCCGGGTGGTTGCTGCCGGTAAGTAGAAGCTTGG |
| | | | GTTGAATCTTTCAATCCGCTGCCATCCGCGGCTTGGGGGTC |
| | | | GAAGGTCCGGGCGGCGCCGGGTGCATTCCTTCCCTAGGTC |
| | | | TCTGGCGGCTACGTATCCGATCCTGTCCTGCGAGCAGGGC |
| | | | CCTCGGGCGGCCTTGACCAAGGCGACAGTGAACACAGTCT |
| | | | GAAGTTCCGGCCCAAGACCTGGGCTTGCTGGCAGGAGTCA |
| | | | GCTCTGGGCATCTTTGGCTTTACGGATTTTTAAGTGGCCCT |
| | | | TTCTCCTTGCTCTCTGCAGAAAGGGGCAAGTTCA |
| 79 | M79 | CK8e- | TGTACGGGCCAGATATACGCGTTTGCCCATGTAAGGAGGC |
| | | GFAPi | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCTCTCTGCGCGGCACGGTGAGC |
| | | | GCGGCCAGGGCAGGACGGGGACGGGCGGAGCAGGGACGG |
| | | | CCACCCCAGGAGATGCCAGGGGAGAAGGGCCAGGGACGA |
| | | | GGACCTGGGTCGAGGGCGGGGCCTGAACGTTACCCCCGGG |
| | | | CTAGAGGCGGGGCCTGGGGCCGGGGGCCGGGCCTCGAAA |
| | | | CCTCCCCAAGTCGGGGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 143 | | GFAPi | TCTCTGCGCGGCACGGTGAGCGCGGCCAGGGCAGGACGGG |
| | | | GACGGGCGGAGCAGGGACGGCCACCCCAGGAGATGCCAG |
| | | | GGGAGAAGGGCCAGGGACGAGGACCTGGGTCGAGGGCGG |
| | | | GGCCTGAACGTTACCCCCGGGCTAGAGGCGGGGCCTGGGG |
| | | | CCGGGGGCCGGGCCTCGAAACCTCCCCAAGTCGGG |
| 80 | M80 | CK8e- | TGTACGGGCCAGATATACGCGTTTGCCCATGTAAGGAGGC |
| | | UCHL1i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCGCTGCGCACGGGGGGTTCGT |
| | | | ACCCATCTGGCCGCGACCGTCCGTTTCCCCCTCGCTTGGTT |
| | | | CTGCCCCTGCTCCCCCTGCACAGGCCTCACAGTGCGTCTGG |
| | | | CCGGCGCTTTATAGCTGCAGCCTGGGCGGCTCCGCTAGCT |
| | | | GTTTTTCGTCTTCCCTAGGCTATTTCTGCCGGGGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 221 | | UCHL1i | CGCTGCGCACGGGGGGTTCGTACCCATCTGGCCGCGACCG |
| | | | TCCGTTTCCCCCTCGCTTGGTTCTGCCCCTGCTCCCCCTGCA |
| | | | CAGGCCTCACAGTGCGTCTGGCCGGCGCTTTATAGCTGCA |
| | | | GCCTGGGCGGCTCCGCTAGCTGTTTTTCGTCTTCCCTAGGC |
| | | | TATTTCTGCCGGG |
| 81 | M81 | CK8e- | TGTACGGGCCAGATATACGCGTTTGCCCATGTAAGGAGGC |
| | | ALDOAi | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCGGGGCTAGTGCTCGGGCAGGT |
| | | | GCCCTGGCTTCCCTTCCCCTCCACACGTCAACGATTCTATT |
| | | | TGAAGTTGGGCAGGGGGGTGGCGCTGCTCACCACACACAA |
| | | | GTGTTATAGGAGGAGTCTGGCCCTTGAGTACCGGGTACGC |
| | | | AGGGGTGCCTCAACCACACTCCGTCCACGGACTCTCCGTT |
| | | | ATTTTAGGAGGTGAGTGTAGTGCCAGTATCTACTCTCCTTC |
| | | | TTAAAAAAAACCAGGGCTCCAGAGAATCAGAACAGCCAC |
| | | | CATCACCGCAGGGAGTCAAGGGAGGAGGGAGATTAGAGA |
| | | | AGGAGCCAGGGAGGGTGGCAGGGAGGCCACGTGATCCGA |
| | | | GTCCCCTCACCCCTTTCCTTCCCACAGGTCCCTGGCCAAAG |
| | | | AGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 123 | | ALDOAi | GGGGCTAGTGCTCGGGCAGGTGCCCTGGCTTCCCTTCCCCT |
| | | | CCACACGTCAACGATTCTATTTGAAGTTGGGCAGGGGGGT |
| | | | GGCGCTGCTCACCACACACAAGTGTTATAGGAGGAGTCTG |
| | | | GCCCTTGAGTACCGGGTACGCAGGGGTGCCTCAACCACAC |
| | | | TCCGTCCACGGACTCTCCGTTATTTTAGGAGGTGAGTGTAG |
| | | | TGCCAGTATCTACTCTCCTTCTTAAAAAAAACCAGGGCTCC |
| | | | AGAGAATCAGAACAGCCACCATCACCGCAGGGAGTCAAG |
| | | | GGAGGAGGGAGATTAGAGAAGGAGCCAGGGAGGGTGGCA |
| | | | GGGAGGCCACGTGATCCGAGTCCCCTCACCCCTTTCCTTCC |
| | | | CACAGGTCCCTGGCCAAAGA |
| 82 | M82 | CK8e- | TGTACGGGCCAGATATACGCGTTTGCCCATGTAAGGAGGC |
| | | CAMK2Ai | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCGCAGTGGAAGGCAGGCAGG |
| | | | TGTTGGGGAGGCAGTTACCGGGGCAACGGGAACAGGGCG |
| | | | TTTTGGAGGTGGTTGCCATGGGGACCTGGATGCTGACGAA |
| | | | GGCTCGCGAGGCTGTGAGCAGCCACAGTGCCCTGCTCAGA |
| | | | AGCCCCGGGCTCGTCAGTCAAACCGGTTCTCTGTTTGCACT |
| | | | CGGCAGCACGGGCAGGCAAGTGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 127 | | CAMK2Ai | CGCAGTGGAAGGCAGGCAGGTGTTGGGGAGGCAGTTACC |
| | | | GGGGCAACGGGAACAGGGCGTTTTGGAGGTGGTTGCCATG |
| | | | GGGACCTGGATGCTGACGAAGGCTCGCGAGGCTGTGAGCA |
| | | | GCCACAGTGCCCTGCTCAGAAGCCCCGGGCTCGTCAGTCA |
| | | | AACCGGTTCTCTGTTTGCACTCGGCAGCACGGGCAGGCAA |
| | | | GT |
| 83 | M83 | CK8e- | TGTACGGGCCAGATATACGCGTTTGCCCATGTAAGGAGGC |
| | | FHL1i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCAGACATTTGGAGAAGTATGC |
| | | | AGAACATCTTGGCAGTTTTCCTTTAACTCTAAGGGTTGTAA |
| | | | ATACAAAGTAGAATGTGTTACCATGAGCCATTTCATAGGA |
| | | | AGTTGACTTTATTTTTTTAGAATTCAGAACTTTGCTTCTAA |
| | | | AAAGGTAACTTTGCCTCTTGATTCTCATTTTTTTCCCTAACT |
| | | | TAGGCACCTTGGAAGTTGGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 139 | | FHL1i | CAGACATTTGGAGAAGTATGCAGAACATCTTGGCAGTTTT |
| | | | CCTTTAACTCTAAGGGTTGTAAATACAAAGTAGAATGTGTT |
| | | | ACCATGAGCCATTTCATAGGAAGTTGACTTTATTTTTTTAG |
| | | | AATTCAGAACTTTGCTTCTAAAAAGGTAACTTTGCCTCTTG |
| | | | ATTCTCATTTTTTTCCCTAACTTAGGCACCTTGGAAGTTG |
| 84 | M84 | CK8e- | TGTACGGGCCAGATATACGCGTTTGCCCATGTAAGGAGGC |
| | | CRYABi | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCGAGGCAGTTGGCTGGTGCTG |
| | | | ACATGTTGACCATCACTGCTCTCTTCCAAGGACTCACAAAG |
| | | | AGTTAATGTCCCTGGGGCTCAGCCTAGGAAGATTCCAGTC |
| | | | CCTGCCCAGGCCCAAGATAGTTGCTGGCCTGATTCCCCTGG |
| | | | CATTCAGGACTGGAAAGGAGGAGGAGGGGCACACTACGC |
| | | | CGGCTCCCATCCTCCCCCCACCCCGCGTGCCTGCTTGGGAT |
| | | | TCCTGACTCTGTACCAGCTTCAGAGAACAGGGGTGGGGGT |
| | | | GGGTGCCATTGGGTGTGGACAGAAAGCTAGTGAAACAAG |
| | | | ACCTTGACAAGTCACTGGCCGGCTCAGACGTGTTTGTGTCT |
| | | | CTCTTTTCTTAGCTCAGTGAGTACTGGGTTTGTGTCACATT |
| | | | GCCAAATCCCGGATCACAAGTCTCCTTGAACTGCTGGTGA |
| | | | GCTAGGATAATAAAACCCCTGACATCACCATTCCAGAAGC |
| | | | TTCACAAGACTGCATATATAAGGGGCTGGCTGTAGCTGCA |
| | | | GCTGAAGGAGCTGACCAGCCAGCTGAGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 136 | | CRYABi | CGAGGCAGTTGGCTGGTGCTGACATGTTGACCATCACTGC |
| | | | TCTCTTCCAAGGACTCACAAAGAGTTAATGTCCCTGGGGCT |
| | | | CAGCCTAGGAAGATTCCAGTCCCTGCCCAGGCCCAAGATA |
| | | | GTTGCTGGCCTGATTCCCCTGGCATTCAGGACTGGAAAGG |
| | | | AGGAGGAGGGGCACACTACGCCGGCTCCCATCCTCCCCCC |
| | | | ACCCCGCGTGCCTGCTTGGGATTCCTGACTCTGTACCAGCT |
| | | | TCAGAGAACAGGGGTGGGGGTGGGTGCCATTGGGTGTGGA |
| | | | CAGAAAGCTAGTGAAACAAGACCTTGACAAGTCACTGGCC |
| | | | GGCTCAGACGTGTTTGTGTCTCTCTTTTCTTAGCTCAGTGA |
| | | | GTACTGGGTTTGTGTCACATTGCCAAATCCCGGATCACAA |
| | | | GTCTCCTTGAACTGCTGGTGAGCTAGGATAATAAAACCCC |
| | | | TGACATCACCATTCCAGAAGCTTCACAAGACTGCATATAT |
| | | | AAGGGGCTGGCTGTAGCTGCAGCTGAAGGAGCTGACCAGC |
| | | | CAGCTGA |
| 85 | M85 | CK8e- | TGTACGGGCCAGATATACGCGTTTGCCCATGTAAGGAGGC |
| | | FXYD1i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCTCTGCCCTGCCAGGGTAGGT |
| | | | CTGGGAATCGGGGGCCTGCTGCGGGAGGTGGAGGCCCAA |
| | | | GGGAGGCCCCCCGGGGACTGTGTGTCTCACCCCCGTCCCT |
| | | | GCTACGTTGTGTTGTTGTGTGATCCCATCGTGGAGGTTGTT |
| | | | TTGGTGACACTGTGTCCCCACGAAGCTGGGGATACCCGTTT |
| | | | CTCTAGCTTGGAGCCACCAAGATAGAGGACAAACACTTCT |
| | | | GTGATTCAGTCCCCAGACTGTCTCTGACTTAATCCCTTGGG |
| | | | TTCAAGCCCTTTGTGGGAGAGCAAGGGCACACACTGCCTA |
| | | | ATCCGTGGTGTCCCCCCCAGGACAATTGCGTCTCTGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 141 | | FXYD1i | CTCTGCCCTGCCAGGGTAGGTCTGGGAATCGGGGGCCTGC |
| | | | TGCGGGAGGTGGAGGCCCAAGGGAGGCCCCCCGGGGACT |
| | | | GTGTGTCTCACCCCCGTCCCTGCTACGTTGTGTTGTTGTGT |
| | | | GATCCCATCGTGGAGGTTGTTTTGGTGACACTGTGTCCCCA |
| | | | CGAAGCTGGGGATACCCGTTTCTCTAGCTTGGAGCCACCA |
| | | | AGATAGAGGACAAACACTTCTGTGATTCAGTCCCCAGACT |
| | | | GTCTCTGACTTAATCCCTTGGGTTCAAGCCCTTTGTGGGAG |
| | | | AGCAAGGGCACACACTGCCTAATCCGTGGTGTCCCCCCCA |
| | | | GGACAATTGCGTCTCT |
| 86 | M86 | CK8e- | TGTACGGGCCAGATATACGCGTTTGCCCATGTAAGGAGGC |
| | | RBP4i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCGTGCCCGGGCTCCGGTGAGT |
| | | | CAGGGCGCGTTATGCAAGTGCCCCCGGCGCCTCCCCTTCG |
| | | | GTCTTTCACCCCGCGCGGTTACGAAAGCGCGACCCCCTCCC |
| | | | CCCGGCGCTATAAAGCAGCGGGGCGGCCGCGGCGCGCTCG |
| | | | CCTCCCTCGCTCCACGCGCGCCCGGACTCGGCGGCCAGGC |
| | | | TTGCGCGCGGTTCCCCTCCCGGTGGTGAGTCGCGGCCGGG |
| | | | GCCTCGGGGCCGGCGGCGGGTTGGGCCGGGGTGGGGTGG |
| | | | GGTGGGGTGAGGTGGGATACGGCGGCGGGGGTCGGCTGC |
| | | | CGGGCACTGACGGAGGCGCGTCTCCCGCAGGGCGGATTCC |
| | | | TGGGCGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 209 | M87 | RBP4i | CGTGCCCGGGCTCCGGTGAGTCAGGGCGCGTTATGCAAGT |
| | | | GCCCCCGGCGCCTCCCCTTCGGTCTTTCACCCCGCGCGGTT |
| | | | ACGAAAGCGCGACCCCCTCCCCCCGGCGCTATAAAGCAGC |
| | | | GGGGCGGCCGCGGCGCGCTCGCCTCCCTCGCTCCACGCGC |
| | | | GCCCGGACTCGGCGGCCAGGCTTGCGCGCGGTTCCCCTCC |
| | | | CGGTGGTGAGTCGCGGCCGGGGCCTCGGGGCCGGCGGCGG |
| | | | GTTGGGCCGGGGTGGGGTGGGGTGGGGTGAGGTGGGATA |
| | | | CGGCGGCGGGGGTCGGCTGCCGGGCACTGACGGAGGCGC |
| | | | GTCTCCCGCAGGGCGGATTCCTGGGC |
| 87 | | CK8e- | TGTACGGGCCAGATATACGCGTTTGCCCATGTAAGGAGGC |
| | | APOC1i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCAGGAAGATTGAGAGGTGAG |
| | | | AGTGCTGAACGGGGAGGGGCTTTGGGGCTAAGGGAAGTG |
| | | | CCCGGGACCCCACCTGACCCCAACGCTCACGGGACAGGGG |
| | | | CAGAGGAGAAAAACGTGGGTGGACAGAGGGAGGCAGGCG |
| | | | GTCAGGGGAAGGCTCAGGAGGAGGGAGATCAACATCAAC |
| | | | CTGCCCCGCCCCCTCCCCAGCCTGATAAAGGTCCTGCGGG |
| | | | CAGGACAGGACCTCCCAACCAAGCCCTCCAGCAAGGATTC |
| | | | AGGTTGGTGCTGAGTGCCTGGGAGGGACACCCGCCTACAC |
| | | | TCTGCAAGAAACTCAAAAAGGGAGTTGAGGGGATCGTGG |
| | | | GAGGGAGGTAGGGAGGGAGGAGGGTGCCACTGATCCCCT |
| | | | GAACCCCTGCCTCTGCCTCCAGAGTGCCCCTCCGGCCGCCA |
| | | | CC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 124 | | APOC1i | CAGGAAGATTGAGAGGTGAGAGTGCTGAACGGGGAGGGG |
| | | | CTTTGGGGCTAAGGGAAGTGCCCGGGACCCCACCTGACCC |
| | | | CAACGCTCACGGGACAGGGGCAGAGGAGAAAAACGTGGG |
| | | | TGGACAGAGGGAGGCAGGCGGTCAGGGGAAGGCTCAGGA |
| | | | GGAGGGAGATCAACATCAACCTGCCCCGCCCCCTCCCCAG |
| | | | CCTGATAAAGGTCCTGCGGGCAGGACAGGACCTCCCAACC |
| | | | AAGCCCTCCAGCAAGGATTCAGGTTGGTGCTGAGTGCCTG |
| | | | GGAGGGACACCCGCCTACACTCTGCAAGAAACTCAAAAAG |
| | | | GGAGTTGAGGGGATCGTGGGAGGGAGGTAGGGAGGGAGG |
| | | | AGGGTGCCACTGATCCCCTGAACCCCTGCCTCTGCCTCCAG |
| | | | AGTGCCCCTCCGGCC |
| 88 | M88 | CK8e- | TGTACGGGCCAGATATACGCGTTTGCCCATGTAAGGAGGC |
| | | ATF5i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCGCTCAGAGGGAAGAGGTCGG |
| | | | ATCCCCAGCTGAGAGGGAGGAGGGTCCCGGACCCTAGGA |
| | | | GTGGGAAGGAAAGGCTCGGATCCCCTGATCCCCAGGAGGA |
| | | | GGGGACCCGGCTGCCTCCCGGTTGGGGCCGCGCGAGGGCG |
| | | | GGGCGCGGAAGGATCCGGGAGGGCCGTGCTCCGCCACCCA |
| | | | GTATATATCTGTCCCCAGTCCCCGGGGCCGCCTCATTCCCT |
| | | | GTCCTCGGATCACAGTCTCTTCTCACTACAGTGTCGCCGCC |
| | | | TCTGCGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 125 | | ATF5i | GCTCAGAGGGAAGAGGTCGGATCCCCAGCTGAGAGGGAG |
| | | | GAGGGTCCCGGACCCTAGGAGTGGGAAGGAAAGGCTCGG |
| | | | ATCCCCTGATCCCCAGGAGGAGGGGACCCGGCTGCCTCCC |
| | | | GGTTGGGGCCGCGCGAGGGCGGGGCGCGGAAGGATCCGG |
| | | | GAGGGCCGTGCTCCGCCACCCAGTATATATCTGTCCCCAGT |
| | | | CCCCGGGGCCGCCTCATTCCCTGTCCTCGGATCACAGTCTC |
| | | | TTCTCACTACAGTGTCGCCGCCTCTGC |
| 89 | M89 | CK8e- | TGTACGGGCCAGATATACGCGTTTGCCCATGTAAGGAGGC |
| | | HDPi | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCCATCCCCCAGGTAGGCCAGG |
| | | | AGGCCTGGGCGGGACCTGCTTGGGGTTAAATACTCCGGCC |
| | | | CAGCACTCCCCAGGCCTCTAGTCCCAGTAGGAGGTTTGAC |
| | | | TAAGATCAATCTTGGTAAGTGCCTTGGCCATCTCCCCTTGG |
| | | | GTGGGACATCTTCAGTAACGCTTCCAGAAGCACCTTGTGTT |
| | | | GGAACCTCCAAGTCTCACTGTCCTTCTTTTTCTCCTTAGAC |
| | | | GACTTACAGTGACGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 152 | | HDPi | CCATCCCCCAGGTAGGCCAGGAGGCCTGGGCGGGACCTGC |
| | | | TTGGGGTTAAATACTCCGGCCCAGCACTCCCCAGGCCTCTA |
| | | | GTCCCAGTAGGAGGTTTGACTAAGATCAATCTTGGTAAGT |
| | | | GCCTTGGCCATCTCCCCTTGGGTGGGACATCTTCAGTAACG |
| | | | CTTCCAGAAGCACCTTGTGTTGGAACCTCCAAGTCTCACTG |
| | | | TCCTTCTTTTTCTCCTTAGACGACTTACAGTGAC |
| 90 | M90 | CK8e- | TGTACGGGCCAGATATACGCGTTTGCCCATGTAAGGAGGC |
| | | FLOT1i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCCCGCGTCCCGGAAGGTGAGT |
| | | | GGGGGAGGGGAGATGGAGCCTGGGGCTGCCGAGAAGCAG |
| | | | GGAGAGAGGGGAAAGGTCTGCAGGCCTCTGGGCGCGGGG |
| | | | GACCTTTATCAAAGGGAGCGGCGAGGGGAAGGGAGGGAC |
| | | | CATACGCAGGCGGAGAAGAGGTAGCTGGGGATCTTAAGG |
| | | | ATTTTGTAGGGAAAAGCCTTAGCGGACCCTTGGGTGTAGA |
| | | | AGGGACGGGCCGTGGCGGTTGCAGACTGGGAGGGCCTGA |
| | | | CGGGAAAGGGAAAGGTTGGGCCCCTGTGGACGCCACAGC |
| | | | GCGCCTTCCCCATCCGCAAAGGCTTTCCCTCCCTCTCCTCC |
| | | | AGCTCCAGCCTGAACCGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 140 | | FLOT1i | CCCGCGTCCCGGAAGGTGAGTGGGGGAGGGGAGATGGAG |
| | | | CCTGGGGCTGCCGAGAAGCAGGGAGAGAGGGGAAAGGTC |
| | | | TGCAGGCCTCTGGGCGCGGGGGACCTTTATCAAAGGGAGC |
| | | | GGCGAGGGGAAGGGAGGGACCATACGCAGGCGGAGAAGA |
| | | | GGTAGCTGGGGATCTTAAGGATTTTGTAGGGAAAAGCCTT |
| | | | AGCGGACCCTTGGGTGTAGAAGGGACGGGCCGTGGCGGTT |
| | | | GCAGACTGGGAGGGCCTGACGGGAAAGGGAAAGGTTGGG |
| | | | CCCCTGTGGACGCCACAGCGCGCCTTCCCCATCCGCAAAG |
| | | | GCTTTCCCTCCCTCTCCTCCAGCTCCAGCCTGAACC |
| 91 | M91 | CK8e- | TGTACGGGCCAGATATACGCGTTTGCCCATGTAAGGAGGC |
| | | HBBi1 | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCTGAGGCCCTGGGCAGGTTGGT |
| | | | ATCAAGGTTACAAGACAGGTTTAAGGAGACCAATAGAAAC |
| | | | TGGGCATGTGGAGACAGAGAAGACTCTTGGGTTTCTGATA |
| | | | GGCACTGACTCTCTCTGCCTATTGGTCTATTTTCCCACCCTT |
| | | | AGGCTGCTGGTGGTCTAGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 145 | | HBBi1 | TGAGGCCCTGGGCAGGTTGGTATCAAGGTTACAAGACAGG |
| | | | TTTAAGGAGACCAATAGAAACTGGGCATGTGGAGACAGA |
| | | | GAAGACTCTTGGGTTTCTGATAGGCACTGACTCTCTCTGCC |
| | | | TATTGGTCTATTTTCCCACCCTTAGGCTGCTGGTGGTCTA |
| 92 | M92 | CK8e- | TGTACGGGCCAGATATACGCGTTTGCCCATGTAAGGAGGC |
| | | HBBi2 | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCCTGAGAACTTCAGGGTGAGT |
| | | | CTATGGGACGCTTGATGTTTTCTTTCCCCTTCTTTTCTATGG |
| | | | TTAAGTTCATGTCATAGGAAGGGGATAAGTAACAGGGTAC |
| | | | AGTTTAGAATGGGAAACAGACGAATGATTGCATCAGTGTG |
| | | | GAAGTCTCAGGATCGTTTTAGTTTCTTTTATTTGCTGTTCAT |
| | | | AACAATTGTTTTCTTTTGTTTAATTCTTGCTTTCTTTTTTTTT |
| | | | CTTCTCCGCAATTTTTACTATTATACTTAATGCCTTAACATT |
| | | | GTGTATAACAAAAGGAAATATCTCTGAGATACATTAAGTA |
| | | | ACTTAAAAAAAAACTTTACACAGTCTGCCTAGTACATTACT |
| | | | ATTTGGAATATATGTGTGCTTATTTGCATATTCATAATCTC |
| | | | CCTACTTTATTTTCTTTTATTTTTAATTGATACATAATCATT |
| | | | ATACATATTTATGGGTTAAAGTGTAATGTTTTAATATGTGT |
| | | | ACACATATTGACCAAATCAGGGTAATTTTGCATTTGTAATT |
| | | | TTAAAAAATGCTTTCTTCTTTTAATATACTTTTTTGTTTATC |
| | | | TTATTTCTAATACTTTCCCTAATCTCTTTCTTTCAGGGCAAT |
| | | | AATGATACAATGTATCATGCCTCTTTGCACCATTCTAAAGA |
| | | | ATAACAGTGATAATTTCTGGGTTAAGGCAATAGCAATATC |
| | | | TCTGCATATAAATATTTCTGCATATAAATTGTAACTGATGT |
| | | | AAGAGGTTTCATATTGCTAATAGCAGCTACAATCCAGCTA |
| | | | CCATTCTGCTTTTATTTTATGGTTGGGATAAGGCTGGATTA |
| | | | TTCTGAGTCCAAGCTAGGCCCTTTTGCTAATCATGTTCATA |
| | | | CCTCTTATCTTCCTCCCACAGCTCCTGGGCAACGTGGCCAC |
| | | | C |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 146 | | HBBi2 | CCTGAGAACTTCAGGGTGAGTCTATGGGACGCTTGATGTTT |
| | | | TCTTTCCCCTTCTTTTCTATGGTTAAGTTCATGTCATAGGAA |
| | | | GGGGATAAGTAACAGGGTACAGTTTAGAATGGGAAACAG |
| | | | ACGAATGATTGCATCAGTGTGGAAGTCTCAGGATCGTTTT |
| | | | AGTTTCTTTTATTTGCTGTTCATAACAATTGTTTTCTTTTGT |
| | | | TTAATTCTTGCTTTCTTTTTTTTTCTTCTCCGCAATTTTTACT |
| | | | ATTATACTTAATGCCTTAACATTGTGTATAACAAAAGGAA |
| | | | ATATCTCTGAGATACATTAAGTAACTTAAAAAAAAACTTT |
| | | | ACACAGTCTGCCTAGTACATTACTATTTGGAATATATGTGT |
| | | | GCTTATTTGCATATTCATAATCTCCCTACTTTATTTTCTTTT |
| | | | ATTTTTAATTGATACATAATCATTATACATATTTATGGGTT |
| | | | AAAGTGTAATGTTTTAATATGTGTACACATATTGACCAAAT |
| | | | CAGGGTAATTTTGCATTTGTAATTTTAAAAAATGCTTTCTT |
| | | | CTTTTAATATACTTTTTTGTTTATCTTATTTCTAATACTTTCC |
| | | | CTAATCTCTTTCTTTCAGGGCAATAATGATACAATGTATCA |
| | | | TGCCTCTTTGCACCATTCTAAAGAATAACAGTGATAATTTC |
| | | | TGGGTTAAGGCAATAGCAATATCTCTGCATATAAATATTTC |
| | | | TGCATATAAATTGTAACTGATGTAAGAGGTTTCATATTGCT |
| | | | AATAGCAGCTACAATCCAGCTACCATTCTGCTTTTATTTTA |
| | | | TGGTTGGGATAAGGCTGGATTATTCTGAGTCCAAGCTAGG |
| | | | CCCTTTTGCTAATCATGTTCATACCTCTTATCTTCCTCCCAC |
| | | | AGCTCCTGGGCAACGTG |
| 93 | M93 | CK8e- | TGTACGGGCCAGATATACGCGTTTGCCCATGTAAGGAGGC |
| | | HBBi2xc | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCCTGAGAACTTCAGGGTGAGT |
| | | | CTATGGGACCCTTGATGTTTTCTTTCCCCTTCTTTTCTATGG |
| | | | TTAAGTTCATGTCATAGGAAGGGGAGAAGTAACAGGGTAC |
| | | | ACATATTGACCAAATCAGGGTAATTTTGCATTTGTAATTTT |
| | | | AAAAAATGCTTTCTTCTTTTAATATACTTTTTTGTTTATCTT |
| | | | ATTTCTAATACTTTCCCTAATCTCTTTCTTTCAGGGCAATAA |
| | | | TGATACAATGTATCATGCCTCTTTGCACCATTCTAAAGAAT |
| | | | AACAGTGATAATTTCTGGGTTAAGGCAATAGCAATATTTCT |
| | | | GCATATAAATATTTCTGCATATAAATTGTAACTGATGTAAG |
| | | | AGGTTTCATATTGCTAATAGCAGCTACAATCCAGCTACCAT |
| | | | TCTGCTTTTATTTTATGGTTGGGATAAGGCTGGATTATTCT |
| | | | GAGTCCAAGCTAGGCCCTTTTGCTAATCATGTTCATACCTC |
| | | | TTATCTTCCTCCCACAGCTCCTGGGCAACGTGGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 147 | | HBBi2xc | CCTGAGAACTTCAGGGTGAGTCTATGGGACCCTTGATGTTT |
| | | | TCTTTCCCCTTCTTTTCTATGGTTAAGTTCATGTCATAGGAA |
| | | | GGGGAGAAGTAACAGGGTACACATATTGACCAAATCAGG |
| | | | GTAATTTTGCATTTGTAATTTTAAAAAATGCTTTCTTCTTTT |
| | | | AATATACTTTTTTGTTTATCTTATTTCTAATACTTTCCCTAA |
| | | | TCTCTTTCTTTCAGGGCAATAATGATACAATGTATCATGCC |
| | | | TCTTTGCACCATTCTAAAGAATAACAGTGATAATTTCTGGG |
| | | | TTAAGGCAATAGCAATATTTCTGCATATAAATATTTCTGCA |
| | | | TATAAATTGTAACTGATGTAAGAGGTTTCATATTGCTAATA |
| | | | GCAGCTACAATCCAGCTACCATTCTGCTTTTATTTTATGGT |
| | | | TGGGATAAGGCTGGATTATTCTGAGTCCAAGCTAGGCCCT |
| | | | TTTGCTAATCATGTTCATACCTCTTATCTTCCTCCCACAGCT |
| | | | CCTGGGCAACGTG |
| 94 | M94 | CK8e- | TGTACGGGCCAGATATACGCGTTTGCCCATGTAAGGAGGC |
| | | IFI27L2i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCTGTTGAGTGAGTCTTGCTTCCC |
| | | | CAGGGGGCTGGCGGGCTGGGCTGGGGGCAGGCTTCCCCAA |
| | | | CCCCAGCCCAGGAAGGGACAGGGAATAGCTTCATTCCCTG |
| | | | ACTTCATCAACCAGCTGACCTACTGACTCCCTCTCCCTCCC |
| | | | TTCCTCCCTCCCCTTCCCTTCCTCCCTCCGCTCCTTGCTTCC |
| | | | CTCCCCCTCCCTCCCTTTCTGCCCTTCTCTCTCCTCTTTCTCC |
| | | | CCTTTCCCATCTCCCTCTGCCTTCCCCTGGCACTCTCTCTCT |
| | | | AGAACGGGCAGCTGCTGGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 153 | | IFI27L2i | TGTTGAGTGAGTCTTGCTTCCCCAGGGGGCTGGCGGGCTG |
| | | | GGCTGGGGGCAGGCTTCCCCAACCCCAGCCCAGGAAGGGA |
| | | | CAGGGAATAGCTTCATTCCCTGACTTCATCAACCAGCTGAC |
| | | | CTACTGACTCCCTCTCCCTCCCTTCCTCCCTCCCCTTCCCTT |
| | | | CCTCCCTCCGCTCCTTGCTTCCCTCCCCCTCCCTCCCTTTCT |
| | | | GCCCTTCTCTCTCCTCTTTCTCCCCTTTCCCATCTCCCTCTG |
| | | | CCTTCCCCTGGCACTCTCTCTCTAGAACGGGCAGCTGCTG |
| 95 | M95 | CK8e- | TGTACGGGCCAGATATACGCGTTTGCCCATGTAAGGAGGC |
| | | RPL26i | AAGGCCTGGGGACACCCGAGATGCCTGGTTATAATTAACC |
| | | | CAGACATGTGGCTGCCCCCCCCCCCCAACACCTGCTGCCCT |
| | | | CTAAAAATAACCCTGCATTCCATGTTCCCGGCGAAGGGCC |
| | | | AGCTGTCCCCCGCCAGCTAGACTCAGCACTTAGTTTAGGA |
| | | | ACCAGTGAGCAAGTCAGCCCTTGGGGCAGCCCATACAAGG |
| | | | CCATGGGGCTGGGCAAGCTGCACGCCTGGGTCCGGGGTGG |
| | | | GCACGGTGCCCGGGCAACGAGCTGAAAGCTCATCTACTCT |
| | | | CAGGGGCCCCTCCCTGGGGACAGCCCCTCCTGGCTAGTCA |
| | | | CACCCTGTAGGCTCCTCTATATAACCCAGGGGCACAGGGG |
| | | | CTGCCCTCATTCTACCACCACCTCCACAGCACAGACAGAC |
| | | | ACTCAGGAGCCAGCCAGCCCGAAGTAACTTCTCGGTAAAG |
| | | | TCTTCACGGAATTTCCAGACCACACTTGCCCACTGGGAGG |
| | | | CTTTTAGGACCCGAGACGTGTGCAGGCTTTTCCAGGTTCGG |
| | | | GTTGGTGGGCCTTATTCATTTTGGCGGGGCTATACGTATTC |
| | | | ACTCTTAAGCCTGTAGAAGATGAACGTTGATTGTACCTTCT |
| | | | AAAACGTGAGGTTATAGTTAAGAGAATTGCGTTGTCGGTC |
| | | | TGCTCAGCAGAGATGTAAGTCCAGTCTTGATTTCCTCAGGC |
| | | | ACTACTTGAGGAGTTGGGAGAGTGTAACTAAAAGGTCTTA |
| | | | TAACTAGGACAAGTGTTTTTGATGCAAAAGTCTTTAGTCCG |
| | | | TAAACATATACAGAAAACACCCGAATAAAACAAAAAATA |
| | | | AAAAGTTTCGAGCTTCGGTTGATAGGCCCTTCCGATCAAA |
| | | | CAGTTTTTGTTGAAGTTGCACAACATGACACTGCCTATGAT |
| | | | CATTCTAGACTATCAAGATAAAATCATTGCGGTTATTTGTT |
| | | | TGGAAGCTGGATGAGATTTTGGTCATTTAAGAGTCTTTTTA |
| | | | ACTTTTTATTTAGCCAAATTGAAGTTTAGCCACC |
| 128 | | CK8e | TGCCCATGTAAGGAGGCAAGGCCTGGGGACACCCGAGATG |
| | | promoter | CCTGGTTATAATTAACCCAGACATGTGGCTGCCCCCCCCCC |
| | | | CCAACACCTGCTGCCCTCTAAAAATAACCCTGCATTCCATG |
| | | | TTCCCGGCGAAGGGCCAGCTGTCCCCCGCCAGCTAGACTC |
| | | | AGCACTTAGTTTAGGAACCAGTGAGCAAGTCAGCCCTTGG |
| | | | GGCAGCCCATACAAGGCCATGGGGCTGGGCAAGCTGCACG |
| | | | CCTGGGTCCGGGGTGGGCACGGTGCCCGGGCAACGAGCTG |
| | | | AAAGCTCATCTACTCTCAGGGGCCCCTCCCTGGGGACAGC |
| | | | CCCTCCTGGCTAGTCACACCCTGTAGGCTCCTCTATATAAC |
| | | | CCAGGGGCACAGGGGCTGCCCTCATTCTACCACCACCTCC |
| | | | ACAGCACAGACAGACACTCAGGAGCCAGCCAGCC |
| 210 | | RPL26i | CGAAGTAACTTCTCGGTAAAGTCTTCACGGAATTTCCAGA |
| | | | CCACACTTGCCCACTGGGAGGCTTTTAGGACCCGAGACGT |
| | | | GTGCAGGCTTTTCCAGGTTCGGGTTGGTGGGCCTTATTCAT |
| | | | TTTGGCGGGGCTATACGTATTCACTCTTAAGCCTGTAGAAG |
| | | | ATGAACGTTGATTGTACCTTCTAAAACGTGAGGTTATAGTT |
| | | | AAGAGAATTGCGTTGTCGGTCTGCTCAGCAGAGATGTAAG |
| | | | TCCAGTCTTGATTTCCTCAGGCACTACTTGAGGAGTTGGGA |
| | | | GAGTGTAACTAAAAGGTCTTATAACTAGGACAAGTGTTTT |
| | | | TGATGCAAAAGTCTTTAGTCCGTAAACATATACAGAAAAC |
| | | | ACCCGAATAAAACAAAAAATAAAAAGTTTCGAGCTTCGGT |
| | | | TGATAGGCCCTTCCGATCAAACAGTTTTTGTTGAAGTTGCA |
| | | | CAACATGACACTGCCTATGATCATTCTAGACTATCAAGAT |
| | | | AAAATCATTGCGGTTATTTGTTTGGAAGCTGGATGAGATTT |
| | | | TGGTCATTTAAGAGTCTTTTTAACTTTTTATTTAGCCAAATT |
| | | | GAAGTTTA |
| 96 | M96 | ACTA1.1 | CGGGCGGGGGCCCAGGCCGCGAACCGGCCGAGGGAGGGG |
| | | | GCTCTAGTGCCCAACACCCAAATATGGCTCGAGAAGGGCA |
| | | | GCGACATTCCTGCGGGGTGGCGCGGAGGGAATGCCCGCGG |
| | | | GCTATATAAAACCTGAGCAGAGGGACAAGCGGCCACCGC |
| | | | AGCGGACAGCGCCAAGTGAAGCCTCGCTTCCCCTCCGCGG |
| | | | CGACCAGGGGCGGATCCGCCACC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 166 | | minACTA1.1 | TCGAGAAGGGCAGCGACATTCCTGCGGGGTGGCGCGGAG |
| | | promoter | GGAATGCCCGCGGGCTATATAAAACCTGAGCAGAGGGAC |
| | | | AAGCGGCCACCGCAGCGGACAGCGCCAAGTGAAGCCTCG |
| | | | CTTCCCCTCCGCGGCGACCAGGG |
| 97 | M97 | ACTA1.2 | CGGGCGGGGGCCCAGGCCGCGAACCGGCCGAGGGAGGGG |
| | | | GCTCTAGTGCCCAACACCCAAATATGGCTCGAGAAGGGCA |
| | | | GCGACATTCCTGCGGGGTGGCGCGGAGGGAATGCCCGCGG |
| | | | GCTATATAAAACCTGAGCAGAGGGACAAGCGGCCACCGC |
| | | | AGCGGACAGCGCCGAGCCGAGTGGTCGTTCCCCTCCGCGG |
| | | | CGACCAGGGGCGGATCCGCCACC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 167 | | minACTA1.2 | TCGAGAAGGGCAGCGACATTCCTGCGGGGTGGCGCGGAG |
| | | promoter | GGAATGCCCGCGGGCTATATAAAACCTGAGCAGAGGGAC |
| | | | AAGCGGCCACCGCAGCGGACAGCGCCGAGCCGAGTGGTC |
| | | | GTTCCCCTCCGCGGCGACCAGGG |
| 98 | M98 | ACTA1.3 | CGGGGGGGGCCCAGGCCGCGAACCGGCCGAGGGAGGGG |
| | | | GCTCTAGTGCCCAACACCCAAATATGGCTCGAGAAGGGCA |
| | | | GCGACATTCCTGCGGGGTGGCGCGGAGGGAATGCCCGGCG |
| | | | CCTATAAAAAGTGTGGGGCGCGGCGGGCGGGTCAGTCCGC |
| | | | CTGGAGACCTCGAGCCGAGTGGTCGTGCCTCCATAGAAGG |
| | | | ATCCGCCACC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 168 | | minACTA1.3 | TCGAGAAGGGCAGCGACATTCCTGCGGGGTGGCGCGGAG |
| | | promoter | GGAATGCCCGGCGCCTATAAAAAGTGTGGGGCGCGGCGG |
| | | variant 1 | GCGGGTCAGTCCGCCTGGAGACCTCGAGCCGAGTGGTCGT |
| | | | GCCTCCATAGAA |
| 99 | M99 | ACTA1.4 | CGGGCGGGGGCCCAGGCCGCGAACCGGCCGAGGGAGGGG |
| | | | GCTCTAGTGCCCAACACCCAAATATGGCTCGAGAAGGGCA |
| | | | GCGACATTCCTGCGGGGTGGCGCGGAGGGAATGCCCGGCG |
| | | | CCTATAAAAAGTGTGGGGCGCGGCGGGCGGGTCAGTCCGC |
| | | | CTGGAGACCTCGAGCCGAGTGGTCGTTGAGCCAAGAGGTA |
| | | | AGGGTTTAAGGGATGGTTGGTTGGTGGGGTATTAATGTTT |
| | | | AATTACCTGGAGCACCTGCCTGAAATCACTTTTTTTCAGGT |
| | | | TGGCCGCGGATCCGCCACC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 171 | | minACTA1.4 | TCGAGAAGGGCAGCGACATTCCTGCGGGGTGGCGCGGAG |
| | | promoter | GGAATGCCCGGCGCCTATAAAAAGTGTGGGGCGCGGCGG |
| | | | GCGGGTCAGTCCGCCTGGAGACCTCGAGCCGAGTGGTCGT |
| | | | TGAGCCAAGAGGTAAGGGTTTAAGGGATGGTTGGTTGGTG |
| | | | GGGTATTAATGTTTAATTACCTGGAGCACCTGCCTGAAATC |
| | | | ACTTTTTTTCAGGTTGGCCGC |
| 200 | | MVM intron | AAGAGGTAAGGGTTTAAGGGATGGTTGGTTGGTGGGGTAT |
| | | (part of | TAATGTTTAATTACCTGGAGCACCTGCCTGAAATCACTTTT |
| | | minACTA1.4 | TTTCAGGTTGG |
| | | promoter) | |
| 100 | M100 | ACTA1.5 | TGCCCATGTAAGGAGGCAAGGCCTGGGGACACCCGAGATG |
| | | | CCTGGTTATAATTAACCCAGACATGTGGCTGCCCCCCCCCC |
| | | | CCAACACCTGCTGCCCTCTAAAAATAACCCTGCGGGCGGG |
| | | | GGCCCAGGCCGCGAACCGGCCGAGGGAGGGGGCTCTAGT |
| | | | GCCCAACACCCAAATATGGCTCGAGAAGGGCAGCGACATT |
| | | | CCTGCGGGGTGGCGCGGAGGGAATGCCCGCGGGCTATATA |
| | | | AAACCTGAGCAGAGGGACAAGCGGCCACCGCAGCGGACA |
| | | | GCGCCGAGCCGAGTGGTCGTTCCCCTCCGCGGCGACCAGG |
| | | | GGCGGATCCGCCACC |
| 154 | | MCK | TGCCCATGTAAGGAGGCAAGGCCTGGGGACACCCGAGATG |
| | | enhancer | CCTGGTTATAATTAACCCAGACATGTGGCTGCCCCCCCCCC |
| | | | CCAACACCTGCTGCCCTCTAAAAATAACCCTG |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 167 | | minACTA1.2 | TCGAGAAGGGCAGCGACATTCCTGCGGGGTGGCGCGGAG |
| | | promoter | GGAATGCCCGCGGGCTATATAAAACCTGAGCAGAGGGAC |
| | | | AAGCGGCCACCGCAGCGGACAGCGCCGAGCCGAGTGGTC |
| | | | GTTCCCCTCCGCGGCGACCAGGG |
| 101 | M101 | ACTA1.6 | TGGCTGATCCTCTCCCCTGCCCTTGGCTCCATGAATGGCCT |
| | | | CGGCAGTCCTAGCGGGTGCCGGGCGGGGGCCCAGGCCGCG |
| | | | AACCGGCCGAGGGAGGGGGCTCTAGTGCCCAACACCCAA |
| | | | ATATGGCTCGAGAAGGGCAGCGACATTCCTGCGGGGTGGC |
| | | | GCGGAGGGAATGCCCGCGGGCTATATAAAACCTGAGCAG |
| | | | AGGGACAAGCGGCCACCGCAGCGGACAGCGCCGAGCCGA |
| | | | GTGGTCGTTCCCCTCCGCGGCGACCAGGGGCGGATCCGCC |
| | | | ACC |
| 122 | | ACTC1 | TGGCTGATCCTCTCCCCTGCCCTTGGCTCCATGAATGGCCT |
| | | enhancer | CGGCAGTCCTAGCGGGTGC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 167 | | minACTA1.2 | TCGAGAAGGGCAGCGACATTCCTGCGGGGTGGCGCGGAG |
| | | promoter | GGAATGCCCGCGGGCTATATAAAACCTGAGCAGAGGGAC |
| | | | AAGCGGCCACCGCAGCGGACAGCGCCGAGCCGAGTGGTC |
| | | | GTTCCCCTCCGCGGCGACCAGGG |
| 102 | M102 | ACTC1.1 | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCAGCGTTCTATAAAGCGGCCCTC |
| | | | CTGGAGCCAGCCACCCAGAGCCCGCTGCCGCCGGAGCCGA |
| | | | GCCGACCCGCCCCGCCGGGATCCGCCACC |
| 122 | | ACTC1 | TGGCTGATCCTCTCCCCTGCCCTTGGCTCCATGAATGGCCT |
| | | enhancer | CGGCAGTCCTAGCGGGTGC |
| 173 | | minACTC1 | GAAGGGGACCAAATAAGGCAAGGTGGCAGACCGGGCCCC |
| | | promoter | CCACCCCTGCCCCCGGCTGCTCCAACTGACCCTGTCCATCA |
| | | | GCGTTCTATAAAGCGGCCCTCCTGGAGCCAGCCACCCAGA |
| | | | GCCCGCTGCCGCCGGAGCCGAGCCGACCCGCCCCGCCG |
| 103 | M103 | ACTC1.2 | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCAGCGTTCTATAAAGCGGCCCTC |
| | | | CTGGAGCCAGCCACCCAGAGCCCGCTGCCGCCGCGAGCCG |
| | | | AGTGGTCGTCCCCGCCGGGATCCGCCACC |
| 122 | | ACTC1 | TGGCTGATCCTCTCCCCTGCCCTTGGCTCCATGAATGGCCT |
| | | enhancer | CGGCAGTCCTAGCGGGTGC |
| 174 | | minACTC1.2 | GAAGGGGACCAAATAAGGCAAGGTGGCAGACCGGGCCCC |
| | | promoter | CCACCCCTGCCCCCGGCTGCTCCAACTGACCCTGTCCATCA |
| | | | GCGTTCTATAAAGCGGCCCTCCTGGAGCCAGCCACCCAGA |
| | | | GCCCGCTGCCGCCGCGAGCCGAGTGGTCGTCCCCGCCG |
| 104 | M104 | ACTC1.3 | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCCGGCGCCTATAAAAAGTGTGG |
| | | | GGCGCGGCGGGCGGGTCAGTCCGCCTGGAGACCTCGAGCC |
| | | | GAGTGGTCGTGCCTCCATAGAAGGATCCGCCACC |
| 122 | | ACTC1 | TGGCTGATCCTCTCCCCTGCCCTTGGCTCCATGAATGGCCT |
| | | enhancer | CGGCAGTCCTAGCGGGTGC |
| 176 | | minACTC1.3 | GAAGGGGACCAAATAAGGCAAGGTGGCAGACCGGGCCCC |
| | | promoter | CCACCCCTGCCCCCGGCTGCTCCAACTGACCCTGTCCATCC |
| | | | GGCGCCTATAAAAAGTGTGGGGCGCGGCGGGCGGGTCAGT |
| | | | CCGCCTGGAGACCTCGAGCCGAGTGGTCGTGCCTCCATAG |
| | | | AA |
| 105 | M105 | ACTC1.4 | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCCGGCGCCTATAAAAAGTGTGG |
| | | | GGCGCGGCGGGCGGGTCAGTCCGCCTGGAGACCTCGAGCC |
| | | | GAGTGGTCGTTGAGCCAAGAGGTAAGGGTTTAAGGGATGG |
| | | | TTGGTTGGTGGGGTATTAATGTTTAATTACCTGGAGCACCT |
| | | | GCCTGAAATCACTTTTTTTCAGGTTGGCCGCGGATCCGCCA |
| | | | CC |
| 122 | | ACTC1 | TGGCTGATCCTCTCCCCTGCCCTTGGCTCCATGAATGGCCT |
| | | enhancer | CGGCAGTCCTAGCGGGTGC |
| 178 | | minACTC1.4 | GAAGGGGACCAAATAAGGCAAGGTGGCAGACCGGGCCCC |
| | | promoter | CCACCCCTGCCCCCGGCTGCTCCAACTGACCCTGTCCATCC |
| | | | GGCGCCTATAAAAAGTGTGGGGCGCGGCGGGCGGGTCAGT |
| | | | CCGCCTGGAGACCTCGAGCCGAGTGGTCGTTGAGCCAAGA |
| | | | GGTAAGGGTTTAAGGGATGGTTGGTTGGTGGGGTATTAAT |
| | | | GTTTAATTACCTGGAGCACCTGCCTGAAATCACTTTTTTTC |
| | | | AGGTTGGCCGC |
| 200 | | MVM intron | AAGAGGTAAGGGTTTAAGGGATGGTTGGTTGGTGGGGTAT |
| | | (part of | TAATGTTTAATTACCTGGAGCACCTGCCTGAAATCACTTTT |
| | | minACTC1.4 | TTTCAGGTTGG |
| | | promoter) | |
| 106 | M106 | ACTC1.5 | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCCGGCGCCTATAAAAAGTGTGG |
| | | | GGCGCGGCGGGGGGTCAGTCCGCCTGGAGACCTCGAGCC |
| | | | GAGTGGTCGTTGAGCCAAGAGGTAAGGGTTTAACGGGCGG |
| | | | GGGCCCAGGCCGCGAACCGGCCGAGGGAGGGGGCTCTAG |
| | | | TGCCCAACACCCAAATATGGCGAGCACCTGCCTGAAATCA |
| | | | CTTTTTTTCAGGTTGGCCGCGGATCCGCCACC |
| 122 | | ACTC1 | TGGCTGATCCTCTCCCCTGCCCTTGGCTCCATGAATGGCCT |
| | | enhancer | CGGCAGTCCTAGCGGGTGC |
| 179 | | minACTC1.5 | GAAGGGGACCAAATAAGGCAAGGTGGCAGACCGGGCCCC |
| | | promoter | CCACCCCTGCCCCCGGCTGCTCCAACTGACCCTGTCCATCC |
| | | | GGCGCCTATAAAAAGTGTGGGGCGCGGCGGGCGGGTCAGT |
| | | | CCGCCTGGAGACCTCGAGCCGAGTGGTCGTTGAGCCAAGA |
| | | | GGTAAGGGTTTAACGGGCGGGGGCCCAGGCCGCGAACCG |
| | | | GCCGAGGGAGGGGGCTCTAGTGCCCAACACCCAAATATGG |
| | | | CGAGCACCTGCCTGAAATCACTTTTTTTCAGGTTGGCCGC |
| 202 | | MVM- | AAGAGGTAAGGGTTTAACGGGCGGGGGCCCAGGCCGCGA |
| | | ACTA1e | ACCGGCCGAGGGAGGGGGCTCTAGTGCCCAACACCCAAAT |
| | | intron | ATGGCGAGCACCTGCCTGAAATCACTTTTTTTCAGGTTGG |
| | | (part of | |
| | | minACTC1.5 | |
| | | promoter) | |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| | | (part of | |
| | | MVM- | |
| | | ACTA1e | |
| | | intron) | |
| 107 | M107 | ACTAC1.1 | CGGGCGGGGGCCCAGGCCGCGAACCGGCCGAGGGAGGGG |
| | | | GCTCTAGTGCCCAACACCCAAATATGGCTCGAGAAGGGCA |
| | | | GCGACATTCCTGCGGGGTGGCGCGGAGGGAATGCCCGGCG |
| | | | CCTATAAAAAGTGTGGGGCGCGGCGGGCGGGTCAGTCCGC |
| | | | CTGGAGACCTCGAGCCGAGTGGTCGTTGAGCCAAGAGGTA |
| | | | AGGGTTTAAGGGATGGTTGGTTGTGGCTGATCCTCTCCCCT |
| | | | GCCCTTGGCTCCATGAATGGCCTCGGCAGTCCTAGCGGGT |
| | | | GCGAAGGGGACCAAATAAGGCAAGGTGGCAGACCGGGCC |
| | | | CCCCACCCCTGCCCCCGGCTGCTCCAACTGACCCTGTCCAT |
| | | | CCGGCGCCTATAAAAAGTGTGGGGCGCGGCGGGCGGGTCA |
| | | | GTCCGCCTGGAGACCTCGAGCCGAGTGGTCGTGTGGGGTA |
| | | | TTAATGTTTAATTACCTGGAGCACCTGCCTGAAATCACTTT |
| | | | TTTTCAGGTTGGCCGCGGATCCGCCACC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| 172 | | minACTAC1.1 | TCGAGAAGGGCAGCGACATTCCTGCGGGGTGGCGCGGAG |
| | | promoter | GGAATGCCCGGCGCCTATAAAAAGTGTGGGGCGCGGCGG |
| | | | GCGGGTCAGTCCGCCTGGAGACCTCGAGCCGAGTGGTCGT |
| | | | TGAGCCAAGAGGTAAGGGTTTAAGGGATGGTTGGTTGTGG |
| | | | CTGATCCTCTCCCCTGCCCTTGGCTCCATGAATGGCCTCGG |
| | | | CAGTCCTAGCGGGTGCGAAGGGGACCAAATAAGGCAAGG |
| | | | TGGCAGACCGGGCCCCCCACCCCTGCCCCCGGCTGCTCCA |
| | | | ACTGACCCTGTCCATCCGGCGCCTATAAAAAGTGTGGGGC |
| | | | GCGGCGGGGGGTCAGTCCGCCTGGAGACCTCGAGCCGAG |
| | | | TGGTCGTGTGGGGTATTAATGTTTAATTACCTGGAGCACCT |
| | | | GCCTGAAATCACTTTTTTTCAGGTTGGCCGC |
| 206 | | MVM- | AAGAGGTAAGGGTTTAAGGGATGGTTGGTTGTGGCTGATC |
| | | ACTC1e | CTCTCCCCTGCCCTTGGCTCCATGAATGGCCTCGGCAGTCC |
| | | intron | TAGCGGGTGCGAAGGGGACCAAATAAGGCAAGGTGGCAG |
| | | (part of | ACCGGGCCCCCCACCCCTGCCCCCGGCTGCTCCAACTGAC |
| | | minACTAC1.1 | CCTGTCCATCCGGCGCCTATAAAAAGTGTGGGGCGCGGCG |
| | | promoter) | GGCGGGTCAGTCCGCCTGGAGACCTCGAGCCGAGTGGTCG |
| | | | TGTGGGGTATTAATGTTTAATTACCTGGAGCACCTGCCTGA |
| | | | AATCACTTTTTTTCAGGTTGG |
| 122 | | ACTC1 | TGGCTGATCCTCTCCCCTGCCCTTGGCTCCATGAATGGCCT |
| | | enhancer | CGGCAGTCCTAGCGGGTGC |
| | | (part of | |
| | | MVM- | |
| | | ACTC1e | |
| | | intron) | |
| 108 | M108 | ACTAC1.2 | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCCGGCGCCTATAAAAAGTGTGG |
| | | | GGCGCGGCGGGCGGGTCAGTCCGCCTGGAGACCTCGAGCC |
| | | | GAGTGGTCGTTGAGCCAAGAGGTAAGGGTTTAAGGGATGG |
| | | | TTGGTTGTGGCTGATCCTCTCCCCTGCCCTTGGCTCCATGA |
| | | | ATGGCCTCGGCAGTCCTAGCGGGTGCGAAGGGGACCAAAT |
| | | | AAGGCAAGGTGGCAGACCGGGCCCCCCACCCCTGCCCCCG |
| | | | GCTGCTCCAACTGACCCTGTCCATCAGCGTTCTATAAAGCG |
| | | | GCCCTCCTGGAGCCAGCCACCCAGAGCCCGCTGCCGCCGC |
| | | | GAGCCGAGTGGTCGTCCCCGCCGGGATCCGCCACCGTGGG |
| | | | GTATTAATGTTTAATTACCTGGAGCACCTGCCTGAAATCAC |
| | | | TTTTTTTCAGGTTGGCCGCGGATCCGCCACC |
| 122 | | ACTC1 | TGGCTGATCCTCTCCCCTGCCCTTGGCTCCATGAATGGCCT |
| | | enhancer | CGGCAGTCCTAGCGGGTGC |
| 188 | | miniACTC1.3 | GAAGGGGACCAAATAAGGCAAGGTGGCAGACCGGGCCCC |
| | | promoter | CCACCCCTGCCCCCGGCTGCTCCAACTGACCCTGTCCATCC |
| | | (partial) | GGCGCCTATAAAAAGTGTGGGGCGCGGCGGGCGGGTCAGT |
| | | | CCGCCTGGAGACCTCGAGCCGAGTGGTCGT |
| 207 | | MVM- | AAGAGGTAAGGGTTTAAGGGATGGTTGGTTGTGGCTGATC |
| | | ACTC1e- | CTCTCCCCTGCCCTTGGCTCCATGAATGGCCTCGGCAGTCC |
| | | ACTC1p | TAGCGGGTGCGAAGGGGACCAAATAAGGCAAGGTGGCAG |
| | | intron | ACCGGGCCCCCCACCCCTGCCCCCGGCTGCTCCAACTGAC |
| | | | CCTGTCCATCAGCGTTCTATAAAGCGGCCCTCCTGGAGCCA |
| | | | GCCACCCAGAGCCCGCTGCCGCCGCGAGCCGAGTGGTCGT |
| | | | CCCCGCCGGGATCCGCCACCGTGGGGTATTAATGTTTAATT |
| | | | ACCTGGAGCACCTGCCTGAAATCACTTTTTTTCAGGTTGG |
| 122 | | ACTC1 | TGGCTGATCCTCTCCCCTGCCCTTGGCTCCATGAATGGCCT |
| | | enhancer | CGGCAGTCCTAGCGGGTGC |
| | | (part of | |
| | | MVM- | |
| | | ACTC1e- | |
| | | ACTC1p | |
| | | intron) | |
| 174 | | minACTC1.2 | GAAGGGGACCAAATAAGGCAAGGTGGCAGACCGGGCCCC |
| | | promoter | CCACCCCTGCCCCCGGCTGCTCCAACTGACCCTGTCCATCA |
| | | (part of | GCGTTCTATAAAGCGGCCCTCCTGGAGCCAGCCACCCAGA |
| | | MVM- | GCCCGCTGCCGCCGCGAGCCGAGTGGTCGTCCCCGCCG |
| | | ACTC1e- | |
| | | ACTC1p | |
| | | intron) | |
| 109 | M109 | DES.2 | GTTCTCCTCTATAAATACCCGCTCTGGTATTTGGGGTTGGC |
| | | | AGCTGTTGCTGCCCTATAAATACGTATTTATAGCTATAAAT |
| | | | ACCTGGCAGGACAGCGGGATCTTGCAGCTGTCAGGGGAGG |
| | | | GGAGGCGGGGGCTGATGTCAGGAGGGATACAAATAGTGC |
| | | | CGACGGCTGGGGGCCCTGTCTCCCCTCGCCGCATCCACTCT |
| | | | CCGGCCGGCCGCCTGCCCGCCGCCTCCTCCGTGCGCCCGCC |
| | | | AGCCTCGCCCGCGCCGGATCCGCCACC |
| 184 | | minDes | GTTCTCCTCTATAAATACCCGCTCTGGTATTTGGGGTTGGC |
| | | enhancer | AGCTGTTGCTGCCCTATAAATACGTATTTATAGCTATAAAT |
| | | | AC |
| 185 | | minDes | GGGATCTTGCAGCTGTCAGGGGAGGGGAGGCGGGGGCTG |
| | | promoter | ATGTCAGGAGGGATACAAATAGTGCCGACGGCTGGGGGCC |
| | | | CTGTCTCCCCTCGCCGCATCCACTCTCCGGC |
| 110 | M110 | DES.3 | GTTCTCCTCTATAAATACCCGCTCTGGTATTTGGGGTTGGC |
| | | | AGCTGTTGCTGCCCTATAAATACGTATTTATAGCTATAAAT |
| | | | ACCTGGCAGGACAGCGGGATCTTGCAGCTGTCAGGGGAGG |
| | | | GGAGGCGGGGGCTGATGTCAGGAGGGATATATAAAGTGC |
| | | | CGACGGCTGGGGGCCCTTCAGTCCGCCTGGAGACCTCGAG |
| | | | CCGAGTGGTCGTGGATCCGCCACC |
| 184 | | minDes | GTTCTCCTCTATAAATACCCGCTCTGGTATTTGGGGTTGGC |
| | | enhancer | AGCTGTTGCTGCCCTATAAATACGTATTTATAGCTATAAAT |
| | | | AC |
| 186 | | minDes.3 | GGGATCTTGCAGCTGTCAGGGGAGGGGAGGCGGGGGCTG |
| | | promoter | ATGTCAGGAGGGATATATAAAGTGCCGACGGCTGGGGGCC |
| | | | CTTCAGTCCGCCTGGAGACCTCGAGCCGAGTGGTCGT |
| 111 | M111 | DES.4 | GTTCTCCTCTATAAATACCCGCTCTGGTATTTGGGGTTGGC |
| | | | AGCTGTTGCTGCCCTATAAATACGTATTTATAGCTATAAAT |
| | | | ACCTGGCAGGACAGCGGGATCTTGCAGCTGTCAGGGGAGG |
| | | | GGAGGCGGGGGCTGATGTCACGGCGCCTATAAAAAGTGTG |
| | | | GGGCGCGGCGGGCGGGTCAGTCCGCCTGGAGACCTCGAGC |
| | | | CGAGTGGTCGTGCCTCCATAGAAGGATCCGCCACC |
| 184 | | minDes | GTTCTCCTCTATAAATACCCGCTCTGGTATTTGGGGTTGGC |
| | | enhancer | AGCTGTTGCTGCCCTATAAATACGTATTTATAGCTATAAAT |
| | | | AC |
| 187 | | minDes.4 | GGGATCTTGCAGCTGTCAGGGGAGGGGAGGCGGGGGCTG |
| | | promoter | ATGTCACGGCGCCTATAAAAAGTGTGGGGCGCGGCGGGCG |
| | | | GGTCAGTCCGCCTGGAGACCTCGAGCCGAGTGGTCGTGCC |
| | | | TCCATAGAA |
| 112 | M112 | CKM.1 | CCACTACGGGTCTAGGCTGCCCATGTAAGGAGGCAAGGCC |
| | | | TGGGGACACCCGAGATGCCTGGTTATAATTAACCCACAAC |
| | | | ACCTGCTGCCCCCCCCCCCCAACACCTGCTGCCTGAGCCTG |
| | | | AGCGGTTACCCCACCCCGGTGCCTGGGTCTTAGGCTCTGTA |
| | | | CACCATGGAGGAGAAGCTCGCTCTAAAAATAACCCTGTCC |
| | | | CTGGTGGATCTCCCTGGGGACAGCCCCTCCTGGCTAGTCAC |
| | | | ACCCTGTAGGCTCCTCTATATAACCCAGGGGCACAGGGGC |
| | | | TGCCCTCAGTCCGCCTGGAGACCTCGAGCCGAGTGGTCGT |
| | | | CTCAGGAGCCGGATCCGCCACC |
| 180 | | minCKM | CCACTACGGGTCTAGGCTGCCCATGTAAGGAGGCAAGGCC |
| | | enhancer | TGGGGACACCCGAGATGCCTGGTTATAATTAACCCACAAC |
| | | | ACCTGCTGCCCCCCCCCCCCAACACCTGCTGCCTGAGCCTG |
| | | | AGCGGTTACCCCACCCCGGTGCCTGGGTCTTAGGCTCTGTA |
| | | | CACCATGGAGGAGAAGCTCGCTCTAAAAATAACCCTGTCC |
| | | | CTGGTGGAT |
| 181 | | minCKM.1 | CTCCCTGGGGACAGCCCCTCCTGGCTAGTCACACCCTGTAG |
| | | promoter | GCTCCTCTATATAACCCAGGGGCACAGGGGCTGCCCTCAG |
| | | | TCCGCCTGGAGACCTCGAGCCGAGTGGTCGTCTCAGGAGC |
| | | | C |
| 113 | M113 | CKM.2 | CCACTACGGGTCTAGGCTGCCCATGTAAGGAGGCAAGGCC |
| | | | TGGGGACACCCGAGATGCCTGGTTATAATTAACCCACAAC |
| | | | ACCTGCTGCCCCCCCCCCCCAACACCTGCTGCCTGAGCCTG |
| | | | AGCGGTTACCCCACCCCGGTGCCTGGGTCTTAGGCTCTGTA |
| | | | CACCATGGAGGAGAAGCTCGCTCTAAAAATAACCCTGTCC |
| | | | CTGGTGGATCTCCCTGGGGACAGCCCCTCCTGGCTAGTCAC |
| | | | ACCCTGTAGCGGCGCCTATAAAAAGTGTGGGGCGCGGCGG |
| | | | GCGGGTCAGTCCGCCTGGAGACCTCGAGCCGAGTGGTCGT |
| | | | CTCAGGAGCCGGATCCGCCACC |
| 180 | | minCKM | CCACTACGGGTCTAGGCTGCCCATGTAAGGAGGCAAGGCC |
| | | enhancer | TGGGGACACCCGAGATGCCTGGTTATAATTAACCCACAAC |
| | | | ACCTGCTGCCCCCCCCCCCCAACACCTGCTGCCTGAGCCTG |
| | | | AGCGGTTACCCCACCCCGGTGCCTGGGTCTTAGGCTCTGTA |
| | | | CACCATGGAGGAGAAGCTCGCTCTAAAAATAACCCTGTCC |
| | | | CTGGTGGAT |
| 182 | | minCKM.2 | CTCCCTGGGGACAGCCCCTCCTGGCTAGTCACACCCTGTAG |
| | | promoter | CGGCGCCTATAAAAAGTGTGGGGCGCGGCGGGGGGTCA |
| | | | GTCCGCCTGGAGACCTCGAGCCGAGTGGTCGTCTCAGGAG |
| | | | CC |
| 114 | M114 | CKM.3 | CCGAGATGCCTGGTTATAATTAACCCACAACACCTGCTGC |
| | | | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| | | | CTCCCTGGGGACAGCCCCTCCTGGCTAGTCACACCCTGTAG |
| | | | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CCGGATCCGCCACC |
| 183 | | minCKM2 | CCGAGATGCCTGGTTATAATTAACCCACAACACCTGCTGC |
| | | enhancer | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| 182 | | minCKM.2 | CTCCCTGGGGACAGCCCCTCCTGGCTAGTCACACCCTGTAG |
| | | promoter | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CC |
| 115 | M115 | CKM.4 | CCGAGATGCCTGGTTATAATTAACCCACAACACCTGCTGC |
| | | | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| | | | CTCCCTGGGGACAGCCCCTCCTGGCTAGTCACACCCTGTAG |
| | | | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CCAAGAGGTAAGGGTTTAAGGGATGGTTGGTTGGTGGGGT |
| | | | ATTAATGTTTAATTACCTGGAGCACCTGCCTGAAATCACTT |
| | | | TTTTTCAGGTTGGGGATCCGCCACC |
| 183 | | minCKM2 | CCGAGATGCCTGGTTATAATTAACCCACAACACCTGCTGC |
| | | enhancer | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| 182 | | minCKM.2 | CTCCCTGGGGACAGCCCCTCCTGGCTAGTCACACCCTGTAG |
| | | promoter | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CC |
| 200 | | MVM intron | AAGAGGTAAGGGTTTAAGGGATGGTTGGTTGGTGGGGTAT |
| | | | TAATGTTTAATTACCTGGAGCACCTGCCTGAAATCACTTTT |
| | | | TTTCAGGTTGG |
| 116 | M116 | CKM- | CCGAGATGCCTGGTTATAATTAACCCACAACACCTGCTGC |
| | | ACTC | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| | | | CTCCCTGGGGACAGCCCCTCCTGGCTAGTCACACCCTGTAG |
| | | | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CCAAGAGGTAAGGGTTTAAGGGATGGTTGGTTGTGGCTGA |
| | | | TCCTCTCCCCTGCCCTTGGCTCCATGAATGGCCTCGGCAGT |
| | | | CCTAGCGGGTGCGAAGGGGACCAAATAAGGCAAGGTGGC |
| | | | AGACCGGGCCCCCCACCCCTGCCCCCGGCTGCTCCAACTG |
| | | | ACCCTGTCCATCCGGCGCCTATAAAAAGTGTGGGGCGCGG |
| | | | CGGGCGGGTCAGTCCGCCTGGAGACCTCGAGCCGAGTGGT |
| | | | CGTGTGGGGTGTTTAATTACCTGGAGCACCTGCCTGAAATC |
| | | | ACTTTTTTTCAGGTTGGGGATCCGCCACC |
| 183 | | minCKM2 | CCGAGATGCCTGGTTATAATTAACCCACAACACCTGCTGC |
| | | enhancer | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| 182 | | minCKM.2 | CTCCCTGGGGACAGCCCCTCCTGGCTAGTCACACCCTGTAG |
| | | promoter | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CC |
| 205 | | MVM- | AAGAGGTAAGGGTTTAAGGGATGGTTGGTTGTGGCTGATC |
| | | ACTC1.3 | CTCTCCCCTGCCCTTGGCTCCATGAATGGCCTCGGCAGTCC |
| | | intron | TAGCGGGTGCGAAGGGGACCAAATAAGGCAAGGTGGCAG |
| | | | ACCGGGCCCCCCACCCCTGCCCCCGGCTGCTCCAACTGAC |
| | | | CCTGTCCATCCGGCGCCTATAAAAAGTGTGGGGCGCGGCG |
| | | | GGCGGGTCAGTCCGCCTGGAGACCTCGAGCCGAGTGGTCG |
| | | | TGTGGGGTGTTTAATTACCTGGAGCACCTGCCTGAAATCAC |
| | | | TTTTTTTCAGGTTGG |
| 122 | | ACTC1 | TGGCTGATCCTCTCCCCTGCCCTTGGCTCCATGAATGGCCT |
| | | enhancer | CGGCAGTCCTAGCGGGTGC |
| | | components | |
| | | (part of | |
| | | MVM- | |
| | | ACTC1.3 | |
| | | intron) | |
| 177 | | minACTC1.3 | GAAGGGGACCAAATAAGGCAAGGTGGCAGACCGGGCCCC |
| | | promoter | CCACCCCTGCCCCCGGCTGCTCCAACTGACCCTGTCCATCC |
| | | (partial) | GGCGCCTATAAAAAGTGTGGGGCGCGGCGGGCGGGTCAGT |
| | | (part of | CCGCCTGGAGACCTCGAGCCGAGTGGTCGT |
| | | MVM- | |
| | | ACTC1.3 | |
| | | intron) | |
| 117 | M117 | CKM-DES | CCGAGATGCCTGGTTATAATTAACCCACAACACCTGCTGC |
| | | | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| | | | CTCCCTGGGGACAGCCCCTCCTGGCTAGTCACACCCTGTAG |
| | | | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CCAAGAGGTAAGGGTTTAAGGGATGGTTGGTTGGTTCTCC |
| | | | TCTATAAATACCCGCTCTGGTATTTGGGGTTGGCAGCTGTT |
| | | | GCTGCCCTATAAATACGTATTTATAGCTATAAATACCTGGC |
| | | | AGGACAGCGGGATCTTGCAGCTGTCAGGGGAGGGGAGGC |
| | | | GGGGGCTGATGTCACGGCGCCTATAAAAAGTGTGGGGCGC |
| | | | GGCGGGCGGGTCAGTCCGCCTGGAGACCTCGAGCCGAGTG |
| | | | GTCGTGCCTCCATAGAAGTGGGGTGTTTAATTACCTGGAG |
| | | | CACCTGCCTGAAATCACTTTTTTTCAGGTTGGGGATCCGCC |
| | | | ACC |
| 183 | | minCKM2 | CCGAGATGCCTGGTTATAATTAACCCACAACACCTGCTGC |
| | | enhancer | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| 182 | | minCKM.2 | CTCCCTGGGGACAGCCCCTCCTGGCTAGTCACACCCTGTAG |
| | | promoter | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CC |
| 208 | | MVM- | AAGAGGTAAGGGTTTAAGGGATGGTTGGTTGGTTCTCCTC |
| | | DES.4 | TATAAATACCCGCTCTGGTATTTGGGGTTGGCAGCTGTTGC |
| | | intron | TGCCCTATAAATACGTATTTATAGCTATAAATACCTGGCAG |
| | | | GACAGCGGGATCTTGCAGCTGTCAGGGGAGGGGAGGCGG |
| | | | GGGCTGATGTCACGGCGCCTATAAAAAGTGTGGGGCGCGG |
| | | | CGGGCGGGTCAGTCCGCCTGGAGACCTCGAGCCGAGTGGT |
| | | | CGTGCCTCCATAGAAGTGGGGTGTTTAATTACCTGGAGCA |
| | | | CCTGCCTGAAATCACTTTTTTTCAGGTTGG |
| 184 | | minDes | GTTCTCCTCTATAAATACCCGCTCTGGTATTTGGGGTTGGC |
| | | enhancer | AGCTGTTGCTGCCCTATAAATACGTATTTATAGCTATAAAT |
| | | (part of | AC |
| | | MVM- | |
| | | DES.4 | |
| | | intron) | |
| 187 | | minDes.4 | GGGATCTTGCAGCTGTCAGGGGAGGGGAGGCGGGGGCTG |
| | | promoter | ATGTCACGGCGCCTATAAAAAGTGTGGGGCGCGGCGGGCG |
| | | (part of | GGTCAGTCCGCCTGGAGACCTCGAGCCGAGTGGTCGTGCC |
| | | MVM- | TCCATAGAA |
| | | DES.4 | |
| | | intron) | |
| 118 | M118 | CKM- | CCGAGATGCCTGGTTATAATTAACCCACAACACCTGCTGC |
| | | ACTA | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| | | | CTCCCTGGGGACAGCCCCTCCTGGCTAGTCACACCCTGTAG |
| | | | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CCAAGAGGTAAGGGTTTAAGGGATGGTTGGTTGCGGGCGG |
| | | | GGGCCCAGGCCGCGAACCGGCCGAGGGAGGGGGCTCTAG |
| | | | TGCCCAACACCCAAATATGGCTCGAGAAGGGCAGCGACAT |
| | | | TCCTGCGGGGTGGCGCGGAGGGAATGCCCGGCGCCTATAA |
| | | | AAAGTGTGGGGCGCGGCGGGGGGTCAGTCCGCCTGGAG |
| | | | ACCTCGAGCCGAGTGGTCGTGTGGGGTGTTTAATTACCTG |
| | | | GAGCACCTGCCTGAAATCACTTTTTTTCAGGTTGGGGATCC |
| | | | GCCACC |
| 183 | | minCKM2 | CCGAGATGCCTGGTTATAATTAACCCACAACACCTGCTGC |
| | | enhancer | CCCCCCCCCCCAACACCTGCTGCCTGAGCCTGAGCGGTTAC |
| | | | CCCACCCCGGTGCCTGGGTCTTAGGCTCTGTACACCATGGA |
| | | | GGAGAAGCTCGCTCTAAAAATAACCCTGTCCCTGGTGGAT |
| 182 | | minCKM.2 | CTCCCTGGGGACAGCCCCTCCTGGCTAGTCACACCCTGTAG |
| | | promoter | CGGCGCCTATAAAAAGTGTGGGGCGCGGCGGGCGGGTCA |
| | | | GTCCGCCTGGAGACCTCGAGCCGAGTGGTCGTCTCAGGAG |
| | | | CC |
| 201 | | MVM- | AAGAGGTAAGGGTTTAAGGGATGGTTGGTTGCGGGCGGGG |
| | | ACTA1 | GCCCAGGCCGCGAACCGGCCGAGGGAGGGGGCTCTAGTG |
| | | intron | CCCAACACCCAAATATGGCTCGAGAAGGGCAGCGACATTC |
| | | | CTGCGGGGTGGCGCGGAGGGAATGCCCGGCGCCTATAAAA |
| | | | AGTGTGGGGCGCGGCGGGCGGGTCAGTCCGCCTGGAGACC |
| | | | TCGAGCCGAGTGGTCGTGTGGGGTGTTTAATTACCTGGAG |
| | | | CACCTGCCTGAAATCACTTTTTTTCAGGTTGG |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| | | (part of | |
| | | MVM- | |
| | | ACTA1 | |
| | | intron) | |
| 169 | | minACTA1.3 | TCGAGAAGGGCAGCGACATTCCTGCGGGGTGGCGCGGAG |
| | | promoter | GGAATGCCCGGCGCCTATAAAAAGTGTGGGGCGCGGCGG |
| | | variant 3 | GCGGGTCAGTCCGCCTGGAGACCTCGAGCCGAGTGGTCGT |
| | | (part of | |
| | | MVM- | |
| | | ACTA1 | |
| | | intron) | |
| 119 | M119 | ATAC1.1b | CGGGCGGGGGCCCAGGCCGCGAACCGGCCGAGGGAGGGG |
| | | | GCTCTAGTGCCCAACACCCAAATATGGCTCGAGAAGGGCA |
| | | | GCGACATTCCTGCGGGGTGGCGCGGAGGGAATGCCCGGCG |
| | | | CCTATAAAAAGTGTGGGGCGCGGCGGGCGGGTCAGTCCGC |
| | | | CTGGAGACCTCGAGCCGAGTGGTCGTTGAGCCAAGAGGTA |
| | | | AGGGTTTAAGGGATGGTTGGTTGTGGCTGATCCTCTCCCCT |
| | | | GCCCTTGGCTCCATGAATGGCCTCGGCAGTCCTAGCGGGT |
| | | | GCGAAGGGGACCAAATAAGGCAAGGTGGCAGACCGGGCC |
| | | | CCCCACCCCTGCCCCCGGCTGCTCCAACTGACCCTGTCCAT |
| | | | CCGGCGCCTATAAAAAGTGTGGGGCGCGGCGGGCGGGTCA |
| | | | GTCCGCCTGGAGACCTCGAGCCGAGTGGTCGTGTGGGGTG |
| | | | TTTAATTACCTGGAGCACCTGCCTGAAATCACTTTTTTTCA |
| | | | GGTTGGCCGCGGATCCGCCACC |
| 121 | | ACTA1 | CGGGCGGGGGCCCAGGCCGCGAACCGGCCGAGGGAGGGG |
| | | enhancer | GCTCTAGTGCCCAACACCCAAATATGGC |
| | | (part of | |
| | | minACTA1.3 | |
| | | promoter | |
| | | (partial)) | |
| 170 | | minACTA1.3 | CGGGCGGGGGCCCAGGCCGCGAACCGGCCGAGGGAGGGG |
| | | promoter | GCTCTAGTGCCCAACACCCAAATATGGCTCGAGAAGGGCA |
| | | variant 2 | GCGACATTCCTGCGGGGTGGCGCGGAGGGAATGCCCGGCG |
| | | | CCTATAAAAAGTGTGGGGCGCGGCGGGCGGGTCAGTCCGC |
| | | | CTGGAGACCTCGAGCCGAGTGGTCGT |
| 203 | | MVM- | AAGAGGTAAGGGTTTAAGGGATGGTTGGTTGTGGCTGATC |
| | | ACTC1 | CTCTCCCCTGCCCTTGGCTCCATGAATGGCCTCGGCAGTCC |
| | | intron | TAGCGGGTGCGAAGGGGACCAAATAAGGCAAGGTGGCAG |
| | | variant 1 | ACCGGGCCCCCCACCCCTGCCCCCGGCTGCTCCAACTGAC |
| | | | CCTGTCCATCCGGCGCCTATAAAAAGTGTGGGGCGCGGCG |
| | | | GGCGGGTCAGTCCGCCTGGAGACCTCGAGCCGAGTGGTCG |
| | | | TGTGGGGTGTTTAATTACCTGGAGCACCTGCCTGAAATCAC |
| | | | TTTTTTTCAGGTTGG |
| 122 | | ACTC1 | TGGCTGATCCTCTCCCCTGCCCTTGGCTCCATGAATGGCCT |
| | | enhancer | CGGCAGTCCTAGCGGGTGC |
| | | part of | |
| | | MVM- | |
| | | ACTC1 | |
| | | intron) | |
| 177 | | minACTC1.3 | GAAGGGGACCAAATAAGGCAAGGTGGCAGACCGGGCCCC |
| | | promoter | CCACCCCTGCCCCCGGCTGCTCCAACTGACCCTGTCCATCC |
| | | (partial) | GGCGCCTATAAAAAGTGTGGGGCGCGGCGGGCGGGTCAGT |
| | | | CCGCCTGGAGACCTCGAGCCGAGTGGTCGT |
| 120 | M120 | ATAC1.2b | TGGCTGATCCTCTCCCCTGCCCTTGGCTCCATGAATGGCCT |
| | | | CGGCAGTCCTAGCGGGTGCGAAGGGGACCAAATAAGGCA |
| | | | AGGTGGCAGACCGGGCCCCCCACCCCTGCCCCCGGCTGCT |
| | | | CCAACTGACCCTGTCCATCCGGCGCCTATAAAAAGTGTGG |
| | | | GGCGCGGGGGGGGTCAGTCCGCCTGGAGACCTCGAGCC |
| | | | GAGTGGTCGTTGAGCCAAGAGGTAAGGGTTTAAGGGATGG |
| | | | TTGGTTGTGGCTGATCCTCTCCCCTGCCCTTGGCTCCATGA |
| | | | ATGGCCTCGGCAGTCCTAGCGGGTGCGAAGGGGACCAAAT |
| | | | AAGGCAAGGTGGCAGACCGGGCCCCCCACCCCTGCCCCCG |
| | | | GCTGCTCCAACTGACCCTGTCCATCAGCGTTCTATAAAGCG |
| | | | GCCCTCCTGGAGCCAGCCACCCAGAGCCCGCTGCCGCCGC |
| | | | GAGCCGAGTGGTCGTCCCCGCCGGTGGGGTGTTTAATTAC |
| | | | CTGGAGCACCTGCCTGAAATCACTTTTTTTCAGGTTGGCCG |
| | | | CGGATCCGCCACC |
| 122 | | ACTC1 | TGGCTGATCCTCTCCCCTGCCCTTGGCTCCATGAATGGCCT |
| | | enhancer | CGGCAGTCCTAGCGGGTGC |
| 188 | | miniACTC1.3 | GAAGGGGACCAAATAAGGCAAGGTGGCAGACCGGGCCCC |
| | | promoter | CCACCCCTGCCCCCGGCTGCTCCAACTGACCCTGTCCATCC |
| | | (partial) | GGCGCCTATAAAAAGTGTGGGGCGCGGCGGGCGGGTCAGT |
| | | | CCGCCTGGAGACCTCGAGCCGAGTGGTCGT |
| 204 | | MVM- | AAGAGGTAAGGGTTTAAGGGATGGTTGGTTGTGGCTGATC |
| | | ACTC1 | CTCTCCCCTGCCCTTGGCTCCATGAATGGCCTCGGCAGTCC |
| | | intron | TAGCGGGTGCGAAGGGGACCAAATAAGGCAAGGTGGCAG |
| | | variant 2 | ACCGGGCCCCCCACCCCTGCCCCCGGCTGCTCCAACTGAC |
| | | | CCTGTCCATCAGCGTTCTATAAAGCGGCCCTCCTGGAGCCA |
| | | | GCCACCCAGAGCCCGCTGCCGCCGCGAGCCGAGTGGTCGT |
| | | | CCCCGCCGGTGGGGTGTTTAATTACCTGGAGCACCTGCCTG |
| | | | AAATCACTTTTTTTCAGGTTGGCCGC |
| 122 | | ACTC1 | TGGCTGATCCTCTCCCCTGCCCTTGGCTCCATGAATGGCCT |
| | | enhancer | CGGCAGTCCTAGCGGGTGC |
| | | (part of | |
| | | MVM- | |
| | | ACTC1 | |
| | | intron) | |
| 175 | | minACTC1.2 | GAAGGGGACCAAATAAGGCAAGGTGGCAGACCGGGCCCC |
| | | promoter | CCACCCCTGCCCCCGGCTGCTCCAACTGACCCTGTCCATCA |
| | | (partial) | GCGTTCTATAAAGCGGCCCTCCTGGAGCCAGCCACCCAGA |
| | | (part of | GCCCGCTGCCGCCGCGAGCCGAGTGGTCGT |
| | | MVM- | |
| | | ACTC1 | |
| | | intron) | |

We claim:

1. A NARE comprising: (i) a sequence that is at least 90% identical to SEQ ID NO:132; and (ii) a sequence that is at least 90% identical to any one of SEQ ID NOs: 156-163.

2. The NARE of claim 1, comprising: (i) a sequence that is at least 95% identical to SEQ ID NO: 132; and (ii) a sequence that is at least 95% identical to any one of SEQ ID NOs: 156-163.

3. The NARE of claim 1 comprising (i) SEQ ID NO:132; and (ii) any one of SEQ ID NOs: 156-163.

4. A NARE comprising: (i) a sequence that is at least 90% identical to SEQ ID NO:164; (ii) a sequence that is at least 90% identical to SEQ ID NO:134; (iii) a sequence that is at least 90% identical to SEQ ID NO:135; and (vi) a sequence that is at least 90% identical to SEQ ID NOs: 148 or 149.

5. The NARE of claim 4 comprising: (i) a sequence that is at least 95% identical to SEQ ID NO: 164; (ii) a sequence that is at least 95% identical to SEQ ID NO:134; (iii) a sequence that is at least 95% identical to SEQ ID NO:135; and (vi) a sequence that is at least 95% identical to SEQ ID NOs: 148 or 149.

6. The NARE of claim 4 comprising: (i) SEQ ID NO:164; (ii) SEQ ID NO:134; (iii) SEQ ID NO: 135; and (vi) SEQ ID NOs: 148 or 149.

7. A NARE comprising: (i) a sequence that is at least 90% identical to SEQ ID NO:218; and (ii) a sequence that is at least 90% identical to any one of SEQ ID NOs: 148 or 149.

8. The NARE of claim 7 comprising: (i) a sequence that is at least 95% identical to SEQ ID NO: 218; and (ii) a sequence that is at least 95% identical to any one of SEQ ID NOs: 148 or 149.

9. The NARE of claim 7 comprising: (i) SEQ ID NO:218; and (ii) SEQ ID NOs: 148 or 149.

10. A NARE comprising: (i) a sequence that is at least 90% identical to any one of SEQ ID NOs: 133, 142, 165 or 217; (ii) optionally, a sequence that is at least 90% identical to SEQ ID NOs: 154 or 189; (iii) optionally, a sequence that is at least 90% identical to SEQ ID NO: 220; and (iv) a sequence that is at least 90% identical to SEQ ID NO:150.

11. The NARE of claim 10, comprising: (i) a sequence that is at least 95% identical to any one of SEQ ID NOs: 133, 142, 165 or 217; (ii) optionally, a sequence that is at least 95% identical to SEQ ID NOs: 154 or 189; (iii) optionally, a sequence that is at least 95% identical to SEQ ID NO:220; and (iv) a sequence that is at least 95% identical to SEQ ID NO: 150.

12. The NARE of claim 10, comprising: (i) any one of SEQ ID NOs: 133, 142, 165 or 217; (ii) optionally, SEQ ID NOs: 154 or 189; (iii) optionally, SEQ ID NO:220; and (iv) SEQ ID NO: 150.

13. A NARE comprising: (i) a sequence that is at least 90% identical to SEQ ID NO:131; (ii) a sequence that is at least 90% identical to SEQ ID NOs: 126 or 213; and (iii) optionally, a sequence that is at least 90% identical to SEQ ID NO:200.

14. The NARE of claim 13, comprising: (i) a sequence that is at least 95% identical to SEQ ID NO: 131; (ii) a sequence that is at least 95% identical to SEQ ID NOs: 126 or 213; and (iii) optionally, a sequence that is at least 95% identical to SEQ ID NO:200.

15. The NARE of claim 13, comprising: (i) SEQ ID NO:131; (ii) SEQ ID NOs: 126 or 213; and (iii) optionally, SEQ ID NO:200.

16. The NARE of claim 13, comprising: (i) a sequence that is at least 90% identical to SEQ ID NO: 131; (ii) a sequence that is at least 90% identical to SEQ ID NO:126; and (iii) optionally, a sequence that is at least 90% identical to SEQ ID NO:200.

17. The NARE of claim 13, comprising: (i) a sequence that is at least 95% identical to SEQ ID NO: 131; (ii) a sequence that is at least 95% identical to SEQ ID NO:126; and (iii) optionally, a sequence that is at least 95% identical to SEQ ID NO:200.

18. The NARE of claim 13, comprising: (i) SEQ ID NO:131; (ii) SEQ ID NO:126; and (iii) optionally, SEQ ID NO:200.

19. A NARE comprising: (i) a sequence that is at least 90% identical to any one of SEQ ID NOs: 190-199; and (ii) a sequence that is at least 90% identical to SEQ ID NO:155.

20. The NARE of claim 19, comprising: (i) a sequence that is at least 95% identical to any one of SEQ ID NOs: 190-199; and (ii) a sequence that is at least 95% identical to SEQ ID NO: 155.

21. The NARE of claim 19, comprising: (i) any one of SEQ ID NOs: 190-199; and (ii) SEQ ID NO: 155.

22. A NARE comprising: (i) a sequence that is at least 90% identical to any one of SEQ ID NOs: 190-199; and (ii) a sequence that is at least 90% identical to any one of SEQ ID NOs: 128-130.

23. The NARE of claim 22, comprising: (i) a sequence that is at least 95% identical to any one of SEQ ID NOs: 190-199; and (ii) a sequence that is at least 95% identical to any one of SEQ ID NOs: 128-130.

24. The NARE of claim 22, comprising: (i) any one of SEQ ID NOs: 190-199; and (ii) any one of SEQ ID NOs: 128-130.

25. A NARE comprising: (i) a sequence that is at least 90% identical to any one of SEQ ID NOs: 128-130; and (ii) a sequence that is at least 90% identical to any one of SEQ ID NOs: 123-125, 127, 136-141, 143, 145-147, 152, 153, 209-212, 219, or 221.

26. The NARE of claim 25, comprising: (i) a sequence that is at least 95% identical to any one of SEQ ID NOs: 128-130; and (ii) a sequence that is at least 95% identical to any one of SEQ ID NOs: 123-125, 127, 136-141, 143, 145-147, 152, 153, 209-212, 219, or 221.

27. The NARE of claim 25, comprising: (i) any one of SEQ ID NOs: 128-130; and (ii) any one of SEQ ID NOs: 123-125, 127, 136-141, 143, 145-147, 152, 153, 209-212, 219, or 221.

28. A NARE comprising: (i) a sequence that is at least 90% identical to SEQ ID NO:121; (ii) a sequence that is at least 90% identical to any one of SEQ ID NOs: 166-169, 171, or 172; and (iii) optionally, a sequence that is at least 90% identical to SEQ ID NOs: 122 or 154.

29. The NARE of claim 28, comprising: (i) a sequence that is at least 95% identical to SEQ ID NO: 121; (ii) a sequence that is at least 95% identical to any one of SEQ ID NOs: 166-169, 171, or 172; and (iii) optionally, a sequence that is at least 95% identical to SEQ ID NOs: 122 or 154.

30. The NARE of claim 28, comprising: (i) SEQ ID NO: 121; (ii) any one of SEQ ID NOs: 166-169, 171, or 172; and (iii) optionally, SEQ ID NOs: 122 or 154.

31. The NARE of claim 28, comprising: (i) a sequence that is at least 90% identical to SEQ ID NO: 121; and (ii) a sequence that is at least 90% identical to any one of SEQ ID NOs: 166-169, 171, or 172.

32. The NARE of claim 28, comprising: (i) a sequence that is at least 95% identical to SEQ ID NO: 121; and (ii) a sequence that is at least 95% identical to any one of SEQ ID NOs: 166-169, 171, or 172.

33. The NARE of claim 28, comprising: (i) SEQ ID NO: 121; and (ii) SEQ ID NOs: 166-169, 171, or 172.

34. The NARE of claim 28, comprising: (i) a sequence that is at least 90% identical to SEQ ID NO: 122 or 154; (ii) a sequence that is at least 90% identical to SEQ ID NO:121; and (iii) a sequence that is at least 90% identical to SEQ ID NO:167.

35. The NARE of claim 28, comprising: (i) a sequence that is at least 95% identical to SEQ ID NO: 122 or 154; (ii) a sequence that is at least 95% identical to SEQ ID NO:121; and (iii) a sequence that is at least 90% identical to SEQ ID NO:167.

36. The NARE of claim 28, comprising: (i) SEQ ID NO: 122 or 154; (ii) SEQ ID NO:121; and (iii) SEQ ID NO:167.

37. A NARE comprising: (i) a sequence that is at least 90% identical to SEQ ID NO:122; (ii) a sequence that is at least 90% identical to any one of SEQ ID NO:173-179, or 188; and (iii) optionally, a sequence that is at least 90% identical to SEQ ID NO:207.

38. The NARE of claim 37, comprising: (i) a sequence that is at least 95% identical to SEQ ID NO: 122; (ii) a sequence that is at least 95% identical to any one of SEQ ID NO:173-179, or 188; and (iii) optionally, a sequence that is at least 95% identical to SEQ ID NO:207.

39. The NARE of claim 37, comprising: (i) SEQ ID NO: 122; (ii) any one of SEQ ID NO: 173-179, or 188; and (iii) optionally, SEQ ID NO:207.

40. The NARE of claim 37, comprising: (i) a sequence that is at least 90% identical to SEQ ID NO: 122; and (ii) a sequence that is at least 90% identical to any one of SEQ ID NO:173-179.

41. The NARE of claim 37, comprising: (i) a sequence that is at least 95% identical to SEQ ID NO: 122; and (ii) a sequence that is at least 95% identical to any one of SEQ ID NO:173-179.

42. The NARE of claim 37, comprising: (i) SEQ ID NO:122; and (ii) any one of SEQ ID NO: 173-179.

43. A NARE comprising: (i) a sequence that is at least 90% identical to SEQ ID NO:184; and (ii) a sequence that is at least 90% identical to any one of SEQ ID NOs: 185-187.

44. The NARE of claim 43, comprising: a sequence that is at least 95% identical to SEQ ID NO: 184; and (ii) a sequence that is at least 95% identical to any one of SEQ ID NOs: 185-187.

45. The NARE of claim 43, comprising: SEQ ID NO: 184; and (ii) any one of SEQ ID NOs: 185-187.

46. A NARE comprising: (i) a sequence that is at least 90% identical to SEQ ID NO:180; and (ii) a sequence that is at least 90% identical to any one of SEQ ID NOs: 181 or 182.

47. The NARE of claim 46, comprising: (i) a sequence that is at least 95% identical to SEQ ID NO: 180; and (ii) a sequence that is at least 95% identical to any one of SEQ ID NOs: 181 or 182.

48. The NARE of claim 46, comprising: (i) SEQ ID NO:180; and (ii) any one of SEQ ID NOs: 181 or 182.

49. A NARE comprising: (i) a sequence that is at least 90% identical to SEQ ID NO:183; (ii) a sequence that is at least 90% identical to SEQ ID NO:182; and (iii) optionally, a sequence that is at least 90% identical to any one of SEQ ID NOs: 200, 201, 205, or 208.

50. The NARE of claim 49, comprising: (i) a sequence that is at least 95% identical to SEQ ID NO: 183; (ii) a sequence that is at least 95% identical to SEQ ID NO:182; and (iii) optionally, a sequence that is at least 95% identical to any one of SEQ ID NOs: 200, 201, 205, or 208.

51. The NARE of claim 49, comprising: (i) SEQ ID NO: 183; (ii) SEQ ID NO:182; and (iii) optionally, any one of SEQ ID NOs: 200, 201, 205, or 208.

52. The NARE of claim 49, comprising: (i) a sequence that is at least 90% identical to SEQ ID NO: 183; (ii) a sequence that is at least 90% identical to SEQ ID NO:182; and (iii) a sequence that is at least 90% identical to any one of SEQ ID NOs: 200, 201, 205, or 208.

53. The NARE of claim 49, comprising: (i) a sequence that is at least 95% identical to SEQ ID NO: 183; (ii) a sequence that is at least 95% identical to SEQ ID NO:182; and (iii) a sequence that is at least 95% identical to any one of SEQ ID NOs: 200, 201, 205, or 208.

54. The NARE of claim 49, comprising: (i) SEQ ID NO: 183; (ii) SEQ ID NO:182; and (iii) any one of SEQ ID NOs: 200, 201, 205, or 208.

55. A NARE comprising: (i) a sequence that is at least 90% identical to SEQ ID NO:154 or 164; and (ii) a sequence that is at least 90% identical to any one of SEQ ID NOs: 128-130.

56. The NARE of claim 55, comprising: (i) a sequence that is at least 95% identical to SEQ ID NO: 154 or 164; and (ii) a sequence that is at least 95% identical to any one of SEQ ID NOs: 128-130.

57. The NARE of claim 55, comprising: (i) SEQ ID NO: 154 or 164; and (ii) any one of SEQ ID NOs: 128-130.

58. A NARE comprising: (i) a sequence that is at least 90% identical to SEQ ID NO:170; (ii) optionally, a sequence that is at least 90% identical to a SEQ ID NO:188; and (iii) a sequence that is at least 90% identical to SEQ ID NOs: 203 or 204.

59. The NARE of claim 58, comprising: (i) a sequence that is at least 95% identical to SEQ ID NO: 170; (ii) optionally, a sequence that is at least 95% identical to a SEQ ID NO:188; and (iii) a sequence that is at least 95% identical to SEQ ID NOs: 203 or 204.

60. The NARE of claim 58, comprising: (i) SEQ ID NO: 170; (ii) optionally, SEQ ID NO:188; and (iii) SEQ ID NOs: 203 or 204.

61. A NARE comprising:
(a) (i) a sequence that is at least 90% identical to SEQ ID NO:218; and (ii) a sequence that is at least 90% identical to SEQ ID NO:149;
(b) (i) a sequence that is at least 90% identical to SEQ ID NO:164; (ii) a sequence that is at least 90% identical to SEQ ID NO:134; (iii) a sequence that is at least 90% identical to SEQ ID NO:135; and (vi) SEQ ID NO:149;
(c) (i) a sequence that is at least 90% identical to SEQ ID NO:165; (ii) a sequence that is at least 90% identical to SEQ ID NO:154; (iii) a sequence that is at least 90% identical to SEQ ID NO:220; and (iv) a sequence that is at least 90% identical to SEQ ID NO:150;
(d) (i) a sequence that is at least 90% identical to SEQ ID NO:142; and (ii) a sequence that is at least 90% identical to SEQ ID NO:150;
(e) (i) a sequence that is at least 90% identical to SEQ ID NO:217; (ii) a sequence that is at least 90% identical to SEQ ID NO:189; and (iii) a sequence that is at least 90% identical to SEQ ID NO:150;
(f) (i) a sequence that is at least 90% identical to SEQ ID NO:196; and (ii) a sequence that is at least 90% identical to SEQ ID NO:155;
(g) (i) a sequence that is at least 90% identical to SEQ ID NO:194; and (ii) a sequence that is at least 90% identical to SEQ ID NO:155; or
(h) (i) a sequence that is at least 90% identical to SEQ ID NO:184; and (ii) a sequence that is at least 90% identical to SEQ ID NO:187.

62. The NARE of claim 61, comprising:
(a) (i) a sequence that is at least 95% identical to SEQ ID NO:218; and (ii) a sequence that is at least 95% identical to SEQ ID NO:149;
(b) (i) a sequence that is at least 95% identical to SEQ ID NO:164; (ii) a sequence that is at least 95% identical to SEQ ID NO:134; (iii) a sequence that is at least 95% identical to SEQ ID NO:135; and (vi) SEQ ID NO:149;
(c) (i) a sequence that is at least 95% identical to SEQ ID NO:165; (ii) a sequence that is at least 95% identical to SEQ ID NO:154; (iii) a sequence that is at least 95% identical to SEQ ID NO:220; and (iv) a sequence that is at least 95% identical to SEQ ID NO:150;
(d) (i) a sequence that is at least 95% identical to SEQ ID NO:142; and (ii) a sequence that is at least 95% identical to SEQ ID NO:150;
(e) (i) a sequence that is at least 95% identical to SEQ ID NO:217; (ii) a sequence that is at least 95% identical to SEQ ID NO: 189; and (iii) a sequence that is at least 95% identical to SEQ ID NO:150;
(f) (i) a sequence that is at least 95% identical to SEQ ID NO:196; and (ii) a sequence that is at least 95% identical to SEQ ID NO:155;
(g) (i) a sequence that is at least 95% identical to SEQ ID NO:194; and (ii) a sequence that is at least 95% identical to SEQ ID NO:155; or
(h) (i) a sequence that is at least 95% identical to SEQ ID NO:184; and (ii) a sequence that is at least 95% identical to SEQ ID NO:187.

63. The NARE of claim 61, comprising:
(a) (i) SEQ ID NO:218; and (ii) SEQ ID NO:149;
(b) (i) SEQ ID NO:164; (ii) SEQ ID NO:134; (iii) SEQ ID NO:135; and (vi) SEQ ID NO: 149;
(c) (i) SEQ ID NO:165; (ii) SEQ ID NO:154; (iii) SEQ ID NO:220; and (iv) SEQ ID NO: 150;
(d) (i) SEQ ID NO:142; and (ii) SEQ ID NO:150;
(e) (i) SEQ ID NO:217; (ii) SEQ ID NO:189; and (iii) SEQ ID NO:150;
(f) (i) SEQ ID NO:196; and (ii) SEQ ID NO:155;
(g) (i) SEQ ID NO:194; and (ii) SEQ ID NO:155; or
(h) (i) SEQ ID NO:184; and (ii) SEQ ID NO:187.

64. A NARE comprising a sequence that is at least 90% identical to any one of SEQ ID NOs: 1-18, 20, 21, 23, 24, 32-49, 51-70, or 74-120.

65. The NARE of claim 64, comprising a sequence that is at least 95% identical to any one of SEQ ID NOs: 1-18, 20, 21, 23, 24, 32-49, 51-70, or 74-120.

66. The NARE of claim 64, comprising any one of SEQ ID NOs: 1-18, 20, 21, 23, 24, 32-49, 51-70, or 74-120.

67. A NARE comprising a sequence that is at least 90% identical to any one of SEQ ID NOs: 1-18 or 32.

68. The NARE of claim 67, comprising a sequence that is at least 95% identical to any one of SEQ ID NOs: 1-18 or 32.

69. The NARE of claim 67, comprising any one of SEQ ID NOs: 1-18 or 32.

70. An expression construct comprising the NARE of any one of the preceding claims and an operatively linked transgene.

71. The expression construct of claim 70, further comprising a polyadenylation sequence.

72. A vector comprising the expression construct of claim 70.

73. The vector of claim 72, wherein the vector is a non-viral vector.

74. The vector of claim 72, wherein the vector is a viral vector.

75. The vector of claim 74, wherein the vector is an adeno-associated virus (AAV) vector.

76. A vector comprising a nucleic acid sequence comprising (i) the expression construct of 70, and (ii) one or more inverted terminal repeats (ITR).

77. The vector of claim 76, wherein the nucleic acid sequence comprises a 5′ ITR and a 3′ ITR.

78. The vector of claim 76, wherein the 5′ ITR and a 3′ ITR are derived from adeno-associated virus (AAV) serotype AAV2.

79. A cell comprising the expression construct of claim 70 or the vector of any one of claims 72-78.

80. The cell of claim 79, wherein the cell is a muscle cell.

81. A pharmaceutical composition comprising: (i) the expression construct of claim 70 or the vector of any one of claims 72-78, and (ii) a pharmaceutically acceptable excipient.

82. A method for expressing a transgene in a cell comprising the expression construct of claim 70 or the vector of any one of claims 72-78.

83. A method for regulating transgene expression in a cell comprising the expression construct of claim 70 or the vector of any one of claims 72-78.

84. The method of claim 82 or 83, wherein the cell is a muscle cell.


SEQUENCE LISTING (222 sequences)

SEQ ID NO: 1 - DNA, 1500 bp, synthetic construct
  source 1..1500: mol_type = other DNA
cctttcaaagcctcaagggtgttctcatccccagcagtgggacaaactggggacagaggaagccccaggccccctcctctcacctggcaccactttctcttgtcctggaccagcccgccagcccactctgcaggccacagttggaactctcatcccaccgcttccagcctgtggtgggagcatgagaccacaaaccaggaagtggggggggggagtgcccagagattgcgcaacccccagcactcagtccagaacatctggagaagtgccggcctctccacctcctggggcaggggccaatctgctctccaagtgattctctttcctcataagaaaggtggggataaaaacaccgtgagttctgcaacatcagaataaatataacaactggggcctcaggctgttcccacaggaaggatgcagataagcagggagggtgtggaatcacatccagggctgctttataaacacggattgatgacacattatgcccccaccccgggaagcgggatggcccctccctccccaggaagtcctggctggggcccatgggggtgggacggctggtcagaggaggagaggcagttggagaccaggtcctgccctgttagcctttctgtaggaccaggtgtcacactcatctgagggctccagacacagggcctgtgcgtctgtcctgcccagcgtctcccacctgacgcggccaggtgcccagttgctcttcaaggccttttcaggtgctcctttccaggaccagggtcacagaagtcaaagctcaggaaaagcccctgattgtgcctgccacagtggaggggccctgcagacccagataaaccttcatggggctgccttgggccgtgggagccagcagtgtgcccagctccaccgtccctgcccctccctaggggctgcctgcgctccagccagcatccctgggcggaaatccttccttcccccgaccaggccaggggcgggggcctccgccctggataaggactccccggcccactccggaccagggctggggcggcctcccaggcgctcactccgctggcaccccaccggaaaacacgtctgcggcccgccccctcccccaaagcacgaccactccgcccgggccccctctggggccgccaggcgaccagggcgcaggcgggggcggacagctcattgggaggggcgccggggcacagtgcggggctcgccccacccccaggtgccccttccccgctctcgcctcgcaggcaccgcatcgggcccgggaatcggtccggacctggcggtgggcgctgggaagaggatccacctccacgtggcccgccccgccccgggggcgcagccagttcccggcgctcactgccccccttctcccggcttccgtccccttctgcgcaggcgccgctccgccccggtcctaggggtgcttccgtggtcggcggctgctgggctccgcgccggggtccgagtcccacgaagccccggcccgagggatccgccacc

SEQ ID NO: 2 - DNA, 512 bp, synthetic construct
  source 1..512: mol_type = other DNA
cccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccggatccgccacc

SEQ ID NO: 3 - DNA, 512 bp, synthetic construct
  source 1..512: mol_type = other DNA
gtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagacagccccccttcagcccagcccagccaggtactgcacggggcgggaatctgggtggggggatccgccacc

SEQ ID NO: 4 - DNA, 512 bp, synthetic construct
  source 1..512: mol_type = other DNA
ggagatgacatatgagcaaagatgcagtgaaggaggtgaaggaaggagccgtgcgatgactgacagaaagacattccaggtagagggcacacaggtgcaaagaccctgaggccagatccaggctgataaaacagagcattttagcagtctcctctccctgccattttttttctcaaaattgacaagcacaagtgtccccggcccaagcaccgcagagagcgcgcagcatctctccccgtgaccatgacccagctactgcctctttaaccttgaatgcctttttgggggctcacgtgtcacccagtggcgagtgagccacccttacttcagaagaacggcatggggtgggggggccttaggtggtgcccgcctcacctatgactgccaaaagcggtcatggggttatttttaaacatggggaggaagtatttattgttcctgggctgcagagagctgggcggagtgtggaattcttctcgggaggcagtgctgggtcctttggatccgccacc

SEQ ID NO: 5 - DNA, 512 bp, synthetic construct
  source 1..512: mol_type = other DNA
tggcaaaggggcagaaggtgcgtgagaggttgcgaatggccaggactgtcctggggccagccggggcacctggtggccaagcttagaaacatgacaggtcctcttgggagggctgaccgcagggagcgttgggtttcaggctgctggcgtcggcttctgtggtgccctttctgtcggctatgagagtccagacagtgcccaacctcctccccttctttccacacgcacaaccaccccaccccctgtggcctgagctgtcctgcctcgccacaatggcacctgccctaaaatagcttcccatgtgagggctagagaaaggaaaagattagaccctccctggatgagagagagaaagtgaaggagggcaggggagggggacagcgagccattgagcgatctttgtcaagcatcccagaaggtataaaaacgcccttgggaccaggcagcctcaaaccccagctgttggggccaggacacccagtgagcccatacttgctcttggatccgccacc

SEQ ID NO: 6 - DNA, 728 bp, synthetic construct
  source 1..728: mol_type = other DNA
tcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccggatccgccacc

SEQ ID NO: 7 - DNA, 592 bp, synthetic construct
  source 1..592: mol_type = other DNA
gctccgcctcccgagagacatttaatccggggggatttgcaggaaacttctaaattaagggtagcggctgctgcagctgagggggggcacgccggtccctgcgcccgggcagctgccgtgagctcacgccccgaaatagccccaggggccccagccgcagctgccactgggtccggctgtcactcagaggaagcacggagcccccagcccaagggtcccctccccttcgcatcgccggggtttttccagccgaccgtcggccactttttcctccgacggctggcagggaagagggggatgggggcgggaccccaagggaggcggtccccagtgggtgggcgaagggggcggccgcaccccccggccgggccgtgcttctgcccctacaaggtttgggccgaggtgggggagggtcctggttgccggccccgcccggtccctccccgccttttaggcgcccgcgtggccgggacgtcccagtcccgctccgtcctcctcgcctgccaccggtgcacccagtccgctcacccagcccagtccgtccggtcctcaccgcctgccggccggcccaccccccaccggatccgccacc

SEQ ID NO: 8 - DNA, 719 bp, synthetic construct
  source 1..719: mol_type = other DNA
ccactgcattccagcctgggtgacagagtgagagtctgtctcaaaaacaaaaccccaaaaaaccaaaaaacaataaaaactaaattggaatcccatgggtctccctcaccctttggaccctgcctgcctgggggaggccttatttagcttggcccagccctgctgagctttccttgggaatgtctatatataggggaagggggcagcccagttgccactgtccatctgcattccttggtgtccagcccccctttcctggccccactgccccttaccatggttcaaagacgggacattcctcttgccaagcaccaacgctgtgtacccagcacaagctaggtgacttatacccgggttcccatttaccatggtctctctgtgagggagaccccacagacaggtccaatcccacagatgaagaaatggctcagagaggcactgctaccggcccaaggtcacgccacctgcttggaggcttaaatttctctgcaagggcccttggctaaattaggtaatgggttcagactgtgggaggggtgggactcgctgaccccaggatctgattgggcagggtctccagtgctggggagcagggaggtgggaggggagggtgcccctacaaatcccgggggctagagcaggccaggtcatctttgggtggtggagtgcaaaggaggcgacctgcaacagaggagtcccggtcaccaggatccgccacc

SEQ ID NO: 9 - DNA, 617 bp, synthetic construct
  source 1..617: mol_type = other DNA
tgggtcagtcacttcctccttcaccagttccacagcatcttactatgagcttggcattcgaggcttctcttggcagggccctgcactcctagcctctccttgcacattgcacccccattccagagaggtttagttaaaggcgggggttaccaagtcagtcagatcttgggcaagtcaccactcctccagagcctcagtttccttatctggaaagtggaggtcatggcaacccgccaacctggttggatgggagcctgagctgttgtgttgcaccttgcctggggcccacgactttgtagctcctgtcctgcactgggcttatgttttcattcattccagaaaccttttcagagagtccctttggggagtgtgggggacaggagggaaagaaacctggtccttgtagccgttcgtctgctccctgccctgggcagaggacgtggggactcaggccagcctgagatcactgggaccagaggaggggctggaggatactacacgcaggggtgggctgggctgggctgggctgggccaggaatgcagcggggcagggctatttaagtcaagggccggctggcaaccccagcaagctgtcctgtgagccgggatccgccacc

SEQ ID NO: 10 - DNA, 365 bp, synthetic construct
  source 1..365: mol_type = other DNA
cctcccacgctgcgcgcgcacctccccgcccccacccctacccgctggcgtgcccagtggaacggagccttgtgtctccgcctcaagtccccggatgctcacctccccgactcgcccccgctgtggccccgcccccgcgcggctcttcgtgccacgtcaccgcctgcgtcgcttccggaggcgcagcgggcgatgacgtagagggacgtgccctctatatgaggttggggagcggctgagtcggccttttccgcccgctcccccctccccccgagcgccgctccggctgcaccgcgctcgctccgagtttcaggctcgtgctaagctagcgccgtcgtcgtctcccttcagtcggatccgccacc

SEQ ID NO: 11 - DNA, 549 bp, synthetic construct
  source 1..549: mol_type = other DNA
tcaaggtcatggcttggctgggaagcgggagggggataccaaaaaagatcagcgttagatggaggtggggagggagggtaggtttcaccctaattgacaccccaccctccccctccccctccccacacctgcttcccggctttagtgcctgttgtcaccaggatggaatcctggcggcagacaaaggggtgggggtgggaggaggagacaaaaagatggacagagacagaggaggagactaagaagtagagacagacacacagagacacacagatggagagccacagagacaacagaaatgcaggggcggggctcctgggccatgcagatggggttggcatggggttggggaggggcccacggaggcttctggactctgagtgtggagccaggggcagcagggctgtctctgaactcccaggcttgtgtttgagcaaaggaattctctccccctccctctcccctcggggcgggcccgagcctccagctataaactccccggagcttcagtgccctcagcaaggctcagcctcaagaggatccgccacc

SEQ ID NO: 12 - DNA, 1006 bp, synthetic construct
  source 1..1006: mol_type = other DNA
gaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaagcagtgaaagggagtccctggctcctggctcgcctgcacgtcccagggcggggagggacttccgccctcacgtcccgctcttcgcaggtttccagatcctgtcctccgcctggggccccagcccggagagagccgctggtgcacacagggccgggattgtctgccctaattatcaggtccaggccccctccccctcaagccgcctcccgagcctccttcctctagatggggagggggaagtgggggctgggaaggaaccacgggcccccgcccgaggcccatgggcccctcctctttcccgcctctcagcctttgaaagaaagaaaggggagggggcaggtcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggggatccgccacc

SEQ ID NO: 13 - DNA, 601 bp, synthetic construct
  source 1..601: mol_type = other DNA
aaagtgactaaaaggaaggaaaaaacctaaaatgtttccaagtggtggggtgtatggggagaaatgtgcccattcagcctctcaagggggcagagccctgtgtctgttctgtccctagaccgaggtggggacggggacagggaccggcaatgcaggaaatccctgcctctcatttgcacacttgggaaatgcaaagctacacacagtggagcgggggctgggggtgtgtagaccgccgggattccgagtttcccggcacgcctaggagagggagaggcaggcaatgtcagggaaattgggcaggcaagacgccagggacgccacgtactgccaggttctcaacgaggtggagccaaaggggcaggccccgcggtgcgcccggcgctgggctcacgggttgctgcacccggcccaggatcgcgggcggtgcagactcagcaggggcgggtgcaaggacgaggcggggcctctgcgcccggccctcttcccggactataaagagagccgccggcttctgggctccaccacgcttttcatctgtcccgctgcgtgttttcctcttgatcgggaactcctgcttctccttgccggatccgccacc

SEQ ID NO: 14 - DNA, 510 bp, synthetic construct
  source 1..510: mol_type = other DNA
taaaaaaaaacctgaactcctggcctcaagcaatcctcctgcctcagcttcccaaagtgctgggattacaggtgtgaaccactgcacccagacgtttactgggtttaaatctgagcccaccccactcaggggtacaggatctggttcatttatctcccagaatgatgccttgggttgcgtcccctctaaactctggcacattccagcccctctttgggagaaagaacatctcttcttgcccaggccctagtgagacatgcatcgcaagacatgctccccccttcctccccgctgggctccagctgctgccagccctccaagaaaggagaggccctgagttgggctattttggtatctggggtgggcacccgcagggctaaggttaccttggtatgttaagggctccctggggcaggactatataaccccagagggactgccccatgctgactccttgcttctttccagccggagccgctgccttgccccccggagactggatccgccacc

SEQ ID NO: 15 - DNA, 514 bp, synthetic construct
  source 1..514: mol_type = other DNA
taaaataaaataaagtagttaaaaaaaaatctatcagctttcctgccaaaataaaaaaaattataaagataacaaggagggaagatgatgaaaagcggattgaaagtgcgctctctgaacttctcctgctatagcagttgccagctcttctgccatcaaacaaataggaagcttcttaaaggaagatgctccaagagaacatcttggaaataaagaccataaaagtaccggcaggcttccatctcactatggctgtctactccaaggttctggtcatctaaaaatagctctcagggtacagatccatctttccctttgccctaagaaagctaaagaactctccaaggggtgtggcaacttatctctgaaacctgatgctagctgtgaggtcaaagcttgcccagaaataaaaggaagcctcagccagggatgaccccactcagggaccggagcagccctcaactcactcttcagcttccctgctgtgttgcagcccagccgcggatccgccacc

SEQ ID NO: 16 - DNA, 559 bp, synthetic construct
  source 1..559: mol_type = other DNA
cactgtccctgtgacctcaacacattgcctgctggtccaaggaaatcagtttattgttcttgccacctggctaggaaatgtccttccagctgccccagtctagctgcctcaccctggggccatgcccccaactctgtcctaccctttccagttggaacactgtggaaatgcggccaggccatcaggggcctcgcactctcctgccagctgtggaatgtgaggcctggcctgggagatatttttgctgcactttgagccaccccgccccctggaactcagaccctgcacagtccatgccataacaatgacgaccacttccaattgtttcctagctggagaggcggggaggggagcactgtttgggaagggggggagcctgggggaaatgcttctagtgacaacagccctttctaaatccggctagggactgggtgccgttgggggtgggggtgccctgctgccccatatatacagcccctgagaccaggtctggctccacagctctgtcctgctctgtgtctttccctgctgctctcaggtaggagcgggatccgccacc

SEQ ID NO: 17 - DNA, 508 bp, synthetic construct
  source 1..508: mol_type = other DNA
gactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccatgacaagtcactggccggctcagacgtgtttgtgtctctcttttcttagctcagtgagtactgggtatgtgtcacattgccaaatcccggatcacaagtctccatgaactgctggtgagctaggataataaaacccctgacatcaccattccagaagcttcacaagactgcatatataaggggctggctgtagctgcagctgaaggagctgaccagccagctgacccctcacactcacctaggatccgccacc

SEQ ID NO: 18 - DNA, 423 bp, synthetic construct
  source 1..423: mol_type = other DNA
ccgactacctgcaaaggggccaaccattgtgggcagccagcctggcgggagaggctagggcacgaagcgccgcccagttaccatggtaacactccctcgtcccaaccccgtcccagtttactggcccaaggtaaggccgggagccgtactccagggcgactgggaggatccactccctgccgcggctctccctccagtggactgccttcccgcctgcctggcggtgggagggcgcggcagagggacgtcacatccgggcgggttggtgagttccggtatttcagggcgtagcaggcggaagtaagggtgagaggaggctgcaacgccgagcggaggaggcaggaaccggagcgcgagcagtagctgggtgggcaccatggctgggatcaccaccatcgaggcggtgaagcgggatccgccacc

SEQ ID NO: 19 - DNA, 716 bp, synthetic construct
  source 1..716: mol_type = other DNA
cacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 20 - DNA, 987 bp, synthetic construct
  source 1..987: mol_type = other DNA
agcttgtcgctgtcagccttccttgacacctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacaccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatcatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcagtccgcctggagacctcgagccgagtggtcgtctcaggagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 21 - DNA, 1652 bp, synthetic construct
  source 1..1652: mol_type = other DNA
agcttgtcgctgtcagccttccttgacacctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacaccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatcatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcagtccgcctggagacctcgagccgagtggtcgtctcaggagccgggcaggttggtatcaaggttacaagcagcccacctgtcccagtgctgacttagtgcaaggcgagccagcaaggagggaggacaggtggcagtggggggtgaggagcatctaaaaatagcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatatataaagtgccgacggctgggggcccttcagtccgcctggagacctcgagccgagtggtcgtactcttgggtttctgataggcactgactctctctgcctattggtctattttcccacccttaggctgctaaaggccgccacc

SEQ ID NO: 22 - DNA, 708 bp, synthetic construct
  source 1..708: mol_type = other DNA
ccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgcccccgggtcacc

SEQ ID NO: 23 - DNA, 945 bp, synthetic construct
  source 1..945: mol_type = other DNA
ccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgccctcagtccgcctggagacctcgagccgagtggtcgtctcaggagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 24 - DNA, 328 bp, synthetic construct
  source 1..328: mol_type = other DNA
gccgtccgccttcggcaccatcctcacgacacccaaatatggcgacgggtgaggaatggtggggagttatttttagagcggtgaggaatggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggcgctcccgcccgcctcgataaaaggctccggggccggcggcggcccacgagctacccggaggagcgggaggcggccacc

SEQ ID NO: 25 - DNA, 1609 bp, synthetic construct
  source 1..1609: mol_type = other DNA
ccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgccctcagtccgcctggagacctcgagccgagtggtcgtctcaggagccgggcaggttggtatcaaggttacaagcagcccacctgtcccagtgctgacttagtgcaaggcgagccagcaaggagggaggacaggtggcagtggggggtgaggagcatctaaaaatagccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatatataaagtgccgacggctgggggcccttcagtccgcctggagacctcgagccgagtggtcgtactcttgggtttctgataggcactgactctctctgcctattggtctattttcccacccttaggctgctaaaggccgccacc

SEQ ID NO: 26 - DNA, 541 bp, synthetic construct
  source 1..541: mol_type = other DNA
gccgtccgccttcggcaccatcctcacgacacccaaatatggcgacgggtgaggaatggtggggagttatttttagagcggtgaggaatggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggcgctcccgcccgcctctatataacccaggggcacaggggctgccctcagtccgcctggagacctcgagccgagtggtcgtctcaggagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 27 - DNA, 580 bp, synthetic construct
  source 1..580: mol_type = other DNA
tggttgctgactaattgagatgcatgctttgcatacttctgcctgctggggagcctggggactttccacaccgggagttatttttagagcgatatggcgacggccacttggcagtacatcaattggaaagtcccgttagtgctattggctggccagccaatagctggttgctgactaattgagatgcatgctttgcatacttctgcctgctggggagcctggggactttccacaccgtgaggaatggtgccaggcatctcgggtttgatgtactgccaagccagccaatagccgctctaaaaataactcccccgtcgccatatttgggtgtccgctctaaaaataactcccggccgtcgccatatgggcccctccctggggacagccccttggaaagtcccgttagtgccaggcgggccatttaccgtcgctctaaaaataactccctagggaaacctgaagcccccattgacgtcaatggggcaggttttttatatagtccggcttcaggtttccctacaaggcctggggacaacccgagatgcctggcaggttttttatatagtccaccaagtcagca

SEQ ID NO: 28 - DNA, 834 bp, synthetic construct
  source 1..834: mol_type = other DNA
tggttgctgactaattgagatgcatgctttgcatacttctgcctgctggggagcctggggactttccacaccgggagttatttttagagcgatatggcgacggccacttggcagtacatcaattggaaagtcccgttagtgctattggctggccagccaatagctggttgctgactaattgagatgcatgctttgcatacttctgcctgctggggagcctggggactttccacaccgtgaggaatggtgccaggcatctcgggtttgatgtactgccaagccagccaatagccgctctaaaaataactcccccgtcgccatatttgggtgtccgctctaaaaataactcccggccgtcgccatatgggcccctccctggggacagccccttggaaagtcccgttagtgccaggcgggccatttaccgtcgctctaaaaataactccctagggaaacctgaagcccccattgacgtcaatggggcaggttttttatatagtccggcttcaggtttccctacaaggcctggggacaacccgagatgcctggcaggttttttatataacccaggggcacaggggctgccctcagtccgcctggagacctcgagccgagtggtcgtctcaggagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 29 - DNA, 577 bp, synthetic construct
  source 1..577: mol_type = other DNA
cactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccgccacc

SEQ ID NO: 30 - DNA, 845 bp, synthetic construct
  source 1..845: mol_type = other DNA
cactacgggtctaggctgcccatgtaaggaggcaaggcctggggacacccgagatgcctggttataattaacccagacatgtggctgccccccccccccaacacctgctgccctctaaaaataaccctgtccctggtggatccacactacgggtctaggctgcccatgtaaggaggcaaggcctggggacacccgagatgcctggttataattaacccagacatgtggctgccccccccccccaacacctgctgccctctaaaaataaccctgtccctggtggatccacactacgggtctaggctgcccatgtaaggaggcaaggcctggggacacccgagatgcctggttataattaacccagacatgtggctgccccccccccccaacacctgctgccctctaaaaataaccctgtccctggtggatccatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagccagccgccacc

SEQ ID NO: 31 - DNA, 443 bp, synthetic construct
  source 1..443: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 32 - DNA, 443 bp, synthetic construct
  source 1..443: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcagtccgcctggagacctcgagccgagtggtcgtctcaggagccagccagccgccacc

SEQ ID NO: 33 - DNA, 639 bp, synthetic construct
  source 1..639: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcagtccgcctggagacctcgagccgagtggtcgtctcaggagccagccagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 34 - DNA, 871 bp, synthetic construct
  source 1..871: mol_type = other DNA
tcgctgtcagccttccttgacacctctgtctcctcaggtgcctggctcccagtccccagaacgcctctcctgtaccttgcttcctagctgggcctttccttctcctctataaataccagctctggtatttcgccttggcagctgttgcccatgtaaggaggcaaggcctggggacacccgagatgcctggttataattaacccagacatgtggctgccccccccccccaacacctgctgccctctaaaaataaccctgcctcccacgctgcgcgcgcacctccccgcccccacccctacccgctggcgtgcccagtggaacggagccttgtgtctccgcctcaagtccccggatgctcacctccccgactcgcccccgctgtggccccgcccccgcgcggctcttcgtgccacgtcaccgcctgcgtcgcttccggaggcgcagcgggcgatgacgtagagggacgtgccctctatataaggttggggaacaggggctgccctcagtccgcctggagacctcgagccgagtggtcgtgcggctgagtcggccttttccgcccgctcccccctccccccgagcgccgctccggctgcaccgcgctcgctccgagtttcaggctcgtgctaagctagcgccgtcgtcgtctcccttcagtcctcaggagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 35 - DNA, 1196 bp, synthetic construct
  source 1..1196: mol_type = other DNA
gaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaagcagtgaaagggagtccctggctcctggctcgcctgcacgtcccagggcggggagggacttccgccctcacgtcccgctcttcgcaggtttccagatcctgtcctccgcctggggccccagcccggagagagccgctggtgcacacagggccgggattgtctgccctaattatcaggtccaggccccctccccctcaagccgcctcccgagcctccttcctctagatggggagggggaagtgggggctgggaaggaaccacgggcccccgcccgaggcccatgggcccctcctctttcccgcctctcagcctttgaaagaaagaaaggggagggggcaggtcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcagtccgcctggagacctcgagccgagtggtcgtatcttcttttgcgtcgccaggtgaagacgggcgggggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 36 - DNA, 1086 bp, synthetic construct
  source 1..1086: mol_type = other DNA
cggccgtccgccttcggcaccatcctcacgacacccaaatatggcgacgggtgaggaatggtggggagttatttttagagcggtgaggaatggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggcgctcccgcccgcctcaaagtgactaaaaggaaggaaaaaacctaaaatgtttccaagtggtggggtgtatggggagaaatgtgcccattcagcctctcaagggggcagagccctgtgtctgttctgtccctagaccgaggtggggacggggacagggaccggcaatgcaggaaatccctgcctctcatttgcacacttgggaaatgcaaagctacacacagtggagcgggggctgggggtgtgtagaccgccgggattccgagtttcccggcacgcctaggagagggagaggcaggcaatgtcagggaaattgggcaggcaagacgccagggacgccacgtactgccaggttctcaacgaggtggagccaaaggggcaggccccgcggtgcgcccggcgctgggctcacgggttgctgcacccggcccaggatcgcgggcggtgcagactcagcaggggcgggtgcaaggacgaggcggggcctctgcgcccggccctcttcccggactataaaaagagccgccggcttctgggctcagttcgcctggagacctcgagccgagtggtcgtcgcttttcatctgtcccgctgcgtgttttcctcttgatcgggaactcctgcttctccttgccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 37 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgaattccgaacgcaacggttcgccttcgcctcaggagccagccagccgccacc

SEQ ID NO: 38 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgacccccgaacgcaacggttcgccttcgcctcaggagccagccagccgccacc

SEQ ID NO: 39 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgaattccgccagcaacggttcgccttcgcctcaggagccagccagccgccacc

SEQ ID NO: 40 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgaattccgaactacacggttcgccttcgcctcaggagccagccagccgccacc

SEQ ID NO: 41 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgacccccgccagcaacggttcgccttcgcctcaggagccagccagccgccacc

SEQ ID NO: 42 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgacccccgaactacacggttcgccttcgcctcaggagccagccagccgccacc

SEQ ID NO: 43 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgaattccgccatacacggttcgccttcgcctcaggagccagccagccgccacc

SEQ ID NO: 44 - DNA, 450 bp, synthetic construct
  source 1..450: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattcgtctcttgacccccgccatacacggttcgccttcgcctcaggagccagccagccgccacc

SEQ ID NO: 45 - DNA, 669 bp, synthetic construct
  source 1..669: mol_type = other DNA
tgcccatgtaaggaggcaaggcctggggacacccgagatgcctggttataattaacccagacatgtggctgccccccccccccaacacctgctgccctctaaaaataaccctgtgcccatgtaaggaggcaaggcctggggacacccgagatgcctggttataattaacccagacatgtggctgccccccccccccaacacctgctgccctctaaaaataaccctgtgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 46 - DNA, 639 bp, synthetic construct
  source 1..639: mol_type = other DNA
agcttgtcgctgtcagccttccttgacacctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacaccactacgggtctaggctgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 47 - DNA, 435 bp, synthetic construct
  source 1..435: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtagcggcgcctatataaggtgtgggcgggtgtggcacagctagttccgtcgcagccggcgagccgaacggtcgtccgcggatccgccacc

SEQ ID NO: 48 - DNA, 533 bp, synthetic construct
  source 1..533: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtagcggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgccacc

SEQ ID NO: 49 - DNA, 616 bp, synthetic construct
  source 1..616: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtagggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgccacc

SEQ ID NO: 50 - DNA, 88 bp, synthetic construct
  source 1..88: mol_type = other DNA
cctccctggggacagcccctcctggctagtcacaccctgtaggctcctctatataacccaggggcacaggggctgccctcattctacc

SEQ ID NO: 51 - DNA, 366 bp, synthetic construct
  source 1..366: mol_type = other DNA
taattcaaggcctggggacaaattctgcattcctactgaattgggcccctccctggggacagccccaattagtgggggcggggcagaattcaaggcctggggacaaattctgcattcctactgaattaccaagtcagcaaattctgccaaatatggactaattctgctaaaaataactccactgaattctgccaaatatggactaattctgccaaatatggactaattacctccctggggacagcccctcctggctagtcacaccctgtaggctcctctatataacccaggggcacaggggctgccctcattctaccaccacctccacagcacagacagacactcaggagccagccagccgccacc

SEQ ID NO: 52 - DNA, 672 bp, synthetic construct
  source 1..672: mol_type = other DNA
taattcaaggcctggggacaaattctgcattcctactgaattgggcccctccctggggacagccccaattagtgggggcggggcagaattcaaggcctggggacaaattctgcattcctactgaattaccaagtcagcaaattctgccaaatatggactaattctgctaaaaataactccactgaattctgccaaatatggactaattctgccaaatatggactaattatgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 53 - DNA, 312 bp, synthetic construct
  source 1..312: mol_type = other DNA
tattctgcattcctactgaattcaaggcctggggacaaatttaaccaggcatctcgggtgtccaattggggctgtccccagggaggggcccaattagtccatatttggcagaattccaggcatatcgggtaattagtgggggcggggcagaatttagggaaacctgaagccaattcctccctggggacagcccctcctggctagtcacaccctgtaggctcctctatataacccaggggcacaggggctgccctcattctaccaccacctccacagcacagacagacactcaggagccagccagccgccacc

SEQ ID NO: 54 - DNA, 618 bp, synthetic construct
  source 1..618: mol_type = other DNA
tattctgcattcctactgaattcaaggcctggggacaaatttaaccaggcatctcgggtgtccaattggggctgtccccagggaggggcccaattagtccatatttggcagaattccaggcatatcgggtaattagtgggggcggggcagaatttagggaaacctgaagccaatttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 55 - DNA, 340 bp, synthetic construct
  source 1..340: mol_type = other DNA
taattttgctaaaaataactccagtgaatccagtaggaatgcagaattatatggtgccggccaattctgctaaaaataactccactgaatttagggaaacctgaagccaatttagggaaacttgaagccaattcaggggagttatttttagcagaattcagtggagttatttttagcagaattctgccccgcccccactaattcctccctggggacagcccctcctggctagtcacaccctgtaggctcctctatataacccaggggcacaggggctgccctcattctaccaccacctccacagcacagacagacactcaggagccagccagccgccacc

SEQ ID NO: 56 - DNA, 646 bp, synthetic construct
  source 1..646: mol_type = other DNA
taattttgctaaaaataactccagtgaatccagtaggaatgcagaattatatggtgccggccaattctgctaaaaataactccactgaatttagggaaacctgaagccaatttagggaaacttgaagccaattcaggggagttatttttagcagaattcagtggagttatttttagcagaattctgccccgcccccactaatttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 57 - DNA, 368 bp, synthetic construct
  source 1..368: mol_type = other DNA
taggcctggggacaaattcaaggcctggggacaaatttgctgacttggtaatttgtccccaggccttgaattggcttcaggtttccctaaattctgcacctgactaattctgctaaaaataactccactgaatttagggaaacctgaagccaattgctattggctggaatttagggaaacctgaagccaattccaggcatatcgggtaattctgccaaatatggactaattcctccctggggacagcccctcctggctagtcacaccctgtaggctcctctatataacccaggggcacaggggctgccctcattctaccaccacctccacagcacagacagacactcaggagccagccagccgccacc

SEQ ID NO: 58 - DNA, 674 bp, synthetic construct
  source 1..674: mol_type = other DNA
taggcctggggacaaattcaaggcctggggacaaatttgctgacttggtaatttgtccccaggccttgaattggcttcaggtttccctaaattctgcacctgactaattctgctaaaaataactccactgaatttagggaaacctgaagccaattgctattggctggaatttagggaaacctgaagccaattccaggcatatcgggtaattctgccaaatatggactaatttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 59 - DNA, 267 bp, synthetic construct
  source 1..267: mol_type = other DNA
taattggcttcaggtttccctaaattggcttcaggtttccctaaattccagccaatagcaatttgctgacttggtaattctgctaaaaataactccactgaatttaaccaggcatctcgggtgtccaattcctccctggggacagcccctcctggctagtcacaccctgtaggctcctctatataacccaggggcacaggggctgccctcattctaccaccacctccacagcacagacagacactcaggagccagccagccgccacc

SEQ ID NO: 60 - DNA, 573 bp, synthetic construct
  source 1..573: mol_type = other DNA
taattggcttcaggtttccctaaattggcttcaggtttccctaaattccagccaatagcaatttgctgacttggtaattctgctaaaaataactccactgaatttaaccaggcatctcgggtgtccaatttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 61 - DNA, 688 bp, synthetic construct
  source 1..688: mol_type = other DNA
taattggacacccgagatgcctggttaaattcagtggagttatttttagcagaattatatggcgacggccaattatatggcgacggccaattccaggcatatcgggtaatttgctgacttggtaatttgtccccaggccttgaattctgccccgcccccactaattagtccatatttggcagaatttattacccgatatacgtggaattccaggcatatcgggtaatttgctgacttggtaatttaaccaggcatctcgggtgtccaattgctattggctggaatttgtccccaggccttgaattctgccaaatatggactaattcagtaggaatgcagaatttgtccccaggccttgaattccaggcatatcgggtaataattggccgtcgccatataattccaggcatatcgggtaattctgcattcctactgaatttgtccccaggccttgaattagtccatatttggcagaatttagggaaacctgaagccaattagtccatatttggcagaattaccaagtcagcaaattctgcacctgactaattcctccctggggacagcccctcctggctagtcacaccctgtaggctcctctatataacccaggggcacaggggctgccctcattctaccaccacctccacagcacagacagacactcaggagccagccagccgccacc

SEQ ID NO: 62 - DNA, 994 bp, synthetic construct
  source 1..994: mol_type = other DNA
taattggacacccgagatgcctggttaaattcagtggagttatttttagcagaattatatggcgacggccaattatatggcgacggccaattccaggcatatcgggtaatttgctgacttggtaatttgtccccaggccttgaattctgccccgcccccactaattagtccatatttggcagaatttattacccgatatacgtggaattccaggcatatcgggtaatttgctgacttggtaatttaaccaggcatctcgggtgtccaattgctattggctggaatttgtccccaggccttgaattctgccaaatatggactaattcagtaggaatgcagaatttgtccccaggccttgaattccaggcatatcgggtaataattggccgtcgccatataattccaggcatatcgggtaattctgcattcctactgaatttgtccccaggccttgaattagtccatatttggcagaatttagggaaacctgaagccaattagtccatatttggcagaattaccaagtcagcaaattctgcacctgactaatttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 63 - DNA, 264 bp, synthetic construct
  source 1..264: mol_type = other DNA
taattagtcaggtgcagaattccagccaatagcaattagtgggggcggggcagaattggggctgtccccagggaggggcccaattcagtggagttatttttagcagaattaccaagtcagcaaattacctccctggggacagcccctcctggctagtcacaccctgtaggctcctctatataacccaggggcacaggggctgccctcattctaccaccacctccacagcacagacagacactcaggagccagccagccgccacc

SEQ ID NO: 64 - DNA, 570 bp, synthetic construct
  source 1..570: mol_type = other DNA
taattagtcaggtgcagaattccagccaatagcaattagtgggggcggggcagaattggggctgtccccagggaggggcccaattcagtggagttatttttagcagaattaccaagtcagcaaattatgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 65 - DNA, 351 bp, synthetic construct
  source 1..351: mol_type = other DNA
taattagtccatatttggcagaattaccaagtcagcaaatttagggaaacctgaagccaattcagtggagttatttttagcagaattgggcccctccctggggacagccccaattccaggcatatcgggtaattcagtaggaatgcagaattcagtggagttatttttagcagaattctgccaaatatggactaattccagccaatagcaattacctccctggggacagcccctcctggctagtcacaccctgtaggctcctctatataacccaggggcacaggggctgccctcattctaccaccacctccacagcacagacagacactcaggagccagccagccgccacc

SEQ ID NO: 66 - DNA, 657 bp, synthetic construct
  source 1..657: mol_type = other DNA
taattagtccatatttggcagaattaccaagtcagcaaatttagggaaacctgaagccaattcagtggagttatttttagcagaattgggcccctccctggggacagccccaattccaggcatatcgggtaattcagtaggaatgcagaattcagtggagttatttttagcagaattctgccaaatatggactaattccagccaatagcaattatgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 67 - DNA, 244 bp, synthetic construct
  source 1..244: mol_type = other DNA
taattagtccatatttggcagaatttgtccccaggccttgaattcagtaggaatgcagaattcagtaggaatgcagaatttaaccaggcatctcgggtgtccaattacctccctggggacagcccctcctggctagtcacaccctgtaggctcctctatataacccaggggcacaggggctgccctcattctaccaccacctccacagcacagacagacactcaggagccagccagccgccacc

SEQ ID NO: 68 - DNA, 550 bp, synthetic construct
  source 1..550: mol_type = other DNA
taattagtccatatttggcagaatttgtccccaggccttgaattcagtaggaatgcagaattcagtaggaatgcagaatttaaccaggcatctcgggtgtccaattatgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 69 - DNA, 224 bp, synthetic construct
  source 1..224: mol_type = other DNA
taattgctattggctggaattatatggcgacggccaattggggctgtccccagggaggggcccaattcaaggcctggggacaaattacctccctggggacagcccctcctggctagtcacaccctgtaggctcctctatataacccaggggcacaggggctgccctcattctaccaccacctccacagcacagacagacactcaggagccagccagccgccacc

SEQ ID NO: 70 - DNA, 530 bp, synthetic construct
  source 1..530: mol_type = other DNA
taattgctattggctggaattatatggcgacggccaattggggctgtccccagggaggggcccaattcaaggcctggggacaaattatgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgccacc

SEQ ID NO: 71 - DNA, 614 bp, synthetic construct
  source 1..614: mol_type = other DNA
gaattcggtaccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccaccccgccacc

SEQ ID NO: 72 - DNA, 767 bp, synthetic construct
  source 1..767: mol_type = other DNA
gaattcggtaccccactacgggtctaggctgcccatgtaaggaggcaaggcctggggacacccgagatgcctggttataattaaccccaacacctgctgcccccccccccccaacacctgctgcctctaaaaataaccctgtccctggtggatcccctgcatgccccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagcggccgatccgccaccccgccacc

SEQ ID NO: 73 - DNA, 777 bp, synthetic construct
  source 1..777: mol_type = other DNA
tagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacccgccacc

SEQ ID NO: 74 - DNA, 801 bp, synthetic construct
  source 1..801: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactagccacc

SEQ ID NO: 75 - DNA, 776 bp, synthetic construct
  source 1..776: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcctcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccgccacc

SEQ ID NO: 76 - DNA, 781 bp, synthetic construct
  source 1..781: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaagccacc

SEQ ID NO: 77 - DNA, 670 bp, synthetic construct
  source 1..670: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtacttgccacc

SEQ ID NO: 78 - DNA, 863 bp, synthetic construct
  source 1..863: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttcagccacc

SEQ ID NO: 79 - DNA, 659 bp, synthetic construct
  source 1..659: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcctctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcggggccacc

SEQ ID NO: 80 - DNA, 642 bp, synthetic construct
  source 1..642: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccggggccacc

SEQ ID NO: 81 - DNA, 848 bp, synthetic construct
  source 1..848: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaagagccacc

SEQ ID NO: 82 - DNA, 667 bp, synthetic construct
  source 1..667: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtgccacc

SEQ ID NO: 83 - DNA, 669 bp, synthetic construct
  source 1..669: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttggccacc

SEQ ID NO: 84 - DNA, 996 bp, synthetic construct
  source 1..996: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctgagccacc

SEQ ID NO: 85 - DNA, 804 bp, synthetic construct
  source 1..804: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctctgccacc

SEQ ID NO: 86 - DNA, 811 bp, synthetic construct
  source 1..811: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggcgccacc

SEQ ID NO: 87 - DNA, 878 bp, synthetic construct
  source 1..878: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccaggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggccgccacc

SEQ ID NO: 88 - DNA, 732 bp, synthetic construct
  source 1..732: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccgctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgcgccacc

SEQ ID NO: 89 - DNA, 703 bp, synthetic construct
  source 1..703: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagccccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgacgccacc

SEQ ID NO: 90 - DNA, 818 bp, synthetic construct
  source 1..818: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccttgggtgtagaagggacgggccgtggcggttgcagactgggagggcctgacgggaaagggaaaggttgggcccctgtggacgccacagcgcgccttccccatccgcaaaggctttccctccctctcctccagctccagcctgaaccgccacc

SEQ ID NO: 91 - DNA, 626 bp, synthetic construct
  source 1..626: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcctgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctagccacc

SEQ ID NO: 92 - DNA, 1346 bp, synthetic construct
  source 1..1346: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtggccacc

SEQ ID NO: 93 - DNA, 972 bp, synthetic construct
  source 1..972: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtggccacc

SEQ ID NO: 94 - DNA, 753 bp, synthetic construct
  source 1..753: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcctgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctggccacc

SEQ ID NO: 95 - DNA, 1042 bp, synthetic construct
  source 1..1042: mol_type = other DNA
tgtacgggccagatatacgcgtttgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcccgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagtttagccacc

SEQ ID NO: 96 - DNA, 221 bp, synthetic construct
  source 1..221: mol_type = other DNA
cgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccaggggcggatccgccacc

SEQ ID NO: 97 - DNA, 221 bp, synthetic construct
  source 1..221: mol_type = other DNA
cgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccgagccgagtggtcgttcccctccgcggcgaccaggggcggatccgccacc

SEQ ID NO: 98 - DNA, 209 bp, synthetic construct
  source 1..209: mol_type = other DNA
cgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgtgcctccatagaaggatccgccacc

SEQ ID NO: 99 - DNA, 299 bp, synthetic construct
  source 1..299: mol_type = other DNA
cgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgttgagccaagaggtaagggtttaagggatggttggttggtggggtattaatgtttaattacctggagcacctgcctgaaatcactttttttcaggttggccgcggatccgccacc

SEQ ID NO: 100 - DNA, 334 bp, synthetic construct
  source 1..334: mol_type = other DNA
tgcccatgtaaggaggcaaggcctggggacacccgagatgcctggttataattaacccagacatgtggctgccccccccccccaacacctgctgccctctaaaaataaccctgcgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccgagccgagtggtcgttcccctccgcggcgaccaggggcggatccgccacc

SEQ ID NO: 101 - DNA, 281 bp, synthetic construct
  source 1..281: mol_type = other DNA
tggctgatcctctcccctgcccttggctccatgaatggcctcggcagtcctagcgggtgccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccgagccgagtggtcgttcccctccgcggcgaccaggggcggatccgccacc

SEQ ID NO: 102 - DNA, 230 bp, synthetic construct
  source 1..230: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatcagcgttctataaagcggccctcctggagccagccacccagagcccgctgccgccggagccgagccgacccgccccgccgggatccgccacc

SEQ ID NO: 103 - DNA, 230 bp, synthetic construct
  source 1..230: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgtccccgccgggatccgccacc

SEQ ID NO: 104 - DNA, 234 bp, synthetic construct
  source 1..234: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgcctccatagaaggatccgccacc

SEQ ID NO: 105 - DNA, 324 bp, synthetic construct
  source 1..324: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgttgagccaagaggtaagggtttaagggatggttggttggtggggtattaatgtttaattacctggagcacctgcctgaaatcactttttttcaggttggccgcggatccgccacc

SEQ ID NO: 106 - DNA, 351 bp, synthetic construct
  source 1..351: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgttgagccaagaggtaagggtttaacgggcgggggcccaggccgcgaaccggccgagggagggggctctagtgcccaacacccaaatatggcgagcacctgcctgaaatcactttttttcaggttggccgcggatccgccacc

SEQ ID NO: 107 - DNA, 509 bp, synthetic construct
  source 1..509: mol_type = other DNA
cgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgttgagccaagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgtggggtattaatgtttaattacctggagcacctgcctgaaatcactttttttcaggttggccgcggatccgccacc

SEQ ID NO: 108 - DNA, 554 bp, synthetic construct
  source 1..554: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgttgagccaagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgtccccgccgggatccgccaccgtggggtattaatgtttaattacctggagcacctgcctgaaatcactttttttcaggttggccgcggatccgccacc

SEQ ID NO: 109 - DNA, 270 bp, synthetic construct
  source 1..270: mol_type = other DNA
gttctcctctataaatacccgctctggtatttggggttggcagctgttgctgccctataaatacgtatttatagctataaatacctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccggatccgccacc

SEQ ID NO: 110 - DNA, 225 bp, synthetic construct
  source 1..225: mol_type = other DNA
gttctcctctataaatacccgctctggtatttggggttggcagctgttgctgccctataaatacgtatttatagctataaatacctggcaggacagcgggatcttgcagctgtcaggggaggggaggcgggggctgatgtcaggagggatatataaagtgccgacggctgggggcccttcagtccgcctggagacctcgagccgagtggtcgtggatccgccacc

SEQ ID NO: 111 - DNA, 237 bp, synthetic construct
  source 1..237: mol_type = other DNA
gttctcctctataaatacccgctctggtatttggggttggcagctgttgctgccctataaatacgtatttatagctataaatacctggcaggacagcgggatcttgcagctgtcaggggaggggaggcgggggctgatgtcacggcgcctataaaaagtgtggggcgcggcgggcgggtcagtccgcctggagacctcgagccgagtggtcgtgcctccatagaaggatccgccacc

SEQ ID NO: 112 - DNA, 345 bp, synthetic construct
  source 1..345: mol_type = other DNA
ccactacgggtctaggctgcccatgtaaggaggcaaggcctggggacacccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtaggctcctctatataacccaggggcacaggggctgccctcagtccgcctggagacctcgagccgagtggtcgtctcaggagccggatccgccacc

SEQ ID NO: 113 - DNA, 345 bp, synthetic construct
  source 1..345: mol_type = other DNA
ccactacgggtctaggctgcccatgtaaggaggcaaggcctggggacacccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtagcggcgcctataaaaagtgtggggcgcggcgggcgggtcagtccgcctggagacctcgagccgagtggtcgtctcaggagccggatccgccacc

SEQ ID NO: 114 - DNA, 296 bp, synthetic construct
  source 1..296: mol_type = other DNA
ccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtagcggcgcctataaaaagtgtggggcgcggcgggcgggtcagtccgcctggagacctcgagccgagtggtcgtctcaggagccggatccgccacc

SEQ ID NO: 115 - DNA, 388 bp, synthetic construct
  source 1..388: mol_type = other DNA
ccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtagcggcgcctataaaaagtgtggggcgcggcgggcgggtcagtccgcctggagacctcgagccgagtggtcgtctcaggagccaagaggtaagggtttaagggatggttggttggtggggtattaatgtttaattacctggagcacctgcctgaaatcactttttttcaggttggggatccgccacc

SEQ ID NO: 116 - DNA, 592 bp, synthetic construct
  source 1..592: mol_type = other DNA
ccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtagcggcgcctataaaaagtgtggggcgcggcgggcgggtcagtccgcctggagacctcgagccgagtggtcgtctcaggagccaagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgtggggtgtttaattacctggagcacctgcctgaaatcactttttttcaggttggggatccgccacc

SEQ ID NO: 117 - DNA, 607 bp, synthetic construct
  source 1..607: mol_type = other DNA
ccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtagcggcgcctataaaaagtgtggggcgcggcgggcgggtcagtccgcctggagacctcgagccgagtggtcgtctcaggagccaagaggtaagggtttaagggatggttggttggttctcctctataaatacccgctctggtatttggggttggcagctgttgctgccctataaatacgtatttatagctataaatacctggcaggacagcgggatcttgcagctgtcaggggaggggaggcgggggctgatgtcacggcgcctataaaaagtgtggggcgcggcgggcgggtcagtccgcctggagacctcgagccgagtggtcgtgcctccatagaagtggggtgtttaattacctggagcacctgcctgaaatcactttttttcaggttggggatccgccacc

SEQ ID NO: 118 - DNA, 567 bp, synthetic construct
  source 1..567: mol_type = other DNA
ccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggatctccctggggacagcccctcctggctagtcacaccctgtagcggcgcctataaaaagtgtggggcgcggcgggcgggtcagtccgcctggagacctcgagccgagtggtcgtctcaggagccaagaggtaagggtttaagggatggttggttgcgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgtgtggggtgtttaattacctggagcacctgcctgaaatcactttttttcaggttggggatccgccacc

SEQ ID NO: 119 - DNA, 503 bp, synthetic construct
  source 1..503: mol_type = other DNA
cgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgttgagccaagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgtggggtgtttaattacctggagcacctgcctgaaatcactttttttcaggttggccgcggatccgccacc

SEQ ID NO: 120 - DNA, 536 bp, synthetic construct
  source 1..536: mol_type = other DNA
tggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgttgagccaagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgtccccgccggtggggtgtttaattacctggagcacctgcctgaaatcactttttttcaggttggccgcggatccgccacc

SEQ ID NO: 121 - DNA, 67 bp, synthetic construct
  source 1..67: mol_type = other DNA
cgggcgggggcccaggccgcgaaccggccgagggagggggctctagtgcccaacacccaaatatggc

SEQ ID NO: 122 - DNA, 60 bp, synthetic construct
  source 1..60: mol_type = other DNA
tggctgatcctctcccctgcccttggctccatgaatggcctcggcagtcctagcgggtgc

SEQ ID NO: 123 - DNA, 382 bp, synthetic construct
  source 1..382: mol_type = other DNA
ggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaaga

SEQ ID NO: 124 - DNA, 412 bp, synthetic construct
  source 1..412: mol_type = other DNA
caggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggcc

SEQ ID NO: 125 - DNA, 266 bp, synthetic construct
  source 1..266: mol_type = other DNA
gctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgc

SEQ ID NO: 126 - DNA, 71 bp, synthetic construct
  source 1..71: mol_type = other DNA
cggcgcctatataaggtgtgggcgggtgtggcacagctagttccgtcgcagccggcgagccgaacggtcgt

SEQ ID NO: 127 - DNA, 201 bp, synthetic construct
  source 1..201: mol_type = other DNA
cgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagt

SEQ ID NO: 128 - DNA, 437 bp, synthetic construct
  source 1..437: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 129 - DNA, 454 bp, synthetic construct
  source 1..454: mol_type = other DNA
ccactacgggtctaggctgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 130 - DNA, 443 bp, synthetic construct; identical to SEQ ID NO: 31
  source 1..443: mol_type = other DNA

SEQ ID NO: 131 - DNA, 348 bp, synthetic construct
  source 1..348: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtag

SEQ ID NO: 132 - DNA, 384 bp, synthetic construct
  source 1..384: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccc

SEQ ID NO: 133 - DNA, 419 bp, synthetic construct
  source 1..419: mol_type = other DNA
tgcccatgtaaggaggcaaggcctggggacacccgagatgcctggttataattaacccagacatgtggctgccccccccccccaacacctgctgccctctaaaaataaccctgcattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcagtccgcctggagacctcgagccgagtggtcgt

SEQ ID NO: 134 - DNA, 206 bp, synthetic construct
  source 1..206: mol_type = other DNA
ccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatc

SEQ ID NO: 135 - DNA, 390 bp, synthetic construct
  source 1..390: mol_type = other DNA
atcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcagtccgcctggagacctcgagccgagtggtcgt

SEQ ID NO: 136 - DNA, 530 bp, synthetic construct
  source 1..530: mol_type = other DNA
cgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctga

SEQ ID NO: 137 - DNA, 335 bp, synthetic construct
  source 1..335: mol_type = other DNA
cgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 138 - DNA, 310 bp, synthetic construct
  source 1..310: mol_type = other DNA
tcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacacc

SEQ ID NO: 139 - DNA, 203 bp, synthetic construct
  source 1..203: mol_type = other DNA
cagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttg

SEQ ID NO: 140 - DNA, 352 bp, synthetic construct
  source 1..352: mol_type = other DNA
cccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccttgggtgtagaagggacgggccgtggcggttgcagactgggagggcctgacgggaaagggaaaggttgggcccctgtggacgccacagcgcgccttccccatccgcaaaggctttccctccctctcctccagctccagcctgaacc

SEQ ID NO: 141 - DNA, 338 bp, synthetic construct
  source 1..338: mol_type = other DNA
ctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctct

SEQ ID NO: 142 - DNA, 994 bp, synthetic construct
  source 1..994: mol_type = other DNA
gaggacacagcctggctctgggtcccagcaggaaaggcaatcccagaaaggcagggtcagggactggagtcctgtgggtgctttttaagcaaagattatcaccaggcaggctaaacttagcaaccggcttttagctagaagggcagggggctggtgtcaggttatgctgggccagcaaagaggcccgggatccccctcccatgcacctgctgatgggccaagcagtgaaagggagtccctggctcctggctcgcctgcacgtcccagggcggggagggacttccgccctcacgtcccgctcttcgcaggtttccagatcctgtcctccgcctggggccccagcccggagagagccgctggtgcacacagggccgggattgtctgccctaattatcaggtccaggccccctccccctcaagccgcctcccgagcctccttcctctagatggggagggggaagtgggggctgggaaggaaccacgggcccccgcccgaggcccatgggcccctcctctttcccgcctctcagcctttgaaagaaagaaaggggagggggcaggtcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcagtccgcctggagacctcgagccgagtggtcgtatcttcttttgcgtcgccaggtgaagacgggcgg

SEQ ID NO: 143 - DNA, 193 bp, synthetic construct
  source 1..193: mol_type = other DNA
tctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcggg

SEQ ID NO: 144 - DNA, 212 bp, synthetic construct
  source 1..212: mol_type = other DNA
ctcaggagccgggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgctaaaggccgccacc

SEQ ID NO: 145 - DNA, 160 bp, synthetic construct
  source 1..160: mol_type = other DNA
tgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtcta

SEQ ID NO: 146 - DNA, 880 bp, synthetic construct
  source 1..880: mol_type = other DNA
cctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 147 - DNA, 506 bp, synthetic construct
  source 1..506: mol_type = other DNA
cctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 148 - DNA, 189 bp, synthetic construct
  source 1..189: mol_type = other DNA
gggcaggttggtatcaaggttacaagcagcccacctgtcccagtgctgacttagtgcaaggcgagccagcaaggagggaggacaggtggcagtggggggtgaggagcatctaaaaatagccactcttgggtttctgataggcactgactctctctgcctattggtctattttcccacccttaggctgct

SEQ ID NO: 149 - DNA, 854 bp, synthetic construct
  source 1..854: mol_type = other DNA
gggcaggttggtatcaaggttacaagcagcccacctgtcccagtgctgacttagtgcaaggcgagccagcaaggagggaggacaggtggcagtggggggtgaggagcatctaaaaatagcccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatatataaagtgccgacggctgggggcccttcagtccgcctggagacctcgagccgagtggtcgtactcttgggtttctgataggcactgactctctctgcctattggtctattttcccacccttaggctgct

SEQ ID NO: 150 - DNA, 189 bp, synthetic construct; identical to SEQ ID NO: 148
  source 1..189: mol_type = other DNA

SEQ ID NO: 151 - DNA, 665 bp, synthetic construct
  source 1..665: mol_type = other DNA
cacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatatataaagtgccgacggctgggggcccttcagtccgcctggagacctcgagccgagtggtcgt

SEQ ID NO: 152 - DNA, 237 bp, synthetic construct
  source 1..237: mol_type = other DNA
ccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgac

SEQ ID NO: 153 - DNA, 287 bp, synthetic construct
  source 1..287: mol_type = other DNA
tgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctg

SEQ ID NO: 154 - DNA, 113 bp, synthetic construct
  source 1..113: mol_type = other DNA
tgcccatgtaaggaggcaaggcctggggacacccgagatgcctggttataattaacccagacatgtggctgccccccccccccaacacctgctgccctctaaaaataaccctg

SEQ ID NO: 155 - DNA, 137 bp, synthetic construct
  source 1..137: mol_type = other DNA
cctccctggggacagcccctcctggctagtcacaccctgtaggctcctctatataacccaggggcacaggggctgccctcattctaccaccacctccacagcacagacagacactcaggagccagccagccgccacc

SEQ ID NO: 156 - DNA, 60 bp, synthetic construct
  source 1..60: mol_type = other DNA
tcattcgtctcttgacccccgccatacacggttcgccttcgcctcaggagccagccagcc

SEQ ID NO: 157 - DNA, 60 bp, synthetic construct
  source 1..60: mol_type = other DNA
tcattcgtctcttgacccccgaacgcaacggttcgccttcgcctcaggagccagccagcc

SEQ ID NO: 158 - DNA, 60 bp, synthetic construct
  source 1..60: mol_type = other DNA
tcattcgtctcttgaattccgccagcaacggttcgccttcgcctcaggagccagccagcc

SEQ ID NO: 159 - DNA, 60 bp, synthetic construct
  source 1..60: mol_type = other DNA
tcattcgtctcttgaattccgaactacacggttcgccttcgcctcaggagccagccagcc

SEQ ID NO: 160 - DNA, 60 bp, synthetic construct
  source 1..60: mol_type = other DNA
tcattcgtctcttgacccccgccagcaacggttcgccttcgcctcaggagccagccagcc

SEQ ID NO: 161 - DNA, 60 bp, synthetic construct
  source 1..60: mol_type = other DNA
tcattcgtctcttgacccccgaactacacggttcgccttcgcctcaggagccagccagcc

SEQ ID NO: 162 - DNA, 60 bp, synthetic construct
  source 1..60: mol_type = other DNA
tcattcgtctcttgaattccgccatacacggttcgccttcgcctcaggagccagccagcc

SEQ ID NO: 163 - DNA, 60 bp, synthetic construct; identical to SEQ ID NO: 156
  source 1..60: mol_type = other DNA

SEQ ID NO: 164 - DNA, 179 bp, synthetic construct
  source 1..179: mol_type = other DNA
agcttgtcgctgtcagccttccttgacacctctgtctcctcaggtgcctggctcccagtccccagaacgcctctcctgtaccttgcttcctagctgggcctttccttctcctctataaataccagctctggtatttcgccttggcagctgttgctgctagggagacggctggcttgaca

SEQ ID NO: 165 - DNA, 145 bp, synthetic construct
  source 1..145: mol_type = other DNA
tcgctgtcagccttccttgacacctctgtctcctcaggtgcctggctcccagtccccagaacgcctctcctgtaccttgcttcctagctgggcctttccttctcctctataaataccagctctggtatttcgccttggcagctgt

SEQ ID NO: 166 - DNA, 140 bp, synthetic construct
  source 1..140: mol_type = other DNA
tcgagaagggcagcgacattcctgcggggtggcgcggagggaatgcccgcgggctatataaaacctgagcagagggacaagcggccaccgcagcggacagcgccaagtgaagcctcgcttcccctccgcggcgaccaggg

SEQ ID NO: 167 - DNA, 140 bp, synthetic construct
  source 1..140: mol_type = other DNA
tcgagaagggcagcgacattcctgcggggtggcgcggagggaatgcccgcgggctatataaaacctgagcagagggacaagcggccaccgcagcggacagcgccgagccgagtggtcgttcccctccgcggcgaccaggg

SEQ ID NO: 168 - DNA, 130 bp, synthetic construct
  source 1..130: mol_type = other DNA
tcgagaagggcagcgacattcctgcggggtggcgcggagggaatgcccggcgcctataaaaagtgtggggcgcggcgggcgggtcagtccgcctggagacctcgagccgagtggtcgtgcctccatagaa

SEQ ID NO: 169 - DNA, 118 bp, synthetic construct
  source 1..118: mol_type = other DNA
tcgagaagggcagcgacattcctgcggggtggcgcggagggaatgcccggcgcctataaaaagtgtggggcgcggcgggcgggtcagtccgcctggagacctcgagccgagtggtcgt

SEQ ID NO: 170 - DNA, 185 bp, synthetic construct
  source 1..185: mol_type = other DNA
cgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgt

SEQ ID NO: 171 - DNA, 220 bp, synthetic construct
  source 1..220: mol_type = other DNA
tcgagaagggcagcgacattcctgcggggtggcgcggagggaatgcccggcgcctataaaaagtgtggggcgcggcgggcgggtcagtccgcctggagacctcgagccgagtggtcgttgagccaagaggtaagggtttaagggatggttggttggtggggtattaatgtttaattacctggagcacctgcctgaaatcactttttttcaggttggccgc

SEQ ID NO: 172 - DNA, 430 bp, synthetic construct
  source 1..430: mol_type = other DNA
tcgagaagggcagcgacattcctgcggggtggcgcggagggaatgcccggcgcctataaaaagtgtggggcgcggcgggcgggtcagtccgcctggagacctcgagccgagtggtcgttgagccaagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgtggggtattaatgtttaattacctggagcacctgcctgaaatcactttttttcaggttggccgc

SEQ ID NO: 173 - DNA, 158 bp, synthetic construct
  source 1..158: mol_type = other DNA
gaaggggaccaaataaggcaaggtggcagaccgggccccccacccctgcccccggctgctccaactgaccctgtccatcagcgttctataaagcggccctcctggagccagccacccagagcccgctgccgccggagccgagccgacccgccccgccg

SEQ ID NO: 174 - DNA, 158 bp, synthetic construct
  source 1..158: mol_type = other DNA
gaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgtccccgccg

SEQ ID NO: 175 - DNA, 150 bp, synthetic construct
  source 1..150: mol_type = other DNA
gaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgt

SEQ ID NO: 176 - DNA, 162 bp, synthetic construct
  source 1..162: mol_type = other DNA
gaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgcctccatagaa

SEQ ID NO: 177 - DNA, 150 bp, synthetic construct
  source 1..150: mol_type = other DNA
gaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgt

SEQ ID NO: 178 - DNA, 252 bp, synthetic construct
  source 1..252: mol_type = other DNA
gaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgttgagccaagaggtaagggtttaagggatggttggttggtggggtattaatgtttaattacctggagcacctgcctgaaatcactttttttcaggttggccgc

SEQ ID NO: 179 - DNA, 279 bp, synthetic construct
  source 1..279: mol_type = other DNA
gaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgttgagccaagaggtaagggtttaacgggcgggggcccaggccgcgaaccggccgagggagggggctctagtgcccaacacccaaatatggcgagcacctgcctgaaatcactttttttcaggttggccgc

SEQ ID NO: 180 - DNA, 211 bp, synthetic construct
  source 1..211: mol_type = other DNA
ccactacgggtctaggctgcccatgtaaggaggcaaggcctggggacacccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggat

SEQ ID NO: 181 - DNA, 122 bp, synthetic construct
  source 1..122: mol_type = other DNA
ctccctggggacagcccctcctggctagtcacaccctgtaggctcctctatataacccaggggcacaggggctgccctcagtccgcctggagacctcgagccgagtggtcgtctcaggagcc

SEQ ID NO: 182 - DNA, 122 bp, synthetic construct
  source 1..122: mol_type = other DNA
ctccctggggacagcccctcctggctagtcacaccctgtagcggcgcctataaaaagtgtggggcgcggcgggcgggtcagtccgcctggagacctcgagccgagtggtcgtctcaggagcc

SEQ ID NO: 183 - DNA, 162 bp, synthetic construct
  source 1..162: mol_type = other DNA
ccgagatgcctggttataattaacccacaacacctgctgccccccccccccaacacctgctgcctgagcctgagcggttaccccaccccggtgcctgggtcttaggctctgtacaccatggaggagaagctcgctctaaaaataaccctgtccctggtggat

SEQ ID NO: 184 - DNA, 84 bp, synthetic construct
  source 1..84: mol_type = other DNA
gttctcctctataaatacccgctctggtatttggggttggcagctgttgctgccctataaatacgtatttatagctataaatac

SEQ ID NO: 185 - DNA, 110 bp, synthetic construct
  source 1..110: mol_type = other DNA
gggatcttgcagctgtcaggggaggggaggcgggggctgatgtcaggagggatacaaatagtgccgacggctgggggccctgtctcccctcgccgcatccactctccggc

SEQ ID NO: 186 - DNA, 116 bp, synthetic construct
  source 1..116: mol_type = other DNA
gggatcttgcagctgtcaggggaggggaggcgggggctgatgtcaggagggatatataaagtgccgacggctgggggcccttcagtccgcctggagacctcgagccgagtggtcgt

SEQ ID NO: 187 - DNA, 128 bp, synthetic construct
  source 1..128: mol_type = other DNA
gggatcttgcagctgtcaggggaggggaggcgggggctgatgtcacggcgcctataaaaagtgtggggcgcggcgggcgggtcagtccgcctggagacctcgagccgagtggtcgtgcctccatagaa

SEQ ID NO: 188 - DNA, 150 bp, synthetic construct; identical to SEQ ID NO: 177
  source 1..150: mol_type = other DNA

SEQ ID NO: 189 - DNA, 617 bp, synthetic construct
  source 1..617: mol_type = other DNA
aaagtgactaaaaggaaggaaaaaacctaaaatgtttccaagtggtggggtgtatggggagaaatgtgcccattcagcctctcaagggggcagagccctgtgtctgttctgtccctagaccgaggtggggacggggacagggaccggcaatgcaggaaatccctgcctctcatttgcacacttgggaaatgcaaagctacacacagtggagcgggggctgggggtgtgtagaccgccgggattccgagtttcccggcacgcctaggagagggagaggcaggcaatgtcagggaaattgggcaggcaagacgccagggacgccacgtactgccaggttctcaacgaggtggagccaaaggggcaggccccgcggtgcgcccggcgctgggctcacgggttgctgcacccggcccaggatcgcgggcggtgcagactcagcaggggcgggtgcaaggacgaggcggggcctctgcgcccggccctcttcccggactataaaaagagccgccggcttctgggctcagttcgcctggagacctcgagccgagtggtcgtcgcttttcatctgtcccgctgcgtgttttcctcttgatcgggaactcctgcttctccttgcc

SEQ ID NO: 190 - DNA, 175 bp, synthetic construct
  source 1..175: mol_type = other DNA
tattctgcattcctactgaattcaaggcctggggacaaatttaaccaggcatctcgggtgtccaattggggctgtccccagggaggggcccaattagtccatatttggcagaattccaggcatatcgggtaattagtgggggcggggcagaatttagggaaacctgaagccaatt

SEQ ID NO: 191 - DNA, 203 bp, synthetic construct
  source 1..203: mol_type = other DNA
taattttgctaaaaataactccagtgaatccagtaggaatgcagaattatatggtgccggccaattctgctaaaaataactccactgaatttagggaaacctgaagccaatttagggaaacttgaagccaattcaggggagttatttttagcagaattcagtggagttatttttagcagaattctgccccgcccccactaatt

SEQ ID NO: 192 - DNA, 231 bp, synthetic construct
  source 1..231: mol_type = other DNA
taggcctggggacaaattcaaggcctggggacaaatttgctgacttggtaatttgtccccaggccttgaattggcttcaggtttccctaaattctgcacctgactaattctgctaaaaataactccactgaatttagggaaacctgaagccaattgctattggctggaatttagggaaacctgaagccaattccaggcatatcgggtaattctgccaaatatggactaatt

SEQ ID NO: 193 - DNA, 130 bp, synthetic construct
  source 1..130: mol_type = other DNA
taattggcttcaggtttccctaaattggcttcaggtttccctaaattccagccaatagcaatttgctgacttggtaattctgctaaaaataactccactgaatttaaccaggcatctcgggtgtccaatt

SEQ ID NO: 194 - DNA, 551 bp, synthetic construct
  source 1..551: mol_type = other DNA
taattggacacccgagatgcctggttaaattcagtggagttatttttagcagaattatatggcgacggccaattatatggcgacggccaattccaggcatatcgggtaatttgctgacttggtaatttgtccccaggccttgaattctgccccgcccccactaattagtccatatttggcagaatttattacccgatatacgtggaattccaggcatatcgggtaatttgctgacttggtaatttaaccaggcatctcgggtgtccaattgctattggctggaatttgtccccaggccttgaattctgccaaatatggactaattcagtaggaatgcagaatttgtccccaggccttgaattccaggcatatcgggtaataattggccgtcgccatataattccaggcatatcgggtaattctgcattcctactgaatttgtccccaggccttgaattagtccatatttggcagaatttagggaaacctgaagccaattagtccatatttggcagaattaccaagtcagcaaattctgcacctgactaatt

SEQ ID NO: 195 - DNA, 127 bp, synthetic construct
  source 1..127: mol_type = other DNA
taattagtcaggtgcagaattccagccaatagcaattagtgggggcggggcagaattggggctgtccccagggaggggcccaattcagtggagttatttttagcagaattaccaagtcagcaaatta

SEQ ID NO: 196 - DNA, 229 bp, synthetic construct
  source 1..229: mol_type = other DNA
taattcaaggcctggggacaaattctgcattcctactgaattgggcccctccctggggacagccccaattagtgggggcggggcagaattcaaggcctggggacaaattctgcattcctactgaattaccaagtcagcaaattctgccaaatatggactaattctgctaaaaataactccactgaattctgccaaatatggactaattctgccaaatatggactaatta

SEQ ID NO: 197 - DNA, 214 bp, synthetic construct
  source 1..214: mol_type = other DNA
taattagtccatatttggcagaattaccaagtcagcaaatttagggaaacctgaagccaattcagtggagttatttttagcagaattgggcccctccctggggacagccccaattccaggcatatcgggtaattcagtaggaatgcagaattcagtggagttatttttagcagaattctgccaaatatggactaattccagccaatagcaatta

SEQ ID NO: 198 - DNA, 107 bp, synthetic construct
  source 1..107: mol_type = other DNA
taattagtccatatttggcagaatttgtccccaggccttgaattcagtaggaatgcagaattcagtaggaatgcagaatttaaccaggcatctcgggtgtccaatta

SEQ ID NO: 199 - DNA, 87 bp, synthetic construct
  source 1..87: mol_type = other DNA
taattgctattggctggaattatatggcgacggccaattggggctgtccccagggaggggcccaattcaaggcctggggacaaatta

SEQ ID NO: 200 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 201 - DNA, 271 bp, synthetic construct
  source 1..271: mol_type = other DNA
aagaggtaagggtttaagggatggttggttgcgggcgggggcccaggccgcgaaccggccgagggagggggctctagtgcccaacacccaaatatggctcgagaagggcagcgacattcctgcggggtggcgcggagggaatgcccggcgcctataaaaagtgtggggcgcggcgggcgggtcagtccgcctggagacctcgagccgagtggtcgtgtggggtgtttaattacctggagcacctgcctgaaatcactttttttcaggttgg

SEQ ID NO: 202 - DNA, 119 bp, synthetic construct
  source 1..119: mol_type = other DNA
aagaggtaagggtttaacgggcgggggcccaggccgcgaaccggccgagggagggggctctagtgcccaacacccaaatatggcgagcacctgcctgaaatcactttttttcaggttgg

SEQ ID NO: 203 - DNA, 296 bp, synthetic construct
  source 1..296: mol_type = other DNA
aagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgtggggtgtttaattacctggagcacctgcctgaaatcactttttttcaggttgg

SEQ ID NO: 204 - DNA, 308 bp, synthetic construct
  source 1..308: mol_type = other DNA
aagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgtccccgccggtggggtgtttaattacctggagcacctgcctgaaatcactttttttcaggttggccgc

SEQ ID NO: 205 - DNA, 296 bp, synthetic construct; identical to SEQ ID NO: 203
  source 1..296: mol_type = other DNA

SEQ ID NO: 206 - DNA, 302 bp, synthetic construct
  source 1..302: mol_type = other DNA
aagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatccggcgcctataaaaagtgtggggcgcggcgggcgggtcagtccgcctggagacctcgagccgagtggtcgtgtggggtattaatgtttaattacctggagcacctgcctgaaatcactttttttcaggttgg

SEQ ID NO: 207 - DNA, 322 bp, synthetic construct
  source 1..322: mol_type = other DNA
aagaggtaagggtttaagggatggttggttgtggctgatcctctcccctgcccttggctccatgaatggcctcggcagtcctagcgggtgcgaaggggaccaaataaggcaaggtggcagaccgggccccccacccctgcccccggctgctccaactgaccctgtccatcagcgttctataaagcggccctcctggagccagccacccagagcccgctgccgccgcgagccgagtggtcgtccccgccgggatccgccaccgtggggtattaatgtttaattacctggagcacctgcctgaaatcactttttttcaggttgg

SEQ ID NO: 208 - DNA, 311 bp, synthetic construct
  source 1..311: mol_type = other DNA
aagaggtaagggtttaagggatggttggttggttctcctctataaatacccgctctggtatttggggttggcagctgttgctgccctataaatacgtatttatagctataaatacctggcaggacagcgggatcttgcagctgtcaggggaggggaggcgggggctgatgtcacggcgcctataaaaagtgtggggcgcggcgggcgggtcagtccgcctggagacctcgagccgagtggtcgtgcctccatagaagtggggtgtttaattacctggagcacctgcctgaaatcactttttttcaggttgg

SEQ ID NO: 209 - DNA, 345 bp, synthetic construct
  source 1..345: mol_type = other DNA
cgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggc

SEQ ID NO: 210 - DNA, 576 bp, synthetic construct
  source 1..576: mol_type = other DNA
cgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagttta

SEQ ID NO: 211 - DNA, 397 bp, synthetic construct
  source 1..397: mol_type = other DNA
gcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttca

SEQ ID NO: 212 - DNA, 204 bp, synthetic construct
  source 1..204: mol_type = other DNA
gcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtactt

SEQ ID NO: 213 - DNA, 160 bp, synthetic construct
  source 1..160: mol_type = other DNA
ggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagcc

SEQ ID NO: 214 - DNA, 95 bp, synthetic construct
  source 1..95: mol_type = other DNA
cagcccacctgtcccagtgctgacttagtgcaaggcgagccagcaaggagggaggacaggtggcagtggggggtgaggagcatctaaaaatagcc

SEQ ID NO: 215 - DNA, 622 bp, synthetic construct
  source 1..622: mol_type = other DNA
tggttgctgactaattgagatgcatgctttgcatacttctgcctgctggggagcctggggactttccacaccgggagttatttttagagcgatatggcgacggccacttggcagtacatcaattggaaagtcccgttagtgctattggctggccagccaatagctggttgctgactaattgagatgcatgctttgcatacttctgcctgctggggagcctggggactttccacaccgtgaggaatggtgccaggcatctcgggtttgatgtactgccaagccagccaatagccgctctaaaaataactcccccgtcgccatatttgggtgtccgctctaaaaataactcccggccgtcgccatatgggcccctccctggggacagccccttggaaagtcccgttagtgccaggcgggccatttaccgtcgctctaaaaataactccctagggaaacctgaagcccccattgacgtcaatggggcaggttttttatatagtccggcttcaggtttccctacaaggcctggggacaacccgagatgcctggcaggttttttatataacccaggggcacaggggctgccctcagtccgcctggagacctcgagccgagtggtcgt

SEQ ID NO: 216 - DNA, 331 bp, synthetic construct
  source 1..331: mol_type = other DNA
cggccgtccgccttcggcaccatcctcacgacacccaaatatggcgacgggtgaggaatggtggggagttatttttagagcggtgaggaatggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggcgctcccgcccgcctctatataacccaggggcacaggggctgccctcagtccgcctggagacctcgagccgagtggtcgt

SEQ ID NO: 217 - DNA, 267 bp, synthetic construct
  source 1..267: mol_type = other DNA
cggccgtccgccttcggcaccatcctcacgacacccaaatatggcgacgggtgaggaatggtggggagttatttttagagcggtgaggaatggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggcgctcccgcccgcctc

SEQ ID NO: 218 - DNA, 743 bp, synthetic construct
  source 1..743: mol_type = other DNA
ccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgccctcagtccgcctggagacctcgagccgagtggtcgtctcaggagcc

SEQ ID NO: 219 - DNA, 315 bp, synthetic construct
  source 1..315: mol_type = other DNA
gaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaa

SEQ ID NO: 220 - DNA, 401 bp, synthetic construct
  source 1..401: mol_type = other DNA
cctcccacgctgcgcgcgcacctccccgcccccacccctacccgctggcgtgcccagtggaacggagccttgtgtctccgcctcaagtccccggatgctcacctccccgactcgcccccgctgtggccccgcccccgcgcggctcttcgtgccacgtcaccgcctgcgtcgcttccggaggcgcagcgggcgatgacgtagagggacgtgccctctatataaggttggggaacaggggctgccctcagtccgcctggagacctcgagccgagtggtcgtgcggctgagtcggccttttccgcccgctcccccctccccccgagcgccgctccggctgcaccgcgctcgctccgagtttcaggctcgtgctaagctagcgccgtcgtcgtctcccttcagtc

SEQ ID NO: 221 - DNA, 176 bp, synthetic construct
  source 1..176: mol_type = other DNA
cgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccggg

SEQ ID NO: 222 - DNA, 324 bp, synthetic construct
  source 1..324: mol_type = other DNA
cattccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgccctcattctaccaccacctccacagcacagacagacactcaggagccagccagcc